(19) United States
(12) Patent Application Publication
(10) Pub. No.: US 2023/0138045 A1
(43) Pub. Date: May 4, 2023

(54) WNT SUPER AGONISTS

(71) Applicant: Surrozen Operating, Inc., South San Francisco, CA (US)

(72) Inventor: Yang LI, Mountain View, CA (US)

(21) Appl. No.: 17/801,624

(22) PCT Filed: Feb. 24, 2021
(86) PCT No.: PCT/US2021/019484

§ 371 Date: Aug. 23, 2022

Related U.S. Application Data
(60) Provisional application No. 63/114,368, filed on Nov. 16, 2020.
(60) Provisional application No. 62/980,870, filed on Feb. 24, 2020.

(51) Int. Cl. C07K 16/28; A61P 43/00

(52) U.S. Cl. CPC C07K 16/2863; A61P 43/00

(57) ABSTRACT

The present invention provides multispecific multivalent antigen binding molecules that can function as WNT agonist, WNT enhancer, and WNT super agonist molecules by binding and activating at least one or two WNT receptors and a WNT enhancer.

### CROSS-REFERENCE TO RELATED APPLICATIONS

This application claims priority to U.S. Provisional Application No. 62/980,870, filed on Feb. 24, 2020 and U.S. Provisional Application No. 63/114,368, filed on Nov. 16, 2020, each of which is herein incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is SRZN_018_02WO_ST25.txt. The text file is 1,179 KB, created on Feb. 24, 2021, and is being submitted electronically vis EFS-Web.

### FIELD OF THE INVENTION

The present invention provides antigen binding formats having both WNT agonist and WNT enhancer activities or WNT agonist or WNT enhancer activities alone.

### BACKGROUND OF THE INVENTION

WNT (“Wingless-related integration site” or “Wingless and Int-1” or “Wingless-Int”) ligands and their signals play key roles in the control of development, homeostasis and regeneration of many essential organs and tissues, including bone, liver, skin, stomach, intestine, kidney, central nervous system, mammary gland, taste bud, ovary, cochlea and many other tissues (reviewed, e.g., by Clevers, Loh, and Nusse (2014) Science; 346:54). Modulation of WNT signaling pathways has potential for treatment of degenerative diseases and tissue injuries.

The seven-pass transmembrane receptor Frizzled (FZD) is critical for nearly all WNT signaling, and the N-terminal FZD cysteine rich domain (CRD) serves as the WNT binding domain. In addition to FZD, the WNT/β-catenin pathway requires the Low-density lipoprotein receptor related proteins 5 and 6 (LRP5/6) to serve as co-receptors. LRP5 and LRP6 are functionally redundant single-pass transmembrane receptors. Biochemical studies of LRP6 indicate that different WNTs may bind to different extracellular domains of the LRP5/6 proteins. The LRP6 extracellular domain contains four repeating sequences of β-propeller and epidermal growth factor-like (βP-E) domains. The crystal structures of the extracellular LRP6 regions indicate that the βP-E repeats represent two discrete, compact, rigid structures, each containing two βP-E pairs. WNT9b binds the first two βP-E repeats on the extracellular domain of LRP6, whereas WNT3a binds the last two βP-E domains.

Non-WNT agonists or enhancers include Norrin and R-Spondin (RSPO), respectively. Norrin is a Fz4-specific ligand that, in conjunction with binding and activation of another WNT receptor, LRP5, forms a WNT surrogate or mimetic molecule.

The four RSPO genes represent a family of conserved secreted proteins that can enhance the WNT pathway signaling. LGR4/5/6 (leucine-rich repeat-containing GPCRs 4, 5, and 6) are receptors for RSPOs.

The role of RSPOs appears to be to stabilize the WNT receptors, FZD and LRP5/6, to promote or enhance WNT signaling. RSPO 1-4 are a family of ligands that amplify WNT signals. Each of the RSPOs work through a receptor complex that contains Zinc and Ring Finger 3 (ZNRF3) or Ring Finger Protein 43 (RNF43) on one end and a Leucine-rich repeat-containing G-protein coupled receptor 4-6 (LGR4-6) on the other (reviewed, e.g., by Knight and Hankenson 2014, Matrix Biology; 37: 157-161). RSPO might also work through additional mechanisms of action (Lebensohn and Rohatgi 2018, eLife, 7:e33126). ZNRF3 and RNF43 are two membrane-bound E3 ligases specifically targeting WNT receptors (FZD1-10 and LRP5 or LRP6) for degradation. Binding of an RSPO to ZNRF3/RNF43 and LGR4-6 causes clearance or sequestration of the ternary complex, which removes E3 ligases from WNT receptors and stabilizes WNT receptors, resulting in enhanced WNT signals. Each RSPO contains two Furin domains (1 and 2), with Furin domain 1 binding to ZNRF3/RNF43, and Furin domain 2 binding to LGR4-6. Fragments of RSPOs containing Furin domains 1 and 2 are sufficient for amplifying WNT signaling.

Antibodies are a well-established and rapidly growing drug class with at least 45 antibody-based products currently marketed for imaging or therapy in the United States and/or Europe with ˜$100 billion in total worldwide sales. This major clinical and commercial success with antibody therapeutics has fueled much interest in developing the next generation antibody drugs including bispecific antibodies. As their name implies, bispecific antibodies or multispecific antibodies (collectively “MsAbs”) bind to at least two different antigens, or at least two different epitopes on the same antigen, as first demonstrated more than 50 years ago. Engineering monospecific antibodies for multispecificity opens up many new potential therapeutic applications as evidenced by >30 BsAb in clinical development.

Bispecific or multispecific antibodies are a class of engineered antibody and antibody-like proteins that, in contrast to ‘regular’ monospecific antibodies, combine two or more different specific antigen binding elements in a single construct. Since bispecific antibodies do not typically occur in nature, they are constructed either chemically or biologically, using techniques such as cell fusion or recombinant DNA technologies. The ability to bind two or more different epitopes with a single molecule offers a number of potential advantages. One approach is to use the specificity of one arm as a targeting site for individual molecules, cellular markers or organisms, such as bacteria and viruses, while the other arm functions as an effector site for the recruitment of effector cells or delivery of molecular payloads to the target, such as drugs, cytokines or toxins. Alternatively, bispecifics can be used to dual target, allowing detection or binding of a target cell type with much higher specificity than monospecific antibodies.

The modular architecture of immunoglobulins has been exploited to create a growing number (>60) of alternative MsAb formats (see, e.g., Spiess et al. (2015) Mol. Immunol. 67:95-106). MsAb are classified here into five distinct structural groups: (i) bispecific IgG (BsIgG) (ii) IgG appended with an additional antigen-binding moiety (iii) MsAb fragments (iv) Multispecific fusion proteins and (v) MsAb conjugates. Each of these different MsAb formats brings different properties in binding valency for each antigen, geometry of antigen-binding sites, pharmacokinetic half-life, and in some cases effector functions.

For antagonistic MsAbs antibodies, which represent the vast majority of the MsAb molecules in development, the geometry of the antigen binding modules is less critical. However, for agonistic MsAbs, these molecules need to faithfully mimic the activity of the natural ligand, the binding geometry could be crucial (see, e.g., Shi, et al. (2018) J. Biol. Chem. 293:5909-5919). Such is true of WNT surrogate molecules, which are required to bind and activate two spatially separated WNT receptors, FZD and LRP.

WNT surrogate molecules which can bind to the hetero-oligomeric WNT/LRP receptor complex have been described previously (see, e.g., WO2019/126398, US 2020-0308287 A1, U.S. Ser. No. 17/257,817 and WO2020/010308) as have WNT enhancers using RSPO (see, e.g., WO2018/140821, US 2020-0048324 A1, WO2018/132572, US 2020-0024338 A1, 17/257,820 and WO2020/014271). However, a combination WNT surrogate molecule and enhancer, e.g., a WNT surrogate molecule that facilitates hetero-oligomerization in specific tissues along with WNT enhancement facilitated by RSPO or a mimetic thereof, has not been previously disclosed, Thus, a need exists to develop different antigen binding formats that mimic the binding of a natural ligand, e.g., FZD and LRP receptors, to hetero-oligomeric complexes that elicit agonistic biological activity, as well as enhancing WNT activity, e.g., using RSPO mimetics. The present invention fulfills this need by providing flexible structures of multispecific antibody (MsAb) formats that bind to different receptors (co-receptors) and acting either as a mimetic or antagonist of the natural ligand.

### BRIEF DESCRIPTION OF THE DRAWINGS

FIGS. 1A-M show the structure-function analysis of different configurations of WNT surrogate molecules in tandem scFv-IgG, Fv-IgG, Fab-IgG, and Fv-Fab formats: (A) schematic drawing of the different structures created using anti-LRP and anti-FZD antibody fragments is shown; (B) shows relative activity of these WNT surrogate molecules or WNT3A on WNT-responsive HEK293 STF reporter cell lines; (C) shows the ability of RSPO to potentiate the activity of these WNT surrogates; and (D-M) show the ability of Fv-IgG structures containing different FZD-binders to stimulate WNT pathways in the presence of RSPO.

FIGS. 2A-E show the structure-function analysis of anti-FZD binders fused with a mutant RPSO (RSPO2-RA) in different configurations: schematic drawing (A), WNT signaling activity (B), and effects on receptor levels (C) of Fv-IgGs fused to RSPOR2A are shown (in (C), at 103, the lines from top to bottom correspond to: anti-GFP, untreated, anti-GFP-RSPO2A, F12578-RSPO2RA, and no stain); (D) shows the activity of additional FZD binders fused to RSPO2RA; and (E) shows the activity of monovalent fusion proteins. “F” indicates anti-FZD binder, and “aGFP” indicates antiGFP antibody serving as negative control.

FIGS. 3A-M shows the activity of trispecific, hexavalent molecules containing FZD, LRP, and E3-ligase binding moieties: (A) shows a schematic drawing of a WNT surrogate (anti-FZD, anti-LRP bispecific antibody) fused to RSPO2-RA; (B-K) show that molecules constructed with RSPO2RA and FZD binders of different specificity all demonstrate both WNT surrogate and RSPO mimetic activities (E, F, G, K are from HEK293 cells transfected with FZD4, FZD9, FZD10, and FZD4, respectively); (J) shows additional attachment sites for RSPO2RA on the WNT mimetic molecule; (K) shows the activity of molecules with the formats shown in (J) (at log −8 of the left graph, the lines from top to bottom correspond to: L6-F4-2+20 nM Rspo, L6-F4-2-RSPO2RA-CH, L6-F4-2-RSPO2RA-NL, L6-F4-2-RSPO2RA-CL, and L6-F4-2; at log −8 of the right graph, the lines from top to bottom correspond to: L6-F4-RSPO2RA-CL, L6-F4+20 nM RSPO2, L6-F4-RSPO2RA-CH, L6-F4-RSPO2RA-NL, and L6-F4); and (L) and (M) show the activity of additional RSPO2RA fusions containing FZD binders with and without an LRP binder and fused at other locations on the IgG.

FIGS. 4A-C show the activity of additional trispecific molecules containing FZD, LRP, and E3-ligase binding moieties: (A) shows a schematic drawing a WNT surrogate (anti-FZD, anti-LRP bispecific antibody in various scFv-IgG configurations, top two structures) fused to RSPO2RA (bottom four structures); and (B-C) show the activity of molecules in (A) in the presence or absence of RSPO. In (B), at log −8, the symbols from top to bottom correspond to: L6-F12578 (Fv-IgG)+RSPO2, HC1-L6-F12578-RSPO2RA-KH+HC2-L6-F12578-HF, HC1-L6-F12578-RSPO2RA-KH+HC2-F12578-HF-RSPO2RA, HC1-L6-F12578-RSPO2RA-KH+HC2-F12578-HF, L6-F12578 (Fv-IgG), and HC1-L6-F12578-KH+HC2-F12578-HF).

FIGS. 5A-H shows WNT super agonist stimulates the expansion of several mouse and human organoids: (A, C, E, G) representative brightfield images of organoid outgrowth after 7 or 14 days. Scale bars, 400 μm. (B, D, F, H) quantification of cell viability using CellTiter-Glo®. Each datapoint represents an independent experiment. A) Outgrowth of mouse small intestinal organoids after 7 days using 0.1 nM of surrogate molecules and B) quantification of cell viability. C) Outgrowth of human small intestinal organoids after 7 days using 1 nM of surrogate molecules and D) quantification of cell viability. E) Outgrowth of mouse hepatocyte organoids after 14 days using 1 nM of surrogate molecules and F) quantification of cell viability. G) Outgrowth of human tubuloids after 7 days using 1 nM of surrogate molecules and H) quantification of cell viability.

FIGS. 6A-H show in vivo effects of WNT mimetic molecules. To test the in vivo effect of WNT mimetics with different FZD specificity, the panel of WNT mimetics were dosed at 3 mg per kg intraperitoneally on day 0, 3, 7 and 10 in C57Bl/6J mice. (A-C) The relative changes (%) of bone mineral densities (BMD) of whole body (A), femur (B) and lumbar (C) of the various treatment groups on day 7 and 13 by DEXA analysis. (D) The temporal body weight changes. (E) The relative changes (%) of body fat content on day 7 and 13. (F-H) The organ weight of salivary gland (F), liver (G), and small intestine (H) at the takedown on day 14. Statistical Analyses: One-way ANOVA, with post hoc Holm-Sidak test (GraphPad Prism). All comparisons made with the anti-GFP group. Data are show as mean standard deviation (SD). *p<0.05, **p<0.01, ***p<0.001, ****p<0.0001.

FIG. 7 shows formats of illustrative WNT surrogate molecules. For the Fab-IgG structure shown (and other structures), the Fab regions of the FZD binding domain and the LRP5/6 binding domain are indicated as being derived from the heavy chain or light chain of a parental antibody, but all other combinations of Fabs are also contemplated, e.g., HC-HC or LC-LC Fabs in either the heavy chain or light chain of the Fab-IgG construct, or switching the order of the two Fabs in either or both arms of the construct.

FIG. 8 shows formats of illustrative WNT super agonist molecules.

### SUMMARY OF THE INVENTION

The present invention provides an WNT super agonist molecule comprising a plurality of antigen binding domains, wherein the binding domains bind to at least one first WNT receptor and at least one second WNT receptor, and a WNT enhancer. In certain embodiments, a surrogate molecule is an agonist mimicking a natural ligand by facilitating the hetero-oligomerization of at least two different receptors in the present of a tissue targeting moiety. In certain embodiments, the binding domains are engineered to mimic a natural WNT ligand. In further embodiments, the binding domains are fused directly together. In yet further embodiments, the binding domains of the super agonist are fused together with a peptide linker. In some embodiments, the peptide linker is about 1 amino acid in length to about 30 amino acids in length. In other embodiments, the peptide linker is about 5 amino acids in length to about 15 amino acids in length. In another embodiment, the peptide linker comprises one or more glycine and/or serine residues. In one embodiment, at least one of the binding domains is selected from the group consisting of: an scFv, a VHH/sdAb, a Fab fragment, a Fab′2 fragment, a diabody, and an Fv fragment. In a further embodiment, at least one of the binding domains is fused to an Fc fragment. In a further embodiment, the structure is an Fv-IgG.

In particular embodiments, the disclosure provides a WNT super agonist molecule, comprising: a) a Frizzled (FZD) binding domain; b) an LRP5/6 binding domain; and c) an E3 ligase binding domain, wherein the super agonist molecule activates the canonical WNT signaling pathway in a cell. In certain embodiments: the FZD binding domain binds one or more FZD receptor; the LRP5/6 binding domain binds one or more of LRP5 and/or LRP6; and the E3 ligase binding domain binds ZNRF3 and/or RNF43. In certain embodiments, the WNT super agonist comprises one or more polypeptides, wherein at least one polypeptide comprises a FZD binding domain fused to an LRP5/6 binding domain, and wherein at least one polypeptide comprises an E3 ligase binding domain fused to a FZD binding domain or an LRP5/6 binding domain. In particular embodiments, the fused binding domains are fused directly together and/or fused via a peptide linker. In some embodiments, the peptide linker is about 1 amino acid in length to about 30 amino acids in length, or about 5 amino acids in length to about 15 amino acids in length, optionally 5, 6, 7, 8, 9, 10, 11, 12, 13, 14 or 15 amino acids in length. In some embodiments, the peptide linker comprises one or more glycine and/or serine residues. In particular embodiments of WNT super agonist molecules, at least one of the binding domains is selected from the group consisting of: an scFv, a VHH/sdAb, a Fab fragment, a Fab′2 fragment, a diabody, and an Fv fragment. In particular embodiments of WNT super agonist molecules, at least one of the binding domains is fused to an Fc fragment, optionally wherein the Fc fragment is from an IgG, IgM, IgA, IgD or IgE antibody isotype or an α, δ, ε, γ, or μ antibody heavy chain. In certain embodiments, the WNT super agonist molecule has ro comprises a structure depicted in Table 3 or Table 4, e.g., the Fv-IgG structure. In certain embodiments, the WNT enhancer domain of the WNT super agonist molecule comprises an E3 ligase binding domain selected from the group consisting of: a mutant R-spondin (RSPO) protein and an antibody or functional fragment thereof. In some embodiments, the mutant RSPO protein has reduced binding to Leucine-rich repeat-containing G-protein receptors 4-6 (LGR4-6) as compared to wild type RSPO. In some embodiments, the E3 ligase binding domain binds a Zinc and Ring Finger 3 (ZNRF3) and/or a Ring Finger Protein 43 (RNF43). In particular embodiments, the E3 ligase binding domain is selected from the group consisting of: an scFv, a VHH/sdAb, a Fab fragment, a Fab′2 fragment, a diabody, and an Fv fragment. In various embodiments, the E3 ligase binding domain is fused to a C-terminus of an Fc fragment of an Fv-IgG, either directly or via a linker, optionally wherein the linker is a peptide linker of about 1 amino acid in length to about 30 amino acids in length, or about 5 amino acids in length to about 15 amino acids in length, or 5, 6, 7, 8, 9, 10, 11, 12, 13, 14 or 15 amino acids in length. In various embodiments, the E3 ligase binding domain is fused to a C-terminus of: a) a light chain or fragment thereof of a FZD binding domain; b) a heavy chain or fragment thereof of a FZD binding domain; c) a light chain or fragment thereof of a LRP5/6 binding domain; or d) a heavy chain or fragment thereof of a LRP5/6 binding domain, either directly or via a linker, optionally wherein the linker is a peptide linker of about 1 amino acid in length to about 30 amino acids in length, or about 5 amino acids in length to about 15 amino acids in length, or 5, 6, 7, 8, 9, 10, 11, 12, 13, 14 or 15 amino acids in length. In various embodiments, the binding domain that binds an E3 ubiquitin ligase is fused to a N-terminus of: a) a light chain or fragment thereof of a FZD binding domain; b) a heavy chain or fragment thereof of a FZD binding domain; c) a light chain or fragment thereof of a LRP5/6 binding domain; or d) a heavy chain or fragment thereof of a LRP5/6 binding domain, either directly or via a linker, optionally wherein the linker is a peptide linker of about 1 amino acid in length to about 30 amino acids in length, or about 5 amino acids in length to about 15 amino acids in length, or 5, 6, 7, 8, 9, 10, 11, 12, 13, 14 or 15 amino acids in length. In some embodiments, a WNT super agonist comprises a polypeptide having at least 90% or 95% sequence identity to a sequence provided in Table 3 or Table 4, or a combination of polypeptides, each having at least 90% or 95% sequence identity to a sequence provided in Table 3 or Table 4.

In a related embodiment, the disclosure provides a WNT enhancer molecule (e.g., an RSPO mimetic) comprising at least one binding domain that binds to at least one WNT receptor; and a WNT enhancer. In certain embodiments, an R-spondin (RSPO) mimetic comprises a first binding composition that binds a WNT receptor and a second binding composition that binds an E3 ubiquitin ligase. In some embodiments, the first binding composition binds a FZD receptor or an LRP receptor, optionally LRP5 and/or LRP6. In some embodiments, the first binding composition is selected from the group consisting of: an scFv, a VHH/sdAb, a Fab fragment, a Fab′2 fragment, a diabody, and an Fv fragment. In certain embodiments, the second binding composition is an RSPO protein, optionally a mutant RSPO protein, or an antibody or fragment thereof that binds an E3 ubiquitin ligase. In certain embodiments, the binding compositions are fused directly together or via a peptide linker. In some embodiments, the peptide linker is about 1 amino acid in length to about 30 amino acids in length, or about 5 amino acids in length to about 15 amino acids in length, optionally 5, 6, 7, 8, 9, 10, 11, 12, 13, 14 or 15 amino acids in length. In certain embodiments of WNT enhancer molecules, the peptide linker comprises one or more glycine and/or serine residues. In some embodiments, the WNT enhancer comprises a polypeptide having at least 90% or 95% sequence identity to a sequence provided in Table 3 or Table 4, or a combination of polypeptides, each having at least 90% or 95% sequence identity to a sequence provided in Table 3 or Table 4.

In yet a further embodiment, the disclosure provides a WNT surrogate molecule comprising at least one binding domain that binds a FZD receptor and at least one binding domain that binds an LRP receptor. In certain embodiments, a WNT surrogate comprises: a) a Frizzled (FZD) binding domain; and b) an LRP5/6 binding domain, wherein the super agonist molecule activates the canonical WNT signaling pathway in a cell. In certain embodiments: a) the FZD binding domain binds one or more FZD receptor; and b) the LRP5/6 binding domain binds LRP5 and/or LRP6. In some embodiments, the FZD binding domain is selected from the group consisting of: an scFv, a VHH/sdAb, a Fab fragment, a Fab′2 fragment, a diabody, and an Fv fragment. In some embodiments, the LRP5/6 binding domain is selected from the group consisting of: an scFv, a VHH/sdAb, a Fab fragment, a Fab′2 fragment, a diabody, and an Fv fragment. In some embodiments, the binding domains are fused directly together or via a peptide linker. In particular embodiments, the peptide linker is about 1 amino acid in length to about 30 amino acids in length, about 5 amino acids in length to about 15 amino acids in length, optionally 5, 6, 7, 8, 9, 10, 11, 12, 13, 14 or 15 amino acids in length. In particular embodiments, the peptide linker comprises one or more glycine and/or serine residues. In some embodiments, the WNT surrogate molecule comprises a polypeptide having at least 90% or 95% sequence identity to a sequence provided in Table 3 or Table 4, or a combination of polypeptides, each having at least 90% or 95% sequence identity to a sequence provided in Table 3 or Table 4.

In various embodiments of any of the molecules disclosed herein that comprise a WNT enhancer domain or an E3 ligase binding domain, the WNT enhancer is selected from the group consisting of: a wild-type RSPO protein, a mutant RSPO protein, and a binding domain that binds to an E3 ubiquitin ligase. In further embodiments, the mutant RSPO protein has reduced binding to Leucine-rich repeat-containing G-protein receptors 4-6 (LGR4-6) as compared to wild type RSPO. In yet further embodiments, the binding domain that binds a E3 ubiquitin ligase binds to a Zinc and Ring Finger 3 (ZNRF3) and/or a Ring Finger Protein 43 (RNF43). In yet a further embodiment, the binding domain that binds to an E3 ubiquitin ligase is selected from the group consisting of: an scFv, a VHH/sdAb, a Fab fragment, a Fab′2 fragment, a diabody, and an Fv fragment. In certain embodiments, the binding domain that binds an E3 ubiquitin ligase is fused to a C-terminus of an Fc fragment of the Fv-IgG. In other embodiments, the binding domain that binds an E3 ubiquitin ligase is fused to a C-terminus of: a) a light chain of a binding domain that binds to a FZD receptor; b) a heavy chain of a binding domain that binds to a FZD receptor; c) a light chain of a binding domain that binds to an LRP receptor; or b) a heavy chain of a binding domain that binds to an LRP receptor. In another embodiment, the binding domain that binds an E3 ubiquitin ligase is fused to a N-terminus of: a) a light chain of a binding domain that binds to a FZD receptor; b) a heavy chain of a binding domain that binds to a FZD receptor; c) a light chain of a binding domain that binds to an LRP receptor; or d) a heavy chain of a binding domain that binds to an LRP receptor. In certain embodiments, the super-agonist comprises a structure depicted in Table 3 or Table 4.

In various embodiments of any of the molecules disclosed herein, one or more of the polypeptides comprises an additional sequence, e.g., a tag, which may, e.g., be used to facilitate purification of the polypeptide. Examples of such tag molecules include, but are not limited to, His tags, Myc tags, and Flag tags.

In one embodiment, the present invention provides a method for treating a subject having a disease or disorder associated with reduced WNT signaling, comprising administering to the subject an effective amount of the WNT super agonist molecule, a WNT enhancer molecule, a WNT surrogate molecule, or a pharmaceutical composition comprising one or more of these molecules. In certain embodiments, the disease or disorder is selected from the group consisting of: oral mucositis, short bowel syndrome, inflammatory bowel diseases (IBD), other gastrointestinal disorders; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; myocardial infarction, coronary artery disease, heart failure; immunodeficiencies, graft versus host diseases, acute kidney injuries, chronic kidney diseases, chronic obstructive pulmonary diseases (COPD), idiopathic pulmonary fibrosis (IPF), cirrhosis, acute liver failure, chronic liver diseases with hepatitis C or B virus infection or post-antiviral drug therapies, alcoholic liver diseases, alcoholic hepatitis, non-alcoholic liver diseases with steatosis or steatohepatitis, treatment of hearing loss, including internal and external loss of auditory hair cells, vestibular hypofunction, macular degeneration, treatment of vitreoretinopathy, diabetic retinopathy, other diseases of retinal degeneration, Fuchs' dystrophy, other corneal diseases, stroke, traumatic brain injury, Alzheimer's disease, multiple sclerosis and other conditions affecting the blood brain barrier; spinal cord injuries, bone related diseases, other spinal cord diseases, and alopecia.

In one embodiment, the present invention provides a method of generating, culturing, or maintaining an organ tissue, cell, or an organoid culture, comprising contacting the organ tissue, cell, or an organoid culture with a WNT super agonist molecule, a WNT enhancer molecule, or a WNT surrogate molecule, or a pharmaceutical composition comprising the WNT super agonist molecule, WNT enhancer molecule, or WNT surrogate molecule. In a further embodiment, the organ tissue obtained is from a donor and contacted with the WNT super agonist molecule, WNT enhancer molecule, or WNT surrogate molecule, optionally by perfusing the organ tissue ex vivo with a composition comprising the WNT super agonist molecule, WNT enhancer molecule, or WNT surrogate molecule. In yet another embodiment, the viability of the organ tissue is maintained by contacting donor organ tissue in vivo, with a composition comprising the WNT super agonist or the WNT enhancer molecule. In another embodiment, the organoid culture is maintained by contacting the organoid culture with the WNT super agonist molecule, WNT enhancer molecule, or WNT surrogate molecule, optionally by culturing the organoid culture in a medium comprising the WNT super agonist or the WNT enhancer.

In a further related embodiment, the disclosure provides a method for inducing bone formation or increasing bone density in a subject, comprising administering to the subject an effective amount of a WNT super agonist molecule, WNT enhancer molecule, or WNT surrogate molecule, or a pharmaceutical composition comprising one or more of these molecules. In some embodiments, the method is performed using a WNT super agonist molecule that binds FZD5, FZD8, and FZD9. In some embodiments, the method is performed using a WNT surrogatet molecule that binds FZD5, FZD8, and FZD9.

In a further related embodiment, the disclosure provides a method for regenerating a salivary gland or inducing salivary gland growth in a subject, comprising administering to the subject an effective amount of a WNT super agonist molecule, WNT enhancer molecule, or WNT surrogate molecule, or a pharmaceutical composition comprising one or more of these molecules. In some embodiments, the methods is performed for treating hyposalivation in the subject. In some embodiments, the method is performed using a WNT super agonist molecule that binds FZD1, FZD2, and FZD7. In some embodiments, the method is performed using a WNT surrogate molecule that binds FZD1, FZD2, and FZD7.

In some embodiments, the present invention provides an RSPO mimetic comprising a first binding composition that binds one WNT receptor and a second binding composition that binds an E3 ubiquitin ligase. In some embodiments, the first binding composition binds a FZD receptor or an LRP receptor. In further embodiments, the first binding composition is selected from the group consisting of: an scFv, a VHH/sdAb, a Fab fragment, a Fab′2 fragment, a diabody, and an Fv fragment. In yet further embodiments, the second binding composition is an RSPO protein or an antibody or fragment thereof that binds an E3 ubiquitin ligase.

### DETAILED DESCRIPTION

As used herein, including the appended claims, the singular forms of words such as “a,” “an,” and “the,” include their corresponding plural references unless the context clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Each embodiment in this specification is to be applied mutatis mutandis to every other embodiment unless expressly stated otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule. As used herein, the term encompasses not only intact polyclonal or monoclonal antibodies, but also fragments thereof (such as dAb, Fab, Fab′, F(ab′)2, Fv), single chain (scFv), VHH, synthetic variants thereof, naturally occurring variants, fusion proteins comprising an antibody or an antigen-binding fragment thereof, humanized antibodies, chimeric antibodies, and any other modified configuration of the immunoglobulin molecule that comprises an antigen-binding site or fragment (epitope recognition site) of the required specificity. “Diabodies”, multivalent or multispecific fragments constructed by gene fusion (WO94/13804; P. Holliger et al (1993), Proc. Natl. Acad. Sci. USA 90 6444-6448) are also a particular form of antibody contemplated herein. Minibodies comprising a scFv joined to a CH3 domain are also included herein (See e.g., S. Hu et al. (1996), Cancer Res., 56:3055-3061; Ward, E. S. et al. (1989) Nature 341:544-546; Bird et al. (1988) Science 242:423-426; Huston et al. (1988) Proc. Natl. Acad. Sci. USA 85:5879-5883; PCT/US92/09965; WO94/13804; P. Holliger et al. (1993) Proc. Natl. Acad. Sci. USA 90:6444-6448; and Y. Reiter et al. (1996) Nature Biotech. 14:1239-1245).

The term “antigen-binding fragment” as used herein refers to a polypeptide fragment that contains at least one CDR of an immunoglobulin heavy and/or light chain, or of a VHH, that binds to the antigen of interest, in particular to one or more FZD receptor or LRP5 or LRP6 receptor. In this regard, an antigen-binding fragment of the herein described antibodies may comprise 1, 2, 3, 4, 5, or all 6 CDRs of a VH and VL sequence set forth herein from antibodies that bind one or more FZD receptor or LRP5 and/or LRP6. In particular embodiments, an antigen-binding fragment may comprise all three VH CDRs or all three VL CDRs. Similarly, an antigen binding fragment thereof may comprise all three CDRs of a VHH binding fragment. An antigen-binding fragment of a FZD-specific antibody is capable of binding to a FZD receptor. An antigen-binding fragment of a LRP5/6-specific antibody is capable of binding to a LRP5 and/or LRP6 receptor. As used herein, the term encompasses not only isolated fragments but also polypeptides comprising an antigen-binding fragment of an antibody disclosed herein, such as, for example, fusion proteins comprising an antigen-binding fragment of an antibody disclosed herein, such as, e.g., a fusion protein comprising a VHH that binds one or more FZD receptors and a VHH that binds LRP5 and/or LRP6.

The term “antigen” refers to a molecule or a portion of a molecule capable of being bound by a selective binding agent, such as an antibody, and additionally capable of being used in an animal to produce antibodies capable of binding to an epitope of that antigen. In certain embodiments, a binding agent (e.g., a WNT surrogate molecule or binding region thereof) is said to specifically bind an antigen when it preferentially recognizes its target antigen in a complex mixture of proteins and/or macromolecules. In certain embodiments, a WNT surrogate molecule or binding region thereof (e.g., an antibody or antigen-binding fragment thereof) is said to specifically bind an antigen when the equilibrium dissociation constant is ≤10⁻⁷ or ≤10⁻⁸ M. In some embodiments, the equilibrium dissociation constant may be ≤10⁻⁹ M or ≤10⁻¹⁰ M.

As used herein, the term “CDR” refers to at least one of the three hypervariable regions of a heavy or light chain variable (V) region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site. In certain embodiments, antibodies and antigen-binding fragments thereof as described herein include a heavy chain and a light chain CDRs, respectively interposed between a heavy chain and a light chain framework regions (FRs)which provide support to the CDRs and define the spatial relationship of the CDRs relative to each other.

As used herein, the term “FRs” refer to the four flanking amino acid sequences which frame the CDRs of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains. The structures and locations of immunoglobulin CDRs and variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), VHH, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody.”

The term “co-receptor” refers to a first cell surface receptor that binds signaling molecule or ligand in conjunction with another receptor to facilitate ligand recognition and initiate a biological process, such as WNT pathway signaling.

The term “agonist activity” refers to the ability of an agonist to mimic the effect or activity of a naturally occurring protein.

As used herein “peptide linker” or “linker moiety” refers to a sequence of sometimes repeating amino acid residues, usually glycine and serine, that are used to join the various antigen binding domains described below. The length of the linker sequence determines the flexibility of the antigen binding domains in MsAbs, in particular, in the binding of epitopes on co-receptors such as FZD receptors, LRP5 and/or LRP6, and/or ZNRF3/RNF43.

As used herein, the term “enhances” refers to a measurable increase in the level of receptor signaling modulated by a ligand or ligand agonist compared with the level in the absence of the agonist, e.g., a WNT surrogate molecule. In particular embodiments, the increase in the level of receptor signaling is at least 10%, at least 20%, at least 50%, at least two-fold, at least five-fold, at least 10-fold, at least 20-fold, at least 50-fold, or at least 100-fold as compared to the level of receptor signaling in the absence of the agonist, e.g., in the same cell type. In certain embodiments, a WNT super agonist molecule increases the level of receptor signaling to a greater degree than a corresponding WNT surrogate molecule comprising the same FZD binding domain and LRP5/6 binding domain, but lacking the E3 ligase binding domain, e.g., by at least 10%, at least 20%, at least 50%, or at least two-fold.

An antigen or epitope that “specifically binds” or “preferentially binds” (used interchangeably herein) to an antibody or antigen-binding fragment thereof is a term well understood in the art, and methods to determine such specific or preferential binding are also well known in the art. A molecule, e.g., a WNT surrogate molecule or WNT super agonist molecule, is said to exhibit “specific binding” or “preferential binding” if it reacts or associates more frequently, more rapidly, with greater duration and/or with greater affinity with a particular cell or substance than it does with alternative cells or substances. A molecule or binding region thereof, e.g., a WNT surrogate molecule or binding region thereof, “specifically binds” or “preferentially binds” to a target antigen, e.g., a FZD receptor, if it binds with greater affinity, avidity, more readily, and/or with greater duration than it binds to other substances. It is also understood by reading this definition that, for example, a surrogate molecule or binding region thereof that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. (1986) Nucl. Acids Res. 14:9081; Stec et al. (1984) J. Am. Chem. Soc. 106:6077; Stein et al. (1988) Nucl. Acids Res. 16:3209; Zon et al. (1991) Anti- Cancer Drug Design, 6:539; Zon et al. (1991) Oligonucleotides and Analogues: A Practical Approach, pp. 87-108 (F. Eckstein, Ed.), Oxford University Press, Oxford England; Stec et al., U.S. Pat. No. 5,151,510; Uhlmann and Peyman (1990) Chem. Rev. 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

The term “host cell” is used to refer to a cell into which has been introduced, or which is capable of having introduced into it, a nucleic acid sequence encoding one or more of the herein described polypeptides, and which further expresses or is capable of expressing a selected gene of interest, such as a gene encoding any herein described polypeptide. The term includes the progeny of the parent cell, whether or not the progeny are identical in morphology or in genetic make-up to the original parent, so long as the selected gene is present. Accordingly there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus.

For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. In one embodiment, the nucleic acid is integrated into the genome (e.g. chromosome) of the host cell. Integration may be promoted by inclusion of sequences which promote recombination with the genome, in accordance-with standard techniques.

“Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, MOLECULAR CLONING, A LABORATORY MANUAL, Cold Spring Harbor Laboratories; Davis et al., 1986, BASIC METHODS IN MOLECULAR BIOLOGY, Elsevier; and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell.

The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

The terms “polypeptide” “protein” and “peptide” and “glycoprotein” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass WNT surrogate molecules, FZD binding regions thereof, LRP5/6 binding regions thereof, antibodies and antigen-binding fragments thereof that bind to a FZD receptor or a LRP5 or LRP6 receptor disclosed herein, or sequences that have deletions from, additions to, and/or substitutions of one or more amino acid of any of these polypeptides. Thus, a “polypeptide” or a “protein” can comprise one (termed “a monomer”) or a plurality (termed “a multimer”) of amino acid chains.

The term “isolated protein,” “or “isolated antibody” referred to herein means that a subject protein, surrogate molecule, or antibody: (1) is free of at least some other proteins with which it would typically be found in nature; (2) is essentially free of other proteins from the same source, e.g., from the same species, (3) is expressed by a cell from a different species; (4) has been separated from at least about 50 percent of polynucleotides, lipids, carbohydrates, or other materials with which it is associated in nature; (5) is not associated (by covalent or noncovalent interaction) with portions of a protein with which the “isolated protein” is associated in nature; (6) is operably associated (by covalent or noncovalent interaction) with a polypeptide with which it is not associated in nature; or (7) does not occur in nature. Such an isolated protein can be encoded by genomic DNA, cDNA, mRNA or other RNA, or may be of synthetic origin, or any combination thereof. In certain embodiments, an isolated protein may comprise naturally-occurring and/or artificial polypeptide sequences. In certain embodiments, the isolated protein is substantially free from proteins or polypeptides or other contaminants that are found in its natural environment that would interfere with its use (therapeutic, diagnostic, prophylactic, research or otherwise).

A “WNT super agonist” is a molecule having enhanced WNT agonist activity. As used herein, the WNT super agonists have both WNT signaling and WNT signal enhancing activity. In some embodiments, the WNT super agonist molecule will bind both at least one FZD receptor and at least one LRP receptor, as well as binding and activating at least one E3 ubiquitin ligase receptor, thereby stabilizing the FZD and/or LRP receptors.

### II. General

The present invention provides combinations of antigen binding molecules that act as WNT super agonist, WNT surrogate and WNT enhancing (RSPO mimetic) molecules by binding to and modulating co-receptor signaling, for example, antigen binding molecules that bind to one or more FZD receptor and one or more LRP5 or LRP6 receptor, and one or more ZNRF3/RNF43 E3 ubiquitin ligase molecules, which in turn modulate a downstream WNT signaling pathway, and methods of preparation and use thereof. In particular embodiments, the surrogate molecules activate or increase a signaling pathway associated with one or both of the co-receptors.

In particular embodiments, the WNT super agonist molecules disclosed herein comprise: (i) one or more antibodies or antigen-binding fragments thereof that specifically bind to one or more first co-receptor, including antibodies or antigen-binding fragments thereof having particular co-receptor specificity and/or functional properties; (ii) one or more antibodies or antigen-binding fragments thereof that specifically bind to one or more second co-receptors; and (iii) one or more polypeptides (e.g., a mutated R-spondin) that specifically bind to one or more E3 ligase, e.g., ZNRF3 and/or RNF43. Certain embodiments encompass specific structural formats or arrangements of the first and second co-receptor binding region(s) of the WNT super agonist molecules advantageous in increasing downstream signaling and related biological effects.

In particular embodiments, the WNT surrogate molecules disclosed herein comprise: (i) one or more antibodies or antigen-binding fragments thereof that specifically bind to one or more first co-receptor, including antibodies or antigen-binding fragments thereof having particular co-receptor specificity and/or functional properties; and (ii) one or more antibodies or antigen-binding fragments thereof that specifically bind to one or more second co-receptors. Certain embodiments encompass specific structural formats or arrangements of the first and second co-receptor binding region(s) of the WNT surrogate molecules advantageous in increasing downstream signaling and related biological effects.

In particular embodiments, the WNT enhancer molecules (also referred to as RSPO mimetics) disclosed herein comprise: (i) one or more antibodies or antigen-binding fragments thereof that specifically bind to one or more first co-receptor (either one or more FZD or LRP5/6), including antibodies or antigen-binding fragments thereof having particular co-receptor specificity and/or functional properties; (ii) one or more polypeptides (e.g., a mutated R-spondin) that specifically bind to one or more E3 ligase, e.g., ZNRF3 and/or RNF43. Certain embodiments encompass specific structural formats or arrangements of the first and second co-receptor binding region(s) of the WNT super agonist molecules advantageous in increasing downstream signaling and related biological effects. In particular embodiments, the WNT enhancer molecules do not bind to both a FZD receptor and an LRP5/6.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Sequences of illustrative antibodies, or antigen-binding fragments, or complementarity determining regions (CDRs) thereof, that bind to one or more FZD receptors, are set forth in WO2019126399. Sequences of illustrative LRP5 and/or LRP6 antibodies, or antigen-binding fragments, or complementarity determining regions (CDRs) thereof, are set forth in WO2019126401. Sequences of antigen binding molecules that bind one or more FZD receptor and LRP5 and/or LRP6 are set forth in U.S. Provisional application Nos. 62/607,875, 62/641,217, and 62/680,522, titled WNT Signaling Pathway Agonists, filed Dec. 19, 2017, Mar. 9, 2018, and Jun. 4, 2018, respectively.

Antibodies and antibody fragments thereof may be prepared by methods well known in the art. For example, the proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present invention can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH:VL heterodimer including an antigen-binding site which retains much of the antigen recognition and binding capabilities of the native antibody molecule. (See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096).

In certain embodiments, single chain Fv or scFV antibodies are contemplated. For example, Kappa bodies (Ill et al. (1997), Prot. Eng. 10: 949-57; minibodies (Martin et al. (1994) EMBO J 13: 5305-9; diabodies (Holliger et al. (1993) PNAS 90: 6444-8; or janusins (Traunecker et al. (1991) EMBO J 10: 3655-59; and Traunecker et al. (1992) Int. J. Cancer Suppl. 7: 51-52), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more FZD receptor through one binding domain and to a second molecule through a second binding domain. These antibodies may be produced through recombinant molecular biological techniques or may be physically conjugated together.

A single chain Fv (scFv) polypeptide is a covalently linked VH::VL heterodimer which is expressed from a gene fusion including VH- and VL-encoding genes linked by an encoded peptide linker. Huston et al. (1988) Proc. Nat. Acad. Sci. USA 85(16):5879-5883. A number of methods have been described to discern chemical structures for converting the naturally aggregated—but chemically separated-light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). A dAb fragment of an antibody consists of a VH domain (Ward, E. S. et al. (1989) Nature 341:544-546).

Where bispecific antibodies are to be used, these may be conventional bispecific antibodies, which can be manufactured in a variety of ways (Holliger, P. and Winter G. (1993) Curr. Op. Biotechnol. 4:446-449), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al. (1996) Protein Eng., 9:616-621).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, the antibodies of the present disclosure may take the form of a single variable domain fragment known as a VHH. The VHH technology was originally developed following the discovery and identification that camelidae (e.g., camels and llamas) possess fully functional antibodies that consist of heavy chains only and therefore lack light chains. These heavy-chain only antibodies contain a single VHH domain and two constant domains (CH2, CH3). The cloned and isolated VHH domains have full antigen binding capacity and are very stable. These VHH domains are encoded by single genes and are efficiently produced in almost all prokaryotic and eukaryotic hosts e.g. E. coli (see e.g. U.S. Pat. No. 6,765,087), molds (for example Aspergillus or Trichoderma) and yeast (for example Saccharomyces, Kluyvermyces, Hansenula or Pichia (see e.g. U.S. Pat. No. 6,838,254). The production process is scalable and multi-kilogram quantities of VHHs have been produced. VHHs may be formulated as a ready-to-use solution having a long shelf life. The Nanoclone® method (see, e.g., WO 06/079372) is a proprietary method for generating VHHs against a desired target, based on automated high-throughput selection of B-cells. VHH antibodies typically have a small size of around 15 kDa.

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al. (1988) Proc Natl Acad Sci USA 86:10029-10033; and Riechmann et al. (1988) Nature 332:323-327). Illustrative methods for humanization of the anti-FZD antibodies disclosed herein include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856. Riechmann, L., et al., (1988) supra; Verhoeyen, M., et al., (1988) Science 239:1534-1536; Kettleborough, C. A., et al., (1991) Protein Engg 4:773-3783; Maeda, H., et al., (1991) Human Antibodies Hybridoma 2:124-134; Gorman, S. D., et al., (1991) Proc Nat!AcadSci USA 88:4181-4185; Tempest, P. R., et al., (1991) Bio Technol. 9:266-271; Co, M. S., et al., (1991) Proc Natl Acad Sci USA 88:2869-2873; Carter, P., et al., (1992) Proc Natd Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

### III. Structures of Receptor Surrogate Ligands (WNT Surrogate Molecules)

The disclosure provides, in certain aspects, surrogate molecules that bind both one or more of a first receptor (e.g., FZD) and one or more of a second receptor (e.g., LRP5 and/or LRP6; also referred to as LRP5/6). For example, a WNT surrogate molecule can bind one or more human FZD receptors and one or both of a human LRP5 and/or a human LRP6.

In certain embodiments, a surrogate molecule is capable of modulating or modulates signaling events associated with at least one of the co-receptors that it binds, in a cell contacted with the surrogate molecule. In certain embodiments, the surrogate molecule increases receptor signaling. As an example, a WNT surrogate molecule specifically modulates the biological activity of a human WNT/β-catenin signaling pathway.

Surrogate molecules of the present invention are biologically active in binding to one or more of a first receptor and to one or more of a second receptor, and as an example, in the activation of WNT signaling, the WNT surrogate molecule is a WNT agonist. The term “agonist activity” refers to the ability of an agonist to mimic the effect or activity of a naturally occurring protein binding to a first and second receptor. The ability of the surrogate molecules and other receptor agonists disclosed herein to mimic the activity of the natural ligand can be confirmed by a number of assays. As an example, WNT surrogate molecules, some of which are disclosed herein activate, enhance or increase the canonical WNT/0-catenin signaling pathway.

In particular embodiments, the structures of the surrogate molecules disclosed herein are bispecific, i.e., they specifically bind to two or more different epitopes, e.g., one or more epitopes of a first receptor, and one or more epitopes of a second receptor.

In particular embodiments, WNT surrogate molecules disclosed herein are multivalent, e.g., they comprise two or more regions that each specifically bind to the same epitope, e.g., two or more regions that bind to an epitope within one or more first co-receptor and/or two or more regions that bind to an epitope within a second co-receptor. In particular embodiments, they comprise two or more regions that bind to an epitope within a first co-receptor and two or more regions that bind to an epitope within a second co-receptor. In certain embodiments, surrogate molecules comprise a ratio of the number of regions that bind one or more first co-receptor to the number of regions that a second co-receptor of or about: 1:1, 2:1, 3:1, 4:1, 5:1, 6:1, 2:3, 2:5, 2:7, 7:2, 5:2, 3:2, 3:4, 3:5, 3:7, 3:8, 8:3, 7:3, 5:3, 4:3, 4:5, 4:7, 4:9, 9:4, 7:4, 5:4, 6:7, 7:6, 1:2, 1:3, 1:4, 1:5, or 1:6. In certain embodiments, the surrogate molecules are bispecific and multivalent.

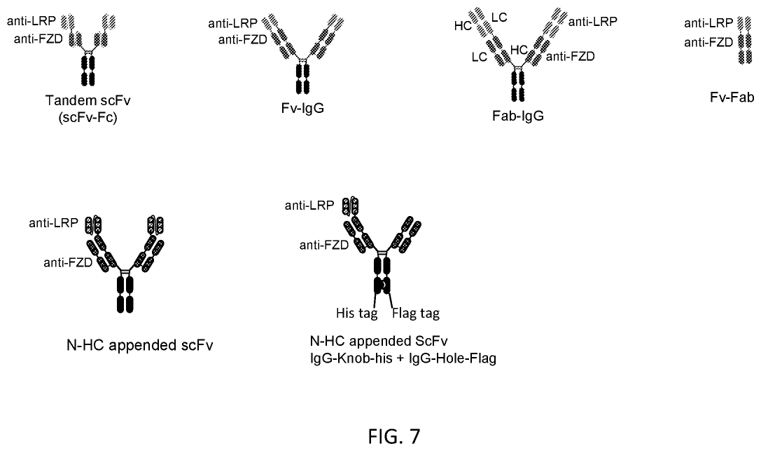

The structures of the WNT surrogate molecules disclosed herein may have any of a variety of different structural formats or configurations. The surrogate molecules may comprise polypeptides and/or non-polypeptide binding moieties, e.g., small molecules. In particular embodiments, the surrogate molecules comprise both a polypeptide region and a non-polypeptide binding moiety. In certain embodiments, the surrogate molecules may comprise a single polypeptide, or they may comprise two or more, three or more, or four or more polypeptides. In certain embodiments, one or more polypeptides of a surrogate molecule are antibodies or antigen-binding fragments thereof. In certain embodiments, surrogates comprise two antibodies or antigen binding fragments thereof, one that binds one or more first co-receptor and one that binds on or more second co-receptor. In certain embodiments, the surrogates comprises one, two, three, or four polypeptides, e.g., linked or bound to each other or fused to each other. Non-limiting examples of WNT surrogate structures contemplated by the disclosure are provided in FIG. 7.

When the surrogate molecules comprise a single polypeptide, they may be a fusion protein comprising one or more first co-receptor binding domain and one or more second co-receptor binding domain. The binding domains may be directly fused or they may be connected via a linker, e.g., a polypeptide linker, including but not limited to any of those disclosed herein.

When the surrogate molecules comprise two or more polypeptides, the polypeptides may be linked via covalent bonds, such as, e.g., disulfide bonds, and/or noncovalent interactions. For example, heavy chains of human immunoglobulin IgG interact at the level of their CH3 domains directly, whereas, at the level of their CH2 domains, they interact via the carbohydrate attached to the asparagine (Asn) N84.4 in the DE turn. In particular embodiments, the surrogate molecules comprise one or more regions derived from an antibody or antigen-binding fragment thereof, e.g., antibody heavy chains or antibody light chains or fragments thereof. In certain embodiments, a surrogate polypeptide comprises two antibody heavy chain regions (e.g., hinge regions) bound together via one or more disulfide bond. In certain embodiments, a surrogate polypeptide comprises an antibody light chain region (e.g., a CL region) and an antibody heavy chain region (e.g., a CH1 region) bound together via one or more disulfide bond.

Surrogate polypeptides may be engineered to facilitate binding between two polypeptides. For example, Knob-into-holes amino acid modifications may be introduced into two different polypeptides to facilitate their binding. Knobs-into-holes amino acid (AA) changes is a rational design strategy developed in antibody engineering, used for heterodimerization of the heavy chains, in the production of bispecific IgG antibodies. AA changes are engineered in order to create a knob on the CH3 of the heavy chains from a first antibody and a hole on the CH3 of the heavy chains of a second antibody. The knob may be represented by a tyrosine (Y) that belongs to the ‘very large’ IMGT volume class of AA, whereas the hole may be represented by a threonine (T) that belongs to the ‘small’ IMGT volume class. Other means of introducing modifications into polypeptides to facilitate their binding are known and available in the art. For example, specific amino acids may be introduced and used for cross-linking, such as Cysteine to form an intermolecular disulfide bond.

Surrogate molecules may have a variety of different structural formats, including but not limited to those as described in WO2019126398 and WO2020010308.

In one embodiment, a surrogate molecule comprises an scFv or antigen-binding fragment thereof fused to a VHH or antigen-binding fragment thereof. In certain embodiments, the scFv specifically binds one or more first receptor, and the VHH specifically binds to one or more second receptor. In certain embodiments, the scFv specifically binds LRP5 and/or LRP6, and the VHH specifically binds one or more FZD receptor. In particular embodiments, the scFv or antigen-binding fragment thereof is fused directly to the VHH or antigen-binding fragment thereof, whereas in other embodiments, the two binding regions are fused via a linker moiety. In particular embodiments, the VHH is fused or linked to the N-terminus of the scFV, while in other embodiments, the VHH is fused to the C-terminus of the scFv.

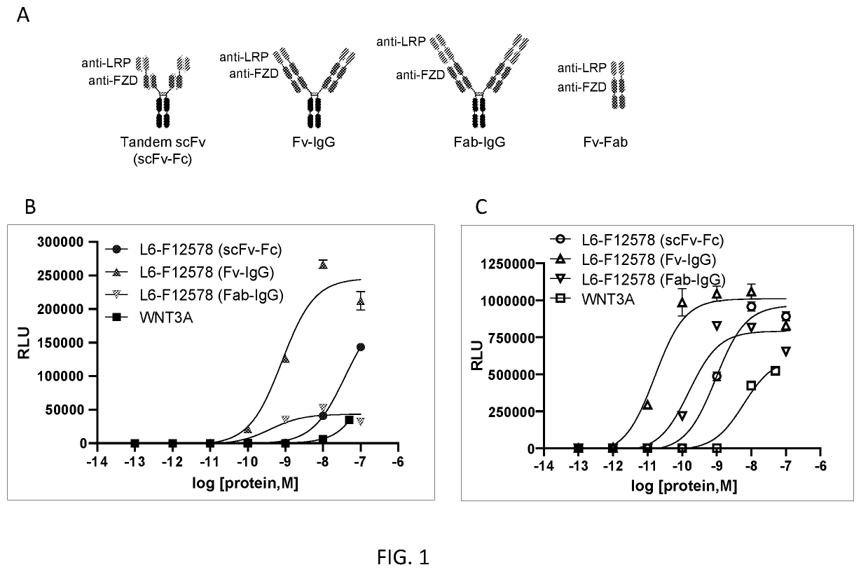

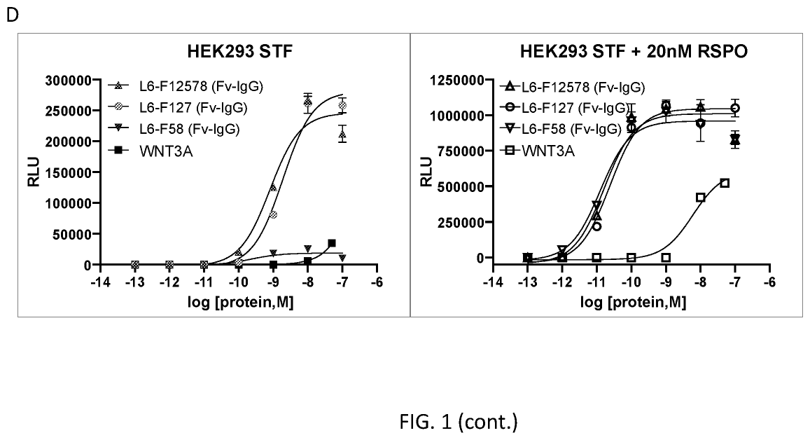

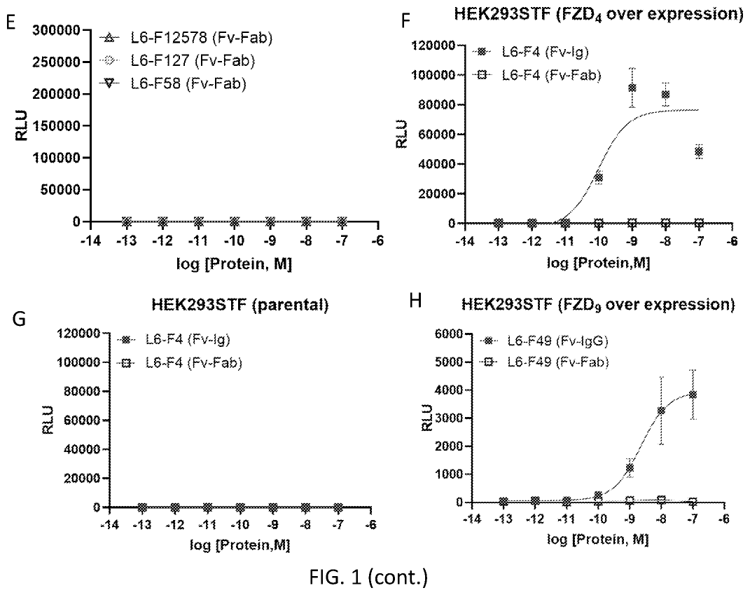

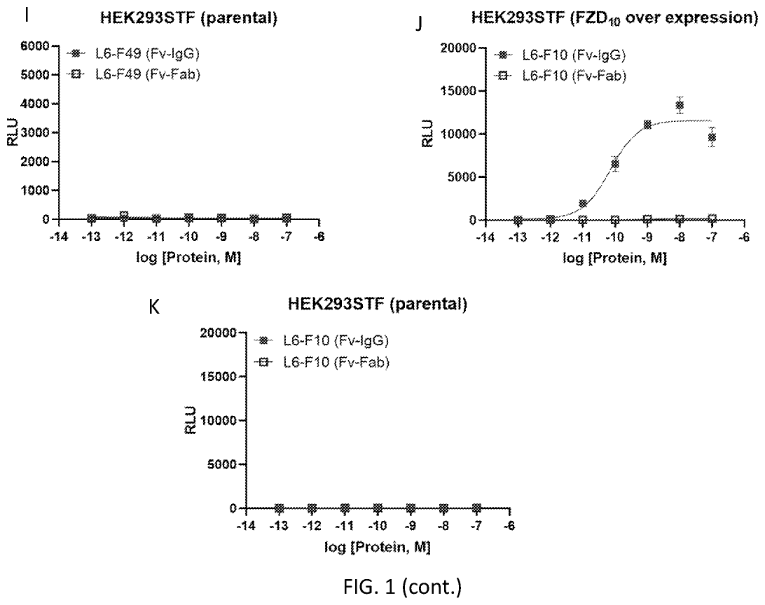

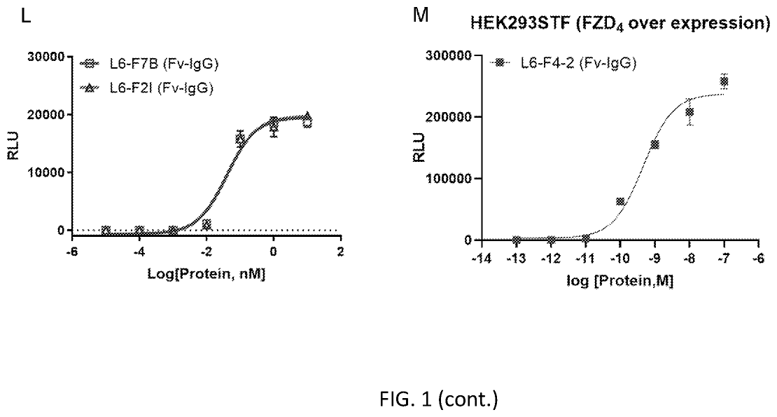

In various embodiments, including but not limited to those depicted in WO2019126398, WO2020010308, Table 3, Table 4, and FIGS. 1-8, a surrogate molecule comprises one or more Fab or antigen-binding fragment thereof and one or more VHH or antigen-binding fragment thereof (or alternatively, one or more scFv or antigen-binding fragment thereof). In certain embodiments, the Fab specifically binds one or more FZD receptor, and the VHH (or scFv) specifically binds LRP5 and/or LRP6. In certain embodiments, the Fab specifically binds LRP5 and/or LRP6, and the VHH (or scFv) specifically binds one or more FZD receptor. In certain embodiments, the VHH (or scFv) is fused to the N-terminus of the Fab, while in some embodiments, the VHH (or scFv) is fused to the C-terminus of the Fab. In particular embodiments, the Fab is present in a full IgG format, and the VHH (or scFv) is fused to the N-terminus and/or C-terminus of the IgG light chain. In particular embodiments, the Fab is present in a full IgG format, and the VHH (or scFv) is fused to the N-terminus and/or C-terminus of the IgG heavy chain. In particular embodiments, two or more VHHs (or scFvs) are fused to the IgG at any combination of these locations.

Fabs may be converted into a full IgG format that includes both the Fab and Fc fragments, for example, using genetic engineering to generate a fusion polypeptide comprising the Fab fused to an Fc region, i.e., the Fab is present in a full IgG format. The Fc region for the full IgG format may be derived from any of a variety of different Fcs, including but not limited to, a wild-type or modified IgG1, IgG2, IgG3, IgG4 or other isotype, e.g., wild-type or modified human IgG1, human IgG2, human IgG3, human IgG4, human IgG4Pro (comprising a mutation in core hinge region that prevents the formation of IgG4 half molecules), human IgA, human IgE, human IgM, or the modified IgG1 referred to as IgG1 LALAPG. The L235A, P329G (LALA-PG) variant has been shown to eliminate complement binding and fixation as well as Fc-7 dependent antibody-dependent cell-mediated cytotoxity (ADCC) in both murine IgG2a and human IgG1. These LALA-PG substitutions allow a more accurate translation of results generated with an “effectorless” antibody framework scaffold between mice and primates. In particular embodiments of any of the IgG disclosed herein, the IgG comprises one or more of the following amino acid substitutions: N297G, N297A, N297E, L234A, L235A, or P236G.

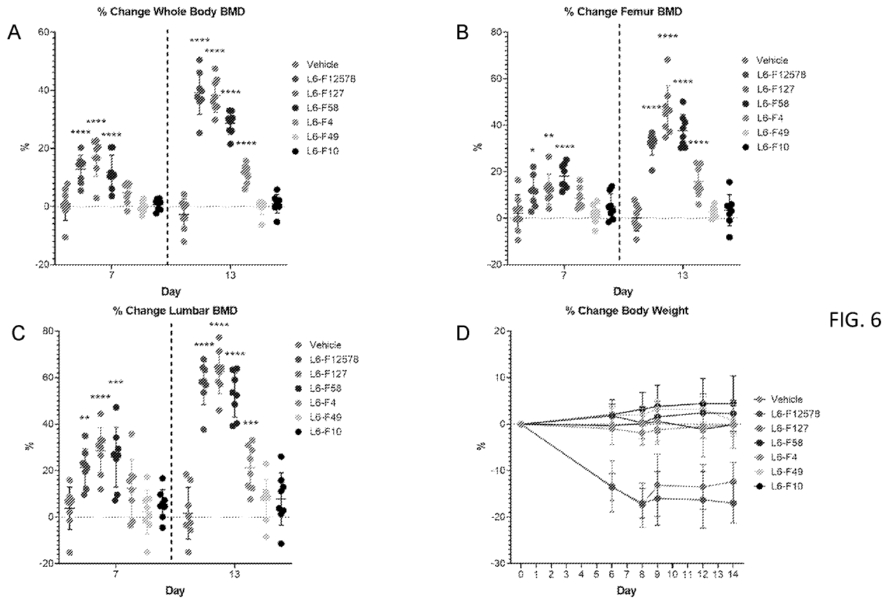

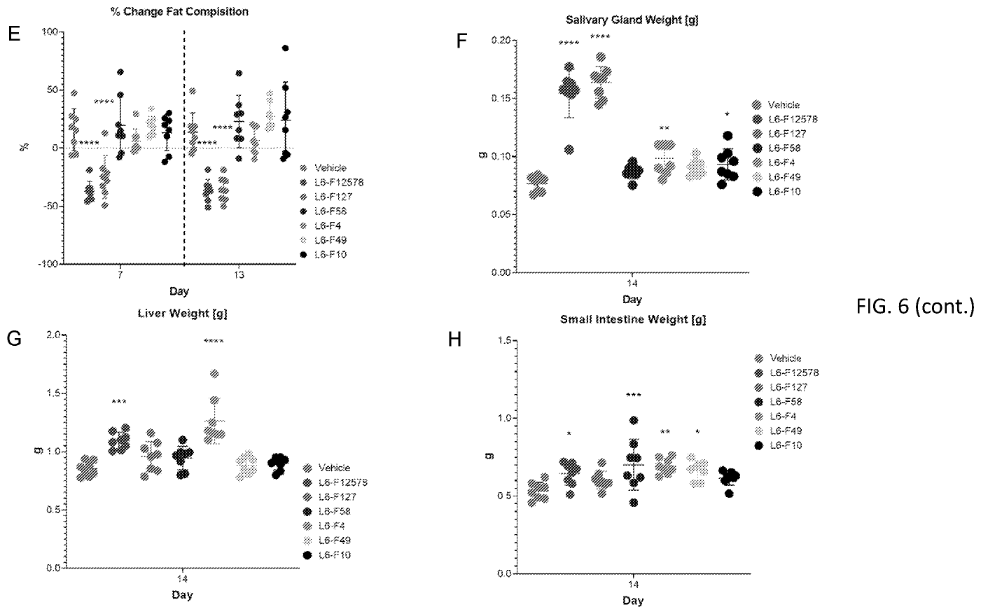

Non-limiting examples of bivalent and bispecific surrogate molecules of co-receptors that are bivalent towards both the one or more first receptor and one or more second receptor (e.g., FZD and LRP) are provided as the top four structures depicted in WO2019126398 and WO2020010308, where the VHH or scFv is depicted in white or grey, and the Fab or IgG is depicted in black. As shown, the VHH (or scFvs) may be fused to the N-termini of both light chains, to the N-termini of both heavy chains, to the C-termini of both light chains, or to the C-termini of both heavy chains. It is further contemplated, e.g., that VHH (or scFvs) could be fused to both the N-termini and C-termini of the heavy and/or light chains, to the N-termini of the light chains and the heavy chains, to the C-termini of the heavy and light chains, to the N-termini of the heavy chains and C-termini of the light chains, or to the C-termini of the heavy chains and the N-termini of the light chains. In other related embodiments, two or more VHH (or scFvs) may be fused together, optionally via a linker moiety, and fused to the Fab or IgG at one or more of these locations. In a related embodiment, the surrogate molecule has a Hetero-IgG format, whereas the Fab is present as a half antibody, and one or more VHH (or scFv) is fused to one or more of the N-terminus of the Fc, the N-terminus of the Fab, the C-terminus of the Fc, or the C-terminus of the Fab. A bispecific but monovalent to each receptor version of this format is depicted at FIG. 6. In certain embodiments, the Fab or antigen-binding fragment (or IgG) thereof is fused directly to the VHH (or scFv) or antigen-binding fragment thereof, whereas in other embodiments, the binding regions are fused via a linker moiety. In particular embodiments, the Fab is described herein or comprises any of the CDR sets described herein.

In various embodiments, including but not limited to those depicted in WO2019126398, WO2020010308, Table 3, Table 4, and FIGS. 1-8, an antigen binding molecule comprises one or more Fab or antigen-binding fragment thereof that binds one or more first receptor (e.g., FZD receptors) and one or more Fab or antigen-binding fragment thereof that binds to at least one or more second receptor (e.g., LRP5 and/or LRP6). In a particular embodiment, it comprises two Fab or antigen-binding fragments thereof that bind one or more first co-receptor and/or two Fab or antigen-binding fragments thereof that bind to one or more second co-receptor. In further embodiments, one or more of the Fab is present in a full IgG format, and in certain embodiments, both Fab are present in a full IgG format. In certain embodiments, the Fab in full IgG format specifically binds one or more first receptor (e.g., one or more FZD receptor), and the other Fab specifically binds at least one second receptor (e.g., LRP5 and/or LRP6). For example, the Fab specifically binds one or more FZD receptor, and the Fab in full IgG format specifically binds LRP5 and/or LRP6. In certain embodiments, the Fab specifically binds LRP5 and/or LRP6, and the Fab in full IgG format specifically binds one or more FZD receptor. In certain embodiments, the Fab is fused to the N-terminus of the IgG, e.g., to the heavy chain or light chain N-terminus, optionally via a linker. In certain embodiments, the Fab is fused to the N-terminus of the heavy chain of the IgG and not fused to the light chain. In particular embodiments, the two heavy chains can be fused together directly or via a linker. An example of such a bispecific and bivalent with respect to both receptors is shown in FIG. 1A. In other related embodiments, two or more VHHs may be fused together, optionally via a linker moiety, and fused to the Fab or IgG at one or more of these locations. In a related embodiment, the WNT surrogate molecule has a Hetero-IgG format, whereas one of the Fab is present as a half antibody, and the other Fab is fused to one or more of the N-terminus of the Fc, the N-terminus of the Fab, or the C-terminus of the Fc. A bispecific but monovalent to each receptor version of this format is depicted at FIG. 6. In certain embodiments, the Fab or antigen-binding fragment thereof is fused directly to the other Fab or IgG or antigen-binding fragment thereof, whereas in other embodiments, the binding regions are fused via a linker moiety. In particular embodiments, the one or both of the two Fabs are described herein or comprise any of the CDR sets described herein.

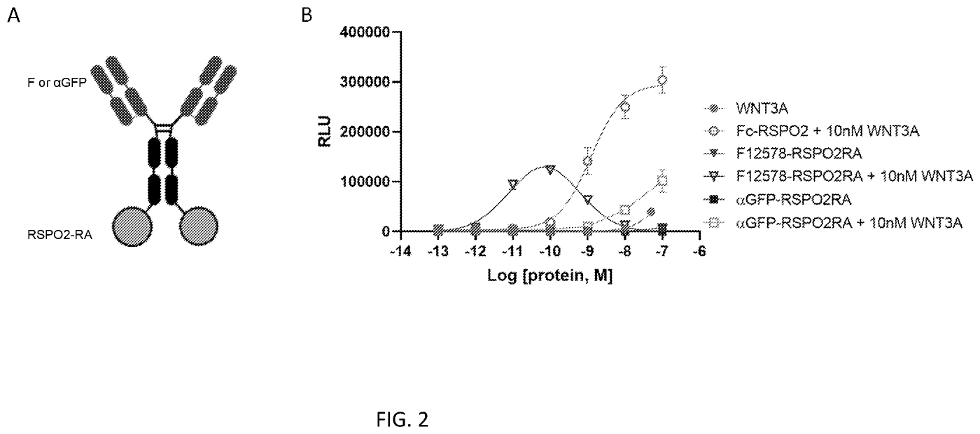

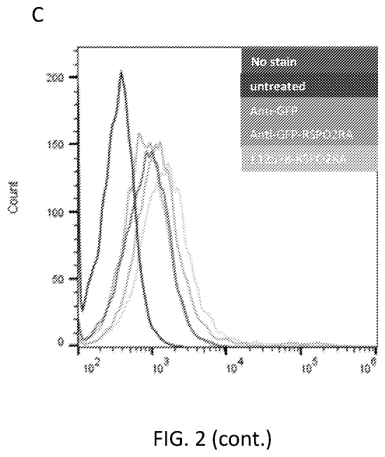

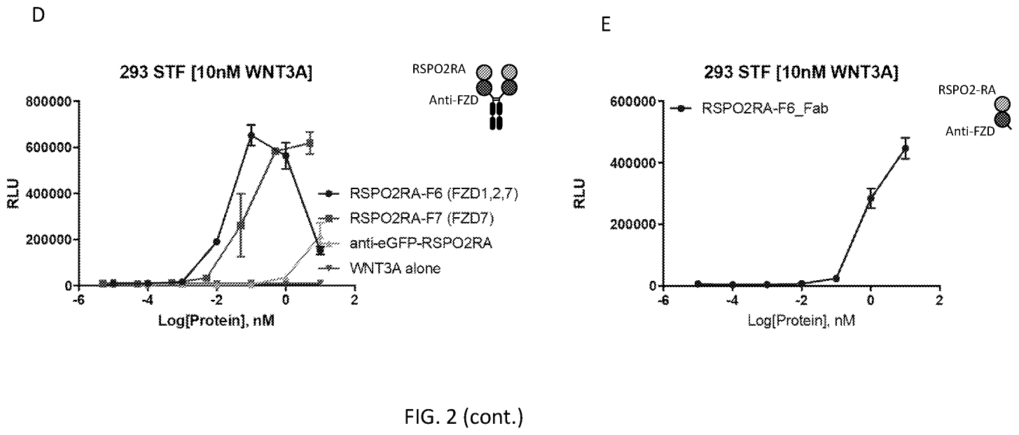

In certain embodiments, the antigen binding molecules have a format as described in PCT Application Publication No. WO2017/136820, e.g., a Fabs-in-tandem IgG (FIT-IG) format. Shiyong Gong, Fang Ren, Danqing Wu, Xuan Wu & Chengbin Wu (2017). FIT-IG also include the formats disclosed in, e.g., Gong, et al (2017) mAbs 9:118-1128. In certain embodiments, FIT-IGs combine the functions of two antibodies into one molecule by re-arranging the DNA sequences of two parental monoclonal antibodies into two or three constructs and co-expressing them in mammalian cells. Examples of FIT-IG formats and constructs are provided in FIGS. 1A and 1B and FIGS. 2A and 2B of PCT Application Publication No. WO2017/136820. In certain embodiments, FIT-IGs require no Fc mutation; no scFv elements; and no linker or peptide connector. The Fab-domains in each arm work “in tandem” forming a tetravalent bi-specific antibody with four active and independent antigen binding sites that retain the biological function of their parental antibodies In particular embodiments, WNT surrogates comprises a Fab and an IgG. In certain embodiments, the Fab binder LC is fused to the HC of the IgG, e.g., by a linker of various length in between. In various embodiment, the Fab binder HC can be fused or unfused to the LC of the IgG. A variation of this format has been called Fabs-in-tandem IgG (or FIT-Ig).

In certain embodiments, the WNT surrogate molecules have a format described in PCT Application Publication No. WO2009/080251 (Klein et al.), e.g. a CrossMab format. CrossMabs formats are also described in Schaefer et al. (2011) Proc. Natl. Acad. Sci USA 108:11187-11192. The CrossMab format allows correct assembly of two heavy chains and two light chains derived from existing antibodies to form a bispecific, bivalent IgG antibodies. The technology is based on the cross over the antibody domain within one Fab-arm of a bispecific IgG antibody in order to enable correct chain association. Various portions of the Fab can be exchanged, e.g., the entire Fab, the variable heavy and light chains, or the CH1-CL chains can be exchanged.

In further embodiments of the present invention, the FiT-Ig and CrossMab technologies are combined to create a multispecific, multivalent antigen binding molecule, Cross-FiT, as depicted in FIG. 1A and Table 2. Also contemplated is a linker between the crossed CL domain of the Fab and the Ig domains rather than between the CH1 and Ig domains. Additional antigen binding fragments, e.g., Fabs, VHH/sdAbs, and/or scFvs, can be appended to the Cross-FiT structure at various sites, e.g., the heavy or light chains and/or the C-terminus of the Fc domain to create multispecific antibodies.

In particular embodiments, surrogate molecules comprise two or more VHHs/sdAbs (or scFvs), including at least one that binds one or more first receptor and at least one that binds at least one second receptor. In certain embodiments, one of the binding regions is a VHH/sdAbs and the other is an scFv. Surrogate molecules comprising two or more VHH/sdAbs (or scFvs) may be formatted in a variety of configurations, including but not limited to those depicted in WO2019126398 and WO2020010308. In certain bispecific, bivalent formats, two or more VHH/sdAbs (or scFvs) are fused in tandem or fused to two different ends of an Fc, optionally via one or more linkers. Where linkers are present, the linker and its length may be the same or different between the VHH/sdAb (or scFv) and the other VHH/sdAb (or scFv), or between the VHH and Fc. For example, in certain embodiments, the VHH/sdAb is fused to the N-terminus, at either the heavy or light chain, and/or C-terminus of the IgG heavy chain. In particular embodiments, two or more VHH/sdAbs are fused to the IgG at any combination of these locations. In various embodiments, both VHH/sdAbs may be fused to the N-termini of the Fc, to the C-termini of the Fc, or one or more VHH/sdAb may be fused to either or both of an N-terminus or C-terminus of the Fc. In a related embodiment, the surrogate molecule has a Hetero-IgG format, whereas one VHH/sdAb is present as a half antibody, and the other is fused to the N-terminus of the Fc or the C-terminus of the Fc. In certain embodiments, the VHH/sdAb is fused directly to the other VHH/sdAb whereas in other embodiments, the binding regions are fused via a linker moiety. In particular embodiments, the VHH/sdAb are described herein or comprises any of the CDR sets described herein. In various embodiments, any of these formats may comprise one or more scFvs in place of one or more VHH/sdAbs.

In certain embodiments, a surrogate molecule is formatted as a diabody. The binders against the two co-receptors can also be linked together in a diabody (or DART) configuration. The diabody can also be in a single chain configuration. If the diabody is fused to an Fc, this will create a bivalent bispecific format. Without fusion to Fc, this would be a monovalent bispecific format. In certain embodiments, a diabody is a noncovalent dimer scFv fragment that consists of the heavy-chain variable (VH) and light-chain variable (VL) regions connected by a small peptide linker. Another form of diabody is a single-chain (Fv)2 in which two scFv fragments are covalently linked to each other.

As discussed, the surrogate molecules, in various embodiments, comprise one or more antibodies or antigen-binding fragments thereof disclosed herein. Thus, in particular embodiments, the surrogate comprises two polypeptides, wherein each polypeptide comprises an Nab or scFv that binds at least one first co-receptor and an Nab or scFv that binds at least one second co-receptor, optionally wherein one of the binding domains is an scFv and the other is an Nab. In certain embodiments, a surrogate comprises three polypeptides, wherein the first polypeptide comprises an antibody heavy chain and the second polypeptide comprises an antibody light chain, wherein the antibody heavy chain and light chain bind either receptor, and wherein the third polypeptide comprises a VHH/sdAb fused to a heavy chain Fc region or the light chain of the antibody, wherein the VHH/sdAb binds to either co-receptor. In other embodiments, the surrogates comprise four polypeptides, including two heavy chain polypeptides and two light chain polypeptides, wherein the two heavy chains and two light chains bind one or more first receptor, and further comprise one or more VHH/sdAb or scFv fused to one or more of the heavy chains and/or light chains, wherein the VHH/sdAb or scFv binds to one or more second co-receptor. In an illustrative embodiment, a WNT surrogate comprises at least four polypeptides, including two heavy chain polypeptides and two light chain polypeptides that bind either LRP5/6 or one or more FZDs, wherein the WNT surrogate further comprises a Fab that binds either LRP5/6 or one or more FZDs. For example, the Fab may comprise two polypeptides, each fused to one of the two heavy chain polypeptides, and two polypeptides, each fused to one of the two light chain polypeptides, or it may comprise two polypeptides each fused to one of the two heavy chain polypeptides and two additional polypeptides, each bound to one of the two polypeptides fused to the heavy chain polypeptides, thus making a second Fab. Other configurations disclosed herein may be used to produce different surrogate molecules.

Also contemplated are Ig molecules where the VL and VH domains of one Ig are appended with the VL and VH domains of a second antibody. This format is call Fv-Ig or 2Fv-Ig for a homodimer. The VL and VH domains from the second Ig are appended to the N-terminus of the VL and VH domains of the first Ig via short peptide linkers. This format preserves the natural antibody's avidity to cell surface receptors or to more than one receptor or co-receptor complexes (see, e.g., Wu, et al (2007) Nature Biotechnol. 25:1290-1297)

In certain embodiments, the antigen binding formats are surrogate molecules that comprise one or more polypeptides comprising two or more binding regions. For illustrative purposes, the two or more binding regions may be a first receptor binding regions or a second receptor binding regions, or they may comprise one or more first receptor binding region and one or more second receptor binding region. The binding regions may be directly joined or contiguous, or may be separated by a linker, e.g. a polypeptide linker, or a non-peptidic linker, etc. The length of the linker, and therefore the spacing between the binding domains can be used to modulate the signal strength, and can be selected depending on the desired use of the surrogate molecule. The enforced distance between binding domains can vary, but in certain embodiments may be less than about 100 angstroms, less than about 90 angstroms, less than about 80 angstroms, less than about 70 angstroms, less than about 60 angstroms, or less than about 50 angstroms. In some embodiments the linker is a rigid linker, in other embodiments the linker is a flexible linker. In certain embodiments where the linker is a peptide linker, it may be from about 1-30 amino acids in length, about 5-15 amino acids in length, or about 1, 2, 3, 4, 5, 6, 7, 8, 9, 10, 11, 12, 13, 14, 15, 16, 17, 18, 19, 20 21, 22, 23, 24, 25, 26, 27, 28, 29, 30 or more amino acids in length, and is of sufficient length and amino acid composition to enforce the distance between binding domains. In some embodiments, the linker comprises or consists of one or more glycine and/or serine residues.

The surrogate molecule can be multimerized, e.g. through an Fc domain, by concatenation, coiled coils, polypeptide zippers, biotin/avidin or streptavidin multimerization, and the like. The surrogate molecules can also be joined to a moiety such as PEG, Fc, etc., as known in the art to enhance stability in vivo.

In certain embodiments, a surrogate molecule enhances or increases the co-receptors pathway signaling, e.g., in the case of WNT—β-catenin signaling, by at least 30%, 35%, 40%, 45%, 50%, 60%, 70%, 75%, 80%, 85%, 90%, 95%, 100%, 110%, 150%, 200%, 250%, 300%, 400% or 500%, as compared to the β-catenin signaling induced by a neutral substance or negative control as measured in an assay described above, for example as measured in the TOPFIash assay (see, e.g., Molinaar (1996) Cell 86:391-399). A negative control may be included in these assays. By way of example, WNT surrogate molecules may enhance β-catenin signaling by a factor of 2×, 5×, 10×, 100×, 1000×, 10000× or more as compared to the activity in the absence of the WNT surrogate molecule when measured, for example when measured in the TOPFIash assay.

In certain embodiments, functional properties of the surrogate molecules (and WNT super agonists and WNT enhancers or RSPO mimetics) may be assessed using a variety of methods known to the skilled person, including e.g., affinity/binding assays (for example, surface plasmon resonance, competitive inhibition assays), cytotoxicity assays, cell viability assays, cell proliferation or differentiation assays in response to the native molecule/ligand, cancer cell and/or tumor growth inhibition using in vitro or in vivo models, including but not limited to any described herein. The surrogate molecules may also be tested for effects on one or both co-receptor internalization, in vitro and in vivo efficacy, etc. Such assays may be performed using well-established protocols known to the skilled person (see e.g., Current Protocols in Molecular Biology (Greene Publ. Assoc. Inc. & John Wiley & Sons, Inc., NY, NY); Current Protocols in Immunology (Edited by: John E. Coligan, Ada M. Kruisbeek, David H. Margulies, Ethan M. Shevach, Warren Strober 2001 John Wiley & Sons, NY, NY); or commercially available kits.

In certain embodiments, a binding region of a surrogate molecule (e.g., an antigen-binding fragment of an anti-FZD antibody) comprises one or more of the CDRs of the anti-co-receptor antibodies. In this regard, it has been shown in some cases that the transfer of only the VHCDR3 of an antibody can be performed while still retaining desired specific binding (Barbas et al., PNAS (1995) 92: 2529-2533). See also, McLane et al., PNAS (1995) 92:5214-5218, Barbas et al., J. Am. Chem. Soc. (1994) 116:2161-2162).

Also disclosed herein is a method for obtaining an antibody or antigen binding domain specific for a co-receptor, the method comprising providing by way of addition, deletion, substitution or insertion of one or more amino acids in the amino acid sequence of a VH domain set out herein or a VH domain which is an amino acid sequence variant of the VH domain, optionally combining the VH domain thus provided with one or more VL domains, and testing the VH domain or VH/VL combination or combinations to identify a specific binding member or an antibody antigen binding domain specific for one or more co-receptors and optionally with one or more desired properties. The VL domains may have an amino acid sequence which is substantially as set out herein. An analogous method may be employed in which one or more sequence variants of a VL domain disclosed herein are combined with one or more VH domains.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. See, generally, Davies et al. (1990) Annual Rev. Biochem. 59:439-473.

In certain embodiments, the surrogate molecules or binding regions thereof described herein have an affinity of less than about 10,000 nM, less than about 1000 nM, less than about 100 nM, less than about 10 nM, less than about 1 nM, or less than about 0.1 nM, and in some embodiments, the antibodies may have even higher affinity for one or more co-receptors.

The constant regions of immunoglobulins show less sequence diversity than the variable regions, and are responsible for binding a number of natural proteins to elicit important biochemical events. In humans, there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region. Molecules disclosed herein may comprise an antibody constant region of any class, subclass, or isotype.

The Fc region of an antibody interacts with a number of Fc receptors and ligands, imparting an array of important functional capabilities referred to as effector functions. For IgG, the Fc region comprises Ig domains CH2 and CH3 and the N-terminal hinge leading into CH2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack.

The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). All FcγRs bind the same region on Fc, at the N-terminal end of the Cg2 (CH2) domain and the preceding hinge. This interaction is well characterized structurally (Sondermann et al., 2001, J Mol Biol 309:737-749), and several structures of the human Fc bound to the extracellular domain of human FcγRIIIb have been solved (pdb accession code 1E4K) (Sondermann et al., 2000, Nature 406:267-273) (pdb accession codes 1IIS and IIIX) (Radaev et al., 2001, J Biol Chem 276:16469-16477.)

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a Kd for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical; however, FcγRIIIb does not have an intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome. A particularly relevant polymorphism with clinical significance is V158/F158 FcγRIIIa. Human IgG1 binds with greater affinity to the V158 allotype than to the F158 allotype. This difference in affinity, and presumably its effect on ADCC and/or ADCP, has been shown to be a significant determinant of the efficacy of the anti-CD20 antibody rituximab (Rituxan®, a registered trademark of IDEC Pharmaceuticals Corporation). Subjects with the V158 allotype respond favorably to rituximab treatment; however, subjects with the lower affinity F158 allotype respond poorly (Cartron et al., 2002, Blood 99:754-758). Approximately 10⁻²⁰% of humans are V158/V158 homozygous, 45% are V158/F158 heterozygous, and 35-45% of humans are F158/F158 homozygous (Lehrnbecher et al., 1999, Blood 94:4220-4232; Cartron et al., 2002, Blood 99:754-758). Thus 80-90% of humans are poor responders, that is, they have at least one allele of the F158 FcγRIIIa.

The Fc region is also involved in activation of the complement cascade. In the classical complement pathway, C1 binds with its C1q subunits to Fc fragments of IgG or IgM, which has formed a complex with antigen(s). In certain embodiments of the invention, modifications to the Fc region comprise modifications that alter (either enhance or decrease) the ability of a FZD-specific antibody as described herein to activate the complement system (see e.g., U.S. Pat. No. 7,740,847). To assess complement activation, a complement-dependent cytotoxicity (CDC) assay may be performed (See, e.g., Gazzano-Santoro et al., J. Immunol. Methods, 202:163 (1996)).

Thus in certain embodiments, the present invention provides the surrogate molecules having a modified Fc region with altered functional properties, such as reduced or enhanced CDC, ADCC, or ADCP activity, or enhanced binding affinity for a specific FcγR or increased serum half-life. Other modified Fc regions contemplated herein are described, for example, in issued U.S. Pat. Nos. 7,317,091; 7,657,380; 7,662,925; 6,538,124; 6,528,624; 7,297,775; 7,364,731; Published U.S. Applications US2009092599; US20080131435; US20080138344; and published International Applications WO2006/105338; WO2004/063351; WO2006/088494; WO2007/024249.

Structurally, the Fc region can be important for proper assembly of the msAb. In particular, modifications to the CH3 domain such as knobs-in-hole (see, e.g., WO1996/027011; and WO1998/050431) or Azymetric mutations (see, e.g., WO2012/58768) can prevent heavy chain mispairing. The present invention utilizes these mutations in certain Fc embodiments.

The surrogate molecules disclosed herein may also be modified to include an epitope tag or label, e.g., for use in purification or diagnostic applications. There are many linking groups known in the art for making antibody conjugates, including, for example, those disclosed in U.S. Pat. No. 5,208,020 or EP Patent 0 425 235 B1, and Chari et al., Cancer Research 52:127-131 (1992). The linking groups include disulfide groups, thioether groups, acid labile groups, photolabile groups, peptidase labile groups, or esterase labile groups, as disclosed in the above-identified patents, disulfide and thioether groups being preferred.

In certain embodiments, and antigen-binding fragments thereof against one co-receptor and/or antibodies and antigen-binding fragments thereof against the other co-receptor present within a surrogate molecule are monoclonal. In certain embodiments, they are humanized.

### IV. WNT Signal Enhancing Molecules (WNT Enhancers)

RSPOs are capable of amplifying WNT signals. The minimal functional unit of RSPO is composed of two Furin domains, Furin domain 1 that binds to ZNRF3/RNF43 E3 ligases, and Furin domain 2 that binds to LGR4-6, bringing together a ternary complex of RSPO, LGR, and the E3 ligases. This results in internalization of the whole complex and removal of ZNRF3/RNF43 away from their targets of destruction. Furin domain 1 alone is not functional, but it is capable of binding to both ZNRF3 and RNF43. In particular embodiments, when used in combination with a WNT or WNT surrogate molecule (e.g., to contact a cell), a WNT signal enhancing molecule increases signaling as compared to if only the WNT or WNT surrogate was used, e.g., by at least 10%, at least 20%, at least 30%, at least 50%, or at least two-fold, at least 3-fold, at least five-fold, or at least 10-fold.

The action module or E3 ligase binding domain responsible for enhancing WNT signaling described herein can be, but is not limited to, any functional moiety that can bind to the ZNRF3/RNF43 ligases, e.g., polypeptides, antibodies or fragments thereof, or organic chemicals. In particular embodiments, the action module, for example a polypeptide comprising the Furin domain 1 of an RSPO, either alone or together with a tissue specific targeting module (which may be substantially inactive in non-target tissues, so as to minimize potential off-target effects). The action module is fused to or bound to at least one WNT receptor or receptor binding domain, and when the E3 ligases ZNRF3/RNF43 are recruited to a ternary leading them to be relocated on the cell surface, sequestered, and/or cleared from the cell surface.

In certain embodiments, the action module or E3 ligase binding domain comprises a fragment or variant of an RSPO polypeptide (e.g., any of RSPOs 1-4), or a functional fragment or variant thereof. In particular embodiments, the action module comprises a fragment of a wild-type RSPO, and in other embodiments, the action module comprises a fragment of an RSPO comprising one or more amino acid modifications. The RSPO may be any RSPO known in the art or a homolog thereof, including RSPOs from any animal species, including but not limited to mammalian species, such as human RSPOs. RSPOs have been identified and described, and their polypeptide and encoding polynucleotide sequences are known and available in the art. In particular embodiments, the RSPO polypeptide is a human RSPO or a homolog found in other vertebrates or non-vertebrates, e.g., a mouse RSPO. Their homologues and variants are available from general database search, such as https://www.dot.ncbi.dot.nlm.dot.nih.dot.gov/protein/. The present invention includes (but is not limited to) action modules comprising or consisting of fragments and variants of any of these or other RSPOs, in particular, RSPO 2. In various embodiments, variants of any of the RSPO polypeptides and fragments thereof comprise one or more amino acid modifications, e.g., deletions, additions, or substitutions as compared to the wild-type RSPO polypeptide. The modification(s) may be present in any region of the variant of RSPO or a fragment thereof, including but not limited to a Furin domain 1 and/or a Furin domain 2. In particular embodiments, the RSPO is RSPO 2 containing mutations in the Furin domain 2, e.g., F105R and F109A, resulting in abrogated LGR4-6 binding. This mutant RSPO is known as “RSPO2RA”. It is understood that amino acid modifications outside of the Furin domain 1 or Furin domain 2 may alter the resulting variant such that the resulting variant has reduced LGR4-6 binding activity as compared to the wild-type RSPO or fragment thereof.

In certain embodiments, the action module comprises or consists of an RSPO sequence, e.g., a full length or wild-type RSPO-1, -2, -3 or -4, optionally a human RSPO-1, -2, -3, or -4, or a variant or fragment thereof. In particular embodiments, it is a variant of any of RSPOs-1-4 having at least 80%, at least 85%, at least 90%, at least 95%, at least 98%, or at least 99% sequence identity to the corresponding wild-type RSPO-1-4 sequence. In certain embodiments, the action module comprises or consists of a full length RSPO (e.g., any of RSPOs-1-4) comprising one or more amino acid modifications, including but not limited to any of those disclosed herein. In certain embodiments, the action module comprises or consists of a fragment of a wild-type or modified RSPO (e.g., any of RSPOs-1-4). In particular embodiments, the fragment is able to bind to ZNRF3 and/or RNF43. In certain embodiments, the action module comprises the Furin domain 1 of an RSPO protein, or fragments or variants of RSPO proteins. In certain embodiments, the action module comprises or consists of one or more (e.g., one, two or three or more Furin domain 1 of an RSPO protein (e.g., RSPO-1-4), or a variant thereof having at least 85%, at least 90%, at least 95%, at least 98% or at least 99% sequence identify to an RSPO Furin domain 1. In certain embodiments, the action module comprises an RSPO Furin 1 domain or variant or fragment thereof and an RSPO Furin 2 domain or variant or fragment thereof. In certain embodiments, the action module comprises an antibody, or antigen binding fragment thereof, that bind ZNRF3/RNF43. In particular embodiments, the action module specifically binds to either ZNRF3 or RNF43. Examples of ZNRF3/RNF43 binding molecules are described in WO2020014271.

In certain embodiments, the action module or E3 ligase binding domain comprises one or more Furin domain 1 of an RSPO, e.g., human RSPO 1 or human RSPO 2, or a variant thereof. In certain embodiments, the action module comprises one or more Furin domain 1 of an RSPO, but it does not comprise a Furin domain 2 of an RSPO. In certain embodiments, the action module comprises one or more Furin domain 1 of an RSPO, and it comprises a modified or variant Furin domain 2 of an RSPO, e.g., a Furin domain 2 with reduced activity as compared to the wild-type Furin domain 2. In certain embodiments, the action module comprises an RSPO protein having a modified or variant Furin domain 2 of an RSPO, e.g., a Furin domain 2 with reduced activity as compared to the wild-type Furin domain 2. In certain embodiments, an action module comprises two or more Furin domains 1, or variants thereof, or multimers of a Furin domain 1 or variant thereof. In certain embodiments, the action module comprises a variant RSPO Furin 1 domain comprising one or more point mutations, e.g., at amino acid residues corresponding to K58, H76, S77, R86, and/or N91 of human RSPO 2. In certain embodiments, the action module comprises a variant RSPO Furin 2 domain comprising one or more point mutations, e.g., at amino acid residues corresponding to F105, F109 (e.g., “RSPO2RA”), and/or K121 of human RSPO 2. In particular embodiments, the action module comprises a modified or variant Furin domain 1 of an RSPO that has increased activity, e.g., binding to ZNRF3/RNF43, as compared to the wild-type Furin domain 1. Action modules or E3 ligase binding domain may further comprise additional moieties or polypeptide sequences, e.g., additional amino acid residues to stabilize the structure of the WNT signal enhancing molecule in which it is present. In certain embodiments, an action module comprises a peptide or polypeptide without obvious/strong sequence homology to RSPOs but has binding affinity to ZNRF3/RNF43 comparable to or higher than the binding affinity of RSPOs to ZNRF3/RNF43.

In certain embodiments, the action module or E3 ligase binding domain comprises a Furin domain 1 of an RSPO polypeptide (e.g., a human RSPO), or a functional fragment or variant thereof, and a modified or variant Furin domain 2 of an RSPO polypeptide (e.g., a human RSPO), wherein the modified Furin domain 2 has reduced binding affinity to LGR4-6 as compared to the corresponding wild-type Furin domain 2. In certain embodiments, the Furin domain 2 comprises one or more point mutations, e.g., at amino acid residues corresponding to F105 and/or F109 of human RSPO 2. The skilled artisan can readily determine the corresponding amino acid residues in other RSPO polypeptides by comparing their amino acid sequences to human RSPO 2. In certain embodiments, the action module or E3 ligase binding domain comprises a Furin domain 1 or variant thereof and a Furin domain 2 or variant thereof, wherein the Furin domain 1 and/or Furin domain 2 comprises one or more point mutations. The one or more point mutations within the action module or E3 ligase binding domain (as compared to the corresponding wild-type RSPO sequence) may occur at any amino acid residues within the Furin domain 1 and/or Furin domain 2, including but not limited to, e.g., at amino acid residues K58, H76, S77, R86, N91, F105, F109, or K121 and other residues that can be modified to reduce the binding affinity to LGR4-6. Regions of the Furin domain 1 and Furin domain 2 of human RSPO 1 that are important for its functional activity have been identified, including conserved hydrophilic residues S48, N51, R66, R70 and Q71, and less conserved, hydrophobic residues, L46, L54, 162 and L64, which are important for binding to the E3 ligases. In addition, in the human RSPO 1 Furin domain 1, amino acid residues K59, S78, D85, R87, N88 and N92 form a hydrophilic interaction surface with LGR5, and the FSHNF amino acid sequence has been identified as a loop important for the hydrophobic surface.

In particular embodiments, action modules or E3 ligase binding domains comprising RSPO Furin domain 1 and/or Furin domain 2 may comprise one or more mutations within any of these regions, surfaces or amino acid residues. In particular embodiments, action modules comprising RSPO Furin domain 1 and/or Furin domain 2 may comprise one or more mutations or other alternations beyond these regions, surfaces or amino acid residues, which indirectly compromise LGR4-6 binding by affecting the structure and/or stability of the binding surface. In certain embodiments, action modules comprising RSPO Furin domain 1 and/or Furin domain 2 may comprise one or more mutations at any amino acid residues, including but not limited to any of those depicted in the accompanying Examples. In particular embodiments, the action module comprises a Furin 1 domain and a modified Furin domain 2 comprising amino acid substitutions at amino acid residues F105 and/or F109 (e.g., RSPO2RA). In particular embodiments, the action module comprises a modified Furin 1 domain and a modified Furin 2 domain, where in certain embodiments, the modified Furin 1 domain comprises one or more amino acid modifications at amino acids R65, R69 and/or Q70, and the modified Furin domain comprises one or more amino acid modification at amino acids F105 and/or F109. In particular embodiments, the modified Furin domain 2 has binding affinity to LGR4-6 less than 80%, less than 50%, less than 20%, or less than 10% the binding of the corresponding wild-type Furin domain 2, e.g., in the context of the full length RSPO protein.

In certain embodiments, the action module or E3 ligase binding domain comprises a Furin domain 1 of an RSPO polypeptide (e.g., a human RSPO), or a functional fragment or variant thereof, and an unmodified Furin domain 2 of an RSPO polypeptide (e.g., a human RSPO). While in certain embodiments, a modified Furin domain 2 having reduced binding affinity to LGR4-6 as compared to the corresponding wild-type Furin domain 2 is more desirable to increase the specificity of tissue targeting, in particular embodiments, the unmodified Furin domain 2 combined with the targeting module has improved tissue targeting over wild-type RSPO without targeting module, and has utility in certain contexts.

In certain embodiments, the action module or E3 ligase binding domain comprises a wild-type or modified RSPO Furin domain 1, e.g., from any of RSPO-1, -2, -3, -4, optionally human RSPOs-1, -2, -3 or -4. In particular embodiments, the action module comprises the RSPO Furin 1 domain and a wild-type or modified RSPO Furin 2 domain, e.g., from any of RSPO-1, -2, -3, -4, optionally human RSPOs-1, -2, -3 or -4. In particular embodiments, the action module comprises the first RSPO Furin 1 domain and a second wild-type or modified RSPO Furin 1 domain, e.g., from any of RSPO-1, -2, -3, -4, optionally human RSPOs-1, -2, -3 or -4. In particular embodiments, the modified Furin domain 2 has comparable binding affinity to LGR4-6 or a binding affinity to LGR4-6 of less than 80%, less than 50%, less than 20%, or less than 10% the binding of the corresponding wild-type Furin domain 2, e.g., in the context of the full length RSPO protein. In certain embodiments, the action module comprises an antibody or antigen-binding fragment thereof that specifically binds ZNRF3 and/or RNF43. In particular embodiments, the action module comprises an antibody or antigen-binding fragment thereof that binds to human RNF43 (hRNF43, NCBI reference sequence XP_011523257.1), or human ZNRF3 (hZNRF3; NCBI reference sequence NP_001193927.1). In particular embodiments, the action module is an antibody or an antigen-binding fragment thereof, comprising: a) CDRH1, CDRH2 and CDRH3 sequences set forth for any of the antibodies of WO2020014271 (e.g., see Table 2A); and/or b) CDRL1, CDRL2 and CDRL3 sequences set forth for any of the antibodies of WO2020014271 (e.g., see Table 2A), or a variant of said antibody, or antigen-binding fragment thereof, comprising one or more amino acid modifications, wherein said variant comprises less than 8 amino acid substitutions in said CDR sequences.

### V. WNT Super Agonist Structures

The present invention encompasses WNT super agonist molecules, in particular, molecules containing a WNT surrogate (e.g., a FZD binder and an LRP binder) in combination with a WNT enhancer (e.g., an RSPO protein or E3 ligase binder). It was surprisingly found that molecules comprising both a WNT surrogate and a WNT enhancer acted as WNT super agonists, and induced a greater level of WNT signaling pathway activity than a WNT surrogate. In particular, it was expected that fusing an E3 ligase binding domain to a WNT surrogate would enhance ubiquitination of the target receptors of the WNT surrogate, and result in antagonism, based on previous studies of PROTAC, i.e., Deshaies, R. J., Nature, Vol 580, 16 Apr. 2020, p. 329). Similarly, it was previously shown that Disheveled binds to WNT receptors and serves as an adaptor to target the ZNRF3 and RNF43 E3 ubiquitin ligases to WNT receptors, leading to their degradation (Jiang, X. et al., 2015, Molecular Cell 58, 522-533). Thus, it was surprising and unexpected to find that the WNT enhancer (or RSPO mimetic, where the E3 ligase binding domain is fused to a FZD or LRP binding domain) and WNT super agonists disclosed herein resulted in increased levels of FZD protein on the cell surface and actually stimulated the WNT signaling pathway.

In certain embodiments, a WNT super agonist molecule enhances or increases the co-receptors pathway signaling, e.g., in the case of WNT—β-catenin signaling, by at least 30%, 35%, 40%, 45%, 50%, 60%, 70%, 75%, 80%, 85%, 90%, 95%, 100%, 110%, 150%, 200%, 250%, 300%, 400% or 500%, as compared to the β-catenin signaling induced by a neutral substance or negative control as measured in an assay described above, for example as measured in the TOPFlash assay (see, e.g., Molinaar (1996) Cell 86:391-399).

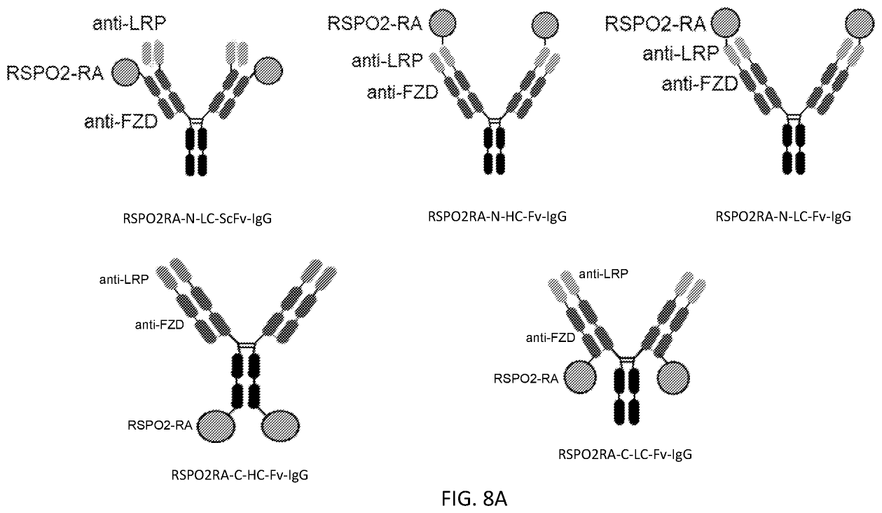

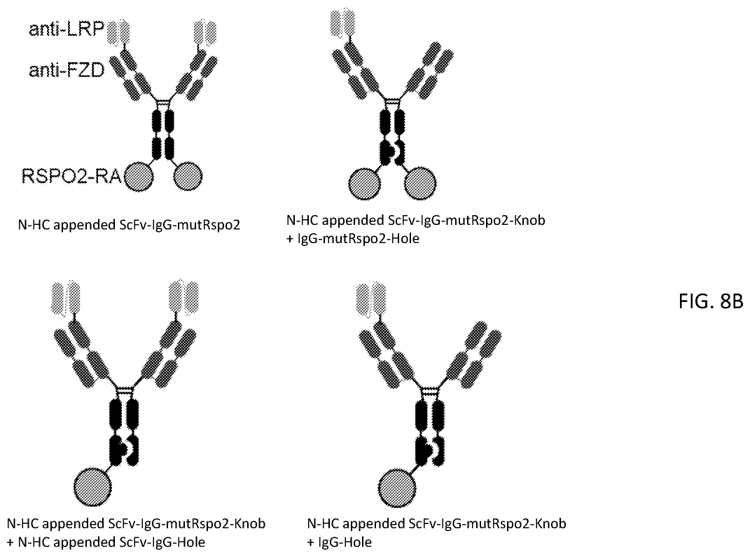

In certain embodiments, a WNT super agonist molecule comprises a first binding domain that binds one or more FZD; a second binding domain that binds LRP5/6; and a third domain comprising a WNT enhancer, e.g., wherein the WNT enhancer comprises an E3 ligase binding domain. These domains may be present on one, two, three, or four polypeptides. When present on more than one polypeptide, the two or more polypeptides may be bound to each other to form the WNT super agonist. Non-limiting examples of various WNT super agonist structures contemplated by the disclosure are provided in Table 4 and FIGS. 8A-8B.

In certain embodiments, a WNT super agonist molecule comprises any of the structures disclosed herein for a WNT surrogate molecule, while further comprising a WNT enhancer domain and, optionally, a targeting module. In some embodiments, the WNT enhancer domain comprises an RSPO protein or functional variant or fragment thereof. In some embodiments, the WNT enhancer domain binds one or more E3 ligase. In particular embodiments, the WNT enhancer domain does not substantially bind to LGR. In particular embodiments, the WNT enhancer domain is a mutant RSPO that lacks an LGR binding region.

In some embodiments, the WNT super agonist molecule further comprises a targeting module, e.g., a targeting module that binds specific cell types or tissue types. In certain embodiments of WNT super agonists, one or more WNT enhancer domain is fused to either end of any polypeptide present within a WNT surrogate molecule. In particular embodiments, one, two, three, or four WNT enhancer domains are present within a WNT super agonist.

In certain embodiments, the WNT surrogates, WNT enhancers, or WNT super agonists comprise or have a structure including, but not limited to, a tandem scFv, scFv-IgG, Fv-IgG, Fab-IgG, VHH-IgG, or Fv-Fab (see, e.g., the general structures of FIG. 1A and the specific structures of FIGS. 2A, 2D, 2E, 3A, 3J, 3L, 3M, 4, 7, 8A, 8B, and Tables 3 and 4).

Tandem scFv super agonists are generated and assembled by linking or directly fusing a first scFv to either the C- or N-terminus of a second scFv molecule. In one format, the first scFv can bind to one or more FZD receptors and the second scFv can bind to one or more LRP receptors. In an alternative format, the first scFv can bind to one or more LRP receptors, and the second scFv can bind to one or more FZD receptors. One of the scFv molecules can also be linked or directly fused at its C-terminus to the N-terminus of an Fc molecule. In certain embodiments of WNT super agonists, the WNT enhancer is linked or fused to the N-terminus of a first scFv, which in turn is linked or fused to the N-terminus of the second scFv, which is linked or fused to the N-terminus of the Fc molecule. In alternative embodiments, the WNT enhancer is linked or fused to the C-terminus of the Fc molecule, which in turn is linked or fused to the C-terminus of one scFv molecule, which is linked or fused at its N-terminus to the C-terminus of a second scFv molecule.

Fab-IgG molecules, where the FZD and LRP binders are both Fabs can be assembled in various approaches, such as charge pairing, knobs-in-holes, crossover of heavy and light chains of the Fabs, etc. In charge pairing the heavy chain (VH-CH1) domain of an anti-LRP6 Fab or an anti-FZD Fab (through direct fusion or a linker, e.g., a linker of 1-30 or 5-15 amino acid, e.g., 5, 10, or 15-mer amino acids) are fused in tandem with the N-terminus of the heavy chain (VH-CH1-CH2-CH3) of an anti-FZD or anti-LRP binder. In certain embodiments, also known as Fabs-in tandem (FiT), both VH-CH1 domains of anti-LRP6 and anti-FZD contain three amino acid mutations (Q39D, Q105D, S183K in the anti-LRP6 Fab; Q39K, Q105K, S183E in anti-FZD Fab) each for proper paring with their own partner light chains, which also contain three complementary amino acid mutations (Q38K, A/S43K, S176E in anti-LRP6 light chain; Q38D, A/S43D, S176K in the anti-FZD light chain). The order of the anti-LRP6 and anti-FZD Fabs could be reversed, where the anti-FZD binder is a Fab and is fused to anti-LRP binder which is in IgG format. In certain embodiments, the WNT enhancer can be attached to the Fab to the N-terminus of either the Vh or Vl the Fab furthest from the IgG domain. In other embodiments, the WNT enhancer is attached to C-terminus of the IgG domain.

HC-LC cross over approach for Fab-on-IgG format: The light chain (VL-CL) domains of anti-LRP6 binder is (through direct fusion or a linker, e.g., a linker of 1-30 or 5-15 amino acid, e.g., 5, 10, or 15-mer amino acids) fused in tandem with the N-terminus of the heavy chain (VH-CH1-CH2-CH3) of an anti-FZD binder. The second construct was VH-CH1 of the anti-LRP6 binder and the third construct was VL-CL of the anti-FZD binder. Similar to the example above, the order of the anti-LRP6 and the anti-FZD binders could be reversed, where anti-FZD binder Fab is fused to the N-terminus of the anti-LRP binder which is in IgG format. Also as above the WNT enhancer can be attached to N-terminus of the VH or VL of the crossover Fab furthest from the IgG domain, or attached to the C-terminus of the IgG domain.

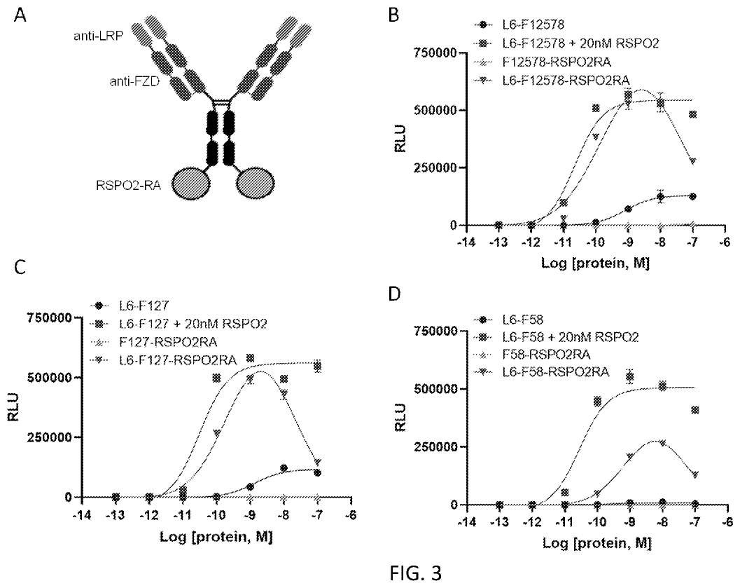

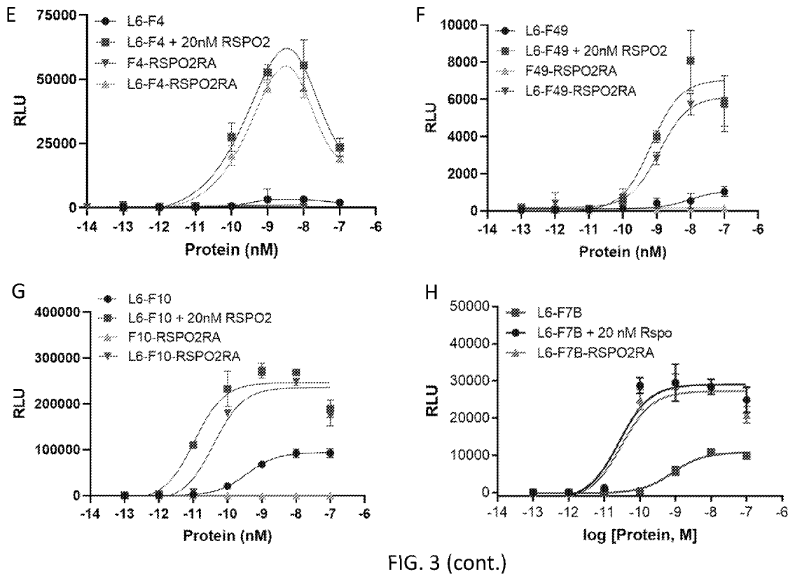

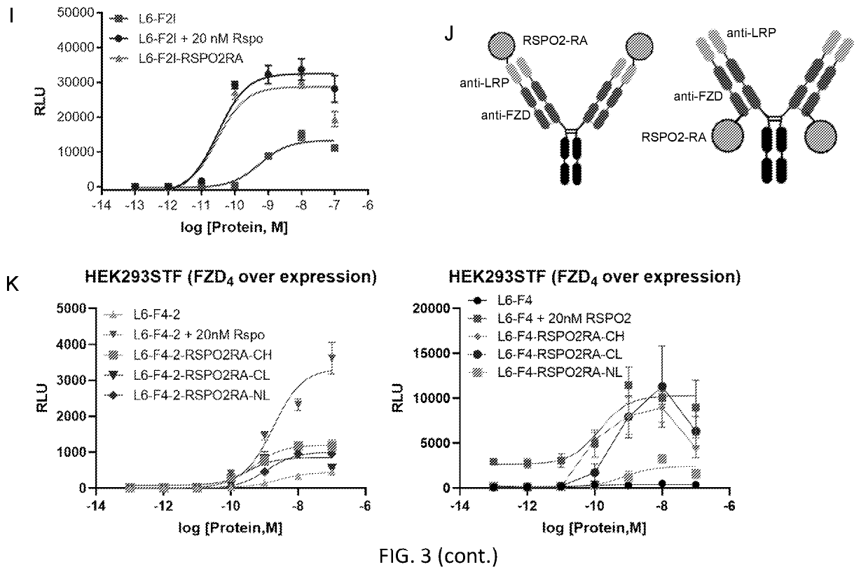

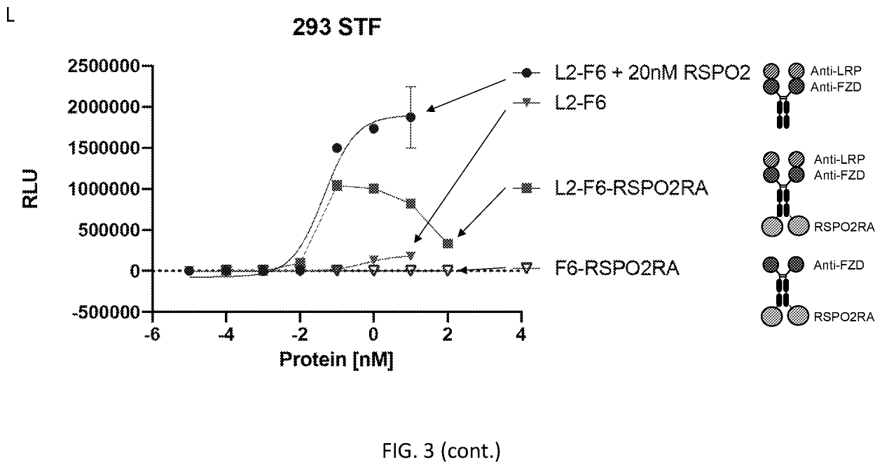

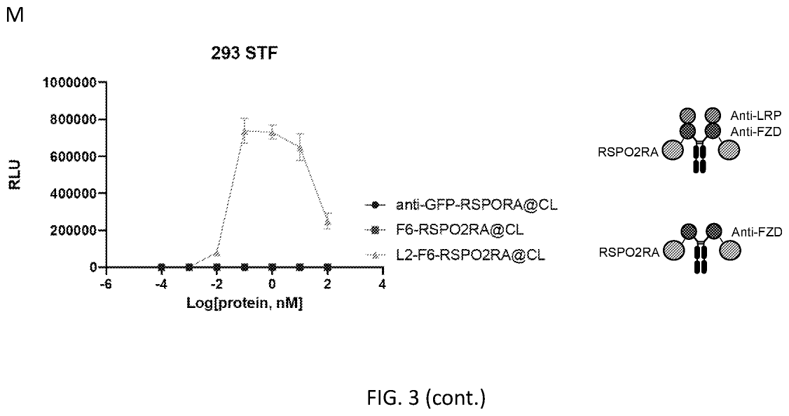

In certain embodiments, the WNT surrogate region of the WNT super agonist is an Fv-IgG. Illustrative examples of various structural formats that may be used are provided in FIG. 1A, and FIGS. 8A and 8B, as well as Table 4. In particular embodiments, the WNT super agonist is an Fv-IgG with the components including at least one binding domain that binds to at least one FZD receptor, at least one binding domain that binds to an LRP receptor, and either at least one RSPO protein (mutant or wild-type) or at least one binding domain that binds to an E3 ubiquitin ligase. In some embodiments, the LRP binding domain is a VHH or Fab fragment linked to the N-terminus of a Fab that binds to a FZD receptor, which is fused to an Fc domain at the C terminus of the FZD Fab (see, e.g., FIGS. 2A, 2D, 2E, 3A, 3J, 3L, 3M, 4A, 8A, 8B, and Table 4). In some embodiments, the FZD binding domain is a VHH or Fab fragment linked to the N-terminus of a Fab that binds to LRP5/6, which is fused to an Fc domain at the C terminus of the LRP5/6 Fab. In further embodiments, the Fv-IgG contains the LRP5/6 VHH and FZD Fab, with an RSPO protein or E3 ligase binder attached to the C-terminus of the Fc domain. Alternatively, the RSPO or E3 ligase binder can be attached to the C-terminus of the heavy or light chain of the Fab (see, e.g., FIG. 3J, FIGS. 8A and 8B, and Table 4).

In certain embodiments, the WNT surrogate region of the WNT super agonist is an Fv-IgG comprising four linked polypeptides, e.g., as depicted in FIG. 8A or FIG. 8B. In certain embodiments, the Fv-IgG comprises two heavy chain polypeptides and two light chain polypeptides. In certain embodiments, each heavy chain polypeptide comprises an Fc region, a variable region of an anti-FZD antibody, and a variable region of an anti-LRP5/6 antibody, wherein the two variable regions are present N-terminal to the Fc region, and wherein the two variable regions may be in either order. In one embodiment, the heavy chain comprises from N-terminus to C-terminus: an anti-LRP5/6 antibody variable region, an anti-FZD variable region, and an Fc region. In particular embodiments, one or both variable region is present within a Fab. The heavy chain may further comprise additional sequences, such as, e.g., a hinge region between the Fc region and the variable regions (or Fab). In particular embodiments, the two light chain polypeptides each comprise a variable region of an anti-FZD antibody, and a variable region of an anti-LRP5/6 antibody, wherein the two variable regions may be in either order, and wherein either or both variable region is present within a Fab. In particular embodiments, an E3 ligase binding domain is fused to the C-terminus or N-terminus of one or both heavy chains. In particular embodiments, an E3 ligase binding domain is fused to the C-terminus or N-terminus of one or both light chains (see, e.g., FIG. 8B). In particular embodiments, the two heavy chain polypeptides of the WNT super agonist molecule are the same, and bind to each other. In certain embodiments, the two heavy chain polypeptides of the WNT super agonist molecule are different, for example, when the WNT super agonist molecule includes only one E3 ligase binding domain. In order to properly combine a heavy chain without an E3 ligase domain with a heavy chain having an E3 ligase domain, the two different heavy chains may be engineered to selectively bind to each other to produce heterodimers, e.g., by introducing Knob-into-holes amino acid modifications into the two different polypeptides to facilitate their binding.

Additionally, the Fv-IgG or other format structure, can include a tissue or cell targeting domain, which can be attached at similar sites as the RSPO or E3 ligase binder or can be an full length antibody that binds a tissue/cell specific target with the WNT receptor binding domains and RSPO/E3 ligase binding domains attached at various sites as described above.

In particular embodiments, any of the domains present in the WNT super agonist are directly joined, or may be separated via a linker, e.g., a polypeptide linker, or a non-peptidic linker, etc. The length of the linker, and therefore the spacing between the binding domains can be used to modulate the signal strength, and can be selected depending on the desired use of the WNT super agonist molecule. The enforced distance between any of the various linked binding domains can vary, but in certain embodiments may be less than about 100 angstroms, less than about 90 angstroms, less than about 80 angstroms, less than about 70 angstroms, less than about 60 angstroms, or less than about 50 angstroms. In some embodiments, the linker is a rigid linker, in other embodiments the linker is a flexible linker. In certain embodiments where the linker is a peptide linker, it may be from about 1-30 amino acids in length, about 5-15 amino acids in length, or about 1, 2, 3, 4, 5, 6, 7, 8, 9, 10, 11, 12, 13, 14, 15, 16, 17, 18, 19, 20 21, 22, 23, 24, 25, 26, 27, 28, 29, 30 or more amino acids in length, and is of sufficient length and amino acid composition to enforce the distance between binding domains. In some embodiments, the linker comprises or consists of one or more glycine and/or serine residues.

In particular embodiments, the WNT super agonist comprises any of the ratios of FZD binding domains and LRP5/6 binding domains disclosed herein for WNT surrogate molecules. In particular embodiments, the WNT super agonist comprises any of the ratios of FZD binding domains and LRP5/6 binding domains disclosed herein for WNT surrogate molecules, and further comprises one or two E3 ligase binding domains.

In certain embodiments, the WNT super agonist molecules or one or more binding regions thereof described herein have an affinity of less than about 10,000 nM, less than about 1000 nM, less than about 100 nM, less than about 10 nM, less than about 1 nM, or less than about 0.1 nM, and in some embodiments, the antibodies may have even higher affinity for one or more co-receptors.

In particular embodiments, a WNT super agonist comprises one or more polypeptide sequence disclosed herein, e.g., in the Examples, or a functional variant or fragment thereof.

### VI. Targeting Molecules

Any of the molecules disclosed herein, e.g., WNT super agonists, WNT surrogates, and WNT enhancers (RSPO mimetics) may further comprise a cell- or tissue-specific binding domain.

Specific cell types and cells within specific tissue may comprise one or more cell- or tissue-specific surface molecule, such as a cell surface receptor. As used herein, the molecule is said to be cell- or tissue-specific if a greater amount of the molecule is present on the specific cell or tissue type as compared to one or more other cell or tissue types, or any other cell or tissue type. In certain embodiments, the greater amount is at least two-fold, at least five-fold, at least 10-fold, at least 20-fold, at least 50-fold, or at least 100-fold as compared to the amount in the one or more other cell or tissue types, or any other cell or tissue type. In particular embodiments, the cell-specific surface molecule has increased or enhanced expression on a target organ, tissue or cell type, e.g., an organ, tissue or cell type in which it is desirous to enhance WNT signaling, e.g., to treat or prevent a disease or disorder, e.g., as compared to one or more other non-targeted organs, tissues or cell types. In certain embodiments, the cell-specific surface molecule is preferentially expressed on the surface of the target organ, tissue or cell type as compared to one or more other organ, tissue or cell types, respectively. For example, in particular embodiments, a cell surface receptor is considered to be a tissue-specific or cell-specific cell surface molecule if it is expressed at levels at least two-fold, at least five-fold, at least 10-fold, at least 20-fold, at least 30-fold, at least 40-fold, at least 50-fold, at least 100-fold, at least 500-fold, or at least 1000-fold higher in the target organ, tissue or cell than it is expressed in one or more, five or more, all other organs, tissues or cells, or an average of all other organs, tissue or cells, respectively. In certain embodiments, the tissue-specific or cell-specific cell surface molecule is a cell surface receptor, e.g., a polypeptide receptor comprising a region located within the cell surface membrane and an extracellular region to which the targeting module can bind. In various embodiments, the methods described herein may be practiced by specifically targeting cell surface molecules that are only expressed on the target tissue or a subset of tissues including the target tissue, or by specifically targeting cell surface molecules that have higher levels of expression on the target tissue as compared to all, most, or a substantial number of other tissues, e.g., higher expression on the target tissue than on at least two, at least five, at least ten, or at least twenty other tissues.

The targeted tissue may be bound by a targeting module, e.g., a binding domain that specifically binds to the tissue specific receptor. The targeted tissue may be any tissue, e.g., any mammalian tissue or cell type. In certain embodiments, the targeted tissue may be present in any organ. In certain embodiments, the target tissue is bone tissue, liver tissue, skin tissue, stomach tissue, intestine tissue, oral mucosa tissue, kidney tissue, central nervous system tissue, mammary gland tissue, taste bud tissue, ovary tissue, inner ear tissue (including cochlear and vestibular tissues), hair follicles, pancreas tissue, retina tissue, cornea tissue, heart tissue or lung tissue, and the targeting module binds to a tissue-specific cell surface molecule (e.g., a cell surface receptor) preferentially expressed on bone tissue, liver tissue, skin tissue, stomach tissue, intestine tissue, oral mucosa tissue, kidney tissue, central nervous system tissue, mammary gland tissue, taste bud tissue, ovary tissue, inner ear tissue (including cochlear and vestibular tissues), hair follicles, pancreas tissue, retina tissue, cornea tissue, heart tissue or lung tissue, respectively.

The targeting module may bind to any cell type, e.g., any cell within any tissue, organ or animal, including but not limited to mammals, such as humans. In certain embodiments, the tissue-specific WNT surrogate-signal enhancing combination molecule binds to specific cell types, e.g., specific cell types associated with a target tissue. For example, in liver tissue, the targeting module may bind to hepatocytes, precursors and stem cells of hepatocytes, biliary tract cells, and/or endothelial or other vascular cells. For example, in bone tissue, the targeting module may bind osteoblasts, precursors of osteoblasts, mesenchymal stem cells, stem cells and precursor cells that give rise to bone, cartilage and/or other cells present in bone tissue. Cell types present in various tissues, including but not limited to the tissues described herein, are known in the art, and in various embodiments, the tissue-specific WNT signal enhancing molecules described herein may bind any of them.

### VII. WNT Enhancer Structures (RSPO Mimetics)

In some embodiments, an RSPO mimetic having the activities of RSPO is desirable. In certain embodiments, the disclosure provides RSPO mimetics, comprising: (i) either a FZD binding domain or an LRP5/6 binding domain (but not both); and an E3 ligase binding domain. The WNT enhancers can operate as RSPO mimetics. In certain embodiments, the RSPO mimetic can have the structures depicted in FIG. 2A, 2D, or 2E. In particular embodiments, an RSPO mimetic will have mutant RSPO (RSPO2RA) and at least one binding domain specific for a WNT receptor (e.g., FZD or LRP). The RSPO mimetic with a FZD binding domain can function as a tissue or cell specific RSPO mimetic if the FZD receptor expression is limited to a particular organ, tissue, or cell.

### VIII. Linkers

In certain embodiments, the WNT surrogate, enhancer, and/or targeting modules are bound or fused directly to each other, whereas in other embodiments, they are separated by a linker, e.g., a polypeptide linker, or a non-peptidyl linker, etc. In particular embodiments, a linker is an Fc linker, e.g., a region of an antibody Fc domain capable of dimerizing with another Fc linker, e.g., via one or more disulfide bonds. In another particular embodiment, a linker is albumin, e.g., human serum albumin, where the targeting and action modules are on the N- and C-termini of albumin.

In certain embodiments, particularly when joining two polypeptides, the linker is made up of amino acids linked together by peptide bonds. In particular embodiments, the linker comprises, in length, from 1 up to about 40 amino acid residues, from 1 up to about 30 amino acid residues, from 1 up to about 20 amino acid residues, from 5 up to about 15 amino acid residues, or from 1 to about 10 amino acid residues, e.g., 3, 4, 5, 6, 7, 8, 9, 10, 11, 12, 13, 14, or 15 amino acids. In certain embodiments, the amino acid residues in the linker are from among the twenty canonical amino acids, and in certain embodiments, selected from cysteine, glycine, alanine, proline, asparagine, glutamine, and/or serine. In certain embodiments, a linker comprises one or more non-natural amino acids. In some embodiments, a peptidyl linker is made up of a majority of amino acids that are sterically unhindered, such as glycine, serine, and alanine linked by a peptide bond. Certain linkers include polyglycines, polyserines, and polyalanines, or combinations of any of these. Some exemplary peptidyl linkers are poly(Gly)1-8, particularly (Gly)3, (Gly)4 (SEQ ID NO: 1), (Gly)5 (SEQ ID NO: 2), (Gly)6 (SEQ ID NO: 3), (Gly)7 (SEQ ID NO: 4), and (Gly)8 (SEQ ID NO: 5) as well as, poly(Gly)4 Ser (SEQ ID NO: 6), poly(Gly-Ala)2 (SEQ ID NO: 7), poly(Gly-Ala)3 (SEQ ID NO: 8), poly(Gly-Ala)4 (SEQ ID NO: 9) and poly(Ala)1-8 (SEQ ID NO: 10-14). Other specific examples of peptidyl linkers include (Gly)5Lys (SEQ ID NO: 15), and (Gly)5LysArg (SEQ ID NO: 16). To explain the above nomenclature, for example, (Gly)3Lys(Gly)4 means Gly-Gly-Gly-Lys-Gly-Gly-Gly-Gly (SEQ ID NO: 17). Other combinations of Gly and Ala are also useful. Additionally, a peptidyl linker can also comprise a non-peptidyl segment such as a 6 carbon aliphatic molecule of the formula —CH2-CH2-CH2-CH2-CH2-CH2-. The peptidyl linkers can be altered to form derivatives as described herein.

Illustrative non-peptidyl linkers include, for example, alkyl linkers such as —NH—(CH2) s-C(O)—, wherein s=2-20. These alkyl linkers may further be substituted by any non-sterically hindering group such as lower alkyl (e.g., C1-C6) lower acyl, halogen (e.g., Cl, Br), CN, NH2, phenyl, etc. Non-peptide portions of the inventive composition of matter, such as non-peptidyl linkers or non-peptide half-life extending moieties can be synthesized by conventional organic chemistry reactions. Chemical groups that find use in linking binding domains include carbamate; amide (amine plus carboxylic acid); ester (alcohol plus carboxylic acid), thioether (haloalkane plus sulfhydryl; maleimide plus sulfhydryl), Schiff s base (amine plus aldehyde), urea (amine plus isocyanate), thiourea (amine plus isothiocyanate), sulfonamide (amine plus sulfonyl chloride), disulfide; hydrazone, lipids, and the like, as known in the art.

The linkage between domains may comprise spacers, e.g. alkyl spacers, which may be linear or branched, usually linear, and may include one or more unsaturated bonds; usually having from one to about 300 carbon atoms; more usually from about one to 25 carbon atoms; and may be from about three to 12 carbon atoms. Spacers of this type may also comprise heteroatoms or functional groups, including amines, ethers, phosphodiesters, and the like. Specific structures of interest include: (CH₂CH₂O)n where n is from 1 to about 12; (CH₂CH₂NH)n, where n is from 1 to about 12; [(CH₂)n(C═O)NH(CH₂)m]_{z}, where n and m are from 1 to about 6, and z is from 1 to about 10; [(CH₂)nOPO₃(CH₂)m]_{z} where n and m are from 1 to about 6, and z is from 1 to about 10. Such linkers may include polyethylene glycol, which may be linear or branched.

In certain embodiments, the domains may be joined through a homo- or heterobifunctional linker. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP); N, N′-(1,3-phenylene) bismaleimide; N, N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. In certain embodiments, the succinimidyl group of these cross-linkers reacts with a primary amine, and the thiol-reactive maleimide forms a covalent bond with the thiol of a cysteine residue.

Other reagents useful include: homobifunctional cross-linking reagents including bismaleimidohexane (“BMH”); p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); disdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like.

### IX. Nucleic Acids and Polypeptides

The present invention further provides in certain embodiments an isolated nucleic acid encoding a polypeptide present in a molecule disclosed herein, e.g., a WNT surrogate, a WNT enhancer, or a WNT super agonist. Nucleic acids include DNA and RNA. These and related embodiments may include polynucleotides encoding antibody fragments that bind one or more co-receptors. The term “isolated polynucleotide” as used herein shall mean a polynucleotide of genomic, cDNA, or synthetic origin, or some combination thereof, which by virtue of its origin, the isolated polynucleotide: (1) is not associated with all or a portion of a polynucleotide in which the isolated polynucleotide is found in nature; (2) is linked to a polynucleotide to which it is not linked in nature, or (3) does not occur in nature as part of a larger sequence. An isolated polynucleotide may include naturally occurring and/or artificial sequences.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

It will be appreciated by those of ordinary skill in the art that, as a result of the degeneracy of the genetic code, there are many nucleotide sequences that encodes an antibody as described herein. Some of these polynucleotides bear minimal sequence identity to the nucleotide sequence of the native or original polynucleotide sequence encoding a polypeptide within a WNT surrogate, a WNT enhancer, or a WNT super agonist. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the invention, a mutagenesis approach, such as site-specific mutagenesis, may be employed for the preparation of variants and/or derivatives of the polypeptides described herein. By this approach, specific modifications in a polypeptide sequence can be made through mutagenesis of the underlying polynucleotides that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the polynucleotide sequences that encode a polypeptide present in a molecule disclosed herein, e.g., a WNT surrogate, a WNT enhancer, or a WNT super agonist, to alter one or more properties of the encoded polypeptide, such as the binding affinity, or the function of a particular Fc region, or the affinity of the Fc region for a particular FcγR. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phages are readily commercially-available and their use is generally well-known to those skilled in the art. Double-stranded plasmids are also routinely employed in site directed mutagenesis that eliminates the step of transferring the gene of interest from a plasmid to a phage.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

In many embodiments, one or more nucleic acids encoding a polypeptide of a molecule disclosed herein, e.g., a WNT surrogate, a WNT enhancer, or a WNT super agonist, are introduced directly into a host cell, and the cell incubated under conditions sufficient to induce expression of the encoded polypeptides.

The surrogate polypeptides of this disclosure may be prepared using standard techniques well known to those of skill in the art in combination with the polypeptide and nucleic acid sequences provided herein. The polypeptide sequences may be used to determine appropriate nucleic acid sequences encoding the particular polypeptide disclosed thereby. The nucleic acid sequence may be optimized to reflect particular codon “preferences” for various expression systems according to standard methods well known to those of skill in the art.

According to certain related embodiments there is provided a recombinant host cell that comprises one or more constructs as described herein, e.g., a vector comprising a nucleic acid encoding a surrogate molecule or polypeptide thereof, and a method of production of the encoded product, which method comprises expression from encoding nucleic acid therefor. Expression may conveniently be achieved by culturing under appropriate conditions recombinant host cells containing the nucleic acid. Following production by expression, an antibody or antigen-binding fragment thereof, may be isolated and/or purified using any suitable technique, and then used as desired.

Polypeptides, and encoding nucleic acid molecules and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli. Polypeptides present within a molecule disclosed herein, e.g., a WNT surrogate, a WNT enhancer, or a WNT super agonist, may be recombinantly produced in prokaryotic or eukaryotic cells.

The expression of polypeptides, e.g., antibodies and antigen-binding fragments thereof, in prokaryotic cells such as E. coli is well established in the art. For a review, see for example Pluckthun, A. Bio/Technology 9: 545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The present invention also provides, in certain embodiments, a method which comprises using a construct as stated above in an expression system in order to express a particular polypeptide present within a molecule disclosed herein, e.g., a WNT surrogate, a WNT enhancer, or a WNT super agonist. The term “transduction” is used to refer to the transfer of genes from one bacterium to another, usually by a phage.

Amino acid sequence modification(s) of any of the polypeptides described herein are contemplated. For example, it may be desirable to improve the binding affinity and/or other biological properties of the surrogate molecule. For example, amino acid sequence variants of a molecule disclosed herein, e.g., a WNT surrogate, a WNT enhancer, or a WNT super agonist, may be prepared by introducing appropriate nucleotide changes into a polynucleotide that encodes the antibody, or a chain thereof, or by peptide synthesis. Such modifications include, for example, deletions from, and/or insertions into and/or substitutions of, residues within the amino acid sequences of the antibody. Any combination of deletion, insertion, and substitution may be made to arrive at the final surrogate molecule, provided that the final construct possesses the desired characteristics (e.g., high affinity binding to one or more co-receptors). The amino acid changes also may alter post-translational processes of the antibody, such as changing the number or position of glycosylation sites. Any of the variations and modifications described above for polypeptides of the present invention may be included in antibodies of the present invention.

The present disclosure provides variants of any of the polypeptides (e.g., polypeptides of surrogate molecules, super agonists, or antibodies or antigen-binding fragments thereof) disclosed herein. In certain embodiments, a variant has at least 90%, at least 95%, at least 98%, or at least 99% identity to a polypeptide disclosed herein. In certain embodiments, such variant polypeptides bind to one or more first co-receptors, and/or to one or more second co-receptors, and/or to an E3 ligase at least about 50%, at least about 70%, and in certain embodiments, at least about 90% as well as a molecule specifically set forth herein. In further embodiments, such variant molecules bind to one or more first co-receptor, and/or to one or more second co-receptor, with greater affinity than the molecules set forth herein, for example, that bind quantitatively at least about 105%, 106%, 107%, 108%, 109%, or 110% as well as an antibody sequence specifically set forth herein.

In particular embodiments, a molecule disclosed herein, e.g., a WNT surrogate, a WNT enhancer, or a WNT super agonist, or a binding region thereof, e.g., a Fab, scFv, or VHH may comprise: a) a heavy chain variable region comprising: i. a CDR1 region that is identical in amino acid sequence to the heavy chain CDR1 region of a selected antibody described herein; ii. a CDR2 region that is identical in amino acid sequence to the heavy chain CDR2 region of the selected antibody; and iii. a CDR3 region that is identical in amino acid sequence to the heavy chain CDR3 region of the selected antibody; and/or b) a light chain variable domain comprising: i. a CDR1 region that is identical in amino acid sequence to the light chain CDR1 region of the selected antibody; ii. a CDR2 region that is identical in amino acid sequence to the light chain CDR2 region of the selected antibody; and iii. a CDR3 region that is identical in amino acid sequence to the light chain CDR3 region of the selected antibody; wherein the antibody specifically binds a selected target. In a further embodiment, the antibody, or antigen-binding fragment thereof, is a variant antibody or antigen-binding fragment thereof wherein the variant comprises a heavy and light chain identical to the selected antibody except for up to 8, 9, 10, 11, 12, 13, 14, 15, or more amino acid substitutions in the CDR regions of the VH and VL regions. In this regard, there may be 1, 2, 3, 4, 5, 6, 7, 8, or in certain embodiments, 9, 10, 11, 12, 13, 14, 15 more amino acid substitutions in the CDR regions of the selected antibody. Substitutions may be in CDRs either in the VH and/or the VL regions. (See e.g., Muller, 1998, Structure 6:1153-1167).

In particular embodiments, a molecule disclosed herein, e.g., a WNT surrogate, a WNT enhancer, or a WNT super agonist, or a binding region thereof, e.g., a Fab, scFv, or VHH/sdAb, may have: a) a heavy chain variable region having an amino acid sequence that is at least 80% identical, at least 95% identical, at least 90%, at least 95% or at least 98% or 99% identical, to the heavy chain variable region of an antibody or antigen-binding fragments thereof described herein; and/or b) a light chain variable region having an amino acid sequence that is at least 80% identical, at least 85%, at least 90%, at least 95% or at least 98% or 99% identical, to the light chain variable region of an antibody or antigen-binding fragments thereof described herein.

A polypeptide has a certain percent “sequence identity” to another polypeptide, meaning that, when aligned, that percentage of amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

In particular embodiments, a molecule disclosed herein, e.g., a WNT surrogate, a WNT enhancer, or a WNT super agonist, or a binding region thereof, e.g., a Fab, scFv, or VHH may comprise: a) a heavy chain variable region comprising: i. a CDR1 region that is identical in amino acid sequence to the heavy chain CDR1 region of a selected antibody described herein; ii. a CDR2 region that is identical in amino acid sequence to the heavy chain CDR2 region of the selected antibody; and iii. a CDR3 region that is identical in amino acid sequence to the heavy chain CDR3 region of the selected antibody; and b) a light chain variable domain comprising: i. a CDR1 region that is identical in amino acid sequence to the light chain CDR1 region of the selected antibody; ii. a CDR2 region that is identical in amino acid sequence to the light chain CDR2 region of the selected antibody; and iii. a CDR3 region that is identical in amino acid sequence to the light chain CDR3 region of the selected antibody; wherein the antibody specifically binds a selected target (e.g., a FZD receptor, such as FZD1). In a further embodiment, the antibody, or antigen-binding fragment thereof, is a variant antibody wherein the variant comprises a heavy and light chain identical to the selected antibody except for up to 8, 9, 10, 11, 12, 13, 14, 15, or more amino acid substitutions in the CDR regions of the VH and VL regions. In this regard, there may be 1, 2, 3, 4, 5, 6, 7, 8, or in certain embodiments, 9, 10, 11, 12, 13, 14, 15 more amino acid substitutions in the CDR regions of the selected antibody. Substitutions may be in CDRs either in the VH and/or the VL regions. (See e.g., Muller, 1998, Structure 6:1153-1167).

Determination of the three-dimensional structures of representative polypeptides (e.g., variant FZD binding regions or LRP5/6 binding regions of WNT surrogate molecules as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of binding regions include VMD which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/. Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261: C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488). A variety of appropriate computational computer programs are also commercially available, such as from Schrödinger (Munich, Germany).

The accompanying Examples set forth a variety of polypeptide sequences that may be present within WNT surrogates, WNT super agonists, and WNT enhancers. In particular, Table 3 provides sequences of polypeptides present in illustrative WNT surrogates and WNT enhancers, and Table 4 provides sequences of polypeptides present in illustrative WNT super agonists and WNT enhancers. These tables also provide the structure of each of the molecules disclosed, which is listed or may be readily discerned from the name of the molecule. Illustrative binding domains present within the various molecules are provided in Table 2 with the full sequences shown in Tables 3 and 4. The various binding domains and molecules described in the Examples may be modified or combined in other orientations or configurations, including but not limited to any of the various configurations shown in the Examples or Figures. For example, the positions of the FZD binding domain and the LRP5/6 binding domain may be switched in any of the polypeptides present within the structures depicted. The disclosure further includes polypeptide variants of any of the polypeptides or binding domains thereof disclosed herein, such polypeptide variants having at least 70%, at least 75%, at least 80%, at least 85%, at least 90%, at least 95%, at least 98%, or at least 99% sequence identity to a polypeptide or binding domain thereof disclosed herein.

### X. Compositions

Pharmaceutical compositions comprising a surrogate molecule described herein and one or more pharmaceutically acceptable diluent, carrier, or excipient are also disclosed.

In further embodiments, pharmaceutical compositions comprising a polynucleotide comprising a nucleic acid sequence encoding a surrogate molecule described herein and one or more pharmaceutically acceptable diluent, carrier, or excipient are also disclosed. In particular embodiments, the pharmaceutical composition further comprises one or more polynucleotides comprising a nucleic acid sequence encoding a naturally occurring co-receptor ligand polypeptide. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail. In other embodiments, the polynucleotides are expression cassettes comprising a promoter operatively linked to the coding sequences. In certain embodiments, the nucleic acid sequence encoding the surrogate molecule and the nucleic acid sequence encoding naturally occurring co-receptor ligand polypeptide are present in the same polynucleotide.

In further embodiments, pharmaceutical compositions comprising an expression vector, e.g., a viral vector, comprising a polynucleotide comprising a nucleic acid sequence encoding a surrogate molecule described herein and one or more pharmaceutically acceptable diluent, carrier, or excipient are also disclosed. In particular embodiments, the pharmaceutical composition further comprises an expression vector, e.g., a viral vector, comprising a polynucleotide comprising a nucleic acid sequence encoding a naturally occurring co-receptor ligand polypeptide. In certain embodiments, the nucleic acid sequence encoding the surrogate molecule and the nucleic acid sequence encoding the naturally occurring co-receptor ligand polypeptide are present in the same polynucleotide, e.g., expression cassette.

The present invention further contemplates a pharmaceutical composition comprising a cell comprising an expression vector comprising a polynucleotide comprising a promoter operatively linked to a nucleic acid encoding a surrogate molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a polypeptide corresponding to the natural ligand of the receptors. In particular embodiments, the cell is a heterologous cell or an autologous cell obtained from the subject to be treated. In particular embodiments, the cell is a stem cell, e.g., an adipose-derived stem cell or a hematopoietic stem cell.

The subject molecules, alone or in combination, can be combined with pharmaceutically-acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the surrogate molecule (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g. syringe, e.g. a prefilled syringe, together with instructions for administration.

The pharmaceutical compositions of the invention encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present invention includes pharmaceutically acceptable salts of a WNT surrogate molecule described herein. The term “pharmaceutically acceptable salt” refers to physiologically and pharmaceutically acceptable salts of the compounds of the invention: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see “Handbook of Pharmaceutical Salts: Properties, Selection, and Use” by Stahl and Wermuth (Wiley-VCH, 2002).

Pharmaceutically acceptable base addition salts are formed with metals or amines, such as alkali and alkaline earth metals or organic amines. Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

In some embodiments, the pharmaceutical composition provided herein comprise a therapeutically effective amount of a WNT surrogate molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### XI. Methods of Use

For illustrative purposes only, the WNT super agonist molecules, WNT surrogate molecules, and WNT enhancer molecules (RSPO mimetics), including those disclosed herein, can be used as to treat various diseases or disorders where tissue regeneration is necessary or beneficial. Subjects that may be treated include, but are not limited to, mammals, e.g., humans. Such diseases include, but are not limited to: increase bone growth or regeneration, bone grafting, healing of bone fractures, treatment of osteoporosis and osteoporotic fractures, vertebral compression fractures, spinal fusion, osseointegration of orthopedic devices, tendon-bone integration, tooth growth and regeneration, dental implantation, periodontal diseases, maxillofacial reconstruction, and osteonecrosis of the jaw. Also contemplated are: treatment of alopecia; enhancing regeneration of sensory organs, e.g. treatment of hearing loss, including internal and external auditory hair cells, treatment of vestibular hypofunction, treatment of macular degeneration, treatment of various retinopathies, including but not limited to vitreoretinopathy, diabetic retinopathy, other diseases of retinal degeneration, wet age-related macular degeneration (AMD), dry AMD, Fuchs' dystrophy, other cornea disease, etc.; treatment of stroke, traumatic brain injury, Alzheimer's disease, multiple sclerosis and other conditions affecting the blood brain barrier; treatment of spinal cord injuries, other spinal cord diseases. The compositions of this invention may also be used in treatment of oral mucositis, treatment of short bowel syndrome, inflammatory bowel diseases (IBD), other gastrointestinal disorders; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; treatment of myocardial infarction, coronary artery disease, heart failure; enhanced growth of hematopoietic cells, e.g. enhancement of hematopoietic stem cell transplants from bone marrow, mobilized peripheral blood, treatment of immunodeficiencies, graft versus host diseases, etc.; treatment of acute kidney injuries, chronic kidney diseases; treatment of lung diseases, chronic obstructive pulmonary diseases (COPD), idiopathic pulmonary fibrosis (IPF) enhanced regeneration of lung tissues. The compositions of the present invention may also be used in enhanced regeneration of liver cells, e.g. liver regeneration, treatment of cirrhosis, enhancement of liver transplantations, treatment of acute liver failure, treatment of chronic liver diseases with hepatitis C or B virus infection or post-antiviral drug therapies, alcoholic liver diseases, alcoholic hepatitis, non-alcoholic liver diseases with steatosis or steatohepatitis, and the like. The compositions of this invention may treat diseases and disorders including, without limitation, conditions in which regenerative cell growth is desired.

In particular embodiments, the WNT super agonist molecules, WNT surrogate molecules, and WNT enhancer molecules (RSPO mimetics), including those disclosed herein, may be used to induce bone formation or increase bone density in a subject. For example, the subject may be administered an effective amount of a WNT super agonist molecule, WNT surrogate molecule, or WNT enhancer molecule. In particular embodiments, the subject is administered a WNT super agonist molecule or a WNT surrogate molecule comprising a FZD binding domain that binds to FZD5, FZD8, and FZD9.

In certain embodiments, the WNT super agonist molecules, WNT surrogate molecules, and WNT enhancer molecules (RSPO mimetics), including those disclosed herein, may be used for regenerating a salivary gland, inducing salivary gland growth or salivary gland tissue growth in a subject. The method may be used to treat hyposalivation or dry mouth in a subject. For example, the subject may be administered an effective amount of a WNT super agonist molecule, WNT surrogate molecule, or WNT enhancer molecule. In particular embodiments, the subject is administered a WNT super agonist molecule or a WNT surrogate molecule comprising a FZD binding domain that binds to FZD1, FZD2, and FZD7.

In certain embodiments, the WNT super agonist molecules, WNT surrogate molecules, and WNT enhancer molecules (RSPO mimetics), including those disclosed herein, may be used to preserve cells, tissues, organs or organoids, e.g., tissue or organs for transplantation. For example, a cell, tissue, organ, or organoid may be contacted with a WNT super agonist molecule, WNT surrogate molecule, or WNT enhancer molecule in vivo or ex vivo. In the context of preserving cells, tissue, or organs for transplantation, the cell, tissue, organ, or organoid may be contacted with a WNT super agonist molecule, WNT surrogate molecule, or WNT enhancer molecule while still in the donor (i.e., before removal from the donor) and/or after removal from the donor. The methods may maintain or enhance viability of the cell, tissue, or organ, for example, during storage or prior to transplantation into a recipient. In particular embodiments, the cells, tissue, or organ is perfused in a composition or solution comprising the WNT super agonist molecule, WNT surrogate molecule, or WNT enhancer molecule. In certain embodiments, certain organ tissue is contacted with a WNT super agonist molecule to maintain viability of that tissue. In particular embodiments, the organ tissue is donor organ tissue to be transplanted to a recipient in need thereof. In certain embodiments, donor organ tissue is perfused in vivo with a solution comprising a WNT super agonist molecule disclosed here, e.g., before the organ tissue is removed from the donor. In certain embodiments, donor organ tissue is perfused ex vivo with a solution comprising a WNT super agonist molecule disclosed here, e.g., during storage or during transport from a donor to a recipient. In particular embodiment, the organ tissue contacted with a Wnt signal enhancing molecule remains viable for transplantation for at least 10%, at least 20%, at least 50%, or at least 100% longer than if it was not contacted with the Wnt signal enhancing molecule. In certain embodiments the organ tissue is liver tissue.

In certain embodiments, the WNT super agonist molecules, WNT surrogate molecules, and WNT enhancer molecules (RSPO mimetics), including those disclosed herein, may be used for the expansion and/or maintenance of ex vivo tissue, e.g., skin tissue. In particular embodiments, the tissue is isolated from a donor or a patient. The tissue may be contacted with (e.g., maintained or cultured in the presence of) a WNT super agonist molecule, WNT surrogate molecule, or WNT enhancer molecule in vivo or ex vivo. In certain embodiments, the tissue is contacted ex vivo, e.g., by perfusion with a composition comprising a WNT super agonist molecule, WNT surrogate molecule, or WNT enhancer molecule.

In another embodiments, the WNT super agonist molecules, WNT surrogate molecules, and WNT enhancer molecules (RSPO mimetics), including those disclosed herein, may be used to generate or maintain an organoid or organoid culture. For example, an organoid culture may be contacted with a WNT super agonist molecule, WNT surrogate molecule, or WNT enhancer molecule, for example, by culturing the organoid in a medium comprising a WNT super agonist molecule, WNT surrogate molecule, or WNT enhancer molecule. In certain embodiments, an organoid culture is generated, grown, or maintained by contacting it with one or more WNT super agonist molecules disclosed herein. In particular embodiments, the WNT super agonist molecule is present in the culture media used to grow or maintain the organoid tissue.

In particular embodiments, a pharmaceutical composition is administered parenterally, e.g., intravenously, orally, rectally, or by injection. In some embodiments, it is administered locally, e.g., topically or intramuscularly. In some embodiments, a composition is administered to target tissues, e.g., to bone, joints, ear tissue, eye tissue, gastrointestinal tract, skin, a wound site or spinal cord.

Methods of the invention may be practiced in vivo or ex vivo. In some embodiments, the contacting of a target cell or tissue with a surrogate molecule is performed ex vivo, with subsequent implantation of the cells or tissues, e.g., activated stem or progenitor cells, into the subject. The skilled artisan can determine an appropriate site of and route of administration based on the disease or disorder being treated.

The dose and dosage regimen may depend upon a variety of factors readily determined by a physician, such as the nature of the disease or disorder, the characteristics of the subject, and the subject's history. In particular embodiments, the amount of a surrogate molecule administered or provided to the subject is in the range of about 0.01 mg/kg to about 50 mg/kg, 0.1 mg/kg to about 500 mg/kg, or about 0.1 mg/kg to about 50 mg/kg of the subject's body weight.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular biology, cell biology and biochemistry can be found in such standard textbooks as “Molecular Cloning: A Laboratory Manual, 3rd Ed.” (Sambrook et al., Harbor Laboratory Press 2001); “Short Protocols in Molecular Biology, 4th Ed.” (Ausubel et al. eds., John Wiley & Sons 1999); “Protein Methods” (Bollag et al., John Wiley & Sons 1996); “Nonviral Vectors for Gene Therapy” (Wagner et al. eds., Academic Press 1999); “Viral Vectors” (Kaplift & Loewy eds., Academic Press 1995); “Immunology Methods Manual” (I. Lefkovits ed., Academic Press 1997); and “Cell and Tissue Culture: Laboratory Procedures in Biotechnology” (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

Recombinant molecules were generated that combine agonists for the WNT receptors, FZD and/or LRP co-receptors, together with agonists for the E3 ligase receptors, ZNRF3 or RNF43, to create WNT signaling ‘super agonists’

Materials and methods employed in the following Examples include the following.

### Protein Production

All recombinant proteins were produced in Expi293F cells (Thermo Fisher Scientific) by transient transfection. The FvFab proteins were first purified using cOmplete® His-tag purification resin (Sigma-Aldrich). The heterodimeric Fc-based proteins were first purified using MiniChrom MabSelect SuRe (Repligen), then polished by cOmplete® His-tag purification resin and anti-Flag M2 affinity gel (Sigma-Aldrich). Other proteins were first purified using MiniChrom MabSelect SuRe unless otherwise specified. All proteins were further polished with Superdex 200 Increase 10/300 GL (GE Healthcare Life Sciences) size-exclusion chromatography (SEC) using IxHBS buffer (20 mM HEPES pH 7.4, 150 mM NaCl). After that, the proteins were examined by SDS-polyacrylamide electrophoresis and estimated to be >90% purity.

### SuperTop Flash (STF) Assay

WNT signaling activity was measured using HEK293 cells containing a luciferase gene controlled by a WNT-responsive promoter (Super Top Flash reporter assay, STF) as previously reported ({Chen, 2020 #65}). In brief, cells were seeded at a density of 10,000 per well in 96-well plates 24 hours prior to treatment at the presence of 3 μM IWP2 to inhibit the production of endogenous WNTs. The recombinant proteins were then added to the cells with or without 20 nM Fc-RSPO2 overnight. Recombinant human WNT3A (R&D systems) was used as a positive control. Cells were lysed with Luciferase Cell Culture Lysis Reagent (Promega) and luciferase activity was measured with Luciferase Assay System (Promega) using vendor suggested procedures.

### Cell Flow Cytometry

HEK293 cells transiently transfected with a plasmid overexpressing ZNRF3 (Gen-Script OHu22977) were treated for 24 h with RSPO derivative molecules at 10 nM final concentration in DMEM supplemented with 10% FBS. Cells were dissociated using Gibco enzyme-free dissociation buffer, washed, and resuspended in FACS buffer (1× PBS with 1% BSA with 0.02% sodium azide). Cells were incubated with 1 nM F12578 IgG for 1 h. After washing, the cells were incubated with goat anti-human IgG Alexa Fluor 647 (Invitrogen, Carlsbad, Calif.) for 40 min. Cells were washed with FACS buffer and subjected to multi-channel analysis using a SONY SH800S flow cytometer (BD Biosciences. Data were processed with FlowJo software (FlowJo, Ashland, Oreg.) and fluorescence signals were displayed in histogram plots.

### Primary Cells and Organoid Expansion

Mouse small intestinal organoids (#70931 STEMCELL Technologies) were maintained and expanded as described in Sato et al., 2009. In short, adapted expansion medium contained Advanced DMEM, 10 mM HEPES, 1× GlutaMAX, 1× Penicillin-Streptomycin, 1× B27, 1.25 mM N-acetylcysteine, 50 ng/mL recombinant human EGF, 50 ng/mL recombinant human Noggin and 500 ng/mL recombinant human R-Spondin 1 (see Table 1).

Human small intestinal organoids were a gift from the Calvin Kuo Lab at Stanford. Organoids were maintained and expanded as described in Sato et al., 2011 (Sato et al., 2011). In short, adapted expansion medium contained Advanced DMEM, 10 mM HEPES, 1× GlutaMAX, 1× Penicillin-Streptomycin, 1× B27, 1× N2, 1.25 mM N-acetylcysteine, 10 mM Nicotinamide, 50 ng/mL recombinant human EGF, 50 ng/mL recombinant human Noggin, 500 ng/mL recombinant human R-Spondin 1, 0.5 nM L6-F12578 surrogate Wnt, 10 nM recombinant Gastrin, 500 nM A83-01 and 10 M SB202190 (see Table 1).

Mouse hepatocyte organoids were grown from primary CD1 murine hepatocytes (#MSCP20 Thermo Fisher) and expanded as described in Hu et al., 2018. In short, adapted expansion medium contained Advanced DMEM, 10 mM HEPES, 1× GlutaMAX, 1× Penicillin-Streptomycin, 1× B27, 1.25 mM N-acetylcysteine, 50 ng/mL recombinant human EGF, 50 ng/mL recombinant human Noggin, 500 ng/mL recombinant human R-Spondin 1, 10 nM recombinant Gastrin, 3 μM CHIR99021, 25 ng/mL recombinant HGF, 50 ng/mL FGF7, 50 ng/mL FGF10, 10 mM Nicotinamide and 500 nM A83-01 (see Table 1).

Human kidney organoids were established from primary human renal proximal tubule epithelial cells (PCS-400-010) and maintained and expanded as described in Schutgens et al., 2019. In short, adapted expansion medium contained Advanced DMEM, 10 mM HEPES, 1× GlutaMAX, 1× Penicillin-Streptomycin, 1× B27, 50 ng/mL recombinant human EGF, 100 ng/mL recombinant human FGF10, 500 nM A83-01 and 500 ng/mL recombinant human R-Spondin 1 (see Table 1).

### Outgrowth Efficiency Assay

For the outgrowth efficiency assay all organoid lines (mouse small intestine, human small intestine and human kidney) were digested to small, near single cell suspension, fragments using 1× TrypLE (12605010 GIBCO) for 10 minutes at 37° C. Mouse hepatocyte outgrowth efficiency was performed with primary single cells. For all cell types the base medium consisted of expansion medium without RSPO1, Surrogate Wnt and/or CHIR99021 and supplemented with 1 μM porcupine inhibitor Wnt-C59 (#5148 Tocris) and 10 μM Y-27632 (#5092280001 MilliporeSigma). Experimental conditions, as in FIG. 3, consisted of one or combination of 500 ng/mL RSPO1, 100 ng/mL recombinant human Wnt-3a, 1 nM Surrogate WNT L6-F12578 (0.1 nM for mouse small intestinal organoids) or 1 nM WNT super agonist L6-F12578-RSPO2RA (0.1 nM for mouse small intestinal organoids. All cells for all conditions were plated in 15 L Matrigel droplets in 96-well plates and submerged in 120 L of the experimental medium. Mouse small intestine, human small intestine and human kidney organoids were expanded for 7 days before measurement and mouse hepatocyte organoids for 14 days before measurements. The medium was changed approximately every 3 days. Each experiment consisted of three technical replicates per plate and was repeated three times. Outgrowth efficiency was quantified using cell viability assay CellTiter-Glo (G9683 Promega) measured on the SpectraMax Paradigm microplate reader (Molecular Devices) according to manufactures protocols.

TABLE 1
Organoid Reagents
Organoid reagents

| Component | Catalog number |
|---|---|
| Advanced DMEM | Thermo Scientific 12634-010 |
| HEPES | Thermo Scientific 15630080 |
| GlutaMAX | Thermo Scientific 35050061 |
| Penicillin-Streptomycin | Thermo Scientific 15140122 |
| B27 | Thermo Scientific 17504044 |
| N2 | Thermo Scientific 17502048 |
| Nicotinamide | Sigma-Aldrich N0636 |
| N-acetylcysteine | Sigma-Aldrich A9165 |
| A83-01 | Tocris 2939 |
| CHIR99021 | Tocris 4423 |
| SB202190 | Tocris 126410 |
| Recombinant Human EGF | Peprotech AF-100-15 |
| Recombinant Human FGF7 | Peprotech 100-19 |
| Recombinant Human FGF10 | Peprotech 100-26 |
| Recombinant Human Noggin | Peprotech 120-10C |
| Recombinant Human HGF | Peprotech 100-39H |
| Human Gastrin I | Tocris 30061 |
| Recombinant Human R-Spondin 1 | R&D 4645RS |
| Recombinant Human Wnt-3a | R&D 5036WN |
| Surrogate WNT L6-F12578 | In-house production |
| WNT super agonist L6-F12578-RSPO2RA | In-house production |
| Matrigel ™ GFR Membrane Matrix | Corning CB40230C |

### Murine Studies and Dual Energy X-Ray Absorptiometry (DEXA):

All animal experiments were performed according to national ethical guidelines in addition to the guidance and approval by the Institutional Animal Care and Use Committee (IACUC) of Surrozen, Inc. Twelve-week-old C57Bl1/6J female mice were obtained from Jackson Laboratories (Bar Harbor, Me., USA) and were housed 4 per cage. Proteins treatments at 3 mg per kg were intraperitoneal dosed on day 0, 3, 7 and 10. Bone mineral density (BMD) and fat content of animals were measured via in vivo DEXA method using a Faxitron UltraFocus (Faxitron Bioptics, Tucson, Ariz.) on day 0, 7 and 13. Animals were anesthetized during imaging through isoflurane and sample ROI included the entire murine skeleton except material above the cervical spine due to increased radiographical intensity of the skull. BMD and fat content were calculated using the accompanying Vision DXA software. Animals were terminated on day 14, and liver, small intestine, and salivary glands were collected for histology.

### Example 1

### WNT Surrogate Formats

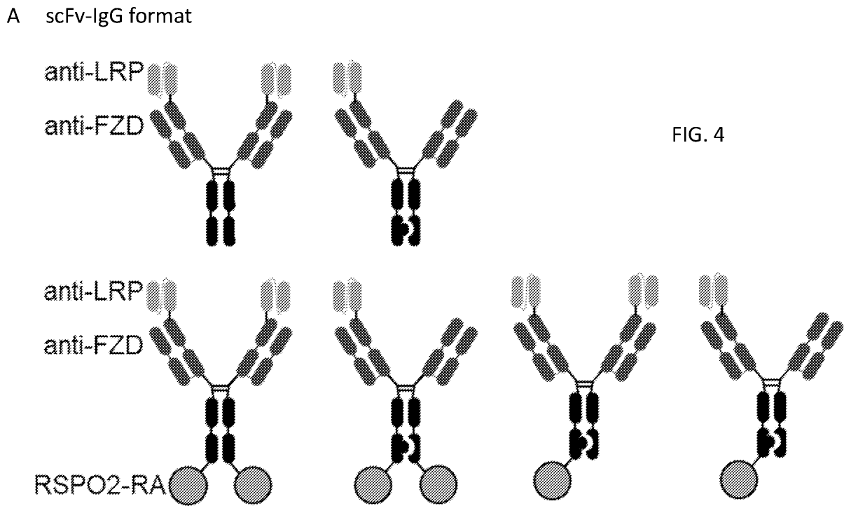

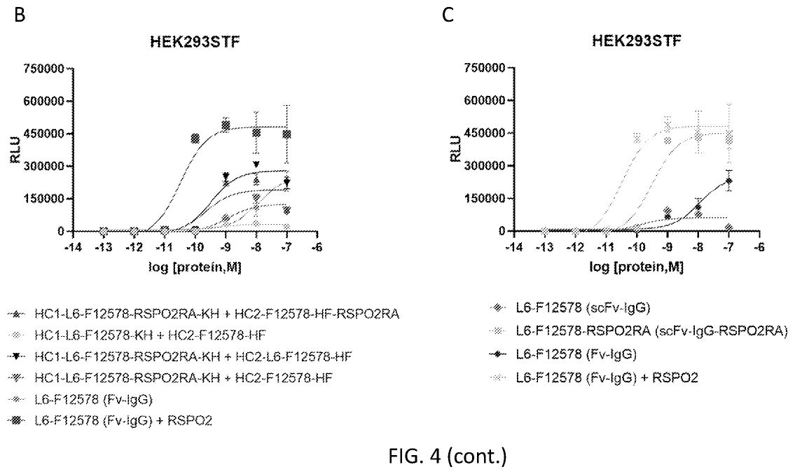

A new modular and flexible platform for potent, selective WNT surrogate generation was created (see, e.g., WO 2020/010308). A key feature of the platform was the requirement for multimerization of FZDs and LRPs, with optimal stoichiometry of two FZD and one or two LRP binders, for maximal WNT/β-catenin activation. This platform was built based on tandem scFv antibody fragment format (See Table 3). To understand whether additional multivalent antibody formats can produce active surrogate WNTs, Fv-IgG, Fab-IgG, scFv-IgG formats as shown in FIG. 1A, FIG. 4A, and Table 3, were tested. These formats also offered different distances and geometries between the different binding arms on the antibody molecule, allowing the assessment of the contribution of format and geometry to activity.

Certain FZD and LRP binders were selected for each construct. Table 2 provides the nomenclature of components used.

TABLE 2
WNT surrogate format components

| Component designation | Clone name | Specificity |
|---|---|---|
| F12578 | 18R5 | FZD 1, 2, 7, 5, 8 |
| F127 | R2H1 | FZD 1, 2, 7 |
| F58 | 2919 | FZD 5, 8 |
| F4 | 5044 | FZD 4 |
| F4-2 | 5063 | FZD 4 |
| F49 | 3SC10 | FZD 4, 9 |
| F10 | hB9L9.3 | FZD 10 |
| F6 | R2M3 | FZD 1, 2, 7, 5, 8 |
| F7 | 1791 | FZD 7 |
| F7B | F7.B | FZD1, 2, 7, 5, 8, 4 |
| F2I | F2.I | FZD1, 2, 7, 5, 8, 4 |
| L6 | YW211.31.57 | LRP6e3e4 |
| L2 | 26 (009S-E04) | LRP6e3e4 |

An LRP6E3E4 binder, YW211.31.57 (see, e.g., U.S. Pat. No. 8,846,041; designated “L1”), and a FZD 1,2,7,5,8 binder, 18R5 (Gurney, et al. (2012) Proc. Natl. Acad. Sci. 109:1171-11722; designated “F1”) were chosen to combine in the formats shown in FIG. 1A, FIG. 4A, and Table 3 to generate the following constructs: L6F12578 (scFv-Fc), L6-F12578 (Fv-IgG), and L6-F12578 (Fab-IgG). As a negative control, anti-GFP binders were used. These proteins were purified via a Protein A-affinity column followed by size-exclusion chromatography (SEC) and tested in WNT responsive HEK293 Super TOP-FLASH (STF) reporter cells.

As shown in FIG. 1B, while all three formats yielded active surrogate WNTs, L6-F12578 (Fv-IgG) gave the highest Emax with EC50 of 0.81 nM, while L6-F12578 (Fab-IgG) gave the lowest Emax with EC50 of 0.39 nM. L6-F12578 (scFv-Fc) was the least potent with Emax similar to Fv-IgG. These surrogate WNTs responded to RSPO treatment, while preserving the relative range in potency and Emax; addition of RSPO increased both Emax and potency of all three surrogates (FIG. 1C).

Since the Fv-IgG format produced the most active molecule, is easier to manufacture, and has more desirable biophysical properties, for example, being a much more stable format compared to tandem scFvs, which are less stable and have propensity for aggregation, we focused on Fv-IgG for additional WNT mimetic generation. To test the general applicability of this format, we chose additional FZD binders of different specificity for mimetic assembly with LRP binder, L6. These additional FZD binders, R2H1 (US 2016/0194394, FZD1,2,7 binder referred herein as F127), 2919 (WO 2017/127933, FZD5,8 binder referred herein as F58), 5044 (US 2016/0194394, FZD₄ binder referred herein as F4), 5063 (US 2016/0194394, FZD4 binder referred herein as F4-2), 3SC10 (WO 2019/126399, FZD_{4,9} binder referred herein as F49), hB9L9.3 (US 2016/0194394, FZD₁₀ binder referred herein as F10), F7.B (Pavlovic, et al. (2018) mAbs 10(8): 1157-1167, FZD_{1,2,4,5,7,8} binder referred herein as F7B), and F2.I (Pavlovic, et al. (2018) mAbs 10(8): 1157-1167, FZD_{1,2,4,5,7,8} binder referred herein as F2I) covers the 8 FZDs that signals through β-catenin. The new WNT mimetics, L6-F127 (Fv-IgG), L6-F58 (Fv-IgG), L6-F7B (Fv-IgG), and L6-F2I (Fv-IgG) which binds FZD_{1,2,7}, FZD_{5,8}, FZD_{1,2,4,5,7,8}, and FZD_{1,2,4,5,7,8}, respectively, are highly active on the WNT responsive HEK293 STF cells (FIG. 1D, 1L). Since HEK293 cells does not express or expresses low levels of FZD₄, FZD₉, and FZD₁₀ (data not shown), the parental cells do not show significant response to L6-F4 (Fv-IgG), L6-F49 (Fv-IgG), and L6-F10 (Fv-IgG) (FIG. 1G, 1I, 1K). However, L6-F4 (Fv-IgG), L6-F49 (Fv-IgG), L6-F10 (Fv-IgG), and L6-F4-2 (Fv-IgG) induced potent signaling in HEK293 STF cells over-expressing FZD₄, FZD₉, FZD₁₀, and FZD₄, respectively, consistent with their binding specificity toward these three receptors (FIG. 1F, 1H, 1J, 1M). These WNT mimetics are a valuable set of molecules that allows studies of β-catenin dependent FZDs.

We have shown previously that in tandem scFv format, multivalent binding to FZDs and LRPs are important for signaling, and bispecific tandem scFv molecules with one FZD and one LRP binding arms are either weak or inactive in inducing Wnt signaling (Chen et al., 2020). To assess the valency requirement in the Fv-IgG format, we generated this set of FZD/LRP binders in the bispecific Fv-Fab format where there is one each of FZD and LRP binding arms (FIG. 1A). As shown in FIG. 1E-1K, none of these bispecific with monovalent binding to FZD and LRP induced significant activation of Wnt signaling, further confirming that the previously observed valency requirement in the tandem scFv, VHH-IgG formats also applies to other antibody formats. As the Fv-IgG molecules are studied further in the subsequent sections, the Fv-IgG designation will be removed from molecule names for brevity.

TABLE 3
Formats and sequences of WNT surrogate molecules; Italic underline = linker; Bold =
VH or VL. Formats are diagrammed in FIG. 7

| | | Polypeptide Sequence(s) Present in Structure |
|---|---|---|
| | | (as depicted in the accompanying figures, WNT surrogate molecules |
| Structure | Structure | typically comprises two of each polypeptide sequence shown for any |
| Type | Name | particular structure) |
| Tandem | L6-F12578 | SEQ ID NO: 18 |
| scFv | (scFv-FC) | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGSGSGGSGSGGSSGGEVQLVESGGGLVQPGGSLRLSCAASGF |
| | | TFSHYTLSWVRQAPGKGLEWVSVISGDGSYTYYADSVKGRFTISSDNS |
| | | KNTLYLQMNSLRAEDTAVYYCARNFIKYVFANWGQGTLVTVSSGGG |
| | | GSGGGGSGGGGSDIELTQPPSVSVAPGQTARISCSGDNIGSFYVHWYQQ |
| | | KPGQAPVLVIYDKSNRPSGIPERFSGSNSGNTATLTISGTQAEDEADYY |
| | | CQSYANTLSLVFGGGTKLTVLGSGSGDKTHTCPPCPAPEAAGGPSVFLFP |
| | | PKPKDTLMISRTPEVTCVVVDVSHEDPEVKFNWYVDGVEVHNAKTKPRE |
| | | EQYNSTYRVVSVLTVLHQDWLNGKEYKCKVSNKALGAPIEKTISKAKGQ |
| | | PREPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGK |
| Fv-IgG | L6-F12578 | Heavy Chain SEQ ID NO: 19 |
| | (Fv-IgG) | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSGGGGSGGGGSGGGGSE |
| | | VQLVESGGGLVQPGGSLRLSCAASGFTFSHYTLSWVRQAPGKGLEW |
| | | VSVISGDGSYTYYADSVKGRFTISSDNSKNTLYLQMNSLRAEDTAVYY |
| | | CARNFIKYVFANWGQGTLVTVSSASTKGPSVFPLAPSSKSTSGGTAALG |
| | | CLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQKSLS |
| | | LSPGK |
| | | Light Chain SEQ ID NO: 20 |
| | | DIQMTQSPSSLSASVGDRVTITCRASQDVSTAVAWYQQKPGKAPKLLI |
| | | YSASFLYSGVPSRFSGSGSGTDFTLTISSLQPEDFATYYCQQSYTTPPTF |
| | | GQGTKVEIKGGGGSGGGGSGGGGSDIELTQPPSVSVAPGQTARISCSGD |
| | | NIGSFYVHWYQQKPGQAPVLVIYDKSNRPSGIPERFSGSNSGNTATLTI |
| | | SGTQAEDEADYYCQSYANTLSLVFGGGTKLTVLGQPKAAPSVTLFPPSS |
| | | EELQANKATLVCLISDFYPGAVTVAWKADSSPVKAGVETTTPSKQSNNK |
| | | YAASSYLSLTPEQWKSHRSYSCQVTHEGSTVEKTVAPTECS |
| Fab(IgG) | L6-F12578 | L6 Light Chain-F12578 Heavy Chain SEQ ID NO: 21 |
| | (Fab-IgG) | DIQMTQSPSSLSASVGDRVTITCRASQDVSTAVAWYQQKPGKAPKLLI |
| | | YSASFLYSGVPSRFSGSGSGTDFTLTISSLQPEDFATYYCQQSYTTPPTF |
| | | GQGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGECGGGGSGGGGSGGGGSEVQLVESGGGLVQPGG |
| | | SLRLSCAASGFTFSHYTLSWVRQAPGKGLEWVSVISGDGSYTYYADSV |
| | | KGRFTISSDNSKNTLYLQMNSLRAEDTAVYYCARNFIKYVFANWGQG |
| | | TLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVLH |
| | | QDWLNGKEYKCKVSNKALGAPIEKTISKAKGQPREPQVYTLPPSREEMTK |
| | | NQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| | | F12578 Light Chain SEQ ID NO: 22 |
| | | DIELTQPPSVSVAPGQTARISCSGDNIGSFYVHWYQQKPGQAPVLVIY |
| | | DKSNRPSGIPERFSGSNSGNTATLTISGTQAEDEADYYCQSYANTLSLV |
| | | FGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQV |
| | | THEGSTVEKTVAPTECS |
| | | L6Variable Heavy Chain - Constant Heavy Chain 1 SEQ ID NO: 23 |
| | | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSASTKGPSVFPLAPSSKST |
| | | SGGTAALGCLVKDYFPEPVTVSWNSGALTSGVHTFPAVLQSSGLYSLSSV |
| | | VTVPSSSLGTQTYICNVNHKPSNTKVDKKVEPKSC |
| Fv-IgG | L6-F127 | Heavy Chain SEQ ID NO: 24 |
| | | EVQLVESGGGLVQPGGSLRLSCAASGFTFTSYYISWVRQAPGKGLEWV |
| | | AEISPYSGSTYYADSVKGRFTISADTSKNTAYLQMNSLRAEDTAVYYCA |
| | | LRARPPIRLHPRGSVMDYWGQGTLVTVSSGGGGSGGGGSGGGGSEVQL |
| | | VESGGGLVQPGGSLRLSCAASGFNISSSYIHWVRQAPGKGLEWVAYIY |
| | | SSYGSTYYADSVKGRFTISADTSKNTAYLQMNSLRAEDTAVYYCARAS |
| | | WYALDYWGQGTLVTVSSASTKGPSVFPLAPSSKSTSGGTAALGCLVKDY |
| | | FPEPVTVSWNSGALTSGVHTFPAVLQSSGLYSLSSVVTVPSSSLGTQTYICN |
| | | VNHKPSNTKVDKKVEPKSCDKTHTCPPCPAPEAAGGPSVFLFPPKPKDTLM |
| | | ISRTPEVTCVVVDVSHEDPEVKFNWYVDGVEVHNAKTKPREEQYNSTYRV |
| | | VSVLTVLHQDWLNGKEYKCKVSNKALGAPIEKTISKAKGQPREPQVYTLP |
| | | PSREEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| | | Light Chain SEQ ID NO: 25 |
| | | DIQMTQSPSSLSASVGDRVTITCRASQDVSTAVAWYQQKPGKAPKLLIY |
| | | SASFLYSGVPSRFSGSGSGTDFTLTISSLQPEDFATYYCQQSYTTPPTFG |
| | | QGTKVEIKGGGGSGGGGSGGGGSDIQMTQSPSSLSASVGDRVTITCRAS |
| | | QSVSSAVAWYQQKPGKAPKLLIYSASSLYSGVPSRFSGSRSGTDFTLTIS |
| | | SLQPEDFATYYCQQYWYGVAPITFGQGTKVEIKRTVAAPSVFIFPPSDEQ |
| | | LKSGTASVVCLLNNFYPREAKVQWKVDNALQSGNSQESVTEQDSKDSTYS |
| | | LSSTLTLSKADYEKHKVYACEVTHQGLSSPVTKSFNRGEC |
| Fv-IgG | L6-F58 | Heavy Chain SEQ ID NO: 26 |
| | | EVQLVESGGGLVQPGGSLRLSCAASGFTFTSYYISWVRQAPGKGLEWV |
| | | AEISPYSGSTYYADSVKGRFTISADTSKNTAYLQMNSLRAEDTAVYYCA |
| | | LRARPPIRLHPRGSVMDYWGQGTLVTVSSGGGGSGGGGSGGGGSEVQL |
| | | VESGGGLVQPGGSLRLSCAASGFNISYSYIHWVRQAPGKGLEWVASIY |
| | | SSSGSTSYADSVKGRFTISADTSKNTAYLQMNSLRAEDTAVYYCARGAI |
| | | DYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHK |
| | | PSNTKVDKKVEPKSCDKTHTCPPCPAPEAAGGPSVFLFPPKPKDTLMISRTP |
| | | EVTCVVVDVSHEDPEVKFNWYVDGVEVHNAKTKPREEQYNSTYRVVSVL |
| | | TVLHQDWLNGKEYKCKVSNKALGAPIEKTISKAKGQPREPQVYTLPPSREE |
| | | MTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| | | Light Chain SEQ ID NO: 27 |
| | | DIQMTQSPSSLSASVGDRVTITCRASQDVSTAVAWYQQKPGKAPKLLIY |
| | | SASFLYSGVPSRFSGSGSGTDFTLTISSLQPEDFATYYCQQSYTTPPTFG |
| | | QGTKVEIKGGGGSGGGGSGGGGSDIQMTQSPSSLSASVGDRVTITCRAS |
| | | QSVSSAVAWYQQKPGKAPKLLIYSASSLYSGVPSRFSGSRSGTDFTLTIS |
| | | SLQPEDFATYYCQQWYSSGHVLITFGQGTKVEIKRTVAAPSVFIFPPSDE |
| | | QLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| Fv-IgG | L6-F4-2 | Heavy Chain SEQ ID NO: 28 |
| | | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSGGGGSGGGGSGGGGSE |
| | | VQLVESGGGLVQPGGSLRLSCAASGFNISYYYIHWVRQAPGKGLEWV |
| | | ASIYPSSGYTYYADSVKGRFTISADTSKNTAYLQMNSLRAEDTAVYYC |
| | | ARSSFYWAMDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQKSLS |
| | | LSPGK |
| | | Light Chain SEQ ID NO: 29 |
| | | DIQMTQSPSSLSASVGDRVTITCRASQDVSTAVAWYQQKPGKAPKLLI |
| | | YSASFLYSGVPSRFSGSGSGTDFTLTISSLQPEDFATYYCQQSYTTPPTF |
| | | GQGTKVEIKGGGGSGGGGSGGGGSDIQMTQSPSSLSASVGDRVTITCR |
| | | ASQSVSSAVAWYQQKPGKAPKLLIYSASSLYSGVPSRFSGSRSGTDFTL |
| | | TISSLQPEDFATYYCQQSYAAYLFTFGQGTKVEIKRTVAAPSVFIFPPSD |
| | | EQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |
| Fv-IgG | L6-F10 | Heavy Chain SEQ ID NO: 30 |
| | | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSGGGGSGGGGSGGGGSE |
| | | VQLVESGGGLVQPGGSLRLSCAASGFTFSSFNMFWVRQAPGKGLEW |
| | | VAGIDDDGSYPNYGSAVKGRATISRDNSKNTLYLQMNSLRAEDTAVY |
| | | YCAKSGYGGSWGGYIADDIDAWGQGTLVTVSSASTKGPSVFPLAPSSK |
| | | STSGGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPAPEA |
| | | AGGPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALGAPIE |
| | | KTISKAKGQPREPQVYTLPPSREEMTKNQVSLTCLVKGFYPSDIAVEWES |
| | | NGQPENNYKTTPPVLDSDGSFFLYSKLTVDKSRWQQGNVFSCSVMHEAL |
| | | HNHYTQKSLSLSPGK |
| | | Light Chain SEQ ID NO: 31 |
| | | DIQMTQSPSSLSASVGDRVTITCRASQDVSTAVAWYQQKPGKAPKLLI |
| | | YSASFLYSGVPSRFSGSGSGTDFTLTISSLQPEDFATYYCQQSYTTPPTF |
| | | GQGTKVEIKGGGGSGGGGSGGGGSELTQPPSVSVSPGQTARITCSGDGS |
| | | YAGSYYYGWYQQKPGQAPVTLIYYNNKRPSGIPERFSGSLSGSTNTLT |
| | | ISGVQAEDEADYYCGSADNSGAAFGGGTKLTVLRTVAAPSVFIFPPSDE |
| | | QLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| Fv-IgG | L6-F4 | Heavy Chain SEQ ID NO: 55 |
| | | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSGGGGSGGGGSGGGGSE |
| | | VQLVESGGGLVQPGGSLRLSCAASGFNLSSYSMHWVRQAPGKGLEW |
| | | VAYISSYYGYTYYADSVKGRFTISADTSKNTAYLQMNSLRAEDTAVYY |
| | | CARPAPGHWGFDYWGQGTLVTVSSASTKGPSVFPLAPSSKSTSGGTAA |
| | | LGCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPEAAGGPSVFL |
| | | FPPKPKDTLMISRTPEVTCVVVDVSHEDPEVKFNWYVDGVEVHNAKTKP |
| | | REEQYNSTYRVVSVLTVLHQDWLNGKEYKCKVSNKALGAPIEKTISKAK |
| | | GQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPGK |
| | | Light Chain SEQ ID NO: 56 |
| | | DIQMTQSPSSLSASVGDRVTITCRASQDVSTAVAWYQQKPGKAPKLLI |
| | | YSASFLYSGVPSRFSGSGSGTDFTLTISSLQPEDFATYYCQQSYTTPPTF |
| | | GQGTKVEIKGGGGSGGGGSGGGGSDIQMTQSPSSLSASVGDRVTITCR |
| | | ASQSVSSAVAWYQQKPGKAPKLLIYSASSLYSGVPSRFSGSRSGTDFTL |
| | | TISSLQPEDFATYYCQQWYYAPITFGQGTKVEIKRTVAAPSVFIFPPSDE |
| | | QLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| Fv-IgG | L6-F49 | Heavy Chain SEQ ID NO: 57 |
| | | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSGGGGSGGGGSGGGGSE |
| | | VQLVQSGAEVKKPGASVKVSCKASGGTFSSYAISWVRQAPGQGLEW |
| | | MGWINAGNGNTTYAQKFQGRVTMTRDTSTSTVYMELSSLRSEDTAV |
| | | YYCARHYYGSGSYPDWGQGTLVTVSSASTKGPSVFPLAPSSKSTSGGTA |
| | | ALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPEAAGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALGAPIEKTISK |
| | | AKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYT |
| | | QKSLSLSPGK |
| | | Light Chain SEQ ID NO: 58 |
| | | DIQMTQSPSSLSASVGDRVTITCRASQDVSTAVAWYQQKPGKAPKLLI |
| | | YSASFLYSGVPSRFSGSGSGTDFTLTISSLQPEDFATYYCQQSYTTPPTF |
| | | GQGTKVEIKRGGGGSGGGGSGGGGSDIQMTQSPSSPSASVGDRVTITCR |
| | | ASQSISSYLNWYQQKPGKAPKLLIYAASSLQSGVPSRFSGSGSGTDFTL |
| | | TISSLQPEDFATYYCQQSYSTPLTFGGGTKVEIKRTVAAPSVFIFPPSDE |
| | | QLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| Fv-IgG | L6-F7B | Heavy Chain SEQ ID NO: 59 |
| | | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSGGGGSGGGGSGGGGSE |
| | | VQLVESGGGLVQPGGSLRLSCAASGFNFSSSSIHWVRQAPGKGLEWV |
| | | AYIYPSYDYTYYADSVKGRFTISADTSKNTAYLQMNSLRAEDTAVYYC |
| | | ARGYFYTWGGMDYWGQGTLVTVSSASTKGPSVFPLAPSSKSTSGGTAA |
| | | LGCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPEAAGGPSVFL |
| | | FPPKPKDTLMISRTPEVTCVVVDVSHEDPEVKFNWYVDGVEVHNAKTKP |
| | | REEQYNSTYRVVSVLTVLHQDWLNGKEYKCKVSNKALGAPIEKTISKAK |
| | | GQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPGK |
| | | Light Chain SEQ ID NO: 60 |
| | | DIQMTQSPSSLSASVGDRVTITCRASQDVSTAVAWYQQKPGKAPKLLI |
| | | YSASFLYSGVPSRFSGSGSGTDFTLTISSLQPEDFATYYCQQSYTTPPTF |
| | | GQGTKVEIKRGGGGSGGGGSGGGGSDIQMTQSPSSLSASVGDRVTITC |
| | | RASQSVSSAVAWYQQKPGKAPKLLIYSASSLYSGVPSRFSGSRSGTDFT |
| | | LTISSLQPEDFATYYCQQYYSLFTFGQGTKVEIKRTVAAPSVFIFPPSDE |
| | | QLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| Fv-IgG | L6-F2I | Heavy Chain SEQ ID NO: 61 |
| | | EVQLVESGGGLVQPGGSLRLSCAASGFTFTSYYISWVRQAPGKGLEW |
| | | VAEISPYSGSTYYADSVKGRFTISADTSKNTAYLQMNSLRAEDTAVYY |
| | | CALRARPPIRLHPRGSVMDYWGQGTLVTVSSGGGGSGGGGSGGGGSE |
| | | VQLVESGGGLVQPGGSLRLSCAASGFNIHSSSIHWVRQAPGKGLEWV |
| | | AATYSSFGSITYADSVKGRFTISADTSKNTAYLQMNSLRAEDTAVYYC |
| | | ARYHHPFGYALDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPEAAGGPSVFLF |
| | | PPKPKDTLMISRTPEVTCVVVDVSHEDPEVKFNWYVDGVEVHNAKTKPR |
| | | EEQYNSTYRVVSVLTVLHQDWLNGKEYKCKVSNKALGAPIEKTISKAKG |
| | | QPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKS |
| | | LSLSPGK |
| | | Light Chain SEQ ID NO: 62 |
| | | DIQMTQSPSSLSASVGDRVTITCRASQDVSTAVAWYQQKPGKAPKLLI |
| | | YSASFLYSGVPSRFSGSGSGTDFTLTISSLQPEDFATYYCQQSYTTPPTF |
| | | GQGTKVEIKRGGGGSGGGGSGGGGSDIQMTQSPSSLSASVGDRVTITC |
| | | RASQSVSSAVAWYQQKPGKAPKLLIYSASSLYSGVPSRFSGSRSGTDFT |
| | | LTISSLQPEDFATYYCQQGVYLFTFGQGTKVEIKRTVAAPSVFIFPPSDE |
| | | QLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| N-HC- | L6-N-HC | L6-N-HC appended ScFv-F12578-IgG HC SEQ ID NO: 63 |
| appended | appended | EVQLVESGGGLVQPGGSLRLSCAASGFTFTSYYISWVRQAPGKGLEW |
| ScFv-IgG | ScFv- | VAEISPYSGSTYYADSVKGRFTISADTSKNTAYLQMNSLRAEDTAVYY |
| | F12578-IgG | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSEVQLVESGGGLVQPGGSLRLSCAASG |
| | | FTFSHYTLSWVRQAPGKGLEWVSVISGDGSYTYYADSVKGRFTISSDN |
| | | SKNTLYLQMNSLRAEDTAVYYCARNFIKYVFANWGQGTLVTVSSAST |
| | | KGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPKSCD |
| | | KTHTCPPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNGKEY |
| | | KCKVSNKALGAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQ |
| | | GNVFSCSVMHEALHNHYTQKSLSLSPGK |
| | | >F12578 LC SEQ ID NO: 64 |
| | | DIELTQPPSVSVAPGQTARISCSGDNIGSFYVHWYQQKPGQAPVLVIY |
| | | DKSNRPSGIPERFSGSNSGNTATLTISGTQAEDEADYYCQSYANTLSLV |
| | | FGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQV |
| | | THEGSTVEKTVAPTECS |
| N-HC- | L6-N-HC | L6-N-HC appended ScFv-F127-IgG HC SEQ ID NO: 65 |
| appended | appended | EVQLVESGGGLVQPGGSLRLSCAASGFTFTSYYISWVRQAPGKGLEW |
| ScFv-IgG | ScFv-F127- | VAEISPYSGSTYYADSVKGRFTISADTSKNTAYLQMNSLRAEDTAVYY |
| | IgG | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSEVQLVESGGGLVQPGGSLRLSCAASG |
| | | FNISSSYIHWVRQAPGKGLEWVAYIYSSYGSTYYADSVKGRFTISADTS |
| | | KNTAYLQMNSLRAEDTAVYYCARASWYALDYWGQGTLVTVSSASTK |
| | | GPSVFPLAPSSKSTSGGTAALGCLVKDYFPEPVTVSWNSGALTSGVHTFPA |
| | | VLQSSGLYSLSSVVTVPSSSLGTQTYICNVNHKPSNTKVDKKVEPKSCDKT |
| | | HTCPPCPAPEAAGGPSVFLFPPKPKDTLMISRTPEVTCVVVDVSHEDPEVK |
| | | FNWYVDGVEVHNAKTKPREEQYNSTYRVVSVLTVLHQDWLNGKEYKC |
| | | KVSNKALGAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGN |
| | | VFSCSVMHEALHNHYTQKSLSLSPGK |
| | | >F127 LC SEQ ID NO: 66 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQYWYGVAP |
| | | ITFGQGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACE |
| | | VTHQGLSSPVTKSFNRGEC |
| N-HC- | L6-N-HC | L6-N-HC appended ScFv-F58-IgG HC SEQ ID NO: 67 |
| appended | appended | EVQLVESGGGLVQPGGSLRLSCAASGFTFTSYYISWVRQAPGKGLEW |
| ScFv-IgG | ScFv-F58- | VAEISPYSGSTYYADSVKGRFTISADTSKNTAYLQMNSLRAEDTAVYY |
| | IgG | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSEVQLVESGGGLVQPGGSLRLSCAASG |
| | | FNISYSYIHWVRQAPGKGLEWVASIYSSSGSTSYADSVKGRFTISADTS |
| | | KNTAYLQMNSLRAEDTAVYYCARGAIDYWGQGTLVTVSSASTKGPSV |
| | | FPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTC |
| | | PPCPAPEAAGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSN |
| | | KALGAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCS |
| | | VMHEALHNHYTQKSLSLSPGK |
| | | >F58 LC SEQ ID NO: 68 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQWYSSGHV |
| | | LITFGQGTKVEIKRTVAAPSVFIFPPSDEQLKSGTASVVCLLNNFYPREAK |
| | | VQWKVDNALQSGNSQESVTEQDSKDSTYSLSSTLTLSKADYEKHKVYAC |
| | | EVTHQGLSSPVTKSFNRGEC |
| N-HC- | L6-N-HC | L6-N-HC appended ScFv-F4-2-IgG HC SEQ ID NO: 69 |
| appended | appended | EVQLVESGGGLVQPGGSLRLSCAASGFTFTSYYISWVRQAPGKGLEW |
| ScFv-IgG | ScFv-F4-2- | VAEISPYSGSTYYADSVKGRFTISADTSKNTAYLQMNSLRAEDTAVYY |
| | IgG | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSEVQLVESGGGLVQPGGSLRLSCAASG |
| | | FNISYYYIHWVRQAPGKGLEWVASIYPSSGYTYYADSVKGRFTISADT |
| | | SKNTAYLQMNSLRAEDTAVYYCARSSFYWAMDYWGQGTLVTVSSAS |
| | | TKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKVEPKSC |
| | | DKTHTCPPCPAPEAAGGPSVFLFPPKPKDTLMISRTPEVTCVVVDVSHEDP |
| | | EVKFNWYVDGVEVHNAKTKPREEQYNSTYRVVSVLTVLHQDWLNGKE |
| | | YKCKVSNKALGAPIEKTISKAKGQPREPQVYTLPPSREEMTKNQVSLTCL |
| | | VKGFYPSDIAVEWESNGQPENNYKTTPPVLDSDGSFFLYSKLTVDKSRWQ |
| | | QGNVFSCSVMHEALHNHYTQKSLSLSPGK |
| | | >F4-2 LC SEQ ID NO: 70 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQSYAAYLFT |
| | | FGQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| N-HC- | L6-N-HC | L6-N-HC appended ScFv-F4-IgG HC SEQ ID NO: 71 |
| appended | appended | EVQLVESGGGLVQPGGSLRLSCAASGFTFTSYYISWVRQAPGKGLEW |
| ScFv-IgG | ScFv-F4- | VAEISPYSGSTYYADSVKGRFTISADTSKNTAYLQMNSLRAEDTAVYY |
| | IgG | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSEVQLVESGGGLVQPGGSLRLSCAASG |
| | | FNLSSYSMHWVRQAPGKGLEWVAYISSYYGYTYYADSVKGRFTISAD |
| | | TSKNTAYLQMNSLRAEDTAVYYCARPAPGHWGFDYWGQGTLVTVSS |
| | | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPK |
| | | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNGK |
| | | EYKCKVSNKALGAPIEKTISKAKGQPREPQVYTLPPSREEMTKNQVSLTCL |
| | | VKGFYPSDIAVEWESNGQPENNYKTTPPVLDSDGSFFLYSKLTVDKSRWQ |
| | | QGNVFSCSVMHEALHNHYTQKSLSLSPGK |
| | | >F4 LC SEQ ID NO: 72 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQWYYAPIT |
| | | FGQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| N-HC- | L6-N-HC | L6-N-HC appended ScFv-F49-IgG SEQ ID NO: 73 |
| appended | appended | EVQLVESGGGLVQPGGSLRLSCAASGFTFTSYYISWVRQAPGKGLEW |
| ScFv-IgG | ScFv-F49- | VAEISPYSGSTYYADSVKGRFTISADTSKNTAYLQMNSLRAEDTAVYY |
| | IgG | CALRARPPIRIHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSQVQLVQSGAEVKKPGASVKVSCKAS |
| | | GGTFSSYAISWVRQAPGQGLEWMGWINAGNGNTTYAQKFQGRVTM |
| | | TRQTSTSTVYMELSSLRSEDTAVYYCARHYYGSGSYPDWGQGTLVTV |
| | | SSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEP |
| | | KSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNG |
| | | KEYKCKVSNKALGAPIEKTISKAKGQPREPQVYTLPPSREEMTKNQVSLT |
| | | CLVKGFYPSDIAVEWESNGQPENNYKTTPPVLDSDGSFFLYSKLTVDKSR |
| | | WQQGNVFSCSVMHEALHNHYTQKSLSLSPGK |
| | | >F49 LC SEQ ID NO: 74 |
| | | DIQMTQSPSSPSASVGDRVTITCRASQSISSYLNWYQQKPGKAPKLLIY |
| | | AASSLQSGVPSRFSGSGSGTDFTLTISSLQPEDFATYYCQQSYSTPLTFG |
| | | GGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGEC |
| N-HC- | L6-N-HC | L6-N-HC appended ScFv-F10-IgG HC SEQ ID NO: 75 |
| appended | appended | EVQLVESGGGLVQPGGSLRLSCAASGFTFTSYYISWVRQAPGKGLEW |
| ScFv-IgG | ScFv-F10- | VAEISPYSGSTYYADSVKGRFTISADTSKNTAYLQMNSLRAEDTAVYY |
| | IgG | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSEVQLVESGGGLVQPGGSLRLSCAASG |
| | | FTFSSFNMFWVRQAPGKGLEWVAGIDDDGSYPNYGSAVKGRATISRD |
| | | NSKNTLYLQMNSLRAEDTAVYYCAKSGYGGSWGGYIADDIDAWGQG |
| | | TLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVLH |
| | | QDWLNGKEYKCKVSNKALGAPIEKTISKAKGQPREPQVYTLPPSREEMTK |
| | | NQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| | | >F10 LC SEQ ID NO: 76 |
| | | ELTQPPSVSVSPGQTARITCSGDGSYAGSYYYGWYQQKPGQAPVTLIY |
| | | YNNKRPSGIPERFSGSLSGSTNTLTISGVQAEDEADYYCGSADNSGAAF |
| | | GGGTKLTVLRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| N-HC- | L6-N-HC- | L6-N-HC appended ScFv-F7B-IgG HC SEQ ID NO: 189 |
| appended | Appended | EVQLVESGGGLVQPGGSLRLSCAASGFTFTSYYISWVRQAPGKGLEW |
| ScFv-IgG | ScFv-F7B- | VAEISPYSGSTYYADSVKGRFTISADTSKNTAYLQMNSLRAEDTAVYY |
| | IgG | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSEVQLVESGGGLVQPGGSLRLSCAASG |
| | | FNFSSSSIHWVRQAPGKGLEWVAYIYPSYDYTYYADSVKGRFTISADT |
| | | SKNTAYLQMNSLRAEDTAVYYCARGYFYTWGGMDYWGQGTLVTVS |
| | | SASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPK |
| | | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNGK |
| | | EYKCKVSNKALGAPIEKTISKAKGQPREPQVYTLPPSREEMTKNQVSLTCL |
| | | VKGFYPSDIAVEWESNGQPENNYKTTPPVLDSDGSFFLYSKLTVDKSRWQ |
| | | QGNVFSCSVMHEALHNHYTQKSLSLSPGK |
| | | F7B LC SEQ ID NO: 190 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQYYSLFTFG |
| | | QGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGEC |
| N-HC- | L6-N-HC- | L6-N-HC appended ScFv-F2I-IgG HC SEQ ID NO: 191 |
| appended | Appended | EVQLVESGGGLVQPGGSLRLSCAASGFTFTSYYISWVRQAPGKGLEW |
| ScFv-IgG | ScFv-F2I- | VAEISPYSGSTYYADSVKGRFTISADTSKNTAYLQMNSLRAEDTAVYY |
| | IgG | CALRARPPIRLHPRGSVMDYWGQGTLVTVSSGSAASGSSGGSSSGADIQ |
| | | MTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQ |
| | | GTKVEIKGGGGSGGGGSGGGGSEVQLVESGGGLVQPGGSLRLSCAASG |
| | | FNIHSSSIHWVRQAPGKGLEWVAATYSSFGSITYADSVKGRFTISADTS |
| | | KNTAYLQMNSLRAEDTAVYYCARYHHPFGYALDYWGQGTLVTVSSA |
| | | STKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKKVEPKS |
| | | CDKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGKE |
| | | YKCKVSNKALGAPIEKTISKAKGQPREPQVYTLPPSREEMTKNQVSLTCL |
| | | VKGFYPSDIAVEWESNGQPENNYKTTPPVLDSDGSFFLYSKLTVDKSRWQ |
| | | QGNVFSCSVMHEALHNHYTQKSLSLSPGK |
| | | F2I LC SEQ ID NO: 192 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQGVYLFTF |
| | | GQGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGEC |
| N-HC | L6-N-HC | L6-N-HC appended ScFv-F12578-IgG-Knob-His HC SEQ ID NO: 77 |
| appended | appended | EVQLVESGGGLVQPGGSLRLSCAASGFTFTSYYISWVRQAPGKGLEW |
| ScFv-IgG- | ScFv- | VAEISPYSGSTYYADSVKGRFTISADTSKNTAYLQMNSLRAEDTAVYY |
| Knob-his + | F12578- | CALRARPPIRLHPRGSVMDYWGQGTLVTVSSGSAASGSSGGSSSGADIQ |
| IgG-Hole- | IgG-Knob- | MTQSPSSLSASVGDRVTITCRASQDVSTAVAWYQQKPGKAPKLLIYSA |
| Flag | his + | SFLYSGVPSRFSGSGSGTDFTLTISSLQPEDFATYYCQQSYTTPPTFGQ |
| | F12578- | GTKVEIKGGGGSGGGGSGGGGSEVQLVESGGGLVQPGGSLRLSCAASG |
| | IgG-Hole- | FTFSHYTLSWVRQAPGKGLEWVSVISGDGSYTYYADSVKGRFTISSDN |
| | Flag | SKNTLYLQMNSLRAEDTAVYYCARNFIKYVFANWGQGTLVTVSSAST |
| | | KGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPKSCD |
| | | KTHTCPPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNGKEY |
| | | KCKVSNKALGAPIEKTISKAKGQPREPQVYTLPPSREEMTKNQVSLWCLV |
| | | KGFYPSDIAVEWESNGQPENNYKTTPPVLDSDGSFFLYSKLTVDKSRWQQ |
| | | GNVFSCSVMHEALHNHYTQKSLSLSPGKGSGSGHHHHHH |
| | | > F12578-IgG-Hole Flag HC SEQ ID NO: 78 |
| | | EVQLVESGGGLVQPGGSLRLSCAASGFTFSHYTLSWVRQAPGKGLE |
| | | WVSVISGDGSYTYYADSVKGRFTISSDNSKNTLYLQMNSLRAEDTAVY |
| | | YCARNFIKYVFANWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPEAAGGPSVFLF |
| | | PPKPKDTLMISRTPEVTCVVVDVSHEDPEVKFNWYVDGVEVHNAKTKPR |
| | | EEQYNSTYRVVSVLTVLHQDWLNGKEYKCKVSNKALGAPIEKTISKAKG |
| | | QPREPQVYTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKS |
| | | LSLSPGKGSGSGDYKDDDDK |
| | | >F12578 LC SEQ ID NO: 79 |
| | | DIELTQPPSVSVAPGQTARISCSGDNIGSFYVHWYQQKPGQAPVLVIY |
| | | DKSNRPSGIPERFSGSNSGNTATLTISGTQAEDEADYYCQSYANTLSLV |
| | | FGGGTKLTVLGPPKAAPSVTLFPPSSEELPANKATLVCLISDFYPGAVTV |
| | | AWKADSSPVKAGVETTTPSKPSNNKYAASSYLSLTPEPWKSHRSYSCPV |
| | | THEGSTVEKTVAPTECS |
| N-HC | L6-N-HC | L6-N-HC appended ScFv-F127-IgG-Knob-His HC SEQ ID NO: 80 |
| appended | appended | EVQLVESGGGLVQPGGSLRLSCAASGFTFTSYYISWVRQAPGKGLEW |
| ScFv-IgG- | ScFv-F127- | VAEISPYSGSTYYADSVKGRFTISADTSKNTAYLQMNSLRAEDTAVYY |
| Knob-his + | IgG-Knob- | CALRARPPIRLHPRGSVMDYWGQGTLVTVSSGSAASGSSGGSSSGADIQ |
| IgG-Hole- | his + F127- | MTQSPSSLSASVGDRVTITCRASQDVSTAVAWYQQKPGKAPKLLIYSA |
| Flag | IgG-Hole- | SFLYSGVPSRFSGSGSGTDFTLTISSLQPEDFATYYCQQSYTTPPTFGQ |
| | Flag | GTKVEIKGGGGSGGGGSGGGGSEVQLVESGGGLVQPGGSLRLSCAASG |
| | | FNISSSYIHWVRQAPGKGLEWVAYIYSSYGSTYYADSVKGRFTISADTS |
| | | KNTAYLQMNSLRAEDTAVYYCARASWYALDYWGQGTLVTVSSASTK |
| | | GPSVFPLAPSSKSTSGGTAALGCLVKDYFPEPVTVSWNSGALTSGVHTFPA |
| | | VLQSSGLYSLSSVVTVPSSSLGTQTYICNVNHKPSNTKVDKKVEPKSCDKT |
| | | HTCPPCPAPEAAGGPSVFLFPPKPKDTLMISRTPEVTCVVVDVSHEDPEVK |
| | | FNWYVDGVEVHNAKTKPREEQYNSTYRVVSVLTVLHQDWLNGKEYKC |
| | | KVSNKALGAPIEKTISKAKGPPREPPVYTLPPSREEMTKNPVSLWCLVKG |
| | | FYPSDIAVEWESNGPPENNYKTTPPVLDSDGSFFLYSKLTVDKSRWPPGN |
| | | VFSCSVMHEALHNHYTQKSLSLSPGKGSGSGHHHHHH |
| | | > F127-IgG-Hole Flag HC SEQ ID NO: 81 |
| | | EVQLVESGGGLVQPGGSLRLSCAASGFNISSSYIHWVRQAPGKGLEW |
| | | VAYIYSSYGSTYYADSVKGRFTISADTSKNTAYLQMNSLRAEDTAVYY |
| | | CARASWYALDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLG |
| | | TPTYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSREEMTKNQVSLSCAVKGFYPSDIAVEWESNGQPENNYKT |
| | | TPPVLDSDGSFFLVSKLTVDKSRWQQGNVFSCSVMHEALHNHYTQKSLS |
| | | LSPGKGSGSGDYKDDDDK |
| | | >F127 LC SEQ ID NO: 82 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQYWYGVAP |
| | | ITFGQGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACE |
| | | VTHQGLSSPVTKSFNRGEC |
| N-HC | L6-N-HC | L6-N-HC appended ScFv-F58-IgG-Knob-His HC SEQ ID NO: 83 |
| appended | appended | EVQLVESGGGLVQPGGSLRLSCAASGFTFTSYYISWVRQAPGKGLEW |
| ScFv-IgG- | ScFv-F58- | VAEISPYSGSTYYADSVKGRFTISADTSKNTAYLQMNSLRAEDTAVYY |
| Knob-his + | IgG-Knob- | CALRARPPIRLHPRGSVMDYWGQGTLVTVSSGSAASGSSGGSSSGADIQ |
| IgG-Hole- | his + F58- | MTQSPSSLSASVGDRVTITCRASQDVSTAVAWYQQKPGKAPKLLIYSA |
| Flag | IgG-Hole- | SFLYSGVPSRFSGSGSGTDFTLTISSLQPEDFATYYCQQSYTTPPTFGQ |
| | Flag | GTKVEIKGGGGSGGGGSGGGGSEVQLVESGGGLVQPGGSLRLSCAASG |
| | | FNISYSYIHWVRQAPGKGLEWVASIYSSSGSTSYADSVKGRFTISADTS |
| | | KNTAYLQMNSLRAEDTAVYYCARGAIDYWGQGTLVTVSSASTKGPSV |
| | | FPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTC |
| | | PPCPAPEAAGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSN |
| | | KALGAPIEKTISKAKGQPREPQVYTLPPSREEMTKNQVSLWCLVKGFYPS |
| | | DIAVEWESNGQPENNYKTTPPVLDSDGSFFLYSKLTVDKSRWQQGNVFSC |
| | | SVMHEALHNHYTQKSLSLSPGKGSGSGHHHHHH |
| | | > F58-IgG-Hole Flag HC SEQ ID NO: 84 |
| | | EVQLVESGGGLVQPGGSLRLSCAASGFNISYSYIHWVRQAPGKGLEW |
| | | VASIYSSSGSTSYADSVKGRFTISADTSKNTAYLQMNSLRAEDTAVYYC |
| | | ARGAIDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNS |
| | | TYRVVSVLTVLHQDWLNGKEYKCKVSNKALGAPIEKTISKAKGQPREPQ |
| | | VYTLPPSREEMTKNQVSLSCAVKGFYPSDIAVEWESNGQPENNYKTTPPV |
| | | LDSDGSFFLVSKLTVDKSRWQQGNVFSCSVMHEALHNHYTQKSLSLSPG |
| | | KGSGSGDYKDDDDK |
| | | >F58 LC SEQ ID NO: 85 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQWYSSGHV |
| | | LITFGQGTKVEIKRTVAAPSVFIFPPSDEQLKSGTASVVCLLNNFYPREAK |
| | | VQWKVDNALQSGNSQESVTEQDSKDSTYSLSSTLTLSKADYEKHKVYAC |
| | | EVTHQGLSSPVTKSFNRGEC |
| N-HC | L6-N-HC | L6-N-HC appended ScFv-F4-2-IgG-Knob-His HC SEQ ID NO: 86 |
| appended | appended | EVQLVESGGGLVQPGGSLRLSCAASGFTFTSYYISWVRQAPGKGLEW |
| ScFv-IgG- | ScFv-F4-2- | VAEISPYSGSTYYADSVKGRFTISADTSKNTAYLQMNSLRAEDTAVYY |
| Knob-his + | IgG-Knob- | CALRARPPIRLHPRGSVMDYWGQGTLVTVSSGSAASGSSGGSSSGADIQ |
| IgG-Hole- | his + F4-2- | MTQSPSSLSASVGDRVTITCRASQDVSTAVAWYQQKPGKAPKLLIYSA |
| Flag | IgG-Hole- | SFLYSGVPSRFSGSGSGTDFTLTISSLQPEDFATYYCQQSYTTPPTFGQ |
| | Flag | GTKVEIKGGGGSGGGGSGGGGSEVQLVESGGGLVQPGGSLRLSCAASG |
| | | FNISYYYIHWVRQAPGKGLEWVASIYPSSGYTYYADSVKGRFTISADT |
| | | SKNTAYLQMNSLRAEDTAVYYCARSSFYWAMDYWGQGTLVTVSSAS |
| | | TKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKVEPKSC |
| | | DKTHTCPPCPAPEAAGGPSVFLFPPKPKDTLMISRTPEVTCVVVDVSHEDP |
| | | EVKFNWYVDGVEVHNAKTKPREEQYNSTYRVVSVLTVLHQDWLNGKE |
| | | YKCKVSNKALGAPIEKTISKAKGQPREPQVYTLPPSREEMTKNQVSLWCL |
| | | VKGFYPSDIAVEWESNGQPENNYKTTPPVLDSDGSFFLYSKLTVDKSRWQ |
| | | QGNVFSCSVMHEALHNHYTQKSLSLSPGKGSGSGHHHHHH |
| | | > F4-2-IgG-Hole Flag HC SEQ ID NO: 87 |
| | | EVQLVESGGGLVQPGGSLRLSCAASGFNISYYYIHWVRQAPGKGLEW |
| | | VASIYPSSGYTYYADSVKGRFTISADTSKNTAYLQMNSLRAEDTAVYY |
| | | CARSSFYWAMDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPEAAGGPSVFLF |
| | | PPKPKDTLMISRTPEVTCVVVDVSHEDPEVKFNWYVDGVEVHNAKTKPR |
| | | EEQYNSTYRVVSVLTVLHQDWLNGKEYKCKVSNKALGAPIEKTISKAKG |
| | | QPREPQVYTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKS |
| | | LSLSPGKGSGSGDYKDDDDK |
| | | >F4-2 LC SEQ ID NO: 88 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQSYAAYLFT |
| | | FGQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| N-HC | L6-N-HC | L6-N-HC appended ScFv-F4-IgG-Knob-His HC SEQ ID NO: 89 |
| appended | appended | EVQLVESGGGLVQPGGSLRLSCAASGFTFTSYYISWVRQAPGKGLEW |
| ScFv-IgG- | ScFv-F4- | VAEISPYSGSTYYADSVKGRFTISADTSKNTAYLQMNSLRAEDTAVYY |
| Knob-his + | IgG-Knob- | CALRARPPIRLHPRGSVMDYWGQGTLVTVSSGSAASGSSGGSSSGADIQ |
| IgG-Hole- | his + F4- | MTQSPSSLSASVGDRVTITCRASQDVSTAVAWYQQKPGKAPKLLIYSA |
| Flag | IgG-Hole- | SFLYSGVPSRFSGSGSGTDFTLTISSLQPEDFATYYCQQSYTTPPTFGQ |
| | Flag | GTKVEIKGGGGSGGGGSGGGGSEVQLVESGGGLVQPGGSLRLSCAASG |
| | | FNLSSYSMHWVRQAPGKGLEWVAYISSYYGYTYYADSVKGRFTISAD |
| | | TSKNTAYLQMNSLRAEDTAVYYCARPAPGHWGFDYWGQGTLVTVSS |
| | | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPK |
| | | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNGK |
| | | EYKCKVSNKALGAPIEKTISKAKGQPREPQVYTLPPSREEMTKNQVSLWC |
| | | LVKGFYPSDIAVEWESNGQPENNYKTTPPVLDSDGSFFLYSKLTVDKSRW |
| | | QQGNVFSCSVMHEALHNHYTQKSLSLSPGKGSGSGHHHHHH |
| | | > F4-IgG-Hole Flag HC SEQ ID NO: 90 |
| | | EVQLVESGGGLVQPGGSLRLSCAASGFNLSSYSMHWVRQAPGKGLE |
| | | WVAYISSYYGYTYYADSVKGRFTISADTSKNTAYLQMNSLRAEDTAV |
| | | YYCARPAPGHWGFDYWGQGTLVTVSSASTKGPSVFPLAPSSKSTSGGT |
| | | AALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEAAGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALGAPIEKTISK |
| | | AKGQPREPQVYTLPPSREEMTKNQVSLSCAVKGFYPSDIAVEWESNGQPE |
| | | NNYKTTPPVLDSDGSFFLVSKLTVDKSRWQQGNVFSCSVMHEALHNHYT |
| | | QKSLSLSPGKGSGSGDYKDDDDK |
| | | >F4 LC SEQ ID NO: 91 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQWYYAPIT |
| | | FGQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| N-HC | L6-N-HC | L6-N-HC appended ScFv-F49-IgG-Knob-His HC SEQ ID NO: 92 |
| appended | appended | EVQLVESGGGLVQPGGSLRLSCAASGFTFTSYYISWVRQAPGKGLEW |
| ScFv-IgG- | ScFv-F49- | VAEISPYSGSTYYADSVKGRFTISADTSKNTAYLQMNSLRAEDTAVYY |
| Knob-his + | IgG-Knob- | CALRARPPIRLHPRGSVMDYWGQGTLVTVSSGSAASGSSGGSSSGADIQ |
| IgG-Hole- | his + F49- | MTQSPSSLSASVGDRVTITCRASQDVSTAVAWYQQKPGKAPKLLIYSA |
| Flag | IgG-Hole- | SFLYSGVPSRFSGSGSGTDFTLTISSLQPEDFATYYCQQSYTTPPTFGQ |
| | Flag | GTKVEIKGGGGSGGGGSGGGGSQVQLVQSGAEVKKPGASVKVSCKAS |
| | | GGTFSSYAISWVRQAPGQGLEWMGWINAGNGNTTYAQKFQGRVTM |
| | | TRDTSTSTVYMELSSLRSEDTAVYYCARHYYGSGSYPDWGQGTLVTV |
| | | SSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEP |
| | | KSCDKTHTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDWLNG |
| | | KEYKCKVSNKALGAPIEKTISKAKGQPREPQVYTLPPSREEMTKNQVSLW |
| | | CLVKGFYPSDIAVEWESNGQPENNYKTTPPVLDSDGSFFLYSKLTVDKSR |
| | | WQQGNVFSCSVMHEALHNHYTQKSLSLSPGKGSGSGHHHHHH |
| | | > F49-IgG-Hole Flag HC SEQ ID NO: 93 |
| | | QVQLVQSGAEVKKPGASVKVSCKASGGTFSSYAISWVRQAPGQGLE |
| | | WMGWINAGNGNTTYAQKFQGRVTMTRDTSTSTVYMELSSLRSEDTA |
| | | VYYCARHYYGSGSYPDWGQGTLVTVSSASTKGPSVFPLAPSSKSTSGGT |
| | | AALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEAAGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALGAPIEKTISK |
| | | AKGQPREPQVYTLPPSREEMTKNQVSLSCAVKGFYPSDIAVEWESNGQPE |
| | | NNYKTTPPVLDSDGSFFLVSKLTVDKSRWQQGNVFSCSVMHEALHNHYT |
| | | QKSLSLSPGKGSGSGDYKDDDDK |
| | | >F49 LC SEQ ID NO: 94 |
| | | DIQMTQSPSSPSASVGDRVTITCRASQSISSYLNWYQQKPGKAPKLLIY |
| | | AASSLQSGVPSRFSGSGSGTDFTLTISSLQPEDFATYYCQQSYSTPLTFG |
| | | GGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGEC |
| N-HC | L6-N-HC | L6-N-HC appended ScFv-F10-IgG-Knob-His HC SEQ ID NO: 95 |
| appended | appended | EVQLVESGGGLVQPGGSLRLSCAASGFTFTSYYISWVRQAPGKGLEW |
| ScFv-IgG- | ScFv-F10- | VAEISPYSGSTYYADSVKGRFTISADTSKNTAYLQMNSLRAEDTAVYY |
| Knob-his + | IgG-Knob- | CALRARPPIRLHPRGSVMDYWGQGTLVTVSSGSAASGSSGGSSSGADIQ |
| IgG-Hole- | his + F10- | MTQSPSSLSASVGDRVTITCRASQDVSTAVAWYQQKPGKAPKLLIYSA |
| Flag | IgG-Hole- | SFLYSGVPSRFSGSGSGTDFTLTISSLQPEDFATYYCQQSYTTPPTFGQ |
| | Flag | GTKVEIKGGGGSGGGGSGGGGSEVQLVESGGGLVQPGGSLRLSCAASG |
| | | FTFSSFNMFWVRQAPGKGLEWVAGIDDDGSYPNYGSAVKGRATISRD |
| | | NSKNTLYLQMNSLRAEDTAVYYCAKSGYGGSWGGYIADDIDAWGQG |
| | | TLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVLH |
| | | QDWLNGKEYKCKVSNKALGAPIEKTISKAKGQPREPQVYTLPPSREEMTK |
| | | NQVSLWCLVKGFYPSDIAVEWESNGQPENNYKTTPPVLDSDGSFFLYSKL |
| | | TVDKSRWQQGNVFSCSVMHEALHNHYTQKSLSLSPGKGSGSGHHHHHH |
| | | > F10-IgG-Hole Flag HC SEQ ID NO: 96 |
| | | EVQLVESGGGLVQPGGSLRLSCAASGFTFSSFNMFWVRQAPGKGLE |
| | | WVAGIDDDGSYPNYGSAVKGRATISRDNSKNTLYLQMNSLRAEDTAV |
| | | YYCAKSGYGGSWGGYIADDIDAWGQGTLVTVSSASTKGPSVFPLAPSS |
| | | KSTSGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPCPAPE |
| | | AAGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALGAP |
| | | IEKTISKAKGQPREPQVYTLPPSREEMTKNQVSLSCAVKGFYPSDIAVEWE |
| | | SNGQPENNYKTTPPVLDSDGSFFLVSKLTVDKSRWQQGNVFSCSVMHEA |
| | | LHNHYTQKSLSLSPGKGSGSGDYKDDDDK |
| | | >F10 LC SEQ ID NO: 97 |
| | | ELTQPPSVSVSPGQTARITCSGDGSYAGSYYYGWYQQKPGQAPVTLIY |
| | | YNNKRPSGIPERFSGSLSGSTNTLTISGVQAEDEADYYCGSADNSGAAF |
| | | GGGTKLTVLRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| N-HC | L6-N-HC | L6-N-HC appended ScFv-F7B-IgG-Knob-His HC SEQ ID NO: 193 |
| appended | appended | EVQLVESGGGLVQPGGSLRLSCAASGFTFTSYYISWVRQAPGKGLEW |
| ScFv-IgG- | ScFv-F7B- | VAEISPYSGSTYYADSVKGRFTISADTSKNTAYLQMNSLRAEDTAVYY |
| Knob-his + | IgG-Knob- | CALRARPPIRLHPRGSVMDYWGQGTLVTVSSGSAASGSSGGSSSGADIQ |
| IgG-Hole- | his + F7B- | MTQSPSSLSASVGDRVTITCRASQDVSTAVAWYQQKPGKAPKLLIYSA |
| Flag | IgG-Hole- | SFLYSGVPSRFSGSGSGTDFTLTISSLQPEDFATYYCQQSYTTPPTFGQ |
| | Flag | GTKVEIKGGGGSGGGGSGGGGSEVQLVESGGGLVQPGGSLRLSCAASG |
| | | FNFSSSSIHWVRQAPGKGLEWVAYIYPSYDYTYYADSVKGRFTISADT |
| | | SKNTAYLQMNSLRAEDTAVYYCARGYFYTWGGMDYWGQGTLVTVS |
| | | SASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPK |
| | | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNGK |
| | | EYKCKVSNKALGAPIEKTISKAKGQPREPQVYTLPPSREEMTKNQVSLWC |
| | | LVKGFYPSDIAVEWESNGQPENNYKTTPPVLDSDGSFFLYSKLTVDKSRW |
| | | QQGNVFSCSVMHEALHNHYTQKSLSLSPGKGSGSGHHHHHH |
| | | > F7B-IgG-Hole Flag HC SEQ ID NO: 194 |
| | | EVQLVESGGGLVQPGGSLRLSCAASGFNFSSSSIHWVRQAPGKGLEW |
| | | VAYIYPSYDYTYYADSVKGRFTISADTSKNTAYLQMNSLRAEDTAVYY |
| | | CARGYFYTWGGMDYWGQGTLVTVSSASTKGPSVFPLAPSSKSTSGGTA |
| | | ALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPEAAGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALGAPIEKTISK |
| | | AKGQPREPQVYTLPPSREEMTKNQVSLSCAVKGFYPSDIAVEWESNGQPE |
| | | NNYKTTPPVLDSDGSFFLVSKLTVDKSRWQQGNVFSCSVMHEALHNHYT |
| | | QKSLSLSPGKGSGSGDYKDDDDK |
| | | >F7B LC SEQ ID NO: 195 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQYYSLFTFG |
| | | QGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGEC |
| N-HC | L6-N-HC | L6-N-HC appended ScFv-F2I-IgG-Knob-His HC SEQ ID NO: 196 |
| appended | appended | EVQLVESGGGLVQPGGSLRLSCAASGFTFTSYYISWVRQAPGKGLEW |
| ScFv-IgG- | ScFv-F2I- | VAEISPYSGSTYYADSVKGRFTISADTSKNTAYLQMNSLRAEDTAVYY |
| Knob-his + | IgG-Knob- | CALRARPPIRLHPRGSVMDYWGQGTLVTVSSGSAASGSSGGSSSGADIQ |
| IgG-Hole- | his + F2I- | MTQSPSSLSASVGDRVTITCRASQDVSTAVAWYQQKPGKAPKLLIYSA |
| Flag | IgG-Hole- | SFLYSGVPSRFSGSGSGTDFTLTISSLQPEDFATYYCQQSYTTPPTFGQ |
| | Flag | GTKVEIKGGGGSGGGGSGGGGSEVQLVESGGGLVQPGGSLRLSCAASG |
| | | FNIHSSSIHWVRQAPGKGLEWVAATYSSFGSITYADSVKGRFTISADTS |
| | | KNTAYLQMNSLRAEDTAVYYCARYHHPFGYALDYWGQGTLVTVSSA |
| | | STKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKKVEPKS |
| | | CDKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGKE |
| | | YKCKVSNKALGAPIEKTISKAKGQPREPQVYTLPPSREEMTKNQVSLWCL |
| | | VKGFYPSDIAVEWESNGQPENNYKTTPPVLDSDGSFFLYSKLTVDKSRWQ |
| | | QGNVFSCSVMHEALHNHYTQKSLSLSPGKGSGSGHHHHHH |
| | | > F2I-IgG-Hole Flag HC SEQ ID NO: 197 |
| | | EVQLVESGGGLVQPGGSLRLSCAASGFNIHSSSIHWVRQAPGKGLEW |
| | | VAATYSSFGSITYADSVKGRFTISADTSKNTAYLQMNSLRAEDTAVYY |
| | | CARYHHPFGYALDYWGQGTLVTVSSASTKGPSVFPLAPSSKSTSGGTA |
| | | ALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPEAAGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALGAPIEKTISK |
| | | AKGQPREPQVYTLPPSREEMTKNQVSLSCAVKGFYPSDIAVEWESNGQPE |
| | | NNYKTTPPVLDSDGSFFLVSKLTVDKSRWQQGNVFSCSVMHEALHNHYT |
| | | QKSLSLSPGKGSGSGDYKDDDDK |
| | | >F2ILC SEQ ID NO: 198 |
| | | DIQMTQSPSSLSASVGDRVTITCRASQSVSSAVAWYQQKPGKAPKLLI |
| | | YSASSLYSGVPSRFSGSRSGTDFTLTISSLQPEDFATYYCQQGVYLFTF |
| | | GQGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGEC |

### Example 2

### Generation of WNT Super Agonist Molecules

To complement the set of surrogate agonists, generation of a set of potent antagonist molecules to study WNT signaling through specific FZDs was also attempted. Previous studies have shown that antibodies that compete with WNT binding to FZD can serve as antagonists (Gurney, et al., supra), however, this approach required the continuous presence of the antibodies at relatively high concentrations. Additionally, not all FZD binding antibodies generated functioned as antagonists, e.g., if they did not compete with WNT binding to receptor. Therefore, a potent and FZD selective antagonist platform would be highly desirable for both research and therapeutic development.

ZNRF3 and RNF43 are membrane-bound E3 ligases that target WNT receptors (FZDs and LRPs) for degradation (Hao et al. (2012) Nature 485:195-200; and Koo et al. (2012) Nature 488:665-669). Based on the activities of the E3 ligases, constructs were made to test if a fusion between FZD and E3 ligase binders would act as an antagonist of WNT signaling.

The E3 ligase binding activity of RSPO2 was utilized by fusing either a control GFP antibody or the FZD binding antibody, F12578, to a mutant RSPO2 fragment. The mutant RSPO2 fragment contained furin domains, Fu1Fu2, that harbor a double F105R/F109A mutation in the Fu2 domain (FIG. 2A, designated “RSPO2RA”). RSPO2RA fragment lost the ability to bind LGR (and therefore lost the WNT signal enhancing activity), but retained ability to bind E3 ligases (Xie et al. (2013) EMBO Rep. 14:1120-1126). Compared to wild type RSPO2Fu1Fu2-Fc fusion (Fc-RSPO2), the RSPO2RA mutant fusion to a negative control anti-GFP antibody had significantly diminished WNT signal enhancing activity. Only modest activities were observed at the highest dose tested (FIG. 2B). Surprisingly, while the fusion of the RSPO2RA mutant to F12578 (F12578-RSPO2RA) had no activity on its own, the fusion protein resulted in a biphasic curve in the presence of WNT3A, where at lower doses, it enhanced rather than inhibited WNT3A signaling (FIG. 2B). To understand the mechanism leading to the enhanced signaling, we performed FACS analysis to assess the FZD receptor levels on cell surface. As shown in FIG. 2C, cell treated with F12578-RSPO2RA showed increased FZD levels as detected by anti-FZD antibody. These results suggest that, instead of acting as an inhibitor to reduce FZD levels, F12578-RSPO2RA acted at least in part in a RSPO mimetic fashion, increased receptor levels and enhanced Wnt signaling.

To understand the general applicability of this observation, additional fusion proteins between RSPO2RA with other FZD binders, R2M3 (“F6”) which binds FZD1,2,7,5,8 and 1791 (“F7”) which binds FZD7, were generated. In these cases, the RSPO2RA was fused to the N-terminus of the FZD binding antibody heavy chain. As shown in FIG. 2D, both RSPO2RA-F6 and RSPO2-F7 also behaved in a RSPO mimetic fashion and enhanced WNT3A activities at low doses. A fusion of RSPO2RA to the N-terminus of F6 Fab on its heavy chain, RSPO2RA-F6_Fab, was also generated. As shown in FIG. 2E, the monovalent fusion protein also enhanced WNT3A activity. These results surprisingly suggested that, instead of acting as a suppressor to reduce FZD levels, FZD binder fusions to RSPO2RA, or generally E3 ligase binders, acted in a RSPO mimetic fashion and enhanced WNT signaling. Structure and sequence of these novel RSPO mimetic molecules are shown in Table 4.

Since this approach yielded the surprising result of enhancers instead of predicted antagonists, it was further investigated whether the surrogate molecules of FIG. 1, in particular the Fv-Ig structure, in combination with the E3 ligase binders would result in a WNT super agonist. To that end, trispecific, hexavalent molecules were generated as shown in FIG. 3A. As shown in FIG. 3B, this type of molecule possesses both WNT surrogate and RSPO activities as exemplified by the construct L6-F12578-RSPO2RA. This WNT super agonist activity translated to different FZD binders, e.g., L6-F127-RSPO2RA, L6-F58-RSPO2RA, L6-F4-RSPO2RA, L6-F49-RSPO2RA, L6-F10-RSPO2RA, L6-F7B-RSPO2RA, L6-F2I-RSPO2RA, L6-F4-2-RSPO2RA (FIG. 3C-3K). Additional formats where RSPO2RA was attached to different locations of the WNT mimetic molecule was also constructed as shown in FIG. 3J, with activities shown in 3K. Table 4 describes the different components/formats tested.

TABLE 4
WNT enhancers and WNT super agonist structures and sequences

| | | Sequences (as depicted in the accompanying |
|---|---|---|
| | | figures, WNT enhancer molecules and WNT super |
| | | agonist molecules typically comprises two of each |
| | | polypeptide sequence shown for any particular |
| Structure | Components | structure) |
| 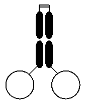 | Fc-RSPO2 | SEQ ID NO: 32 DKTHTCPPCPAPEAAGGPSVFLFPPKPKDTLMISRTPEVTCV VVDVSHEDPEVKFNWYVDGVEVHNAKTKPREEQYNSTYRVVS VLTVLHQDWLNGKEYKCKVSNKALGAPIEKTISKAKGQPREP QVYTLPPSREEMTKNQVSLTCLVNKGFYPSDIAVEWESNGQPE NNYKTTPPVLDSDGSFFLYSKLTVDKSRWQQGNVFSCSVMHE |
| | | 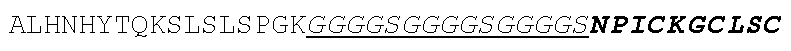 |
| | | 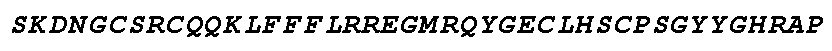 |
| | | 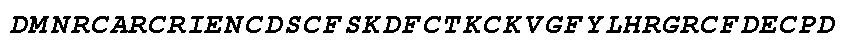 |
| | | 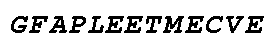 |
| 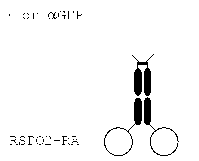 | F12578- RSPO2RA | F12578 Light Chain SEQ ID NO: 22 DIELTQPPSVSVAPGQTARISCSGCNIGSFYVHWYQQKPGQA PVLVIYDKSNRPSGIPERFSGSNSGNTATLTISGTQAEDEAD YYCQSYANTLSLVFGGGTKLTVLGQPKAAPSVTLFPPSSEEL QANKATLVCLISDFYPGAVTVAWKADSSPVKAGVETTTPSKQ SNNKYAASSYLSLTPEQWKSHRSYSCQVTHEGSTVEKTVAPT ECS F12578-RSPO2RA Heavy Chain SEQ ID NO: 34 EVGLVESGGGLVQPGGGSLTLSCAASGFTFSHTLSWVRQAPG KGLEWVSVISGDGSYTYYADSVKGRFTISSDNSKNTLYLQMN |
| | | SLRAEDTAVYYCARNFIKYVFANWGQGTLVTVSSASTKGPSV |
| | | FPLAPSSKSTSGGTAALGCLVKDYFPEVTVSWNSGALTSGV |
| | | HTFPAVLQSSGLYSLSSVVTVPSSSLGTQTYICVNHKPSNT |
| | | KVDKKVEPKSCDKTHTCPPCPAPEAAGGPSVFLFPPKPKDTL |
| | | MISRTPEVTCVVVDVSHEDPEVKFNWYVDGVEVHNAKTKPRE |
| | | EQYNSTYRVVSVLTVLHQDWLNGKEYKCKVSNKALGAPIEKT |
| | | ISKAKGQPREPQVYTLPPSREEMTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGKGGGGSGSGGSGGGG |
| | | 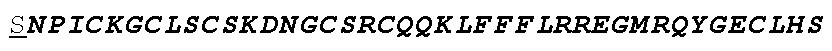 |
| | | 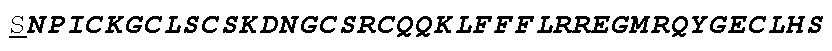 |
| | | 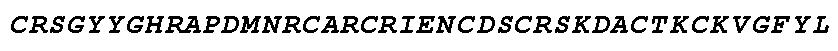 |
| 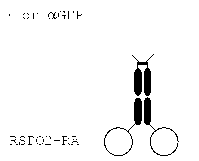 | αGFP- RSPO2RA | αGFP Light Chain SEQ ID NO: 35 DIELTQPPSVSVAPGQTARISCSGDNLGKKYVVWYQQKPGQA PVLVIYGDDERPSGIPERFSGSNSGNTATLTISGTQAEDEAD YYCASYDSSHILIVFGGGTKLTVLGQPKAAPSVTLFPPSSEE LQANKATLVCLISDFYPGAVTVAWKADSSPVKAGVETTTPSK QSNNKYAASSYLSLTPEQWKSHRESYSCQVTHEGSTVEKTVAP TECS αGFP Heavy Chain RSPO2RA SEQ ID NO: 36 QVQLVESGGGLVQPGGSLRLSCAASGFTFSRYGMHWVRQAPG KGLEWVSGISSIGSNTYYADSVKGRFTISRDNSKNTLYLQMN |
| | | SLRAEDTAVYYCARWYKTYIDVWGQGTLVTVSSASTKGPSVF |
| | | PLAPSSKSTSGGTAALGCLVKDYFPEPVTVSWNSGALTSGVH |
| | | TFPAVLQSSGLYSLSSVVTVPSSSLGTQTYICNVNHKPSNTK |
| | | VDKKVEPKSCDKTHTCPPCPAPEAAGGPSVFLFPPKPKDTLM |
| | | ISRTPEVTCVVVDVSHEDPEVKFNWYVDGVEVHNAKTKPREE |
| | | QYNSTKYRVVSVLTVLHQDWLNGKEYKCKVSNKALGAPIEKTI |
| | | SKAKGQPREPQVYTLPPSREEMTKNQVLTCLVKGFYPSIDA |
| | | VEWESNQPENNYKTTPPVLDSDGSFFLYSKLTVDKSRWQQG |
| | | NVFSCSVMHEALHNHYTQKSLSLSPGKGGGGSGSGGSGGGGS |
| | | 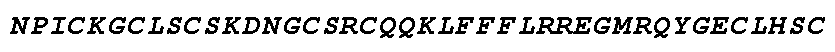 |
| | | 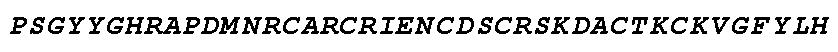 |
| | | 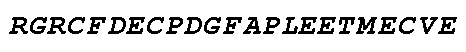 |
| 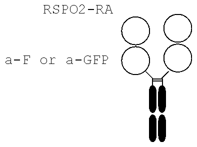 | RSPO2RA-F6 | F6 Light Chain SEQ ID NO: 37 QAVVLQEPSLSVSPGGTVTLTCGLSSGSVSTNYYPSWYQQTP GQAPRTLIYYTNTRSSDVPERFSGSIVGNKAALTITGAQPDD ESVYFCLLYLGRGIWVFGGGTKLTVLGQPKAAPSVTLFPPSS EELQANKATLVCLISDFYPGAVTVAWKADSSPVKAGVETTTP SKQSNNKYAASSYLSLTPEQWKSHRSYSCQVTHEGSTVEKTV APTECS RSPO2RA-F6 Heavy Chain SEQ ID NO: 38 |
| | | 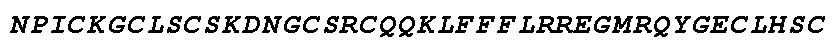 |
| | | 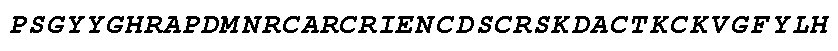 |
| | | 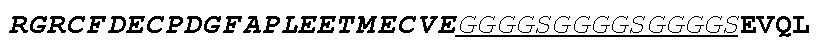 |
| | | VQSGAEVKKPGASVKVSCKASGYTFTSYGISWVRQAPGQGLE |
| | | WMGWISAYNGNTNYAQKLQGRVTMTTDTSTSTAYMELRSLRS |
| | | DDTAVYYCASSKEKATYYYGMDVWGQGTTVTVSSASTKGPSV |
| | | FPLAPSSKSTSGGTAALGCLVKDYFPEPVTVSWNSGALTSGV |
| | | HTFPAVLSSGLYSLSSVVTVPSSSLGTQTYICNVNHKPSNT |
| | | KVDKKVEPKSCDKTHTCPPCPAPEAAGGPSVFLFPPKPKDTL |
| | | MISRTPEVTCVVVDVSHEDPEVKFNWYDGVEVHNAKTKPRE |
| | | EQYNSTYRVVSVLTVLHQDWLNGKEYKCKVSNKALGAPIEKT |
| | | ISKAKGQPREPQVYTLPPSREEMTKNWVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 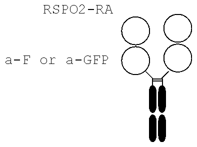 | RSPO2RA-F7 | F7 Light Chain SEQ ID NO: 39 DIVMTQSPKSMSMSVGERVTLRCKASENVLNYVSWYQQKPEQ SPKLLIYGASNRYTGVPDRFTGSGSATDFTLTISSVQAEDLA DYHCGQSYRYPTFGAGTKLELKRTVAAPSVFIFPPSDEQLKS GTASVVCLLNNFYPREAKVQWKVDNALQSGNSQESVTEQDSK DSTYSLSSTLTLSKADYEKHKVYACEVTHQGLSSPVTKSFNR GEC RSPO2RA-F7 Heavy Chain SEQ ID NO: 40 |
| | | 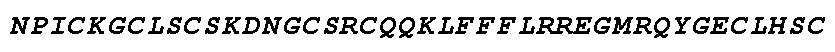 |
| | | 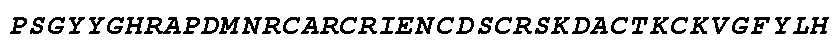 |
| | | 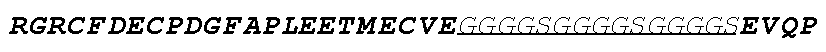 |
| | | VESGGGLVQPKGSLKLSCAASGFTFNTYAMHWVRQAPGKGLE |
| | | WVARIRSKSNNYAKNYDDSVKDRFTISRDDSQSMLYLQMNNL |
| | | KTEDTAMYYCVRENYGGRFDYWGQGTTLTVSSASTKGPSVFP |
| | | LAPSSKSTSGGTAALGCLVKDYFPEPVTVSWNSGALTSGVHT |
| | | FPAVLQSSGLYSLSSVVTVPSSSLGTQTYICNVNHKPSNTKV |
| | | DKKVEPKSCDKTHTCPPCPAPEAAGGPSVFLFPPKPKDTLMI |
| | | SRTPEVTCVVVDVSHEDPEVKFNWYVDGVEVHNAKTKPREEQ |
| | | YNSTYRVVSVLTVLHQDWLNGKEYKCKVSNKALGAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQGN |
| | | VFSCSVMHEALHNHYTQKSLSLSPGK |
| 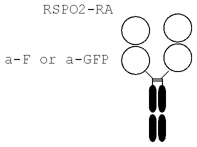 | RSPO2RA- αGFP | αGFP Light Chain SEQ ID NO: 35 DIELTQPPSVSVAPGQTAISCSGDNLGKKYVYWYQQKPGQAPV LVIYGDDERPSGIPERFSGSNSGNTATLTISGTQAEDEADYYCA SYDSSHILIVFGGGTKLTVLGQPKAAPSVTLFPPSSEELQANKA TLVCLISDFYPGAVTVAWKADSSPVKAGVETTTPSKQSNNKYAA SSYLSLTPEQWKSHRSYSCQVTHEGSTVEKTVAPTECS RSPO2RA-αGFP Heave Chain SEQ ID NO: 41 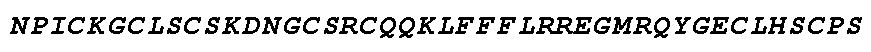 |
| | | 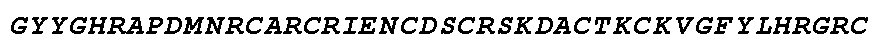 |
| | | 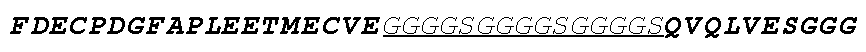 |
| | | LVQPGGSLRLSCAASGFTFSRYGMHWVRQAPGKGLEWVSGISSI |
| | | GSNTYYADSVKGRFTISRDNSKNTLYLQUMNSLRAEDTAVYYCAR |
| | | WYKTYIDVWGQGTLVTVSSASTKGPSVFPLAPCSRSTSESTAA |
| | | GCLVKDYFPEPVTVSWNSGALTSGVHTFPAVLQSSGLYSLSSVV |
| | | TVPSSNFGTQTYTCNVDHKPSNTKVDKTVERKCCVECPPCPAPP |
| | | VAGPSVFLFPPKPKDTLMISRTPEVTCVVVDVSHEDPEVQFNWY |
| | | VDGVEVHNAKTKPREEQFNSTFRVVSVLTVVHQDWLNGKEYKCK |
| | | VSNKGLPAPIEKTISKTKGQPREPQVYTLPPSREEMTKNQVSLT |
| | | CLVKGFYPSDIAVEWESNGQPENNYKTTPPMLDSDGSFFLYSKL |
| | | TVDKSRWQQGNVFSCSVMHEALHNHYTQKSLSLSPGK |
| 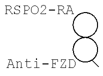 | RSPO2RA-F6 Fab | F6 Light Chain SEQ ID NO: 37 QAVVLQEPSLSVSPGGTVTLTCGLSSGSVSTNYYPSWYQQTPGQ APRTLIYYTNTRSSDVPERFSGSIVGNKAALTITGAQPDDESVY FCLLYLGRGIWVFGGGTKLTVLGQPKAAPSVTLFPPSSEELQAN KATLVCLISDFYPGAVTVAWKADSSPVKAGVETTTPSKQSNNKY |
| | | AASSYLSLTPEQWKSHRSYSCQVTHEGSTVEKTVAPTECS |
| | | RSPO2RA-F6-VH-CH1 SEQ ID NO: 42 |
| | | 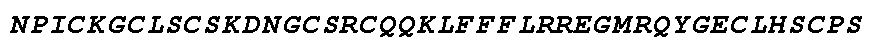 |
| | | 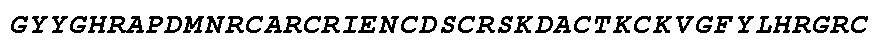 |
| | | 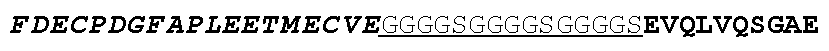 |
| | | VKKPGASVKVSCKASGYTFTSYGISWVRQAPGQGLEWMGWISAY |
| | | NGNTNYAQKLQGRVTMTTDTSTSTAYMELRSLRSDDTAVYYCAS |
| | | SKEKATYYYGMDVWGQGTTVTVSSASTKGPSVFPLAPSSKSTSG |
| | | GTAALGCLVKDYFPEPVTVSWNSGALTSGVHTFPAVLQSSGLYS |
| | | LSSVVTVPSSSLGTQTYICNVNHKPSNTKVDKKVEPKSCGSGSG |
| | | HHHHHH |
| 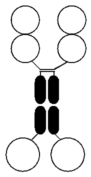 | L6-F12578- RSPO2RA | L6-F12578 Light Chain SEQ ID NO: 20 DIQMTQSPSSLSASVGDRVTITCRASQDVSTAVAWYQQKPGKA PKLLIYSASFLYSGVPSRFSGSGSGTDFTLTISSLQPEDFATY YCQQSYTTPPTFGQGTKVEIKGGGGSGGGGSGGGGSDIELTQP PSVSVAPGQTARISCSGDNIGSFYVHWYQQKPGQAPVLVIYDK SNRPSGIPERFSGSNSGNTATLTISGTQAEDEADYYCQSYANT LSLVFGGGTKLTVLGQPKAAPSVTLFPPSSEELQANKATLVCL ISDFYPGAVTVAWKADSSPVKAGVETTTPSKQSNNKYAASSYL SLTPEQWKSHRSYSCQVTHEGSTVEKTVAPTECS L6-F12478-RSPO2RA heavy Chain SEQ ID NO: 48 EVQLVESGGGLVQPGGSLRLSCAASGFTFTSYYISWVRQAPGK |
| | | GLEWVAEISPYSGSTYYADSVKGRFTISADTSKNTAYLQMNSL |
| | | RAEDTAVYYCALRARPPIRLHPRGSVMDYWGQGTLVTVSSGGG |
| | | GSGGGGSGGGGSEVQLVESGGGLVQPGGSLRLSCAASGFTFSH |
| | | YTLSWVRQAPGKGLEWVSVISGDGSYTYYADSVKGRFTISSDN |
| | | SKNTLYLQMNSLRAEDTAVYYCARFIKYVFANWGQGTLVTVS |
| | | SASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPEAAGGPSVFLFPP |
| | | KPKDTLMISRTPEVTCVVVDVSHEDPEVKFNWYVDGVEVHNAK |
| | | TKPREEQYNSTYRVVSVLTVLHQDWLNGKEYKCKVSNKALGAP |
| | | IEKTISKAKGQPREPQVYTLPPSREEMTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPGKGGGGSGSGGSGGG |
| | | 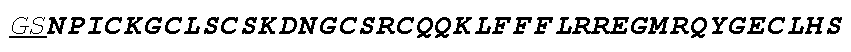 |
| | | 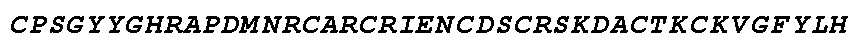 |
| | | 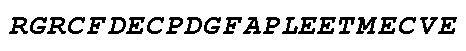 |
| 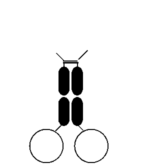 | F127-RSPO2RA | F127 Light Chain SEQ ID NO: 49 DIQMTQSPSSLSASVGDRVTITCRASQSVSSAVAWYQQ KPGKAPKLLIYSASSLYSGVPSRFSGSRSGTDFTLTISSL QPEDFATYYCQQYWYGVAPITFGQGTKVEIKRTVAAP SVFIFPPSDEQLKSGTASVVCLLNNFYPREAKVQWKVDNA LQSGNSQESVTEQDSKDSTYSLSSTLTLSKADYEKHKVYA CEVTHQGLSSPVTKSFNRGEC F127-RSPO2RA Heavy Chain SEQ ID NO: 50 EVQLVESGGGLVQPGGSLTLSCAASGFNISSSYIHWVR QAPGKGLEWVAYIYSSYGSTYYADSVKGRFTISADTSK |
| | | NTAYLQMNSLRAEDTAVYYCARASWYALDYWGQGTL |
| | | VTVSSASTKGPSASTKGPSVFPLAPSSKSTSGGTAALGCLV |
| | | KDYFPEPVTVSWNSGALTSGVHTFPAVLQSSGLYSLSSVV |
| | | TVPSSSLGTQTYICNVNHKPSNTKVDKKVEPKSCDKTHTC |
| | | PPCPAPEAAGGPSVFLFPPKPKDTLMISRTPEVTCVVVDVS |
| | | HEDPEVKFNWYVDGVEVHNAKTKPREEQYNSTYRVVSV |
| | | LTVLHQDWLNGKEYKCKVSNKALGAPIEKTISKAKGQPR |
| | | EPQVYTLPPSREEMTKNQVSLTCLVKGFYPSDIAVEWESN |
| | | GQPENNYKTTPPVLDSDGSFFYSKLTVDKSRWQQGNVF |
| | | SCSVMHEALHNHYTQKSLSLSPGKGGGGSGSGGSGGGGS |
| | | 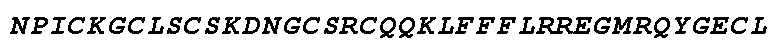 |
| | | 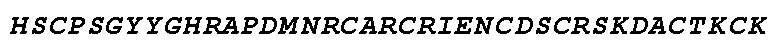 |
| | | 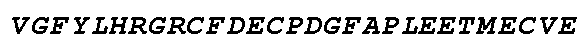 |
| 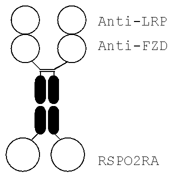 | L6-F127- RSPO2RA | L6-F127 Light Chain SEQ ID NO: 25 DIQMTQSPSSLSASVGDRVTITCRASQDVSTAVAWYGGKPGKA PKLLIYSASFLYSGVPSRFSGSGSGTDFTLTISSLQPEDFATY YCQQSYTTPPTFGQGTKVEIKGGGGSGGGGSGGGGSDIQMTQS PSSLSASVGDRVTITCRASQSVSSAVAWYGGKPGKAPKLLIYS ASSLYSGVPSRFSGSRSGTDFTLTISSLQPEDFATYYCQQYWY GVAPITFGQGTKVEIKRTVAAPSVFIFPPSDEQLKSGTASVVC LLNNFYPREAKVQWKVDNALQSGNSQESVTEQDSKDSTYSLSS TLTLSKADYEKHKVYACEVTHQGLSSPVTKSFNRGEC L6-F127-RSPO2RA Heavy Chain DEQ ID NO: 51 |
| | | EVQLVESGGGLVQPGGSLRLSCAASGFTFTSYYISWVRQAPGK |
| | | GLEWVAEISPYSGSTYYADSVKGRFTISADTSKNTAYLQMNSL |
| | | RAEDTAVYYCALRARPPIRLHPRGSVMDYWGQGTLVTVSSGGG |
| | | GSGGGGSGGGGSEVQLVESGGGLVQPGGSLRLSCAASGFNISS |
| | | SYIHWRVQAPGKGLEWVAYIYSSYGSTYYADSVKGRFTISADT |
| | | SKNTAYLQMNSLRAEDTAVYYCARASWYALDYWGQGTLVTVSS |
| | | ASTKGPSVFPLAPSSKSTSGGTAALGCLVKDYFPEPVTVSWNS |
| | | GALTSGVHTFPAVLQSSGLYSLSSVVTVPSSSLGTQTYICNVN |
| | | HKPSNTKVDKKVEPKSCDKTHTCPPCPAPEAAGGPSVFLFPPK |
| | | PKDTLMISRTPEVTCVVVDVSHEDPEVFKFNWYDGVEVHNAKT |
| | | KPREEQYNSTYRVVSVLTVLHQDWLNGKEYKCKVSNKALGAPI |
| | | EKTISKAKGQPREPQVYTLPPSREEMTKNQVSLTCLVKGFYPS |
| | | DIAVEWESNGQPENNYKTTPPVLDSDGSFFLYSKLTVDSRWQ |
| | | QGNVFSCSVMHEALHNHYTQKSLSLSLSPGKGGGGSGSGGSGGGG |
| | | 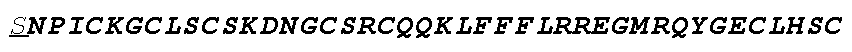 |
| | | 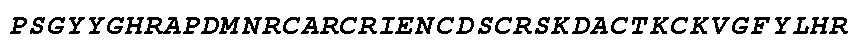 |
| | | 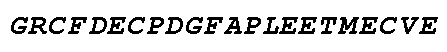 |
| 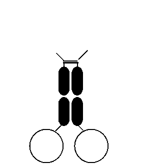 | F58-RSPO2RA | F58 Light Chain SEQ ID NO: 52 DIQMTQSPSSLSASVGDRVTITCRASQSVSSAVAWYQQKPGKA PKLLIYSASSLYSGVPSRFSGSRSGTDFTLTISSLQPEDFATY YCQQWYSSGHVLITFGQGTKVEIKRTVAAPSVFIFPPSDEQLK SGTASVVCLLNNFYPREAKVQWKVDNALQSGNSQESVTEQDSK DSTYSLSSTLTLSKADYEKHKVYACEVTHQGLSSPVTKSFNRG EC F8-RSPO2RA Heavy Chain SEQ ID NO: 53 EVQLESGGGLVQPGGSLRLSCAASGFNISYSIHVWVRQAPGK GLEWVASIYSSSGSTSYADSVKGRFTISADTSKNTAYLQMNSL |
| | | RAEDTAVYYCARGAIDYWGQGTLVTVSSASTKGPSVFPLAPSS |
| | | KSTSGGTAALGCLVKDYFPEPVTVSWNSGALTSGVHTFPAVLQ |
| | | SSGLYSLSSVVTVPSSSLGTQTYICNVNHKPSNTKVDKKVEPK |
| | | SCDKTHTCPPCPAPEAAGGSVFLFPPKPKDTLMISRTPEVTC |
| | | VVVDVSHEDPEVKFNWYVDGVEVHNAKTKPREEQYNSTYRVVS |
| | | VLTVLHQDWLNGKEYKCKVSNKALGAPIEKTISKAKGQPREPQ |
| | | VYTLPPSREEMTKNQVSLTCLVKGFYPSDIAVEWESNGQPENN |
| | | YKTTPPVLDSDGSFFLYSKLTVDKSRWQQGNVFSCSVMHEALH |
| | | 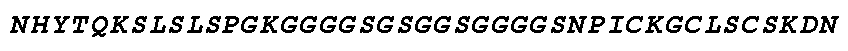 |
| | | 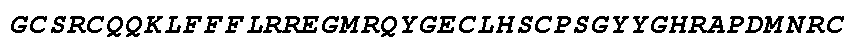 |
| | | 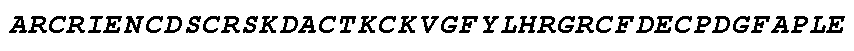 |
| | | 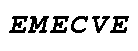 |
| 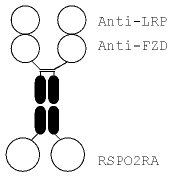 | L6-F58- RSPO2RA | L6-F58 Light Chain SEQ ID NO: 27 DIQMTQSPSSLSASVGDRVTITCRASQDVSTAVAWYQQKPGKA PKLLIYSASFLYSGVPSRFSGSGSGTDFTLTISSLQPEDFATY YCQQSYTTPPTFGQGTKVEIKGGGGSGGGGSGGGGSDIQMTQS PSSLSASVGDRVTITCRASQSVSSAVAWYQQKPGKAPKLLIYS ASSLYSGVPSRFSGSRSGTDFTLTISSLQPEDFATYYCQQWYS SGHVLITFGQGTKVEIKRTVAAPSVFIFPPSDEQLKSGTASVV CLLNNFYPREAKVQWKVDNALQSGNSQESVTEQDSKDSTYSLS STLTLSKADYEKHHVYACEVTHQGLSSPVTKSFVRGEC L6-58_RSPO2RA Heavy Chain SEQ ID NO: 54 EVQLVESGGGLVQPGGSLRLSCAASGFTFTSYYISWVRQAPGK |
| | | GLEWVAEISPYSGSTYYADSVKGRFTISADTSKNTAYLQMNSL |
| | | RAEDTAVYYCALRARPPIRLHPRGSVMDYWGQGTLVTVSSGGG |
| | | GSGGGGSGGGGSEVQLESGGGLVQPGGSLRLSCAASGFNISY |
| | | SYIHWVRQAPGKGLEWVASIYSSSGSTSYADSVKGRFTISADT |
| | | SKNTAYLQMNSLRAEDTAVYYCARGAIDYWGQGTLVTVSSAST |
| | | KGPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKKVEPKSCDKTHTCPPCPAPEAAGGPSVFLFPPKPKD |
| | | TLMISRTPEVTCVVVDVSHEDPEVKFNWYVDGVEVHNAKTKPR |
| | | EEQYNSTYRVVSVLTVLHQDWLNGKEYKCKVSNKALGAPIEKT |
| | | ISKAKGQPREPQVYTLPPSREEMTKNQVSLTCLVKGFYPSDIA |
| | | VEWESNGQPENNYKTTPPVLDSDGSFFLYSKLTVDKSRWGGGN |
| | | 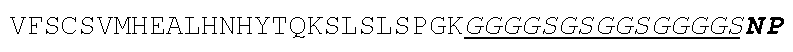 |
| | | 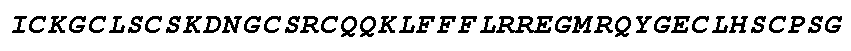 |
| | | 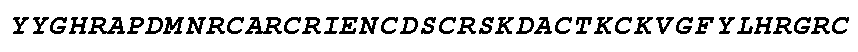 |
| | | 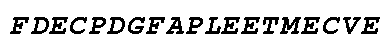 |
| 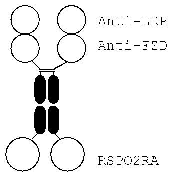 | L2-F6- RSPO2RA | L2-F6 Light Chain SEQ ID: NO: 43 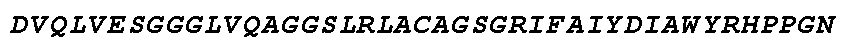 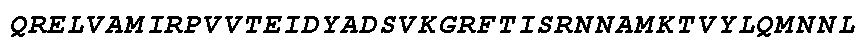 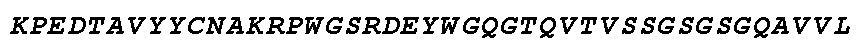 QEPSLSVSPGGTVTLTCGLSSGSVSTNYYPSWYQQTPGQAPRT LIYYTNTRSSDVPERFSGSIVGNKAALTITCAQPDDESVYFCL LYLGRGIWVFGGGTKLTVLGQPKAAPSVTLFPPSSEELQANKA TLVCLISDFYPGAVTVAWKADSSPVKAGVETTTPSKQSNNKYA ASSYLSLTPEQWKSHRSYSCQVTHEGSTVEKTVAPTECS |
| | | F6-RSPO2RA Heavy Chain SEQ ID: NO: 44 |
| | | EVQLVQSGAEVKKPGASVKVSCKASGYTFTSYGISWVRQAPGQ |
| | | GLEWMGWISAYNGNTNYAQKLQGRVTMTTDTSTSTAYMELRSL |
| | | RSDDTAVYYCASSKEKATYYYGMDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKKVEPKSCDKTHTCPPCPAPEAAGGPSVFLFPPKPKDTLMI |
| | | SRTPEVTCVVVDVSHEDPEVKFNWYVDGVEVHNAKTKPREEQY |
| | | NSTYRVVSVLTVLHQDWLNGKEYKCKVSNKALGAPIEKTISKA |
| | | KGQPREPQVYTLPPSREEMTKNQVSLTCLVKGFYPSDIAVEWE |
| | | SNGQPENNYKTTPPVLDSDGSFFLYSKLTVDKSRWQQGNVFSC |
| | | 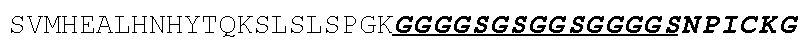 |
| | | 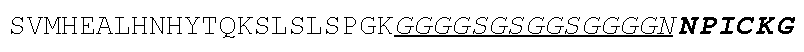 |
| | | 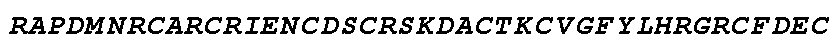 |
| | | 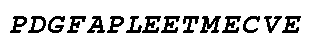 |
| 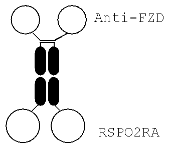 | F6-RSPO2RA | F6 Light Chain SEQ ID NO: 37 QAVVLQEPSLSVSPGGTVTLTCGLSSGSVSTNYYPSWYQQTPG QAPRTLIYYTNTRSSDVPERFSGSIVGNKAALTITGAQPDDES VYFCLLYLGRGIWVFGGGTKLTVLGQPKAAPSVTLFPPSSEEL QANKATLVCLISDFYPGAVTVAWKADSSPVKAGVETTTPSKQS NNKYAASSYLSLTPEQWKSHRSYSCQVTHEGSTVEKTVAPTEC S F6-RSPO2RA Heavy Chain SEQ ID NO: 44 EVQLVQSGAEVKKPGASVKVSCKASGYTFTSYGISWVRQAPGQ |
| | | GLEWMGMISAYNGNTNYAQKLQGRVTMTTDTSTSTAYMELREL |
| | | RSDDTAVYYCASSKEKATYYYGMDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKKVEPKSCDKTHTCPPCPAPEAAGGPSVFLFPPKPKDTLMI |
| | | SRTPEVTCVVVDVSHEDPEVKFNWYVDGVEVHNAKTKPREEQY |
| | | NSTYRVVSVLTVLHQWLNGKEYKCKVSNKALGAPIEKTISKA |
| | | KGQPREQVYTLPPSREEMTKNQSLTCLVKGFYPSIAVEWE |
| | | SNQPENNYKTTPPVLDSDGSFFLYSKLTVDKSRWQQGNVFSC |
| | | 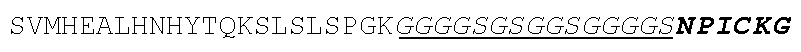 |
| | | 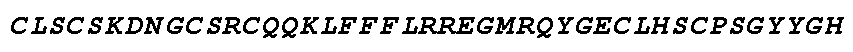 |
| | | 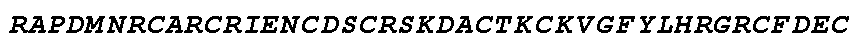 |
| | | 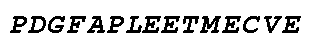 |
| 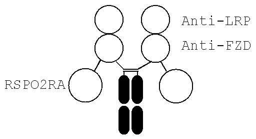 | L2-F6- RSPO2R@ CL | L2-F6-FSPO2RA Light Chain SEQ ID NO: 45 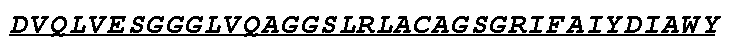 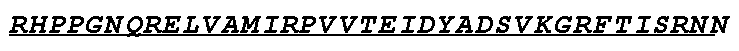 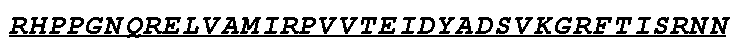 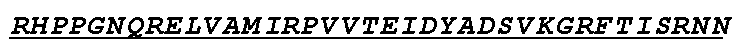 SSGSVSTNYYPSWYQQTPGQAPRTLIYYTNTRSSDVP |
| | | ERFSGSIVGNKAALTITGAQPDDESVYFCLLYLGRGI |
| | | WVFGGGTKVTVLGQPKAAPSVTLFPPSSEELQANKAT |
| | | LVCLISDFYPGAVTVAWKADSSPVKKAGVETTTPSKQS |
| | | NNKYAASSYLSLTPEQWKSHRSYSCQVTHESGSTVEKT |
| | | 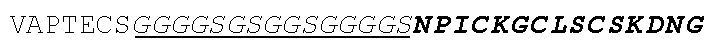 |
| | | 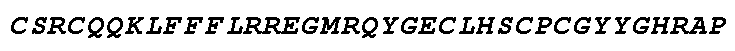 |
| | | 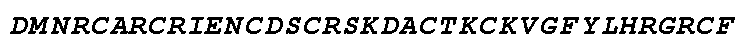 |
| | | 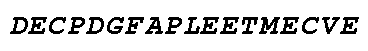 |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLMIS |
| | | RTPEVTCVVVDVSHEDPEKFNWYVDGVEVHNAKTKP |
| | | REEQYNSTYRVVSVLTVLHQDWLNGKEYKCKVSNKAL |
| | | GAPIEKTISKAKGQPREPQVYTLPPSREEMTKNQVSL |
| | | TCLVKGFYPSDIAVEWESNGQPENNYKTTPPVLDSDG |
| | | SFFLYSKLTVDKSRWQQGNFSCSVMHEALHNHYTQK |
| | | SLSLSPGK |
| 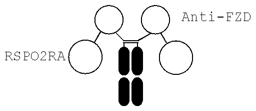 | F6- RSO2RA@ CL | F6-RSPO2RA Light Chain SEQ ID NO: 47 QAVVLQEPSLSVSPGGTVTLTCGLSSGSVSTNYYPSW YQQTPGQAPRTLIYYTNTRSSDVPERFSGSIVGNKAA LTITGAQPDDESVYFCLLYLGRGIWVFGGGTKLTVLG QPKAAPSVTLFPPSSEELQANKATLVCLISDFYGAV TVAWKADSSPVKAGVETTTPSKQSNNKYAASSYLSLT PEQWKSHRSYSCQVTHEGSTVEKTVAPTECSGGGGSG |
| | | 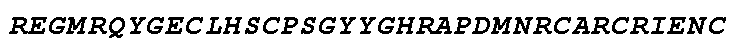 |
| | | 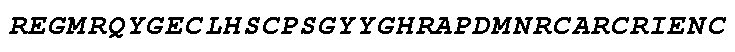 |
| | | 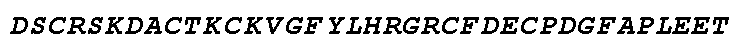 |
| | | 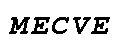 |
| | | F6 Heavy Chain SEQ ID NO: 46 |
| | | EVQLVQSGAEVKKPGASVKVSCKASGYTTSGISWV |
| | | RQAPGQGLEWMGWISAYNGNTNYAQKLQGRVTMTTDT |
| | | STSTAYMELRSLRSDDTAVYYCASSKEKATYYYGMDV |
| | | WGQGTTVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDTHTCPPCPAPEAAGGPSVFLFPPKPKDTIMIS |
| | | RTPEVTCVVVDVSHEDPEVKFNWYVDGVEVHNAKTKP |
| | | REEQYNSTYRVVSVLTVLHQDWLNGKEYKCKVSNKAL |
| | | GAPIEKTISKAKGQPREPQVYTLPPSREEMTKNQVSL |
| | | TCLVKGFYPSDIAVEWESNGQPENNYKTTPPVLDSDG |
| | | SFFLYSKLTVDKSRWQQGNVFSCSVMHEALHNHYTQK |
| | | SLSLSPGK |
| | F4-2- | R4-2_LC SEQ ID NO: 98 |
| | RSPO2RA | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQSYAAYLFT |
| | | FGQGTKVEIKRTVAAPSVFIFPPSDEQLKSGTAS |
| | | VVCLLNNFYPREAKVQWKVDNALQSQNSQESV |
| | | TEQDSKDSTYSLSSTLTLSKADYEKHKVYACEVT |
| | | HQGLSSVTKSFNRGEC |
| | | >F4-2_RSPO2RA_HC SEQ ID NO: 99 |
| | | EVQLVESGGGLVQPGGSLRTSCAASGFNISYY |
| | | YIHWVRQAPGKGLEWVASIYPSSGYTYYADSV |
| | | KGRFTISADTSKNTAYLQMNSLRAEDTAVYYC |
| | | ARSSFYWAMDYWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSGTQ |
| | | TYICNVNHKPSNTKVDKKVEPKSCDKTHTCPPCP |
| | | APEAAGGPSVFLPPKPKDTLMISRTPEVTCVVV |
| | | DVSHEDPEVKFNWYBDVEVHNAKTKREEQY |
| | | NSTYRVVSVLTVLHQDWLNGKEYKCKVSNKAL |
| | | GAPIEKTISKAKGQPREPQVYTLPPSEEMTKNQ |
| | | VSLTCLVKGFYPSDIAVEWESNGQPENNYKTTPP |
| | | VLDSDGFFLYSKLTVDKSRWQQGNVFSCSVMH |
| | | EALHNHYTQKSLSLSPGKGGGGSGSGGSGGGGS |
| | | 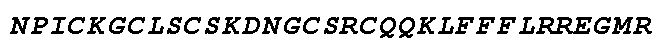 |
| | | 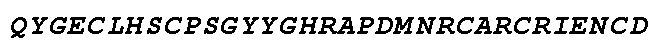 |
| | | 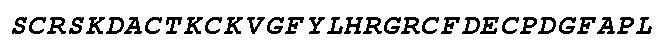 |
| | | 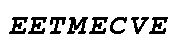 |
| | F10- | F10_LC SEQ ID NO: 100 |
| | RSPO2RA | ELTQPPSVSVSPGQTARITCSGDGSYAGSYYYG |
| | | WYQQKPGQAPVTLIYNNKRPSGIPERFSGSL |
| | | SGSTNTLTISGVQAEDEADYYCGSADNSGAAF |
| | | GGGTKLTVLRTVAAPSVFIFPPSDEQLKSGTASV |
| | | VCLLNNFYPREAKVQWKVDNALQSGNSQESVTE |
| | | QDKDSTYSLSSTLTLSKADYEKHKVYACEVTH |
| | | QGLSSPVTKSFNRGEC |
| | | >F10_RSPO2RA_HC SEQ ID NO: 101 |
| | | EVQLVESGGGLVQPGGSLRLSCAASGFTFSSF |
| | | NMFWVRQAPGKGLEWVAGIDDDGSYPNYGS |
| | | AVKGRATISRDNSKNTLYLQMNSLRAEDTAVY |
| | | YCAKSGYGGSWGGYIADDIDAWGQGTLVTVS |
| | | SASTKGPSVFPLAPSSKSTSGGTAALGCLVKDYF |
| | | PEPVTVSWNSGALTSGVHTFPAVLQSSGLYSLSS |
| | | VVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVH |
| | | NAKTKPREEQYNSTYRVVSVLTVLHQDWLNGK |
| | | EYKCKVSNKALGAPIEKTISKAKGQPREPQVYTL |
| | | PPSREEMTKNQVSLTCLVKGFYPSDIAVEWESNG |
| | | QPENNYKTTPPVLDSDGSFFLYSKLTVDKSRWQ |
| | | QGNVSCSYMHEALHNHYTQKSLSLSPGKGGGG |
| | | 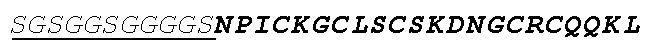 |
| | | 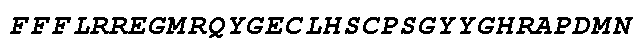 |
| | | 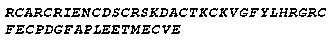 |
| | anti-GFP- | anti-GFP_LC SEQ ID NO NO: 102 |
| | RSPO2RA | DIELTQPPSVSVAPGQTARISCSGDNLGKKYVY |
| | | WYQQKPGQAPVLVIYGDDERPSGIPERFSGSN |
| | | SGNTATLTISGTQAEDEADYYCASYDSSHILIV |
| | | FGGGTKLTVLGQPKAAPSVTLFPPSSEELQANK |
| | | ATLVCLISDFYPGAVTVAWKADSSPVKAGVETT |
| | | TPSKQSNNKYAASSYLSLTPEQWKSHRSYSCQVT |
| | | HEGSTVEKTVAPTECS |
| | | >anti-GFP_RSPO2RA_HC SEQ ID NO: 103 |
| | | QVQLVESGGGLVQPGGSLRLSCAASGFTFSRY |
| | | GMHWVRQAPGKGLEWVSGISSIGSNTYYADS |
| | | VKGRFTISRDNSKNTLYLQMNSLRAEDTAVYY |
| | | CARWYKTYIDVWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPSREEMTKNQ |
| | | VSLTCLVKGFYPSDIAVEWESNGQPENNYKTTPP |
| | | VLDSDGSFFLYSKLTVDKSRWQQGNVFSCVMH |
| | | EALHNHYTQKSLSLSPGKGGGGSGSGGSGGGGS |
| | | 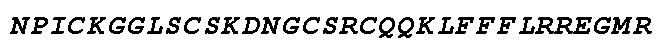 |
| | | 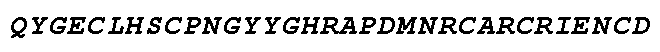 |
| | | 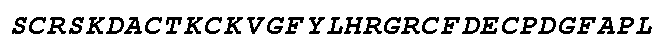 |
| | | 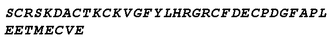 |
| Fv-IgG | L6-F2-2- | L6-F4-2-RSPO2RA_HC SEQ ID NO: 103 |
| | RSPO2RA | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSG |
| | | GGGSGGGGSGGGGSEVQLVESGGGLVQPGGSL |
| | | RLSCAASGFNISYYYIHWVRQAPGKGLEWVAS |
| | | IYPSSGYTYYADSVKGRFTISADTSKNTAYLQM |
| | | NSLRAEDTAVYYCARSSFYWAMDYWGQGTL |
| | | VTVSSASTKGPSVFPLAPSSKSTSGGTAALGCLV |
| | | KDYFPEPVTVSWNSGALTSGVHTFPAVLQSSGLY |
| | | SLSSVVTVPSSSLGTQTYICNVNHKPSNTKVDKK |
| | | VEPKSCDKTHTCPPCPAPEAAGGPSVFLFPPKPK |
| | | DTLMISRTPEVTCVVVDVSHEDPEVKFNWYVDG |
| | | VEVHNAKTKPREEQYNSTYRVVSVLTVLHQDW |
| | | LNGKEYKCKVSNKALGAPIEKTISKAKGQPREPQ |
| | | VYTLPPSREEMTKNQVSLTCLVKGFYPSDIAVEW |
| | | ESNGQPENNYKTTPPVLDSDGSFFLYSKLTVDKS |
| | | RWQQGNVFSCSVMHEALHNHYTQKSLSLSPGK |
| | | 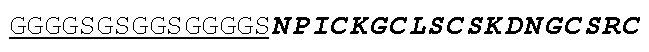 |
| | | 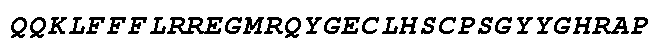 |
| | | 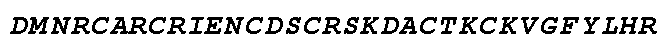 |
| | | 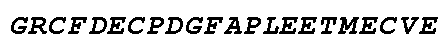 |
| | | >L6-F4-2_LC SEQ ID NO: 104 |
| | | DIQMTQSPSSLSASVGDRVTITCRASQDVSTAV |
| | | AWYQQKPGKAPKLLIYSASFLYSGVPSRFSGS |
| | | GSGTDFTLTISSLQPEDFATYYCQQSYTTPPTF |
| | | GQGTYKVEIKGGGGSGGGGSGGGGSDIQMTQSP |
| | | SSLSASVGDRVTITCRASQSVSSAVAWYQQKP |
| | | GKAPKLLIYSASSLYSGVPSRFSGSRSGTDFTL |
| | | TISSLQPEDFATYYCQQSYAAYLFTFGQGTKV |
| | | EIKRTVAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| Fv-IgG | L6-F10- | L6-F10-RSPO2RA_HC SEQ ID NO: 105 |
| | RSPO2RA | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSG |
| | | GGGSGGGGSGGGGSEVQLVESGGGLVQPGGSL |
| | | RLSCAASGFTFSSFNMFWVRQAPGKGLEWVA |
| | | GIDDDGSYPNYGSAVKGRATISRDNSKNTLYL |
| | | QMNSLRAEDTAVYYCAKSGYGGSWGGYIADD |
| | | IDAWGQGTLVTVSSASTKGPSVFPLAPSSKSTSG |
| | | GTAALGCLVKDYFPEPVTVSWNSGALTSGVHTF |
| | | PAVLQSSGLYSLSSVVTVPSSSLGTQTYICNVNH |
| | | KPSNTKVDKKVEPKSCDEKTHTCPPCPAPEAAGGP |
| | | SVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTIS |
| | | KAKGQPREPQVYTLPPSREEMTKNQVSLTCLVK |
| | | GFYPSDIAVEWESNGQPENNYKTTPPVLDSDGSF |
| | | FLYSKLTVDKSRWQQGNVFSCSBMHEALHNHY |
| | | 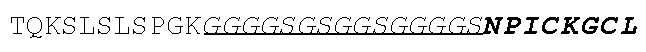 |
| | | 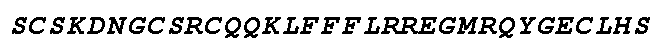 |
| | | 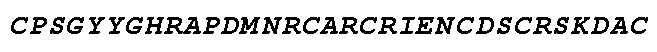 |
| | | 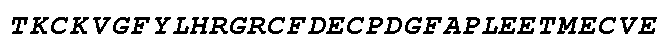 |
| | | >L6-F10_LC SEQ ID NO: 106 |
| | | DIQMTQSPSSLSASVGDRVTITCRASQDVSTAV |
| | | AWYQQKPGKAPKLLIYSASFLYSGVPSRFSGS |
| | | GSGTDFTLTISSLQPEDFATYYCQQSYTTPPTF |
| | | GQGTKVEIKGGGGSGGGGSGGGGSELTQPPSVS |
| | | VSPGQTARITCSGDGSYAGSYYYGWYQQKPG |
| | | QAPVTLIYYNNKRPSGIPERFSGSLSGSTNTLTI |
| | | SGVQAEDEADYYCGSADNSGAAFGGGTKLTV |
| | | LRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| (F4)Fv-IvG | L6-F4- | L6-F4-RSPO2RA HC SEQ ID NO: 107 |
| | RSPO2RA | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNLSSYSMHWVRQAPGKG |
| | | LEWVAYISSYYGYTTYYADSVKGRFTISADTSKNTAYL |
| | | QMNSLRAEDTAVYYCARPAPGHWGFDYWGQGTLVTVS |
| | | SASTKGPSVFPLAPSSKSTSGGTAALGCLVKDYFPEP |
| | | VTVSWNSGALTSGVHTFPAVLQSSGLYSLSSVVTVPS |
| | | SSLGTQTYICNVNHKPSNTKYVDKKVEPKSCDKTHTCP |
| | | PCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGKGG |
| | | 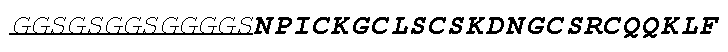 |
| | | 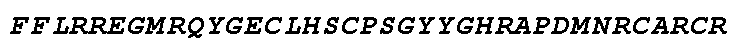 |
| | | 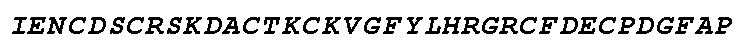 |
| | | 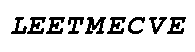 |
| | | L6-F4-RSPO2RA LC SEQ ID NO: 108 |
| | | DIQMTQSPSSLSASVGDRVTITCRASQDVSTAVAWQ |
| | | QKPGKAPKLLIYSASFLYSGVPSRFSGSGSGTDFTLT |
| | | ISSLQPEDFATYYCQQSYTTPPTFGQGTKVEIKGGGG |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQWYYAPITFGQ |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHKVYACEVTHQGLSSPVTKSFNR |
| | | GEC |
| (F49) Fv-IgG | L6-F49- | L6-F49-RSPO2RA HC SEQ ID NO: 109 |
| | RSPO2RA | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVQS |
| | | GAEVKKPGASVKVSCKASGGTFSSYAISWVRQAPGQG |
| | | LEWMGWINAGNGNTTYAQKFQGRVTMTRDTSTSTVYM |
| | | ELSSRLSEDTAVYYCARHYYGSGSYPDWGQGTLVTVS |
| | | SASTKGPSVFPLAPSSKSTSGGTAALGCLVDEKYFPEP |
| | | PCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGKGG |
| | | 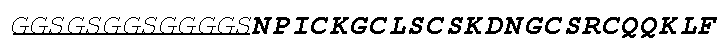 |
| | | 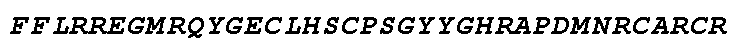 |
| | | 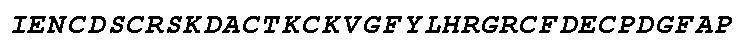 |
| | | 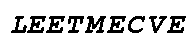 |
| | | L6-F49-RSPO2RA LC SEQ ID NO: 110 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKRGGG |
| | | GSGGGGSGGGGSDIQMTQSPSSPSASVGDRVTITCRA |
| | | SQSISSYLNWYQQKPGKAPKLLIYAASSLQSGVPSRF |
| | | SGSGSGTDFTLTISSLQPEDFATYYCQQSYSTPLTFG |
| | | GGTKVEIKRTVAAPSVFIFPPSDEQLKSGTASVVCLL |
| | | NNFYPREAKVQWKVDNALQSGNSQESVTEQDSKDSTY |
| | | SLSSTLTLSKADYEKHKVYACEVTHQGLSSPVTKSFN |
| | | RGEC |
| (F10) Fv-IgG | L6-F10- | L6-F10-RSPO2RA HC SEQ ID NO: 111 |
| | RSPO2RA | EQQLVESGGGLVQPGGSLRLSCAASGFTFTSYYISWV |
| | | RQAPGKGLEWVAEISPYSGSTYYADSVKGRFTISADY |
| | | SKNTAYLQMNSLRAEDTAVYYCALRRPPIRLHPRGS |
| | | VMDYWGQGTLVTVSSGGGGSGGGGSGGGGSEVQLVES |
| | | GGGGLVQPGGSLRLSCAASGFTFSSFNMFWVRQAPGKG |
| | | LEWVAGIDDDGSYPNYGSAVKGRATISRDNSKNTLYL |
| | | QMNSLRAEDTAVYYCAKSGYGGSWGGYIADDIDAWGQ |
| | | GTLVTVSSASTKGPSVFPLAPSSKSTSGGTAALGCLV |
| | | KDYFPEPVTVSWSGALTSGVHTFPAVLQSSGLYSLS |
| | | SVVTVPSSSLGTQTYICNVNHKPSNTKVDKKVEPKSC |
| | | DKTHTCPPCPAPEAAGGPSVFLFPPKPKDTLMISRTP |
| | | EVTCVVVDVSHEDPEVKFNWYDGVEVHNAKTKPREE |
| | | QYNSTYRVVSVLTVLHQDWLNGKEYKCKVSNKALGAP |
| | | IEKTISKAKGQPREPQVYTLPPSREEMTKNQVSLTCL |
| | | VKGFYPSDIAVEWESNGQPENNYKTTPPVLDSDGSFF |
| | | LYSKLTVDKSRWQQGNVFSCSVMHEALHNHYTQKSLS |
| | | 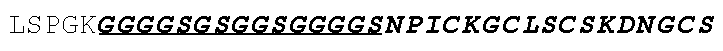 |
| | | 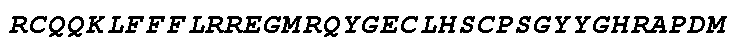 |
| | | 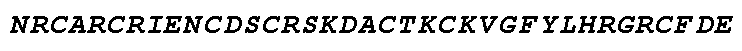 |
| | | 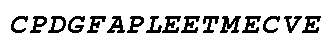 |
| | | L6-F10-FSPO2RA LC SEQ ID NO: 112 |
| | | DIQMTQSPSSLSASVGDRVTITCRASQDVSTAVAWYQ |
| | | QKPGDAPKLLIYSASFLYSGVPSRFSGSGSGTDFTLT |
| | | ISSLQPEDFATYYCQQSYTTPPTFGQGTKVEIKGGGG |
| | | SGGGGSGGGGSELTQPPSVSVSPGQTARITCSGDGSY |
| | | AGSYYYGWYQQKPGQAPVTLIYYNNKRPSGIPERFSG |
| | | SLSGSTNTLTISGVQAEDEADYYCGSADNSGAAFGGG |
| | | TKLTVLRTVAAPSVFIFPPSDEQLKSGTASVVCLLNN |
| | | FYPREAKVQWKVDNALQSGNSQESVTEQDSKDSTYSL |
| | | SSTLTLSKADYEKHKVYACEVTHQGLSSPVTKSFNRG |
| | | EC |
| Fv-IgG | L6-F7B- | L6-F7B-RSPO2RA HC SEQ ID NO: 113 |
| | RSPO2RA | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNFSSSSIHWVRQAPGKG |
| | | LEWVAYIYPSYDYTYYADSVKGRFTISADTSKNTAYL |
| | | QMNSLRAEDTAVYYCARGYFYTWGGMDYWGQGTLVTV |
| | | SSASTKGPSVFPLAPSSKSTSGGTAALGCLVKDYFPE |
| | | PVTVSWNSGALTSGVHTFPAVLQSSGLYSLSSVVTVP |
| | | SSSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALGAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGKG |
| | | 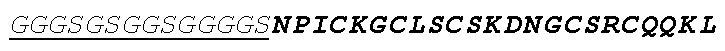 |
| | | 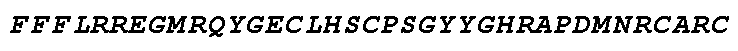 |
| | | 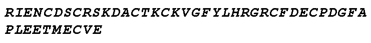 |
| | | -L-6-F7B-RSPO2RA LC SEQ ID NO: 114 |
| | | DIQMTQSPSSLSASVGDRVTITCRASQDVSTAVAWQ |
| | | QKPGKAPKLLIYSASFLYSGVPSRFSGSGSGTDFTLT |
| | | ISSLQPEDFATYYCQQSYTTPPTFGQGTKVEIKRGGG |
| | | GSGGGGSGGGGSDIQMTQSPSSLSASVGDRVTITCRA |
| | | SQSVSSAVAWYQQKPGKAPKLLIYSASSLYSGVPSRF |
| | | SGSRSGTDFTLTISSLQPEDFATYYCQQYYSLFTFGQ |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHKVYACEVTHQGLSSPVTKSFNR |
| | | GEC |
| FV-IgG | L6-F2I- | L6-F2I-RSPO2RA HC SEQ ID NO: 115 |
| | RSPO2RA | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNIHSSSIHWVRQAPGKG |
| | | LEWVAATYSSFGSITYADSVKGRFTISADTSKNTAYL |
| | | QMNSLRAEDTAVYYCARYHHPFGYALDYWGQGTLVTV |
| | | SSASTKGPSVFPLAPSSKSTSGGTAALGCLVKDYFPE |
| | | PVTVSWNSGALTSGVHTFPAVLQSSGLYSLSSVVTVP |
| | | SSSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYDGVEVHNAKTKPREEQYNSTY |
| | | RVVSVLTVLHQDWLNGKEYKCKVSNKALGAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGKG |
| | | 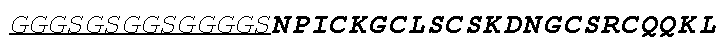 |
| | | 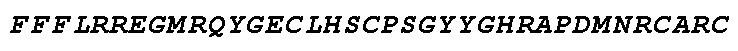 |
| | | 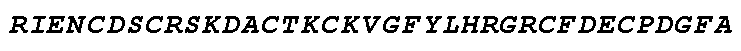 |
| | | 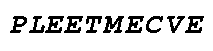 |
| | | -L6-F2I-RSPO2RA LC SEQ ID NO: 117 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKRGGG |
| | | GSGGGGSGGGGSDIQMTQSPSSLSASVGDRVTITCRA |
| | | SQSVSSAVAWYQQKPGKAPKLLIYSASSLYSGVPSRF |
| | | SGSRSGTDFTLTISSLQPEDFATYYCQQGVYLFTFGQ |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHKVYACEVTHQGLSSPVTKSFNR |
| | | GEC |
| 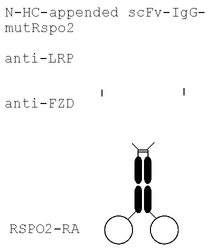 | L6-N-HC appended ScFv- F12578-IgG- RSPO2Ra | L6-N-HC appended ScFv-F12578-IgG-RSPO2RA HC SEQ ID NO: 118 EVQLVESGGGLVQPGGSLRLSCAASGFTFTSY YISWVRQAPGKGLEWVAEISPYSGSTYYADSV KGRFTISADTSKNTAYLQMNSLRAEDTAVYYC ALRARPPIRLHPRGSVMDYWGQGTLVTVSSGS AASGSSGGSSSGADIQMTQSPSSLSASVGDRVTIT CRASQDVSTAVAWYQQKPGKAPKLLIYSASFL YSGVPSRFSGSGSGTDFTLTISSLQPEDFATYY CQQSYTTPPTFGQGTKVEIKGGGGSGGGGSGG GGSEVQLVESGGGLVQPGGSLRLSCAASGFTF SHYTLSWVRQAPGKGLEWVSVISGDGSYTYY ADSVKGRFTISSDNSKNTLYLQMNSLRAEDTA VYYCARNFIKYVFANWGQGTLVTVSSASTKGP SVFPLAPSSKSTSGGTAALGCLVKDYFPEPVTVS |
| | | WNSGALTSGVHTFPAVLQSSGLYSLSSVVTVPSS |
| | | SLGTQTYICNVNHKPSNTKVDKKVEPKSCDKTH |
| | | TCPPCPAPEAAGGPSVFLFPPKPKDTLMISRTPEV |
| | | TCVVVDVSHEDPEVKFNWYYVDGVEVHNAKTKP |
| | | REEQYNSTYRVVSVLTVLHQDWLNGKEYKCKV |
| | | SNKALGAPIEKTISKAKGQPREPQVYTLPPSREEM |
| | | TKNQVSLTCLVKGFYPSDIAVEWESNGQPENNY |
| | | KTTPPVLDSDGSFFLYSKLTVDKSRWQQGNVFSC |
| | | SVMHEALHNHYTQKSLSLSPGKGGGGSGSGGSG |
| | | 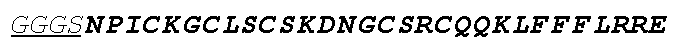 |
| | | 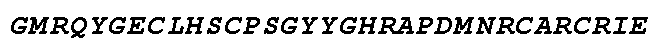 |
| | | 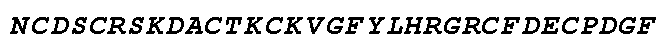 |
| | | 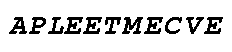 |
| | | >F12578 LC SEQ ID NO: 119 |
| | | DIELTQPPSVSVAPGQTARISCSGDNIGSFYVH |
| | | WYQQKPGQAPVLVIYDKSNRPSGIPERFSGSN |
| | | SGNTATLTISGTQAEDEADYYCQSYANTLSLV |
| | | FGGGTKLTVLGQPKAAPSVTLFPPSSEELQANK |
| | | ATLVCLISDFYPGAVTVAWKADSSPVKAGVETT |
| | | TPSKQSNNKYAASSYLSLTPEQWKSHRSYSCQVT |
| | | HEGSTVEKTVAPTECS |
| N-HC-appended scFv-IgG- | L6-HC | L6-N-HC appended ScFv-F127-IgG-RSPO2RA HC |
| mutRspo2 | appended | SEQ ID NO: 120 |
| | ScFv-F127- | EVQLVESGGGLVQPGGSLRLSCAASGFTFTSY |
| | IgG- | YISWVRQAPGKGLEWVAEISPYSGSTYYADSV |
| | RSPO2RA | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | SSYIHWVRQAPGKGLEWVAYIYSSYGSTYYAD |
| | | SVKGRFTISADTSKNTAYLQMNSLRAEDTAVY |
| | | YCARASWYALDYWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLGT |
| | | QTYICNVNHKPSNTKVDKKVEPKSCDKTHTCPP |
| | | CPAPEAAGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LGAPIEKTISKAKGQPREPQVYTLPPSREEMTKN |
| | | QVSLTCLVKGFYPSDIAVEWESNGQPENNYKTTP |
| | | PVLDSDGSFFLYSKLTVDKSRWQQGNVFSCSVM |
| | | HEALHNHYTQKSLSLSPGKGGGGSGSGGSGGGG |
| | | 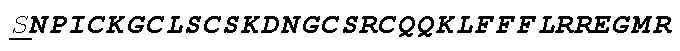 |
| | | 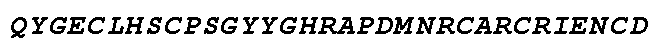 |
| | | 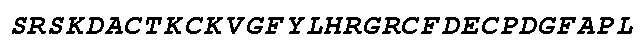 |
| | | 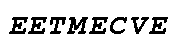 |
| | | >F127LC SEQ ID NO: 121 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQYWYGVAP |
| | | ITFGQGTKVEIKRTVAAPSVFIFPPSDEQLKSGT |
| | | ASVVCLLNNFYPREAKVQWKVDNALQSGNSQE |
| | | SVTEQDSKDSTYSLSSTLTLSKADYEKHKVTACE |
| | | VTHQGLSSPVTKSFNRGEC |
| N-HC-appended scFv-IgG- | L6-N-HC | L6-N-HC appended ScFv-F58-IgG-RSPO2RA HC |
| mutRspo2 | appended | SEQ ID NO: 122 |
| | ScFv-F58- | EVQLVESGGGLVQPGGGSLRLSCAASGFTFTSY |
| | IgG- | YISWVRQAPGKGLEWVAEISPYSGSTYYADSV |
| | RSPO2RA | KGRFTISADTSKNTAYLQMNSLRAEDTAVYYC |
| | | ALRARPPIRLHPRGSVMDYWGQGTLVTVSSGS |
| | | AASGSSGGSSSSGADIQMTQSPSSLSASVGDRVTIT |
| | | CRASQDVSTAVAWYQQKPGKAPKKLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | YSYIHWVRQAPGKGLEWVASIYSSSGSTSYAD |
| | | SVKGRFTISADTSKNTAYLQMNSLRAEDTAVY |
| | | YCARGAIDYWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAP |
| | | EAAGGPSVFLFPPKPKDTLMISRTPEVTCVVVDV |
| | | SHEDPEVKFNWYCDGVEHNAKTKPREEQYNS |
| | | TYRVVSVLTVLHQDWLNGKEYKCKVSNKALGA |
| | | PIEKTISKAKGQPREPQVYTLPPSREEMTKNQVSL |
| | | TCLVKGFYPSIAVEWESNGQPENNYKTTPPVLD |
| | | SDGSFFLYSKLTVDKSRWQQGNVFSCSVMHEAL |
| | | 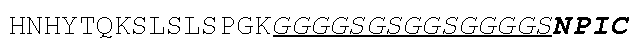 |
| | | 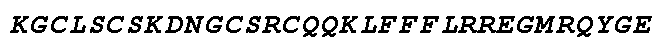 |
| | | 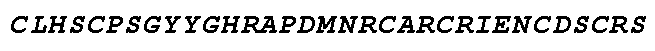 |
| | | 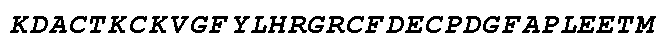 |
| | | 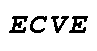 |
| | | >F58 LC SEQ ID NO: 123 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQWYSSGHV |
| | | LITFGQGTKVEIKRTVAAPSVFIFPPSDEQLKSGT |
| | | ASVVCLLNNFYPREAKVQWKVDNALQSGNSQE |
| | | SVTEQDSKDSTYSLSSTLTLSKADYEKHKVYACE |
| | | VTHQGLSSPVTKSFNRGEC |
| N-HC-appended scFv-IgG- | L6-N-HC | L6-N-HC appended ScFv-F4-2-IgG-RSPO2RA HC |
| mutRspo2 | appended | SEQ ID NO: 124 |
| | ScFv-F4-2- | EVQLVESGGGLVQPGGSLRLSCAASGFTFTSV |
| | IgG- | YISWVRQAPGKGLEWVAEISPYSGSTYYADSV |
| | RSPO2RA | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | YYYIHWVRQAPGKGLEWVASIYPSSGYTYYA |
| | | DSVKGRFTISADTSKNTAYLQMNSLRAEDTAV |
| | | YYCARSSFYWAMDYWGQGTLVTVSSASTKGP |
| | | SVFPLAPSSKSTSGGTAALGCLVKDYFPEPVTVS |
| | | WNSGALTSGVHTFPAVLQSSGLYSLSSVVTVPSS |
| | | SLGTQTYICNVNHKPSNTKVDKKVEPKSCDKTH |
| | | TCPPCPAPEAAGGPSVFLFPPKPKDTLMISRTPEV |
| | | TCVVVDVSHEDPEVKFNWYVDGVEVHNAKTKP |
| | | REEQYNSTYRVVSVLTVLHQDWLNGKEYKCKV |
| | | SNKALGAPIEKTISKAKGQPREPQVYTLPPSREEM |
| | | TKNQVSLTCLVKGFYPSDIAVEWESNGQPENNY |
| | | KTTPPVLDSDGSFFLYSKLTVDKSRWQQGNVFSC |
| | | SVMHEALHNHYTQKSLSLSPGKGGGGSGSGGSG |
| | | 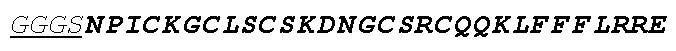 |
| | | 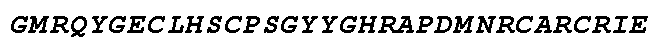 |
| | | 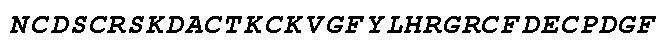 |
| | | 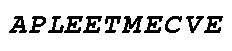 |
| | | >F4-2 LC SEQ ID NO: 125 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQSYAAYLFT |
| | | FGQGTKVEIKRTVAAPSVFIFPPSDEQLKSGTAS |
| | | VVCLLNNFYPREAKVQWKVDNALQSGNSQESV |
| | | TEQDSKDSTYSLSSTLTLSKADYEKHKVYACEVT |
| | | HQGLSSPVTKSFNRGEC |
| N-HC-appended scFv-IgG- | L6-N-HC | L6-N-HC appended ScFv-F4-IgG-RSPO2RA HC SEQ |
| mutRspo2 | appended | ID NO: 126 |
| | ScFv-F4- | EVQLVESGGGLVQPGGSLRLSCAASGFTFTSY |
| | IgG- | YISWVRQAPGKGLEWVAEISPYSGSTYYADSV |
| | RSPO2RA | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNL |
| | | SSYSMHWVRQAPGKGLEWVAYISSYYGYTYY |
| | | ADSVKGRFTISADTSKNTAYLQMNSLRAEDTA |
| | | VYYCARPAPGHWGFDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLTCLVKGFYPSDIAVEWESNGQPENN |
| | | YKTTPPVLDSDGSFFLYSKLTVDKSRWQQGNVF |
| | | SCSVMHEALHNHYTQKSLSLSPGKGGGGSGSGG |
| | | 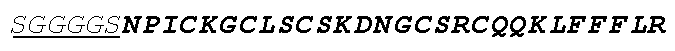 |
| | | 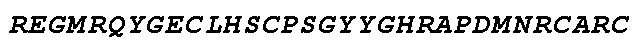 |
| | | 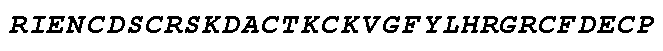 |
| | | 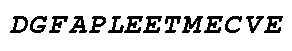 |
| | | >F4 LC SEQ ID NO: 127 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQWYYAPITF |
| | | GQGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| N-HC-appended scFv-IgG- | L6-N-HC | L6-N-HC appended ScFv-F49-IgG-RSPO2$A HC |
| mutRspo2 | appended | SEQ ID NO: 128 |
| | ScFv-F49- | EVQLVESGGGLVQPGGSLRLSCAASGFTFTSY |
| | IgG- | YISWVRQAPGKGLEWVAEISPYSGSTYYADSV |
| | RSPO2RA | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSQVQLVQSGAEVKKPGASVKVSCKASGGT |
| | | FSSYAISWVRQAPGQGLEWMGWINAGNGNTT |
| | | YAQKFQGRVTMTRDTSTSTVYMELSSLRSEDT |
| | | AVYYCARHYYGSGSYPDWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLTCLVKGFYPSDIAVEWESNGQPEN |
| | | NYKTTPPVLDSDGSFFLYSKLTVDKSRWQQGNV |
| | | FSCSVMHEALHNHYTQKSLSLSPGKGGGGSGSG |
| | | 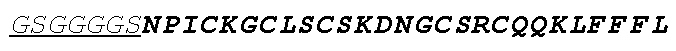 |
| | | 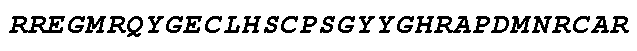 |
| | | 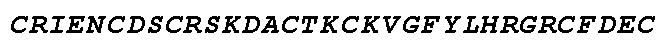 |
| | | 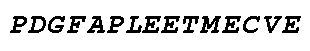 |
| | | >F49 LC SEQ ID NO: 129 |
| | | DIQMTQSPSSPSASVGDRVTITCRASQSISSYLN |
| | | WYQQKPGKAPKLLIYAASSLQSGVPSRFSGSG |
| | | SGTDFTLTISSLQPEDFATYYCQQSYSTPLTFG |
| | | GGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| N-HC-appended scFv-IgG- | L6-N-HC | L6-N-HC appended ScFv-F10-IgG-RSPO2RA HC |
| mutRspo2 | appended | SEQ ID NO: 130 |
| | ScFv-F10- | EVQLVESGGGLVQPGGSLRLSCAASGFTFTSY |
| | IgG- | YISWVRQAPGKGLEWVAEISPYSGSTYYADSV |
| | RSPO2RA | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFTF |
| | | SSFNMFWVRQAPGKGLEWVAGIDDDGSYPNY |
| | | GSAVKGRATISRDNSKNTLYLQMNSLRAEDTA |
| | | VYYCAKSGYGGSWGGYIADDIDAWGQGTLVT |
| | | VSSASTKGSVFPLAPSSKSTSGGTAALGCLVKD |
| | | YFPEPVTVSWNSGALTSGVHTFPAVLQSSGLYSL |
| | | SSVVTVPSSSLGTQTYICNVNHKPSNTKVDKKVE |
| | | PKSCDKTHTCPPCPAPEAAGGPSVFLFPPKPKDTL |
| | | MISRTPEVTCVVVDVSHEDPEVKFNWYVDGVEV |
| | | HNAKTKPREEQYNSTYRVVSVLTVLHQDWLNG |
| | | KEYKCKVSNKALGAPIEKTISKAKGQPREPQVYT |
| | | LPPSREEMTKNQVSLTCLVKGFYPSDIAVEWESN |
| | | GQPENNYKTTPPVLDSDGSFFLYSKLTVDKSRW |
| | | QQGNVFSVMHEALHNHYTQKSLSLSPGKGGG |
| | | 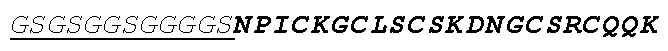 |
| | | 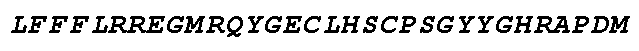 |
| | | 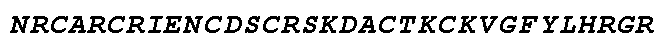 |
| | | 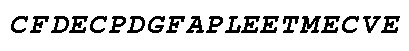 |
| | | >F10 LC SEQ ID NO: 131 |
| | | ELTQPPSVSVSPGQTARITCSGDGSYAGSYYYG |
| | | WYQQKPGQAPVTLIYYNNKRPSGIPERFSGSL |
| | | SGSTNTLTISGVQAEDEADYYCGSADNSGAAF |
| | | GGGTKLTVLRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| 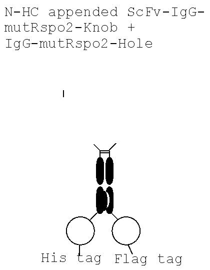 | L6-N-Hc appended ScFv- F12578-IgG- RSPO2RA- Knob-his + F12578-IgG- RSPO2RA Hole-Flag | L6-N-HC appended ScFv-F12578-IgG-RSPO2RA- Knob-His HC SEQ ID NO: 132 EVQLVESGGGLVQPGGSLRLSCAASGFTFTSY YISWVRQAPGKGLEWVAEISPYSGSTYYADSV KGRFTISADTSKNTAYLQMNSLRAEDTAVYYC ALRARPPIRLHPRGSVMDYWGQGTLVTVSSGS AASGSSGGSSSGADIQMTQSPSSLSASVGDRVTIT CRASQDVSTAVAWYQQKPGKAPKLLIYSASFL YSGVPSRFSGSGSGTDFTLTISSLQPEDFATYY CQQSYTTPPTFGQGTKVEIKGGGGSGGGGSGG GGSEVQLVESGGGLVQPGGSLRLSCAASGFTF SHYTLSWVRQAPGKGLEWVSVISGDGSYTYY ADSVKGRFTISSDNSKNTLYLQMNSLRAEDTA VYYCARNFIKYVFANWGQGTLVTVSSASTKGP SVFPLAPSSKSTSGGTAALGCLVKDYFPEPVTVS WNSGALTSGVHTFPAVLQSSGLYSLSSVVTVPSS |
| | | SLGTQTYICNVNHKPSNTKVDKKVEPKSCDKTH |
| | | TCPPCPAPEAAGGPSVFLFPPKPKDTLMISRTPEV |
| | | TCVVVDVSHEDPEVKFNWYVDGVEVHNAKTKP |
| | | REEQYNSTYRVVSVLTVLHQDWLNGKEYKCKV |
| | | SNKALGAPIEKTISKAKGQPREPQVYTLPPSREEM |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPGKGGGGSGGGGSG |
| | | 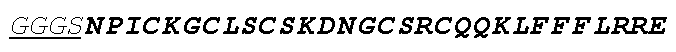 |
| | | 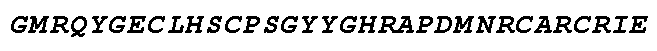 |
| | | 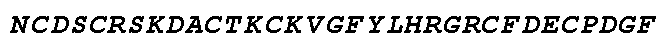 |
| | | 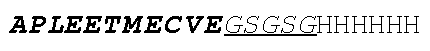 |
| | | >F12578-IgG-RSPO2RA-Hole Flag HC SEQ ID |
| | | NO: 133 |
| | | EVQLVESGGGLVQPGGSLRLSCAASGFTFSHY |
| | | TLSWVRQAPGKGLEWVSVISGDGSYTYYADS |
| | | VKGRFTISSDNSKNTLYLQMNSLRAEDTAVYY |
| | | CARNFIKYYFANWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPSREEMTKNQ |
| | | VSLSCAVKGFYPSDIAVEWESNGQPENNYKTTPP |
| | | VLDSDGSFFLVSKLTVDKSRWQQGNVFSCSVMH |
| | | EALHNHYTQKSLSLSPGKGGGGSGGGGSGGGGS |
| | | 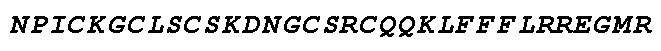 |
| | | 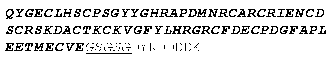 |
| | | >F12578 LC SEQ ID NO: 134 |
| | | DIELTQPPSVSVAPGQTARISCSGDNIGSFYVH |
| | | WYQQKPGQAPVLVIYDKSNRPSGIPERFSGSN |
| | | SGNTATLTISGTQAEDEADYYCQSYANTLSLV |
| | | FGGGTKLTVLGQPKAAPSVTLFPPSSEELQANK |
| | | ATLVCLISDFYPGAVTVAWKADSSPVKAGVETT |
| | | TPSKQSNNKYAASSYLSLTPEQWKSHRSYSCQVT |
| | | HEGSTVEKTVAPTECS |
| N-HC appended ScFv-IgG- | L6-N-HC | L6-N-HC appended ScFv-F127-IgG-RSPO2RA-Knob- |
| mutRspo2-Knob + | appended | His HC SEQ ID NO: 135 |
| IgG-mutRspo2-Hole | ScFv-F127- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F127-IgG- | AASGSSGGSSSGADIQMTQSPSSLSASVGDRVTIT |
| | RSPO2RA | CRASQDVSTAVAWYQQKPGKAPKLLIYSASFL |
| | Hole-Flag | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | SSYIHWVRQAPGKGLEWVAYIYSSYGSTYYAD |
| | | SVKGRFTISADTSKNTAYLQMNSLRAEDTAVY |
| | | YCARASWYALDYWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLGT |
| | | QTYICNVNHKPSNTKVDKKVEPKSCDKTHTCPP |
| | | CPAPEAAGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LGAPIEKTISKAKGQPREPQVYTLPPSREEMTKN |
| | | QVSLWCLVKGFYPSDIAVEWESNGQPENNYKTT |
| | | PPVLDSDGSFFLYSKLTVDKSRWQQGNVFCSV |
| | | MHEALHNHYTQKSLSLSPGKGGGGSGGGGSGGG |
| | | 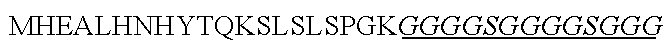 |
| | | 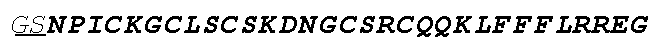 |
| | | 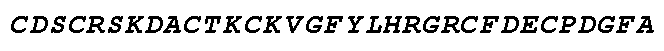 |
| | | 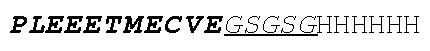 |
| | | >F127-IgG-RSPO2RA-Hole Flag HC SEQ ID |
| | | NO: 136 |
| | | EVQLVESGGGLVQPGGSLRLSCAASGFNISSSY |
| | | IHWVRQAPGKGLEWVAYIYSSYGSTYYADSV |
| | | KGRFTISADTSKNTAYLQMNSLRAEDTAVYYC |
| | | ARASWYALDYWGQGTLVTVSSASTKGPSVFPL |
| | | APSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPSREEMTKNQ |
| | | VSLSCAVKGFYPSDIAVEWESNGQPENNYKTTPP |
| | | VLDSDGSFFLVSKLTVDKSRWQQGNVFSCSVMH |
| | | EALHNHYTQKSLSLSPGKGGGGSGGGGSGGGGS |
| | | 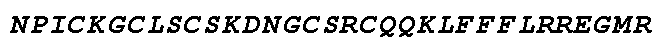 |
| | | 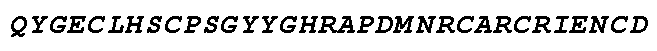 |
| | | 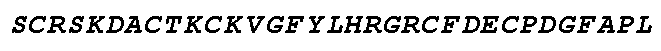 |
| | | 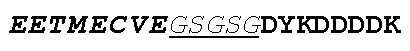 |
| | | >F127 LC SEQ ID NO: 137 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQYWYGVAP |
| | | ITFGQGTKVEIKRTVAAPSVFIFPPSDEQLKSGT |
| | | ASVVCLLNNFYPREAKVQWKVDNALQSGNSQE |
| | | SVTEQDSKDSTYSLSSTLTLSKADYEKHKVYACE |
| | | VTHQGLSSPVTKSFNRGEC |
| N-HC appended ScFv-IgG- | L6-N-HC | L6-N-HC appended ScFv-F58-IgG-RSPO2RA-Knob- |
| mutRspo2-Knob + | appended | His HC SEQ ID NO: 138 |
| IgG-mutRspo2-Hole | ScFv-F58- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F58-IgG- | AASGSSGGSSSGADIQMTQSPSSLSASVGDRVTIT |
| | RSPO2RA | CRASQDVSTAVAWYQQKPGKAPKLLIYSASFL |
| | Hole-Flag | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | YSYIHWVRQAPGKGLEWVASIYSSSGSTSYAD |
| | | SVKGRFTISADTSKNTATYLQMNSLRAEDTAVY |
| | | YCARGAIDYWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAP |
| | | EAAGGPSVFLFPPKPKDTLMISRTPEVTCVVVDV |
| | | SHEDPEVKFNWYVDGVEVHNAKTKPREEQYNS |
| | | TYRVVSVLTVLHQDWLNGKEYKCKVSNKALGA |
| | | PIEKTISKAKGQPREPQVYTLPPSREEMTKNQVSL |
| | | WCLVKGFYPSDIAVEWESNGQPENNYKTTPPVL |
| | | DSDGSFFLYSKLTVDKSRWQQGNVFSCSVMHEA |
| | | 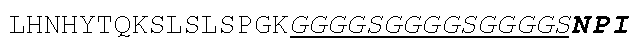 |
| | | 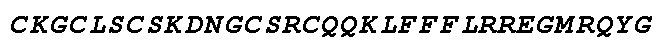 |
| | | 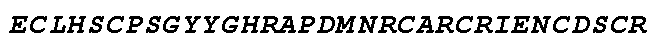 |
| | | 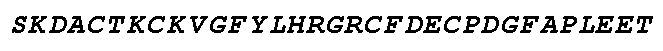 |
| | | 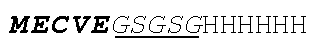 |
| | | >F58-IgG-RSPO2RA-Hole Flag HC SEQ ID NO: 139 |
| | | EVQLVESGGGLVQPGGSLRLSCAASGFNISYS |
| | | YIHWVRQAPGKGLEWVASIYSSSGSTSYADSV |
| | | KGRFTISADTSKNTAYLQMNSLRAEDTAVYYC |
| | | ARGAIDYWGQGTLVTVSSASTKGPSVFPLAPSS |
| | | KSTSGGTAALGCLVKDYFPEPVTVSWNSGALTS |
| | | GVHTFPAVLQSSGLYSLSSVVTVPSSSLGTQTYIC |
| | | NVNHKPSNTKVDKKVEPKSCDKTHTCPPCPAPE |
| | | AAGGPSVFLFPPKPKDTLMISRTPEVTCVVVDVS |
| | | HEDPEVKFNWYVDGVEVHNAKTKPREEQYNST |
| | | YRVVSVLTVLHQDWLNGKEYKCKVSNKALGAP |
| | | IEKTISKAKGQPREPQVYTLPPSREEMTKNQVSLS |
| | | CAVKGFYPSDIAVEWESNGQPENNYKTTPPVLDS |
| | | DGSFFLVSKLTVDKSRWQQGNVFSCSVMHEALH |
| | | 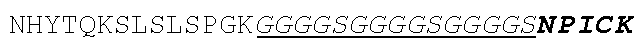 |
| | | 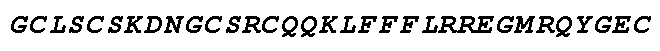 |
| | | 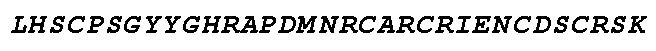 |
| | | 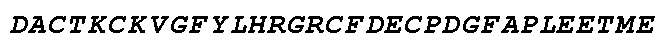 |
| | | 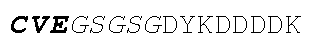 |
| | | >F58 LC SEQ ID NO: 140 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQWYSSGHV |
| | | LITFGQGTKVEIKRTVAAPSVFIFPPSDEQLKSGT |
| | | ASVVCLLNNFYPREAKVQWKVDNALQSGNSQE |
| | | SVTEQDSKDSTYSLSSTLTLSKADYEKHKVYACE |
| | | VTHQGLSSPVTKSFNRGEC |
| N-HC appended ScFv-IgG- | L6-N-HC | L6-N-HC appended ScFv-F4-2-IgG-RSPO2RA-Knob- |
| mutRspo2-Knob + | appended | His HC SEQ ID NO: 141 |
| IgG-mutRspo2-Hole | ScFv-F4-2- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F4-2-IgG- | AASGSSGGSSSGADIQMTQSPSSLSASVGDRVTIT |
| | RSPO2RA | CRASQDVSTAVAWYQQKPGKAPKLLIYSASFL |
| | Hole-Flag | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | YYYIHWVRQAPGKGLEWVASIYPSSGYTYYA |
| | | DSVKGRFTISADTSKNTAYLQMNSLRAEDTAV |
| | | YYCARSSFYWAMDYWGQGTLVTVSSASTKGP |
| | | SVFPLAPSSKSTSGGTAALGCLVKDYFPEPVTVS |
| | | WNSGALTSGVHTFPAVLQSSGLYSLSSVVTVPSS |
| | | SLGTQTYICNVNHKPSNTKVDKKVEPKSCDKTH |
| | | TCPPCPAPEAAGGPSVFLFPPKPKDTLMISRTPEV |
| | | TCVVVDVSHEDPEVKFNWYVDGVEVHNAKTKP |
| | | REEQYNSTYRVVSVLTVLHQDWLNGKEYKCKV |
| | | SNKALGAPIEKTISKAKGQPREPQVYTLPPSREEM |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPGKGGGGSGGGGSG |
| | | 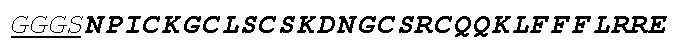 |
| | | 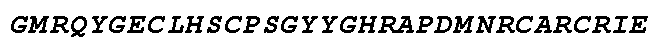 |
| | | 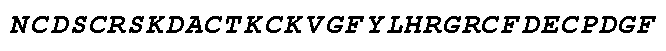 |
| | | 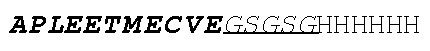 |
| | | >F4-2-IgG-RSPO2RA-Hole Flag HC SEQ ID |
| | | NO: 142 |
| | | EVQLVESGGGLVQPGGSLRLSCAASGFNISYY |
| | | YIHWVRQAPGKGLEWVASIYPSSGYTYYADSV |
| | | KGRFTISADTSKNTAYLQMNSLRAEDTAVYYC |
| | | ARSSFYWAMDYWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPSREEMTKNQ |
| | | VSLSCAVKGFYPSDIAVEWESNGQPENNYKTTPP |
| | | VLDSDGSFFLVSKLTVDKSRWQQGNVFSCSVMH |
| | | EALHNHYTQKSLSLSPGKGGGGSGGGGSGGGGS |
| | | 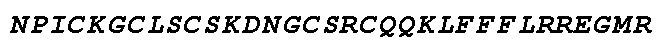 |
| | | 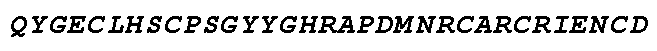 |
| | | 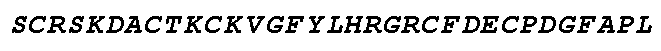 |
| | | 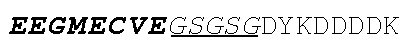 |
| | | >F4-2 LC SEQ ID NO: 143 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQSYAAYLFT |
| | | FGQGTKVEIKRTVAAPSVFIFPPSDEQLKSGTAS |
| | | VVCLLNNFYPREAKVQWKVDNALQSGNSQESV |
| | | TEQDSKDSTYSLSSTLTLSKADYEKHKVYACEVT |
| | | HQGLSSPVTKSFNRGEC |
| N-HC appended ScFv-IgG- | L6-N-HC | L6-N-HC appended ScFv-F4-IgG-RSPO2RA-Knob- |
| mutRspo2-Knob + | appended | his HC SEQ ID NO: 144 |
| IgG-mutRspo2-Hole | ScFv-F4- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F4-IgG- | AASGSSGGSSSGADIQMTQSPSSLSASVGDRVTIT |
| | RSPO2RA | CRASQDVSTAVAWYQQKPGKAPKLLIYSASFL |
| | Hole-Flag | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNL |
| | | SSYSMHWVRQAPGKGLEWVAYISSYYGYTYY |
| | | ADSVKGRFTISADTSKNTAYLQMNSLRAEDTA |
| | | VYYCARPAPGHWGFDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLWCLVKGFYPSDIAVEWESNGQPENN |
| | | YKTTPPVLDSDGSFFLYSKLTVDKSRWQQGNVF |
| | | SCSVMHEALHNHYTQKSLSLSPGKGGGGSGGGG |
| | | 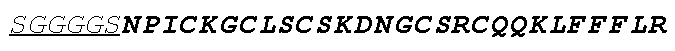 |
| | | 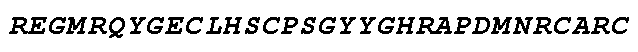 |
| | | 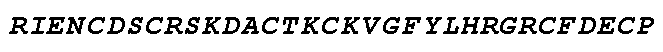 |
| | | 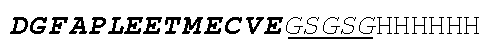 |
| | | >F4-IgG-RSPO2RA-Hole Flag HC SEQ ID NO: 145 |
| | | EVQLVESGGGLVQPGGSLRLSCAASGFNLSSY |
| | | SMHWVRQAPGKGLEWVAYISSYYGYTYYADS |
| | | VKGRFTISADTSKNTAYLQMNSLRAEDTAVYY |
| | | CARPAPGHWGFDYWGQGTLVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPSREEMT |
| | | KNQVSLSCAVKGFYPSDIAVEWESNGQPENNYK |
| | | TTPPVLDSDGSFFLVSKLTVDKSRWQQGNVFSCS |
| | | VMHEALHNHYTQKSLSLSPGKGGGGSGGGGSGG |
| | | 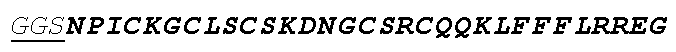 |
| | | 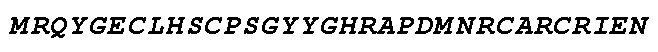 |
| | | 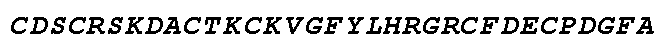 |
| | | 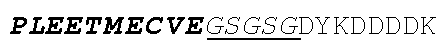 |
| | | >F4 LC SEQ ID NO: 146 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQWYYAPITF |
| | | GQGTKVEIKRTVAAPSVFIPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| N-HC appended ScFv-IgG- | L6-N-HC | L6-N-HC appended ScFv-F49-IgG-RSPO2RA-Knob- |
| mutRspo2-Knob + | appended | His HC SEQ ID NO: 147 |
| IgG-mutRspo2-Hole | ScFv-F49- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F49-IgG- | AASGSSGGSSSGADIQMTQSPSSLSASVGDRVTIT |
| | RSPO2RA | CRASQDVSTAVAWYQQKPGKAPKLLIYSASFL |
| | Hole-Flag | YSGVPSRFSGSGSGTDFTLTISSLQPEDFATYY |
| | | CQQSYTTPPTFGQGTKVEIKGGGGSGGGGSGG |
| | | GGSQVQLVQSGAEVKKPGASVKVSCKASGGT |
| | | FSSYAISWVRQAPGQGLEWMGWINAGNGNTT |
| | | YAQKFQGRVTMTRDTSTSTVYMELSSLRSEDT |
| | | AVYYCARHYYGSGSYPDWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLWCLVKGFYPSDIAVEWESNGQPEN |
| | | NYKTTPPVLDSDGSFFLYSKLTVDKSRWQQGNV |
| | | FSCSVMHEALHNHYTQKSLSLSPGKGGGGSGGG |
| | | 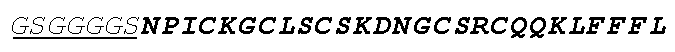 |
| | | 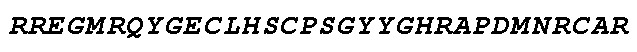 |
| | | 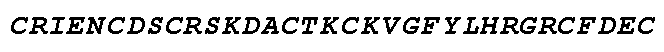 |
| | | 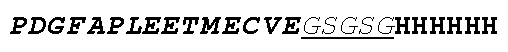 |
| | | >F49-IgG-RSPO2RA-Hole Flag HC SEQ ID NO: 148 |
| | | QVQLVQSGAEVKKPGASVKVSCKASGGTFSSY |
| | | AISWVRQAPGQGLEWMGWINAGNGNTTYAQ |
| | | KFQGRVTMTRDTSTSTVYMELSSLRSEDTAVY |
| | | YCARHYYGSGSYPDWGQGTLVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPSREEMT |
| | | KNQVSLSCAVKGFYPSDIAVEWESNGQPENNYK |
| | | TTPPVLDSDGSFFLVSKLTVDKSRWQQGNVFSCS |
| | | VMHEALHNHYTQKSLSLSPGKGGGGSGGGGSGG |
| | | 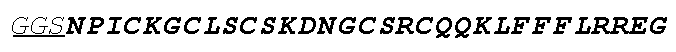 |
| | | 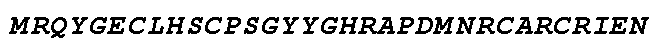 |
| | | 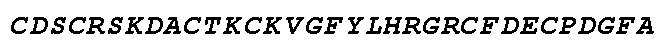 |
| | | 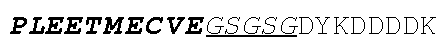 |
| | | >F49 LC SEQ ID NO: 149 |
| | | DIQMTQSPSSPSASVGDRVTITCRASQSISSYLN |
| | | WYQQKPGKAPKLLIYAASSLQSGVPSRFSGSG |
| | | SGTDFTLTISSLQPEDFATYYCQQSYSTPLTFG |
| | | GGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| N-HC appended ScFv-IgG- | L6-N-HC | L6-N-HC appended ScFv-F10-IgG-RSPO2RA-Knob- |
| mutRspo2-Knob + | appended | His HC SEQ ID NO: 150 |
| IgG-mutRspo2-Hole | ScFv-F10- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F10-IgG- | AASGSSGGSSSGADIQMTQSPSSLSASVGDRVTIT |
| | RSPO2RA | CRASQDVSTAVAWYQQKPGKAPKLLIYSASFL |
| | Hole-Flag | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFTF |
| | | SSFNMFWVRQAPGKGLEWVAGIDDDGSYPNY |
| | | GSAVKGRATISRDNSKNTLYLQMNSLRAEDTA |
| | | VYYCAKSGYGGSWGGYIADDIDAWGQGTLVT |
| | | VSSASTKGPSVFPLAPSSKSTSGGTAALGCLVKD |
| | | YFPEPVTVSWNSGALTSGVHTFPAVLQSSGLYSL |
| | | SSVVTVPSSSLGTQTYICNVNHKPSNTKVDKKVE |
| | | PKSCDKTHTCPPCPAPEAAGGPSVFLFPPKPKDTL |
| | | MISRTPEVTCVVVDVSHEDPEVKFNWYVDGVEV |
| | | HNAKTKPREEQYNSTYRVVSVLTVLHQDWLNG |
| | | KEYKCKVSNKALGAPIEKTISKAKGQPREPQVYT |
| | | LPPSREEMTKNQVSLWCLVKGFYPSDIAVEWES |
| | | NGQPENNYKTTPPVLDSDGSFFLYSKLTVDKSR |
| | | WQQGNVFSCSVMHEALHNHYTQKSLSLSPGKG |
| | | 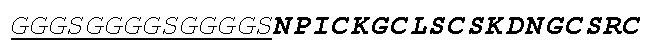 |
| | | 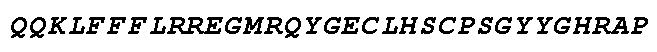 |
| | | 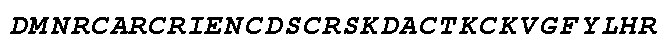 |
| | | 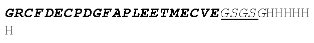 |
| | | >F10-IgG-RSPO2RA-Hole Flag HC SEQ ID NO: 151 |
| | | EVQLVESGGGLVQPGGSLRLSCAASGFTFSSF |
| | | NMRWVRQAPGKGLEWVAGIDDDGSYPNYGS |
| | | AVKGRATISRDNSKNTLYLQMNSLRAEDTAVY |
| | | YCAKSGYGGSWGGYIADDIDAWGQGTLVTVS |
| | | SASTKGPSVFPLAPSSKSTSGGTAALGCLVKDYF |
| | | PEPVTVSWNSGALTSGVHTFPAVLQSSGLYSLSS |
| | | VVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVH |
| | | NAKTKPREEQYNSTYRVVSVLTVLHQDWLNGK |
| | | EYKCKVSNKALGAPIEKTISDKAKGQPREPQVYTL |
| | | PPSREEMTKNQVSLSCAVKGFYPSDIAVEWESNG |
| | | QPENNYKTTPPVLDSDGSFFLVSKLTVDKSRWQ |
| | | QGNVFSCSVMHEALHNHYTQKSLSLSPGKGGGG |
| | | 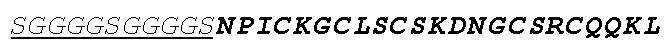 |
| | | 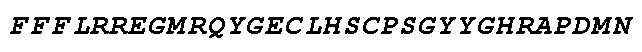 |
| | | 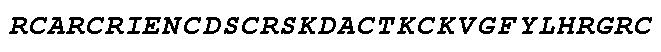 |
| | | 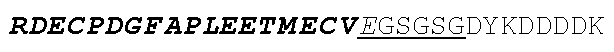 |
| | | >F10 LC SEQ ID NO: 152 |
| | | ELTQPPSVSVSPGQTARITCSGDGSYAGSYYYG |
| | | WYQQKPGQAPVTLIYYNNKRPSGIPERFSGSL |
| | | SGSTNTLTISGVQAEDEADYYCGSADNSGAAF |
| | | GGGTKLTVLRTVAAPSVFIFPPSDEQLKSGTASV |
| | | VDLLNNFYPREAKVQWKVDNALQSGNSQESVTE |
| | | QDSKDSTYSLSSTLTLSKADYEKHKVYACEVTH |
| | | QGLSSPVTKSFNRGEC |
| 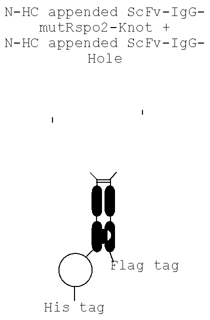 | L6-N-HC appended ScFv- F12578-IgG- RSPO2RA- Knob-his + L6-N-HC appended ScFv- F12478-IgG- Hole-Flag | L6-N-HC appended Scfv-F12578-IgG-RSPO2RA- Knob-His HC SEQ ID NO: 153 EVQLVESGGGLVQPGGSLRLSCAASGFTFTSY YISWVRQAPGKGLEWVAEISPYSGSTYYADSV KGRFTISADTSKNTAYLQMNSLRAEDTAVYYC ALRARPPIRLHPRGSVMDYWGQGTLVTVSSGS AASGSGGSSGADIQMTQSPSSLSASVGDRVTIT CRASQDVSTAVAWYQQKPGKAPKLLIYSASFL YSGVPSRFSGSGSGTDFTLTISSLQPEDFATYY CQQSYTTPPTFGQGTKVEIKGGGGSGGGGSGG GGSEVQLVESGGGLVQPGGSLRLSCAASGFTF SHYTLSWVRQAPGKGLEWVSVISGDGSYTYY ADSVKGRFTISSDNSKNTLYLQMNSLRAEDTA VYYCARNFIKYVFANWGQGTLVTVSSASTKGP SVFPLAPSSKSTSGGTAALGCLVKDYFPEPVTVS WNSGALTSGVHTFPAVLQSSGLYSLSSVVTVPSS SLGTQTYICNVNHKPSNTKVDKKVEPKSCDKTH TCPPCPAPEAAGGPSVFLFPPKPKDTLMISRTPEV TCVVVDVSHEDPEVKFNWYDGVEVHNAKTKP REEQYNSTYRVVSVLTVLHQDWLNGKEYKCKV |
| | | SNKALGAPIEKTISKAKGQPREPQVYTLPPSREEM |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPGKGGGGSGGGGSG |
| | | 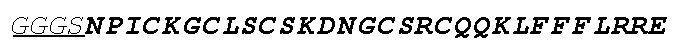 |
| | | 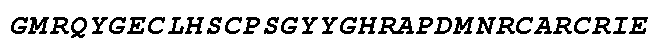 |
| | | 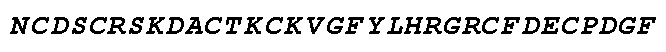 |
| | | 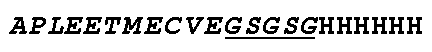 |
| | | >L6-N-HC appended ScFv-F12578-IgG-Hole Flag HC |
| | | SEQ ID NO: 154 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFTF |
| | | SHYTLSWVRQAPGKGLEWVSVISGDGSYTYY |
| | | ADSVKGRFTISSDNSKNTLYLQMNSLRAEDTA |
| | | VYYCARNFIKYVFANWGQGTLVTVSSASTKGP |
| | | SVFPLAPSSKSTSGGTAALGCLVKDYFPEPVTVS |
| | | WNSGALTSGVHTFPAVLQSSGLYSLSSVVTVPSS |
| | | SLGTQTYICNVNHKPSNTKVDKKVEPKSCDKTH |
| | | TCPPCPAPEAAGGPSVFLFPPKPKDTLMISRTPEV |
| | | TCVVVDVSHEDPEVKFNWYVDGVEVHNAKTKP |
| | | REEQYNSTYRVVSVLTVLHQDWLNGKEYKCKV |
| | | SNKALGAPIEKTISKAKGQPREPQVYTLPPSREEM |
| | | TKNWVSLSCAVKGFYPSDIAVEWESNGQPENNY |
| | | KTTPPVLDSDGSFFLVSKLTVDKSRWQQGNVFSC |
| | | SVMHEALHNHYTQKSLSLSPGKGSGSGDYKDDD |
| | | DK |
| | | >F12578 LC SEQ ID NO: 155 |
| | | DIELTQPPSVSVAPGQTARISCSGDNIGSFYVH |
| | | WYQQKPGQAPVLVIYDKSNRPSGIPERFSGSN |
| | | SGNTATLTISGTQAEDEADYKYCQSYANTLSLV |
| | | FGGGTKLTVLGQPKAAPSVTLFPPSSEELQANK |
| | | ATLVCLISDFYPGAVTVAWKADSSPVKAGVETT |
| | | TPSKQSNNKYASSYLSLTPEQWKSHRSYSCQVT |
| | | HEGSTVEKTVAPTECS |
| N-HC appended ScFv-IgG- | L6-N-HC | L6-N-HC appended ScFv-F127-IgG-RSPO2RA-Knob- |
| mutRspo2-Knob + | appended | His HC SEQ ID NO: 156 |
| N-HC appended ScFv-IgG- | ScFv-F127- | EVQLVESGGGLVQPGGSLRLSCAASGFTFTSY |
| Hole | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | L6-N-HC | AASGSSGGSSSGADIQMTQSPSSLSASVGDRVTIT |
| | appended | CRASQDVSTAVAWYQQKPGKAPKLLIYSASFL |
| | ScFv-F127- | YSGVPSRFSGSGSGTDFTLTISSLQPEDFATYY |
| | IgG-Hole- | CQQSYTTPPTFGQGTKVEIKGGGGSGGGGSGG |
| | Flag | GGSEVQLVESGGGLVQPGGSLRLSCAASGFNIS |
| | | SSYIHWVRQAPGKGLEWVAYIYSSYGSTYYAD |
| | | SVKGRFTISADTSKNTAYLQMNSLRAEDTAVY |
| | | YCARASWYALDYWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLGT |
| | | QTYICNVNHKPSNTKVDKKVEPKSCDKTHTCPP |
| | | CPAPEAAGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LGAPIEKTISKAKGQPREPQVYTLPPSREEMTKN |
| | | QVSLWCLVKGFYPSDIAVEWESNGQGPENNYKTT |
| | | MHEALHNHYTQKSLSLSPGKGGGGSGGGGSGGG |
| | | 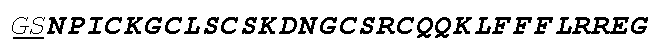 |
| | | 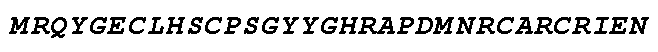 |
| | | 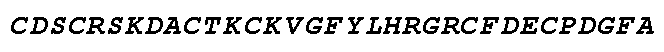 |
| | | 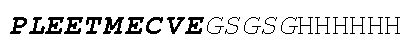 |
| | | >L6-N-HC appended ScFv-F127-IgG-Hole Flag HC |
| | | SEQ ID NO: 157 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | SSYIHWVRQAPGKGLEWVAYIYSSYGSTYYAD |
| | | SVKGRFTISADTSKNTAYLQMNSLRAEDTAVY |
| | | YCARASWYALDYWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLGT |
| | | QTYICNVNHKPSNTKVDKKVEPKSCDKTHTCPP |
| | | CPAPEAAGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LGAPIEKTISKAKGQPREPQVYTLPPSREEMTKN |
| | | QVSLSCAVKGFYPSDIAVEWESNGQPENNYKTTP |
| | | PVLDSDGSFFLVSKLTVDKSRWQQGNVFSCSVM |
| | | HEALHNHYTQKSLSLSPGKGSGSGDYKDDDDK |
| | | >F127 LC SEQ ID NO: 158 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQYWYGVAP |
| | | ITFGQGTKVEIKRTVAAPSVFIFPPSDEQLKSGT |
| | | ASVVCLLNNFYPREAKVQWKVDNALQSGNSQE |
| | | SVTEQDSKDSTYSLSSTLTLSKADYEKHKVYACE |
| | | VTHQGLSSPVTKSFNRGEC |
| N-HC appended ScFc-IgG- | L6-N-HC | L6-N-HC appended ScFv-F58-IgG-RSPO2RA-Knob- |
| mutRspo2-Knob + | appended | his HC SEQ ID NO: 159 |
| N-HC appended ScFv-IgG- | ScFv-F58 | EVQLVESGGGLVQPGGSLRLSCAASGFTFTSY |
| Hole | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | L6-N-HC | AASGSSGGSSSGADIQMTQSPSSLSASVGDRVTIT |
| | appended | CRASQDVSTAVAWYQQKPGKAPKLLIYSASFL |
| | ScFv-F58- | YSGVPSRFSGSGSGTDFTLTISSLQPEDFATYY |
| | IgG-Hole | CQQSYTTPPTFGQGTKVEIKGGGGSGGGGSGG |
| | Flag | GGSEVQLVESGGGLVQPGGSLRLSCAASGFNIS |
| | | YSYIHWVRQAPGKGLEWVASIYSSSGSTSYAD |
| | | SVKGRFTISADTSKNTAYLQMNSLRAEDTAVY |
| | | YCARGAIDYWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAP |
| | | EAAGGPSVFLFPPKPKDTLMISRTPEVTCVVVDV |
| | | SHEDPEVKFNWYVDGVEVHNAKTKPREEQYNS |
| | | TYRVVSVLTVLHQDWLNGKEYKCKVSNKALGA |
| | | PIEKTISKAKGQPREPQVYTLPPSREEMTKNQVSL |
| | | WCLVKGFYPSDIAVEWESNGQPENNYKTTPPVL |
| | | DSDGSFFLYSKLTVDKSRWQQGNVFSCSVMHEA |
| | | 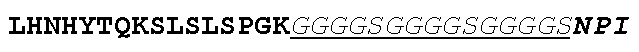 |
| | | 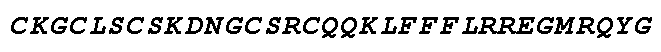 |
| | | 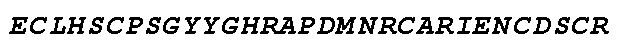 |
| | | 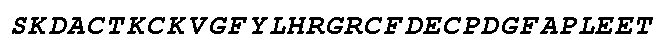 |
| | | 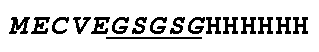 |
| | | >L6-N-HC appended ScFv-F58-IgG-Hole Flag HC |
| | | SEQ ID NO: 160 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | YSYIHWVRQAPGKGLEWVASIYSSSGSTSYAD |
| | | SVKGRFTISADTSKNTAYLQMNSLRAEDTAVY |
| | | YCARGAIDYWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAP |
| | | EAAGGPSVFLFPPKPKDTLMISRTPEVTCVVVDV |
| | | SHEDPEVKFNWYVDGVEVHNAKTKPREEQYNS |
| | | TYRVVSVLTVLHQDWLNGKEYKCKVSNKALGA |
| | | PIEKTISKAKGQPREPQVYTLPPSREEMTKNQVSL |
| | | SCAVKGFYPSDIAVEWESNGQPENNYKTTPPVLD |
| | | SDGSFFLVSKLTVDKSRWQQGNFSCSVMHEAL |
| | | HNHYTQKSLSLSPGKGSGSGDYKDDDDK |
| | | >F58 LC SEQ ID NO: 161 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQWYSSGHV |
| | | LITFGQGTKVEIKRTVAAPSVFIFPPSDEQLKSGT |
| | | ASVVCLLNNFYPREAKVQWKVDNALQSGANQE |
| | | SVTEQDSKDSTYSLSSTLTLSKADYEKHKVYACE |
| | | VTHQGLSSPVTKSFNRGEC |
| N-HC appended ScFc-IgG- | L6-N-HC | L6-N-HC appended ScFv-F4-2-IgG-RSPO2RA-Knob- |
| mutRspo2-Knob + | appended | His HC SEQ ID NO: 162 |
| N-HC appended ScFv-IgG- | ScFv-F4-2- | EQVLVESGGGLVQPGGSLRLSCAASGFTFTSY |
| Hole | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | L6-N-HC | AASGSSGGSSSGADIQMTQSPSSLSASVGDRVTIT |
| | appended | CRASQDVSTAVAWYQQKPGKAPKLLIYSASFL |
| | ScFv-F4-2- | YSGVPSRFSGSGSGTDFTLTISSLQPEDFATYY |
| | IgG-Hole- | CQQSYTTPPTFGQGTKVEIKGGGGSGGGGSGG |
| | Flag | GGSEVQLVESGGGLVQPGGSLRLSCAASGFNIS |
| | | YYYIHWVRQAPGKGLEWVASIYPSSGYTYYA |
| | | DSVKGRFTISADTSKNTAYLQMNSLRAEDTAV |
| | | YYCARSSFYWAMDYWGQGTLVTVSSASTKGP |
| | | SVFPLAPSSKSTSGGTAALGCLVDKYFPEPVTVS |
| | | WNSGALTSGVHTFPAVLQSSGLYSLSSVVTVPSS |
| | | SLGTQTYICNVNHKPSNTKVDKKVEPKSCDKTH |
| | | TCPPCPAPEAAGGPSVFLFPPKPKDTLMISRTPEV |
| | | TCVVVDVSHEDPEVKFNWYVDGVEVHNAKTKP |
| | | REEQYNSTYRVVSVLTVLHQDWLNGKEYKCKV |
| | | SNKALGAPIEKTISKAKGQPREPQVYTLPPSREEM |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPGKGGGGSGGGGSG |
| | | 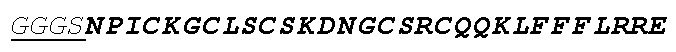 |
| | | 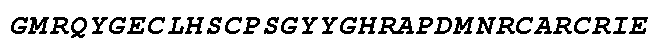 |
| | | 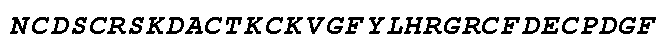 |
| | | 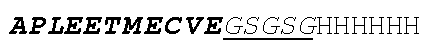 |
| | | >L6-N-HC appended ScFv-F4-2-IgG-Hole Flag HC |
| | | SEQ ID NO: 163 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | YYYIHWVRQAPGKGLEWVASIYPSSGYTYYA |
| | | DSVKGRFTISADTSKNTAYLQMNSLRAEDTAV |
| | | YYCARSSFYWAMDYWGQGTLVTVSSASTKGP |
| | | SVFPLAPSSKSTSGGTAALGCLVKDYFPEPVTVS |
| | | WNSGALTSGVHTFPAVLQSSGLYSLSSVVTVPSS |
| | | SLGTQTYICNVNHKPSNTKVDDVEPKSCDKTH |
| | | TCPPCPAPEAAGGPSVFLFPPKPKDTLMISRTPEV |
| | | TCVVVDVSHEDPEVKFNWYVDGVEVHNAKTKP |
| | | REEQYNSTYRVVSVLTVLHQDWLNGKEYKCKV |
| | | SNKALGQPIEKTISKAKGQPREPQVYTLPPSREEM |
| | | TKNQVSLSCAVKGFYPSDIAVEWESNGQPENNY |
| | | KTTPPVLDSDGSFFLVSKLTVDKSRWQQGNVFSC |
| | | SVMHEALHNHYTQKSLSLSPGKGSGSGDYKDDD |
| | | DK |
| | | >F4-2 LC SEQ ID NO: 164 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQSYAAYLFT |
| | | FGQGTKVEIKRTVAAPSVFIFPPSDEQLKSGTAS |
| | | VVCLLNNFYPREAKVQWKVDNALQSGNSQESV |
| | | TEQDSKDSTYSLSSTLTLSKADYEKHKVYACEVT |
| | | HQGLSSPVTKSFNRGEC |
| N-HC appended ScFv-IgG- | L6-N-HC | L6-N-HC appended ScFv-F4-IgG-RSPO2RA-Knob- |
| mutRspo2-Knob + | appended | His HC SEQ ID NO: 165 |
| N-HC appended ScFv-IgG- | ScFv-F4- | EVQLVESGGGLVQPGGSLRLSCAASGFTFTSYL |
| Hole | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | L6-N-HC | AASGSSGGSSSGADIQMTQSPSSLSASVGDRVTIT |
| | appended | CRASQDVSTAVAWYQQKPGKAPKLLIYSASFL |
| | ScFv-F4- | YSGVPSRFSGSGSGTDFTLTISSLQPEDFATYY |
| | IgG-Hole | CQQSYTTPPTFGQGTKVEIKGGGGSGGGGSGG |
| | Flag | GGSEVQLVESGGGLVQPGGSLRLSCAASGFNL |
| | | SSYSMHWVRQAPGKGLEWVAYISSYYGYTYY |
| | | ADSVKGRFTISADTSKNTAYLQMNSLRAEDTA |
| | | VYYCARPAPGHWGFDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLWCLVKGFYPSDIAVEWESNGQPENN |
| | | YKTTPPVLDSDGSFFLYSKLTVDKSRWQQGNVF |
| | | SCSVMHEALHNHYTQKSLSLSPGKGGGGSGGGG |
| | | 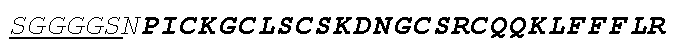 |
| | | 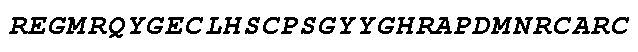 |
| | | 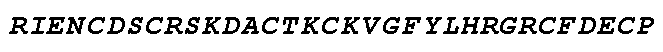 |
| | | 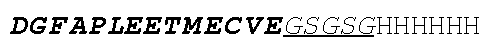 |
| | | >L6-N-HC appended ScFv-F4-IgG-Hole Flag HC |
| | | SEQ ID NO: 166 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNL |
| | | SSYSMHWVRQAPGKGLEWVAYISSYYGYTYY |
| | | ADSVKGRFTISADTSKNTAYLQMNSLRAEDTA |
| | | VYYCARPAPGHWGFDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLGQDWLNGKEYKCK |
| | | VSNKALGAPIEKTISKAKGQPREPQVYTLPPSREE |
| | | MTKNQVSLSCAVKGFYPSDIAVEWESNGQPENN |
| | | YKTTPPVLDSDGSFFLVSKLTVDKSRWQQGNVF |
| | | SCSVMHEALHNHYTQKSLSLSPGKGSGSGDYKD |
| | | DDDK |
| | | >F4 LC SEQ ID NO: 167 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQWYYAPITF |
| | | GQGTKVEIKRTVAPPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| N-HC appended ScFv-IgG- | L6-N-HC | L6-N-HC appended ScFv-F49-IgG-RSPO2RA-Knob- |
| mutRspo2-Inob + | appended | His HC SEQ ID NO: 168 |
| N-HC appended ScFv-IgG- | ScFv-F49- | EQQLVESGGGLVQPGGSLRLSCAASGFTFTSY |
| Hole | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | L6-N-HC | AASGSSGGSSSGADIQMTQSPSSLSASVGDRVTIT |
| | appended | CRASQDVSTAVAWYQQKPGKAPKLLIYSASFL |
| | ScFv-F49- | YSGVPSRFSGSGSGTDFTLTISSLQPEDFATYY |
| | IgG-Hole- | CQQSYTTPPTFGQGTKVEIKGGGGSGGGGSGG |
| | Flag | GGSQVQLVQSGAEVKKPGASVKVSCKASGGT |
| | | FSSYAISWVRQAPGQGLEWMGWINAGNGNTT |
| | | YAQKFQGRVTMTRDTSTSTVYMELSSLRSEDT |
| | | AVYYCARHYYGSGSYPDWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLWCLVKGFYPSDIAVEWESNGQPEN |
| | | NYKTTPPVLDSDGSFFLYSKLTVDKSRWQQGNV |
| | | FSCSVMHEALHNHYTQKSLSLSPGKGGGGSGGG |
| | | 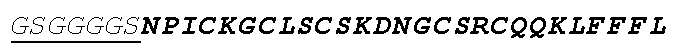 |
| | | 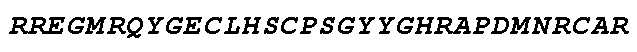 |
| | | 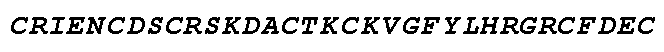 |
| | | 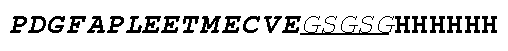 |
| | | >L6-N-HC appended ScFv-F49-IgG-Hole Flag HC |
| | | SEQ ID NO: 169 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRQSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSQVQLVQSGAEVKKPGASVKVSCKASGGT |
| | | FSSYAISWVRQAPGQGLEWMGWINAGNGNTT |
| | | YAQKFQGRVTMTRDTSTSTVYMELSSLRSEDT |
| | | AVYYCARHYYGSGSYPDWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLSCAVKGFYPSDIAVEWESNGQPEN |
| | | NYKTTPPVLDSDGSFFLVSKLTVDKSRWQQGNV |
| | | FSCSVMHEALHNHYTQKSLSLSPGKGSGSGDYK |
| | | DDDK |
| | | >F49 LC SEQ ID NO: 170 |
| | | DIQMTQSPSSPSASVGDRVTITCRASQSISSYLN |
| | | WYQQKPGKAPKLLIYAASSLQSGVPSRFSGSG |
| | | SGTDFTLTISSLQPEDFATYYCQQSYSTPLTFG |
| | | GGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| N-HC appended ScFv-IgG- | L6-N-HC | L6-N-HC appended ScFv-F10-IgG-RSPO2RA-Knob- |
| mutRspo2-Knob + | appended | His HC SEQ ID NO: 171 |
| N-HC appended Scfv-IgG- | ScFv-F10- | EVQLVESGGGLVQPGGSLRLSCAASGFTFTSY |
| Hole | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | L6-N-HC | AASGSSGGSSSGADIQMTQSPSSLSASVGDRVTIT |
| | appended | CRASQDVSTAVAWYQQKPGKAPKLLIYSASFL |
| | ScFv-F10- | YSGVPSRFSGSGSGTDFTLTISSLQPEDFATYY |
| | IgG-Hole- | CQQSYTTPPTFGQGTKVEIKGGGGSGGGGSGG |
| | Flag | GGSEVQLVESGGGLVQPGGSLRLSCAASGFTF |
| | | SSFNMFWVRQAPGKGLEWVAGIDDDGSYPNY |
| | | GSAVKGRATISRDNSKNTLYLQMNSLRAEDTA |
| | | VYYCAKSGYGGSWGGYIADDIDAWGQGTLVT |
| | | VSSASTKGPSVFPLAPSSKSTSGGTAALGCLVKD |
| | | YFPEPVTVSWNSGALTSGVHTFPAVLQSSGLYSL |
| | | SSVVTVPSSSLGTQTYICNVNHKPSNTKVDKKVE |
| | | PKSCDKTHTCPPCPAPEAAGGPSVFLFPPKPKDTL |
| | | MISRTPEVTCVVVDVSHEDPEVKFNWYDGVEV |
| | | HNAKTKPREEQYNSTYRVVSVLTVLHQDWLNG |
| | | KEYKCKVSNKALGAPIEKTISKAKGQPREPQVYT |
| | | LPPSREEMTKNQVSLWCLVKGFYPSDIAVEWES |
| | | NGQPENNYKTTPPVLDSDGSFFLYSKLTVDKSR |
| | | WQQGNVFSCSVMHEALHNHYTQKSLSLSPGKG |
| | | GGGSGGGGSGGGGSNPICKGCLSCSKDNGCSRC |
| | | 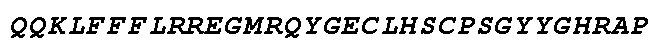 |
| | | 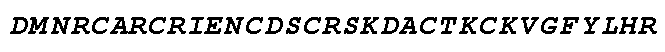 |
| | | 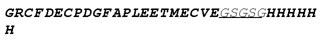 |
| | | >L6-N-HC appended ScFv-F10-IgG-Hole Flag HC |
| | | SEQ ID NO: 172 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFTF |
| | | SSFNMFWVRQAPGKGLEWVAGIDDDGSYPNY |
| | | GSAVKGRATISRDNSKNTLYLQMNSLRAEDTA |
| | | VYYCAKSGYGGSWGGYIADDIDAWGQGTLVT |
| | | VSSASTKGPSVFPLAPSSKSTSGGTAALGCLVDK |
| | | YFPEPVTVSWNSGALTSGVHTFPAVLQSSGLYSL |
| | | SSVVTVPSSSLGTQTYICNVNHKPSNTKVDKKVE |
| | | PKSCDKTHTCPPCPAPEAAGGPSVFLFPPKPKDTL |
| | | MISRTPEVTCVVVDVSHEDPEVKFNWYVDGVEV |
| | | HNAKTKPREEQYNSTYRVVSVLTVLHQDWLNG |
| | | KEYKCKVSNKALGAPIEKTISKAKGQPREPQVYT |
| | | LPPSREEMTKNQVSLSCAVKGFYPSDIAVEWESN |
| | | GQPENNYKTTPPVLDSDGSFFLVSKLTVDKSRW |
| | | QQGNVFSCSVMHEALHNHYTQKSLSLSPGKGSG |
| | | SGDYKDDDDK |
| | | >F10 LC SEQ ID NO: 173 |
| | | ELTQPPSVSVSPGQTARITCSGDGSYAGSYYYG |
| | | WYQQKPGQAPVTLIYYNNKRPSGIPERFSGSL |
| | | SGSTNTLTISGVQAEDEADYYCGSADNSGAAF |
| | | GGGTKLTVLRTVAAPSVFIPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| 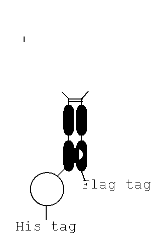 | L6-N-HC appended ScFv- F12578-IgG- RSPO2RA- Knob-his + F12578-IgG- Hole-Flag | L6-N-HC appended ScFv-F12578-IgG-RSPO2RA- Knob-His HC SEQ ID NO: 174 EVQLVESGGGLVQPGGSLRLSCAASGFTFTSY YISWVRQAPGKGLEWVAEISPYSGSTYYADSV KGRFTISADTSKNTAYLQMNSLRAEDTAVYYC ALRARPPIRLHPRGSVMDYWGQGTLVTVSSGS AASGSSGGSSSGADIQMTQSPSSLSASVGDRVTIT CRASQDVSTAVAWYQQKPGKAPKLLIYSASFL YSGVPSRFSGSGSGTDFTLTISSLQPEDFATYY CQQSYTTPPTFGQGTKVEIKGGGGSGGGGSGG GGSEVQLVESGGGLVQPGGSLRLSCAASGFTF SHYTLSWVRQAPGKGLEWVSVISGDGSYTYY ADSVKGRFTISSDNSKNTLYLQMNSLRAEDTA VYYCARNFIKYVFANWGQGTLVTVSSASTKGP |
| | | SVFPLAPSSKSTSGGTAALGCLVKDYFPEPVTVS |
| | | WNSGALTSGVHTFPAVLQSSGLYSLSSVVTVPSS |
| | | SLGTQTYICNVNHKPSNTKVDKKVEPKSCDKTH |
| | | TCPPCPAPEAAGGPSVFLFPPKPKDTLMISRTPEV |
| | | TCVVVDVSHEDPEVKFNWYVDGVEVHNAKTKP |
| | | REEQYNSTYRVVSVLTVLHQDWLNGKEYKCKV |
| | | SNKALGAPIEKTISKAKGQPREPQVYTLPPSREEM |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPGKGGGGSGGGGSG |
| | | 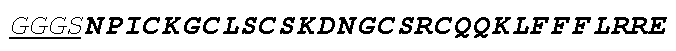 |
| | | 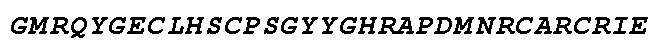 |
| | | 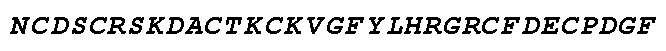 |
| | | 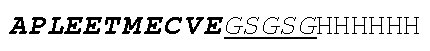 |
| | | >F12578-IgG-Hole Flag HC SEQ ID NO: 175 |
| | | EVQLVESGGGLVQPGGSLRLSCAASGFTFSHY |
| | | TLSWVRQAPGKGLEWVSVISGDGSYTYYADS |
| | | VKGRFTISSDNSKNTLYLQMNSLRAEDTAVYY |
| | | CARNFIKYVFANWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPSREEMTKNQ |
| | | VSLSCAVKGFYPSDIAVEWESNGQPENNYKTTPP |
| | | VLDSDGSFFLVSKLTVDKSRWQQGNVFSCSVMH |
| | | EALHNHYTQKSLSLSPGKGSGSGDYKDDDDK |
| | | >F12578 LC SEQ ID NO: 176 |
| | | DIELTQPPSVSVAPGQTARISCSGDNIGSFYVH |
| | | WYQQKPGQAPVLVIYDKSNRPSGIPERFSGSN |
| | | SGNTATLTISGTQAEDEADYYCQSYANTLSLV |
| | | FGGGTKLTVLGQPKAAPSVTLFPPSSEELQANK |
| | | ATLVCLISDFYPGAVTVAWKADSSPVKAGVETT |
| | | TPSKQSNNKYAASSYLSLTPEQWKSHRSYSCQVT |
| | | HEGSTVEKTVAPTECS |
| | L6-N-HC | L6-N-HC appended ScFv-F127-IgG-RSPO2RA-Knob- |
| | appended | His HC SEQ ID NO: 1199 |
| | ScFv-F127- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F127-IgG- | AASGSSGGSSSGADIQMTQSPSSLSASVGDRVTIT |
| | Hole-Flag | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | SSYIHWVRQAPGKGLEWVAYIYSSYGSTYYAD |
| | | SVKGRFTISADTSKNTAYLQMNSLRAEDTAVY |
| | | YCARASWYALDYWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLGT |
| | | QTYICNVNHKPSNTKVDKKVEPKSCDKTHTCPP |
| | | CPAPEAAGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LGAPIEKTISKAKGQPREPQVYTLPPSREEMTKN |
| | | QVSLWCLVKGFYPSDIAVEWESNGQPENNYKTT |
| | | PPVLDSDGSFFLYSKLTVDKSRWQQGNVFSCSV |
| | | MHEALHNHYTQKSLSLSPGKGGGGSGGGGSGGG |
| | | 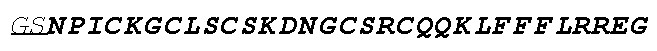 |
| | | 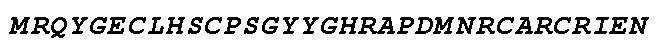 |
| | | 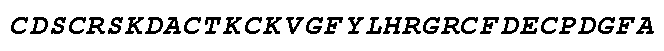 |
| | | 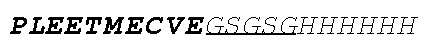 |
| | | >F127-IgG-Hole Flag HC SEQ ID NO: 200 |
| | | EVQLVESGGGLVQPGGSLRLSCAASGFNISSSY |
| | | IHWVRQAPGKGLEWVAYIYSSYGSTYYADSV |
| | | KGRFTISADTSKNTAYLQMNSLRAEDTAVYYC |
| | | ARASWYALDYWGQGTLVTVSSASTKGPSVFPL |
| | | APSSKSTSGGTAALGGLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPSREEMTKNQ |
| | | VSLSCAVKGFYPSDIAVEWESNGQPENNYKTTPP |
| | | VLDSDGSFFLVSKLTVDKSRWQQGNFSCSVMH |
| | | EALHNHYTQKSLSLSPGKGSGSDYKDDDDK |
| | | >F127 LC SEQ ID NO: 201 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQYWYGVAP |
| | | ITFGQGTKVEIKRTVAAPSVFIFPPSDEQLKSGT |
| | | ASVVCLLNNFYPREAKVQWKVDNALQSGNSQE |
| | | SVTEQDSKDSTYSLSSTLTLSKADYEKHKVYACE |
| | | VTHQGLSSPVTKSFNRGEC |
| | L6-N-HC | L6-N-HC appended ScFv-F58-IgG-RSPO2RA-Inob- |
| | appended | His HC SEQ ID NO: 202 |
| | ScFv-F58- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F58-IgG- | AASGSSGGSSSGADIQMTQSPSSLSASVGDRVTIT |
| | Hole-Flag | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | YSYIHWVRQAPGKGLEWVASIYSSSGSTSYAD |
| | | SVKGRFTISADTSKNTAYLQMNSLRAEDTAVY |
| | | YCARGAIDYWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAP |
| | | EAAGGPSVFLFPPKPKDTLMISRTPEVTCVVVDV |
| | | SHEDPEVKFNWYVDGVEVHNAKTKPREEQYNS |
| | | TYRVVSVLTVLHQDWLNGKEYKCKVSNKALGA |
| | | PIEKTISKAKGQPREPQVYTLPPSREEMTKNQVSL |
| | | WCLVKGFYPSDIAVEWESNGQPENNYKTTPPVL |
| | | DSDGSFFLYSKLTVDKSRWQQGNFSCSVMHEA |
| | | 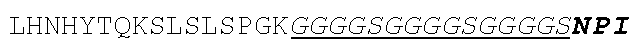 |
| | | 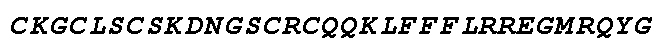 |
| | | 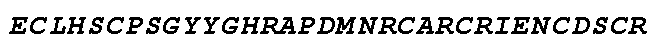 |
| | | 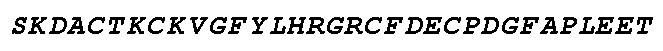 |
| | | 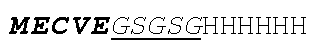 |
| | | >F58-IgG-Hole Flag HC SEQ ID NO: 203 |
| | | EVQLVESGGGLVQPGGSLRLSCAASGFNISYS |
| | | YIHWVRQAPGKGLEWVASIYSSSGSTSYADSV |
| | | KGRFTISADTSKNTAYLQMNSLRAEDTAVYYC |
| | | ARGAIDYWGAGTLVTVSSASTKGPSVFPLAPSS |
| | | KSTSGGTAALGCLVDKDYFPEVTVSWNSGALTS |
| | | GVHTFPAVLQSSGLYSLSSVVTVPSSSLGTQTYIC |
| | | NVNHKPSNTKVDKKVEPKSCDKTHTCPPCPAPE |
| | | AAGGSVFLFPPKPKDTLMISRTPEVTCVVVDVS |
| | | HEDPEVKFNWYVDGVEVHNAKTKPREEQYNST |
| | | YRVVSVLTVLHQDWLNGKEYKCKVSNKALGAP |
| | | IEKTISKAKGQPREPQVYTLPPSREEMTKNQVSLS |
| | | CAVKGFYPSDIAVEWESNGQPENNYKTTPPVLDS |
| | | DGSFFLVSKLTVDKSRWQQGNVFSCSVMHEALH |
| | | NHYTQKSLSLSPGKGSGSGDYKDDDDK |
| | | >F58 LC SEQ ID NO: 204 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQWYSSGHV |
| | | LITFGQGTKVEIKRTVAAPSVFIFPPSDEQLKSGT |
| | | ASVVCLLNNFYPREAKVQWKVDNALQSQNSQE |
| | | SVTEQDSKDSTYSLSSTLTLSKADYEKHKVYACE |
| | | VTHQGLSSPVTKSFNRGEC |
| | L6-N-HC | L6-N-HC appended ScFv-F4-2-IgG-RSPO2RA-Knob- |
| | appended | His HC SEQ ID NO: 177 |
| | ScFv-F4-2- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F4-2-IgG- | AASGSSGGSSSGADIQMTQSPSSLSASVGDRVTIT |
| | Hole-Flag | CRASQDVSTAVAWYQQKPGKAPKLLIYSASFL |
| | | YSTVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNIS |
| | | YYYIHWVRQAPGKGLEWVASIYPSSGYTYYA |
| | | DSVKGRFTISADTSKNTAYLQMNSLRAEDTAV |
| | | YYCARSSFYWAMDYWGQGTLVTVSSASTKGP |
| | | SVFPLAPSSKSTSGGTAALGCLVKDYFPEPVTVS |
| | | WNSGALTSGVHTFPAVLQSSGLYSLSSVVTVPSS |
| | | SLGTQTYICNVNHKPSNTKVDKKVEPKSCDKTH |
| | | TCPPCPAPEAAGGPSVFLFPPKPKDTLMISRTPEV |
| | | REEQYNSTYRVVSVLTVLHQDWLNGKEYKCKV |
| | | SNKALGAPIEKTIASKAKGQPREPQVYTLPPSREEM |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPGKGGGGSGGGGSG |
| | | 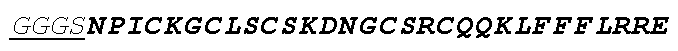 |
| | | 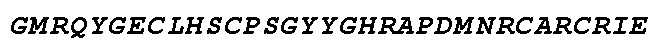 |
| | | 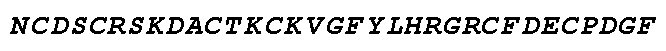 |
| | | 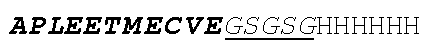 |
| | | >F4-IgG-Hole Flag HC SEQ ID NO: 178 |
| | | EVQLVESGGGLVQPGGSLRLSCAASGFNISYY |
| | | YIHWVRQAPGKGLEWVASIYPSSGYTYYADSV |
| | | KGRFTISADTSKNTAYLQMNSLRAEDTAVYYC |
| | | ARSSFYWAMDYWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPSREEMTKNQ |
| | | VSLSCAVKGFYPSDIAVEWESNGQPENNYKTTPP |
| | | VLDSDGSFFLVSKLTVDKSRWQQGNVFSCSVMH |
| | | EALHNHYTQKSLSLSPGKGSGSGDYKDDDDK |
| | | >F4-2 LC SEQ ID NO: 179 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQSYAAYLFT |
| | | FGQGTKVEIKRTVAAPSVFIFPPSDEQLKSGTAS |
| | | VVCLLNNFYPREAKVQWKVDNALQSGNSQESV |
| | | TEQDSKDSTYSLSSTLTLSKADYEKHKVYACEVT |
| | | HQGLSSPVTKSFNRGEC |
| | L6-N-HC | L6-N-HC appended ScFv-F4-IgG-RSPO2RA-Knob- |
| | appended | His HC SEQ ID NO: 180 |
| | ScFv-F4- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F4-IgG- | AASGSSGGSSSGADIQMTQSPSSLSASVGDRVTIT |
| | Hole-Flag | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNL |
| | | SSYSMHWVRQAPGKGLEWVAYISSYYGYTYY |
| | | ADSVKGRFTISADTSKNTAYLQMNSLRAEDTA |
| | | VYYCARPAPGHWGFDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLWCLVKGFYPSDIAVEWESNGQPENN |
| | | YKTTPPVLDSDGSFFLYSKLTVDKSRWQQGNVF |
| | | SCSVMHEALHNHYTQKSLSLSPGKGGGGSGGGG |
| | | 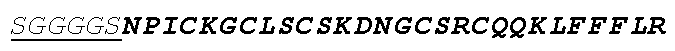 |
| | | 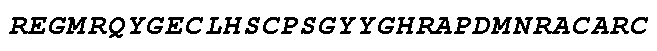 |
| | | 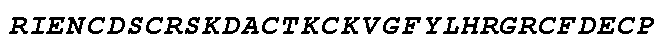 |
| | | 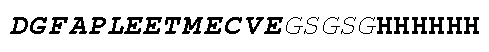 |
| | | >F4-IgG-Hole Flag HC SEQ ID NO: 181 |
| | | EVQLVESGGGLVQPGGSLRLSCAASGFNLSSY |
| | | SMHWVRQAPGKGLEWVAYISSYYGYTYYADS |
| | | VKGRFTISADTSKNTAYLQMNSLRAEDTAVYY |
| | | CARPAPGHWGFDYWGQGTLVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPSREEMT |
| | | KNQVSLSCAVKGFYPSDIAVEWESNGQPENNYK |
| | | TTPPVLDSDGSFFLVSKLTVDKSRWQQGNVFSCS |
| | | VMHEALHNHYTQKSLSLSPGKGSGSGDYKDDDD |
| | | K |
| | | >F4 LC SEQ ID NO: 182 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQWYYAPITF |
| | | GQGTKVEIKRTVAAPSVFIFPPSDEQLKSGTASV |
| | | VDLLNNFYPREAKVQWKVDNALQSGNSQESVTE |
| | | QDSKDSTYSLSSTLTLSKADYEKHKVYACEVTH |
| | | QGLSSPVTKSFNRGEC |
| | L6-N-HC | L6-N-HC appended ScFv-F49-IgG-RSPO2RA-Knob- |
| | appended | His HC SEQ ID NO: 183 |
| | ScFv-F49- | EQVLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F49-IgG- | AASGSSGGSSSGADIQMTQSPSSLSASVGDRVTIT |
| | Hole-Flag | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSQVQLVQSGAEVKKPGASVKVSCKASGGT |
| | | FSSYAISWVRQAPGQGLEWMGWINAGNGNTT |
| | | YAQKFQGRVTMTRDTSTSTVYMELSSLRSEDT |
| | | AVYYCARHYYGSGSYPKDWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLWCLVKGFYPSIAVEWESNGQPEN |
| | | NYKTTPPVLDSDGSFFLYSKLTVDKSRWQQGNV |
| | | FSCSVMHEALHNHYTQKSLSLSPGKGGGGSGGG |
| | | 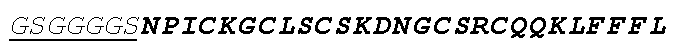 |
| | | 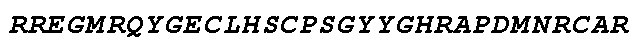 |
| | | 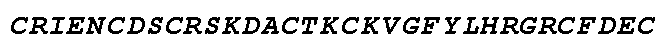 |
| | | 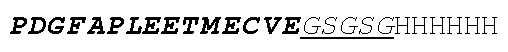 |
| | | >F49-IgG-Hole Flag HC SEQ ID NO: 184 |
| | | QVQLVQSGAEVKKPGASVKVSCKASGGTFSSY |
| | | AISWVRQAPGQGLEWMGWINAGNGNTTYAQ |
| | | KFQGRVTMTRDTSTSTVYMELSSLRSEDTAVY |
| | | YCARHYYGSGSYPDWGQGTLVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPSREEMT |
| | | KNQVSLSCAVKGFYPSDIAVEWESNGQPENNYK |
| | | TTPPVLDSDGSFFLVSKLTVDKSRWQQGNVFSCS |
| | | VMHEALHNHYTQKSLSLSPGKGSGSGDYKDDDD |
| | | K |
| | | >F49 LC SEQ ID NO: 185 |
| | | DIQMTQSPSSPSASVGDRVTITCRASQSISSYLN |
| | | WYQQKPGKAPKLLIYAASSLQSGVPSRFSGSG |
| | | SGTDFTLTISSLQPEDFATYYCQQSYSTPLTFG |
| | | GGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| | L6-N-HC | >L6-N-HC appended ScFv-F10-IgG-RSPO2RA- |
| | appended | Knob-His HC SEQ ID NO: 186 |
| | ScFv-F10- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F10-IgG- | AASGSSGGSSSGADIQMTQSPSSLSASVGDRVTIT |
| | Hole-Flag | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFTF |
| | | SSFNMFWVRQAPGKGLEWVAGIDDDGSYPNY |
| | | GSAVKGRATISRDNSKNTLYLQMNSLRAEDTA |
| | | VYYCAKSGYGGSWGGYIADDIDAWGQGTLVT |
| | | VSSASTKGPSVFPLAPSSKSTSGGTAALGSCLVKD |
| | | YFPEPVTVSWNSGALTSGVHTFPAVLQSSGLYSL |
| | | SSVVTVPSSSLGTQTYICNVNHKPSNTKVDKKVE |
| | | PKSCDKTHTCPPCPAPEAAGGPSVFLFPPKPKDTL |
| | | MISRTPEVTCVVVDVSHEDPEVKFNWYVDGVEV |
| | | HNAKTKPREEQYNSTYRVVSVLTVLHQDWLNG |
| | | KEYKCKVSNKALGAPIEKTISKAKGQPREPQVYT |
| | | LPPSREEMTKNQVSLWCLVKGFYPSDIAVEWES |
| | | NGQPENNYKTTPPVLDSDGSFFLYSKLTVDKSR |
| | | WQQGNFSCSVMHEALHNHYTQKSLSLSPGKG |
| | | 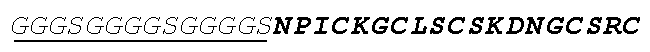 |
| | | 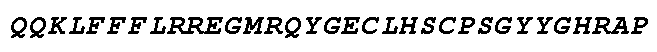 |
| | | 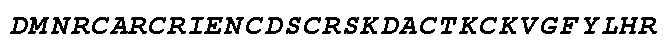 |
| | | 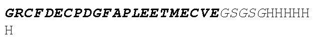 |
| | | >F10-IgG-Hole Flag HC SEQ ID NO: 187 |
| | | EVQLVESGGGLVQPGGSLRLSCAASGFTFSSF |
| | | NMFWVRQAPGKGLEWVAGIDDDGSYPNYGS |
| | | AVKGRATISRDKNSKNTLYLQMNSLRAEDTAVY |
| | | YCAKSGYGGSWGGYIADDIDAWGQGTLVTVS |
| | | SASTKGPSVFPLAPSSKSTSGGTAALGCLVKDYF |
| | | PEPVTVSWNSGALTSGVHTFPAVLQSSGLYSLSS |
| | | VVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVH |
| | | NAKTKPREEQYNSTYRVVSVLTVLHQDWLNGK |
| | | EYKCKVSNKALGAPIEKTISKAKGQPREPQVYTL |
| | | PPSREEMTKNQVSLSCAVKGFYPSDIAVEWESNG |
| | | QPENNYKTTPPVLDSDGSFFLVSKLTVDKSRWQ |
| | | QGNVFSCSVMHEALHNHYTQKSLSLSPGKGSGS |
| | | GDYKDDDDK |
| | | >F10 LC SEQ ID NO: 188 |
| | | ELTQPPSVSVSPGQTARITCSGDGSYAGSYYYG |
| | | WYQQKPGQAPVITLIYYNNKRPSGIPERGSGSL |
| | | SGSTNTLTISGVQAEDEADYYCGSADNSGAAF |
| | | GGGTKLTVLRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| | L6-N-HC | L6-N-HC appended ScFv-F7B-IgG-RSPO2RA HC |
| | appended | SEQ ID NO: 205 |
| | ScFv-F7B- | EVQLVESGGGLVQPGGSLRLSCAASGFTFTSY |
| | IgG- | YISWVRQAPGKGLEWVAEISPYSGSTYYADSV |
| | RSPO2RA | KGRFTISADTSKNTAYLQMNSLRAEDTAVYYC |
| | | ALRARPPIRLHPRGSVMDYWGQGTLTVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGASTKGPSVFPLAPSSKSTSGGTAALGCLVKDY |
| | | FPEPVTVSWNSGALTSGVHTFPAVLQSSGLYSLS |
| | | SVVTVPSSSLGTQTYICNVNHKPSNTKVDKKVEP |
| | | KSCDKTHTCPPCPAPEAAGGPSVFLFPPKPKDTL |
| | | MISRTPEVTCVVVDVSHEDPEVKFNWYVDGVEV |
| | | HNAKTKPREEQYNSTYRVVSVLTVLHQDWLNG |
| | | KEYKCKVSNKALGAPIEKTISDKAKGQPREPQVYT |
| | | LPPSREEMTKNQVSLTCLVKGFYPSDIAVEWESN |
| | | GQPENNYKTTPPVLDSDGSFFLYSKLTVDKSRW |
| | | QQGNVFSCSVMHEALHNHYTQKSLSLSPGKGGG |
| | | 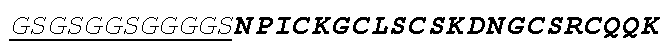 |
| | | 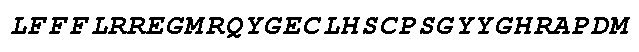 |
| | | 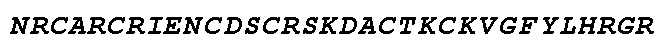 |
| | | 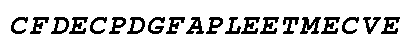 |
| | | F7B LC SEQ ID NO: 206 |
| | | DIQMTQSPSSLSASVGDRVTITCRASQSVSSAV |
| | | AWYQQKPGKAPKLLIYSASSLYSGVPSRFSGS |
| | | RSGTDTLTISSLQPEDFATYYCQQYYSLFTFG |
| | | QGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| | L6-N-HC | >L6-N-HC appended ScFv-F21-IgG-RSPO2RA HC |
| | appended | SEQ ID NO: 207 |
| | ScFv-F2I- | EVQLVESGGGLVQPGGSLRLSCAASGFTFTSY |
| | IgG- | YISWVRQAPGKGLEWVAEISPYSGSTYYADSV |
| | RSPO2RA | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNI |
| | | HSSSIHWVRQAPGKGLEWVAATYSSFGSITYA |
| | | DSVKGRFTISADTSKNTAYLQMNSLRAEDTAV |
| | | YYCARYHHPFGYALDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLTCLVKGFYPSDIAVEWESNGQPENN |
| | | YKTTPPVLDSDGSFFLYSKLTVDKSRWQQGNVF |
| | | SCSVMHEALHNHYTQKSLSLSPGKGGGGSGG |
| | | 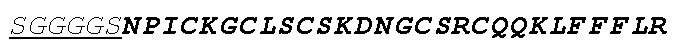 |
| | | 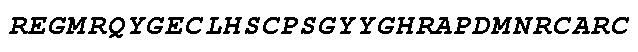 |
| | | 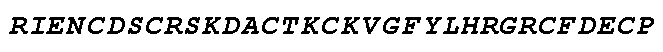 |
| | | 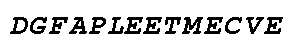 |
| | | >F21 LC SEQ ID NO: 208 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQGVYLFTFG |
| | | QGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| | L6-N-HC | >L6-N-HC appended ScFv-F7B-IgG-RSPO2RA- |
| | appended | Knob-His HC SEQ ID NO: 209 |
| | ScFv-F7B- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F7B-IgG- | AASGSSGGSSGADIQMTQSPSSLSASVGDRVTIT |
| | RSPO2RA | CRASQDVSTAVAWYQQKPGKAPKLLIYSASFL |
| | Hole-Flag | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNF |
| | | SSSSIHWVRQAPGKGLEWVAYIYPSYDYTYYA |
| | | DSVKGRFTISADTSKNTAYLQMNSLRAEDTAV |
| | | YYCARGYFYTWGGMDYWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLWCLVKGFYPSDIAVEWESNGQPEN |
| | | NYKTTPPVLDSDGSFFLYSKLTVDKSRWQQGNV |
| | | FSCSVMHEALHNHYTQKSLSLSPGKGGGGSGGG |
| | | 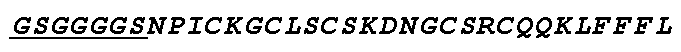 |
| | | 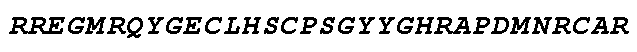 |
| | | 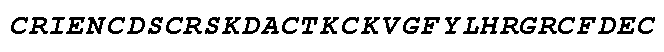 |
| | | 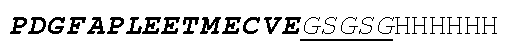 |
| | | >F7B-IgG-RSPO2RA-Hole Flag HC SEQ ID NO: 210 |
| | | EVQLVESGGGLVQPGGSLRLSCAASGFNFSSSS |
| | | IHWVRQAPGKGLEWVAYIYPSYDYTYYADSV |
| | | KGRFTISADTSKNTAYLQMNSLRAEDTAVYYC |
| | | ARGYFYTWGGMDYWGQGTLVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTC |
| | | PPCPAPEAAGGPSVFLFPKKPKDTLMISRTPEVTC |
| | | VVVDVSHEDPEVKFNWYVDGVEVHNAKTKPRE |
| | | EQYNSTYRVVSVLTVLHQDWWLNGKEYKCKVSN |
| | | KALGAPIEKTISKAKGQPREPQVYTLPPSREEMT |
| | | KNWVSLSCAVKGFYPSDIAVEWESNGQPENNYK |
| | | TTPPVLDSDGSFFLVSKLTVDKSRWQQGNVFSCS |
| | | VMHEALHNHYTQKSLSLSPGKGGGGSGGGGSGG |
| | | 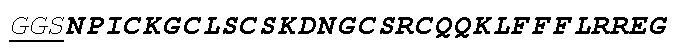 |
| | | 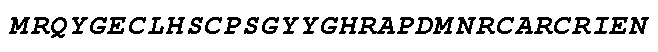 |
| | | 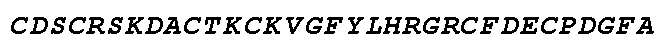 |
| | | 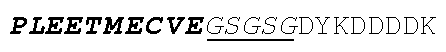 |
| | | >F7B LC SEQ ID NO: 211 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQYYSLFTFG |
| | | QGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYCEVTHQ |
| | | GLSSPVTKSFNRGEC |
| | L6-N-HC | >L6-N-HC appended ScFv-F21-IgG-RSPO2RA-Knob- |
| | appended | His HC SEQ ID NO: 212 |
| | ScFv-F2I- | EVQLVESGGGLVQPGGGSLRLSCAASGFTFTSY |
| | IgG- | YIDWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F21-IgG- | AASGSSGGSSSGADIQMTQSPSSLSASVGDRVTIT |
| | RSPO2RA | CRASQDVSTAVAWYQQKPGKAPKLLIYSASFL |
| | Hole-Flag | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNI |
| | | HSSSIHWVRQAPGKGLEWVAATYSSFGSITYA |
| | | DSVKGRFTISADTSKNTAYLQMNSLRAEDTAV |
| | | YYCARYHHPFGYALDYWGQGTLVTVSSASTK |
| | | GPSVFPLAPSSKSTSGGTAALGCLVDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLWCLVKGFYPSDIAVEWESNGQPENN |
| | | YKTTPPVLDSDGSFFLYSKLTVDKSRWQQGNVF |
| | | SCSVMHEALHNHYTQKSLSLSPGKGGGGSGGGG |
| | | 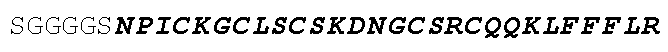 |
| | | 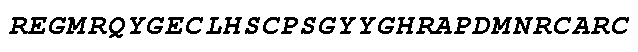 |
| | | 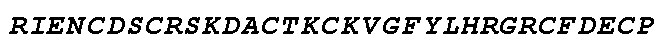 |
| | | 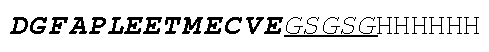 |
| | | >F2I-IgG-RSPO2RA-Hole Flag HC SEQ ID NO: 213 |
| | | EVQLVESGGGLVQPGGSLRLSCAASGFNIHSSS |
| | | IHWVRQAPGKGLEWVAATYSSFGSITYADSVK |
| | | GRFTISADTSKNTAYLQMNSLRAEDTAVYYCA |
| | | RYHHPFGYALDYWGQGTLVTVSSASTKGPSVF |
| | | PLAPSSKSTSGGTAALGCLVKDYFPEVTVSWNS |
| | | GALTSGVHTFPAVLQSSGLYSLSSVVTVPSSSLGT |
| | | QTYICNVNHKPSNTKVDKKVEPKSCDKTHTCPP |
| | | CPAPEAAGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LGAPIEKTISDKAKGQPREPQVYTLPPSREEMTKN |
| | | QVSLSCAVKGFYPSDIAVEWESNGQPENNYKTTP |
| | | PVLDSDGSFFLVSKLTVDKSRWQQGNFSCSVM |
| | | HEALHNHYTQKSLSLSPGKGGGGSGGGGSGGGG |
| | | 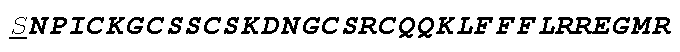 |
| | | 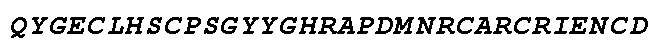 |
| | | 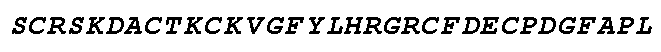 |
| | | 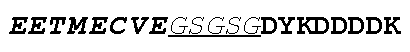 |
| | | >FI LC SEQ ID NO: 214 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQGVYLFTFG |
| | | QGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| | L6-N-HC | >L6-N-HC appended ScFv-F7B-IgG-RSPO2RA- |
| | appended | Knob-His HC SEQ ID NO: 215 |
| | ScFv-F7B- | EVQLVESGGGLVQPGGSLRLSCAASGFTFTSY |
| | IgG- | YISWRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | L6-N-HC | AASGSSGGSSSGADIQMTQSPSSLSASVGDRVTIT |
| | appended | CRASQDVSTAVAWYQQKPGKAPKLLIYSASFL |
| | ScFv-F7B- | YSGVPSRFSGSGSGTDFTLTISSLQPEDFATYY |
| | IgG-Hole- | CQQSYTTPPTFGQGTKVEIKGGGGSGGGGSGG |
| | Flag | GGSEVQLVESGGGLVQPGGSLRLSCAASGFNF |
| | | SSSSIHWVRQAPGKGLEWVAYIYPSYDYTYYA |
| | | DSVKGRFTISADTSKNTAYLQMNSLRAEDTAV |
| | | YYCARGYFYTWGGMDYWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLWCLVKGFYPSDIAVEWESNGQPEN |
| | | NYKTTPPVLDSDGSFFLYSKLTVDKSRWQQGNV |
| | | FSCSVMHEALHNHYTQKSLSLSPGKGGGGSGGG |
| | | 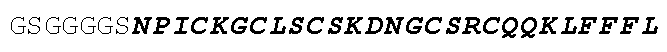 |
| | | 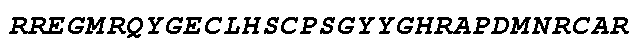 |
| | | 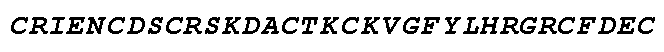 |
| | | 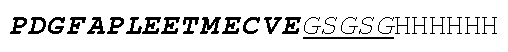 |
| | | >L6-N-HC appended ScFv-F7B-IgG-Hole Flag HC |
| | | SEQ ID NO: 216 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGKVEIKGGGGSGGGGSGG |
| | | GGSEVQLVESGGGLVQPGGSLRLSCAASGFNF |
| | | SSSSIHWVRQAPGKGLEWVAYIYPSYDYTYYA |
| | | DSVKGRFTISADTSKNTAYLQMNSLRAEDTAV |
| | | YYCARGYFYTWGGMDYWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLSCAVKGFYPSDIAVEWESNGQPEN |
| | | NYKPPTTVLDSDGSFFLVSKLTVDKSRWQQGNV |
| | | FSCSVMHEALHNHYTQKSLSLSPGKGSGSGDYK |
| | | DDDDK |
| | | >F7B LC SEQ ID NO: 217 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQYYSLFTFG |
| | | QGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| | L6-N-HC | >L6-N-HC appended ScFv-F2I-IgG-RSPO2RA-Knob- |
| | appended | HIS HC SEQ ID NO: 218 |
| | ScFv-F2I- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-hi + | ALRARPPIRLHPRGSVMDYWGQGTLVTVSSGS |
| | L6-N-HC | AASGSSGGSSSGADIQMTQSPSSLSASVGDRVTIT |
| | appended | CRASQDVSTAVAWYQQKPGKAPKLLIYSASFL |
| | ScFv-F2I- | YSGVPSRFSGSGSGTDFTLTISSLQPEDFATYY |
| | IgG-Hole- | CQQSYTTPPTFGQGTKVEIKGGGGSGGGGSGG |
| | Flag | GGSEVQLVESGGGLVQPGGSLRLSCAASHGNI |
| | | HSSSIHWVRQAPGKGLEWVAATYSSFGSITYA |
| | | DSVKGRFTISADTSKNTAYLQMNSLRAEDTAV |
| | | YYCARYHHPFGYALDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLWCLVKGFYPSIAVEWESNGQPENN |
| | | YKTTPPVLDSDGSFFLYSKLTVDKSRWQQGNVF |
| | | SCVMHEALHNHYTQKSLSLSPGKGGGGSGGGG |
| | | 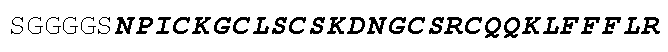 |
| | | 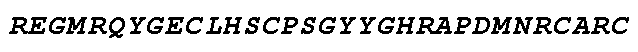 |
| | | 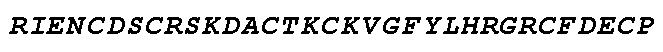 |
| | | 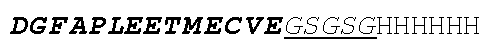 |
| | | >L6-N-HC appended ScFv-F2I-IgG-Hole Flag HC |
| | | SEQ ID NO: 219 |
| | | EVQLVESGGGLVQPGGSLRLSCAASGFTFTSY |
| | | YISWVRQAPGKGLEWVAEISPYSGSTYYADSV |
| | | KGRFTISADTSKNTAYLQMNSLRAEDTAVYYC |
| | | ALRARPPIRLHPRGSVMDYWGQGTLVTVSSGS |
| | | AASGSSGGSSSGADIQMTQSPSSLSASVGDRVTIT |
| | | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNI |
| | | HSSSIHWVRQAPGKGLEWVAATYSSFGSITYA |
| | | DSVKGRFTISADTSKNTAYLQMNSLRAEDTAV |
| | | YYCARYHHPFGYALDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGTQTYICNVHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLSCAVKGFYPSDIAVEWESNGQPENN |
| | | YKTTPPVLDSDGSFFLVSKLTVDKSRWQQGNVF |
| | | SCSVMHEALHNHYTQKSLSLSPGKGSGSGDYKD |
| | | DDDK |
| | | >F2I LC SEQ ID NO: 220 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQGVVLFTFG |
| | | QGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLASSPVTKSFNRGEC |
| | L6-N-HC | >L6-N-HC appended ScFv-F7B-IgG-RSPO2RA- |
| | appended | Knob-His HC SEQ ID NO: 221 |
| | ScFv-F7B- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F7B-IgG- | AASGSSGGSSSGADIQMTQSPSSLSASVGDRVTIT |
| | Hole-Flag | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNF |
| | | SSSSIHWVRQAPGKGLEWVAYIYPSYDYTYYA |
| | | DSVKGRFTISADTSKNTAYLQMNSLRAEDTAV |
| | | YYCARGYFYTWGGMDYWGQGTLVTVSSAST |
| | | KGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTP |
| | | EVTCVVVDVSHEDPEVKFNWYVDGVEVHNAKT |
| | | KPREEQYNSTYRVVSVLTVLHQDWLNGKEYKC |
| | | KVSNKALGAPIEKTISKAKGQPREPQVYTLPPSRE |
| | | EMTKNQVSLWCLVKGFYPSDIAVEWESNGPQPEN |
| | | NYKTTPPVLDSDGSFFLYSKLTVDKSRWQQGNV |
| | | FSCSVMHEALHNHYTQKSLSLSPGKGGGGSGGG |
| | | 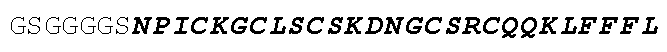 |
| | | 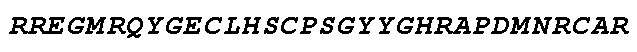 |
| | | 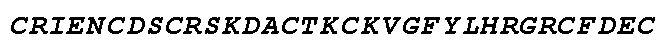 |
| | | 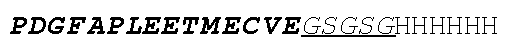 |
| | | >F7B-IgG-Hole Flag HC SEQ ID NO: 222 |
| | | EVQLVESGGGLVQPGGSLRLSCAASGFNFSSSS |
| | | IHWVRQAPGKGLEWVAYIYPSYDYTYYADSV |
| | | KGRFTISADTSKNTAYLQMNSLRAEDTAVYYC |
| | | ARGYFYTWGGMDYWGQGTLVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPSREEMT |
| | | KNQVSLSCAVKGFYPSDIAVEWESNGQPENNYK |
| | | TTPPVLDSDGSFFLVSKLTVDKSRWQQGNVFSCS |
| | | VMHEALHNHYTQKSLSLSPGKGSGSDYKDDDD |
| | | K |
| | | >F7B LC SEQ ID NO: 223 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQYYSLFTFG |
| | | QGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |
| | L6-N-HC | >L6-N-HC appended ScFv-F2I-IgG-RSPO2RA-Knob- |
| | appended | His HC SEQ ID NO: 224 |
| | ScFv-F2I- | EVQLVESGGGLVQPGGSLRLSCAASGFTFTSY |
| | IgG- | YISWVRQAPGKGLEWVAEISPYSGSTYYADSV |
| | RSPO2RA- | KGRFTISADTSKNTAYLQMNSLRAEDTAVYYC |
| | Knob-his + | ALRARPPIRLHPRGSVMDYWGQGTLVTVSSGS |
| | F2I-IgG- | AASGSSGGSSSGADIQMTQSPSSLSASVGDRVTIT |
| | Hole-Flag | CRASQDVSTAVAWYQQKPGKAPKLLIYSASFL |
| | | YSGVPSRFSGSGSGTDFTLTISSLQPEDFATYY |
| | | CQQSYTTPPTFGQGTKVEIKGGGGSGGGGSGG |
| | | GGSEVQLVESGGGLVQPGGSLRLSCAASGFNI |
| | | HSSSIHWVRQAPGKGLEWVAATYSSFGSITYA |
| | | DSVKGRFTISADTSKNTAYLQMNSLRAEDTAV |
| | | YYCARYHHPFGYALDYWGQGTLVTVSSASTK |
| | | GPSVFPLAPSSKSTSGGTAALGCLVKDYFPEPVT |
| | | VSWNSGALTSGVHTFPAVLQSSGLYSLSSVVTVP |
| | | SSSLGQGTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCK |
| | | VSNKALGAPIEKTISKAKGQPREPQVYTLPPSREE |
| | | MTKNQVSLWCLVKGFYPSDIAVEWESNGQPENN |
| | | YKTTPPVLDSDGSFFLYSKLTVDKSRWQQGNVF |
| | | SCSVMHEALHNHYTQKSLSLSPGKGGGGSGGGG |
| | | 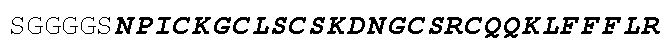 |
| | | 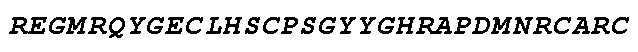 |
| | | 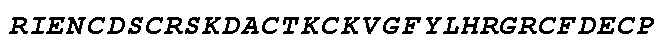 |
| | | 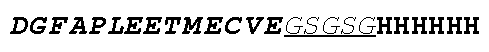 |
| | | >F2I-IgG-Hole Flag HC SEQ ID NO: 225 |
| | | EVQLVESGGGLVQPGGSLRLSCAASGFNIHSSS |
| | | IHWVRQAPGKGLEWVAATYSSFGSITYADSVK |
| | | GRFTISADTSKNTAYLQMNSLRAEDTAVYYCA |
| | | RYHHPFGYALDYWGQGTLVTVSSASTKGPSVF |
| | | PLAPSSKSTSGGTAALGCLVKDYFPEPVTVSWNS |
| | | GALTSGVHTFPAVLQSSGLYSKSSVVTVPSSSLGT |
| | | QTYICNVNHKPSNTKVDKKVEPKSCDKTHTCPP |
| | | CPAPEAAGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LGAPIEKTISKAKGQPREPQVYTLPPSREEMTKN |
| | | QVSLSCAVKGFYPSDIAVEWESNGQPENNYKTTP |
| | | PVLDSDGSFFLVSKLTVDKSRWQQGNVFSCSVM |
| | | HEALHNHYTQKSLSLSPGKGSGSGDYKDDDDK |
| | | >F2I LC SEQ ID NO: 226 |
| | | DIQMTQSPSSLSASVGDRVTITCRASQSVSSAV |
| | | AWYQQKPGKAPKLLIYSASSLYSGVPSRFSGS |
| | | RSGTDFTLTISSLQPEDFATYYCQQBVYLFTFG |
| | | QGTKVEIKRTVAAPSVFIFPPSDEQLKGTASVV |
| | | CLLNNFYPREAKVQWKVDNALQSGNSQESVTEQ |
| | | DSKDSTYSLSSTLTLSKADYEKHKVYACEVTHQ |
| | | GLSSVTKSFNRGEC |
| RSPO2RA-N-LC-ScFv-IgG | RSPO2RA- | RSPO2RA-F12578 LC SEQ ID NO: 227 |
| | L6-F12578 | 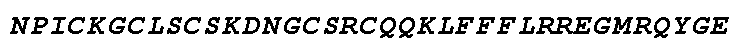 |
| | | 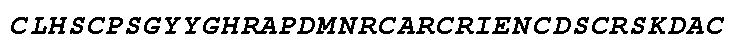 |
| | | 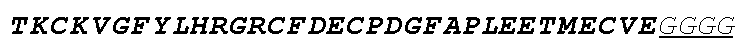 |
| | | SGGGGSGGGGSDIELTQPPSVSVAPGQTARISCSGDN |
| | | IGSFYVHWYQQKPGQAPVLVIYDKSNRPSGIPERFSG |
| | | SNSGNTATLTISGTQAEDEADYYCQSYANTLSLVFGG |
| | | GTKLTVLGQPKAAPSVTLFPPSSEELQANKATLVCLI |
| | | SDFYPGAVTVAWKADSSPVKAGVETTTPSKQSNNKYA |
| | | ASSYLSLTPEQWKSHRSYSCQVTHEGSTVEKTVAPTE |
| | | CS |
| | | L6_F12578 HC SEQ ID NO: 228 |
| | | EVQLVESGGGGLVQPGGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSEVQLVESGGGLVQPGGSLRLSCAASGFTFSHYT |
| | | LSWVRQAPGKGLEWVSVISGDGSYTYYADSVKGRFTI |
| | | SSDNSKNTLYLQMNSLRAEDTAVYYCARNFIKYVFAN |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLMIS |
| | | RTPEVTCVVVDVSHEDPEVKFNWYVDGVEVHNAKTKP |
| | | REEQYNSTYRVVSVLTVLHQDWLNGKEYKCKVSNKAL |
| | | GAPIEKTISKAKGQPREPQVYTLPPSREEMTKNQVSL |
| | | TCLVKGFYPSDIAVEWESNGQPENNYKTTPPVLDSDG |
| | | SFFLYSKLTVDKSRWQQGNVSCSVMHEALHNHYTQK |
| | | SLSLSPGK |
| RSPO2RA-N-LC-ScFv-IgG | RSPO2RA- | RSPO2RA-F127 LC SEQ ID NO: 229 |
| | L6-F127 | 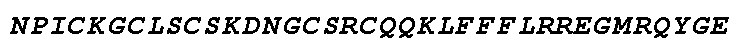 |
| | | 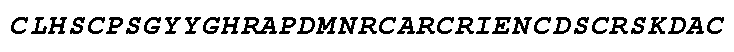 |
| | | 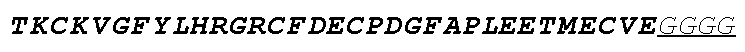 |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQYWYGVAPITF |
| | | GQGTKVEIKRTVAAPSVFIFPPSDEQLKSGTASVVCL |
| | | LNNFYPREAKVQWKVDNALQSGNSQESVTEQDSKDST |
| | | YSLSSTLTLSKADYEKHKVYACEVTHQGLGGPVTKSF |
| | | NRGEC |
| | | L6_F127 HC SEQ ID NO: 230 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSEVQLVESGGGLVQPGGSLRLSCAASGFNISSSY |
| | | IHWVRQAPGKGLEWVAYIYSSYGSTYYADSVKGRFTI |
| | | SADTSKNTAYLQMNSLRAEDTAVYYCARASWYALDYW |
| | | GQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPK |
| | | SCDKTHTCPPCPAPEAAGGPSVFLFPPKPKDTLMISR |
| | | TPEVTCVVVDVSHEDPEVKFNWYVDGVEVHNAKTKPR |
| | | EEQYNSTYRVVSVLTVLHQDWLNGKEYKCKVSNKALG |
| | | APIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNHYTQKS |
| | | LSLSPGK |
| RSPO2RA-N-LC-ScFv-IgG | RSPO2RA- | RSPO2RA-F58 LC SEQ ID NO: 231 |
| | L6-F58 | 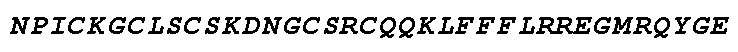 |
| | | 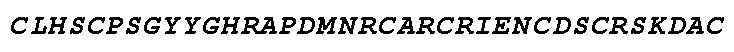 |
| | | 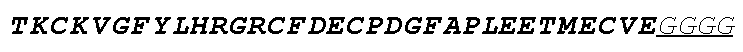 |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQWYSSGHVLIT |
| | | FGQGTKVEIKRTVAAPSVFIFPPSDEQLKSGTASVVC |
| | | LLNNFYPREAKVQWKVDNALQSGNSQESVTEQDSKDS |
| | | TYSLSSTLTLSKADYEKHKVYACEVTHQGLSSPVTKS |
| | | FNREC |
| | | L6_F58 HC SEQ ID NO: 232 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSEVQLVESGGGLVQPGGSLRLSCAASGFNISYSY |
| | | IHWVRQAPGKGLEWVASIYSSSGSTSYADSVKGRFTI |
| | | SADTSKNTAYLQMNSLRAEDTAVYYCARGAIDYWGQG |
| | | TLVTVSSASTKGPSVFPLAPSSKSTSGGTAALGCLVK |
| | | DYFPEPVTVSWNSGALTSGVHTFPAVLQSSGLYSLSS |
| | | VVTVPSSSLGTQTYICNVNHKPSNTKVDKKVEPKSCD |
| | | KTHTCPPCPAPEAAGGPSVFLFPPKPKDTLMISRTPE |
| | | VTCVVVDVSHEDPEVKFNWYVDGVEVHNAKTKPREEQ |
| | | YNSTYRVVSVLTVLHQDWLNGKEYKCKVSNKALGAPI |
| | | EKTISKAKGQPREPQVYTLPPSREEMTKNQVSLTCLV |
| | | KGFYPSDIAVEWESNGQPENNYKTTPPVLDSDGSFFL |
| | | YSKLTVDKSRWQQGNVFSCSVMHEALHNHYTQKSLSL |
| | | SPGK |
| RSPO2RA-N-LC-ScFv-IgG | RSPO2RA- | RSPO2RA-F4-2 LC SEQ ID NO: 233 |
| | L6-F4-2 | 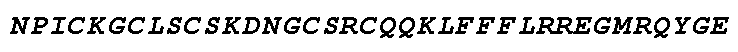 |
| | | 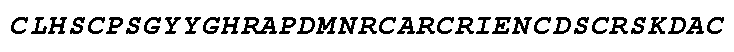 |
| | | 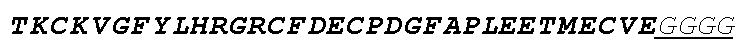 |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQSYAAYLFTFG |
| | | QGTKVEIKRTVAAPSVFIFPPSDEQLKSGTASVVCLL |
| | | NNFYPREAKVQWKVDNALQSGNSQESVTEQDSKDSTY |
| | | SLSSTLTLSKADYEKHKVYACEVTHQGLSSPVTKSFN |
| | | RGEC |
| | | L6-F4-2 HC SEQ ID NO: 234 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSEVQLVESGGGLVQPGGSLRLSCAASGFNISYYY |
| | | IHWVRQAPGKGLEWVASIYPSSGYTYYADSVKGRFTI |
| | | SADTSKNTAYLQMNSLRAEDTAVYYCARSSFYWAMDY |
| | | WGQGTLVTVSSASTKGPSVFPLAPSSKSTSGGTAALG |
| | | CLVDKDYFPEPVTVSWNSGALTSGVHTFPAVLQSSGLY |
| | | SLSSVVTVPSSSLGTQTLYICNVNHKPSNTKVDKKVEP |
| | | KSCDKTHTCPPCPAPEAAGGPSVFLFPPKPKDTLMIS |
| | | RTPEVTCVVVDVSHEDPEVKFNWYDGVEVHNAKTKP |
| | | REEQYNSTYRVVSVLTVLHQDWLNGKEYKCKVSNKAL |
| | | GAPIEKTISKAKGQPREPQVYTLPPSREEMTKNQVSL |
| | | TCLVKGFYPSDIAVEWESNGQPENNYKTTPPVLDSDG |
| | | SFFLYSKLTVDKSRWQQGNVFSCSVMHEALHNHYTQK |
| | | SLSLSPGK |
| RSPO2RA-N-LC-ScFv-IgG | RSPO2RA- | RSPO2RA-F4 LC SEQ ID NO: 235 |
| | L6-F4 | 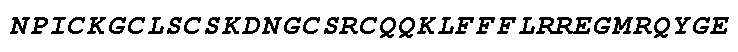 |
| | | 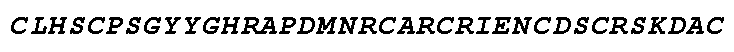 |
| | | 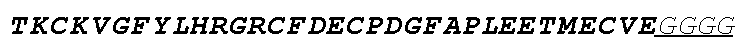 |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQWYYAPITFGQ |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHKVYACEVTHQGLSSPVTKSFNR |
| | | GEC |
| | | L6-F4 HC SEQ ID NO: 236 |
| | | EVQLVESGGGGLVQPGGSL4LSCAASGFTFTSYYISWY |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSEVQLVESGGGLVQPGGSLRLSCAASGFNLSSYS |
| | | MHWVRQAPGKGLEWVAYISSYYGYTYYADSVKGRFTI |
| | | SADTSKNTAYLQMNSLRAEDTAVYYCARPAPGHWGFD |
| | | YWGQGTLVTVSSASTKGPSVFPLAPSSKSTSGGTAAL |
| | | GCLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPEAAGGPSVFLFPPKPKDTLMI |
| | | SRTPEVTCVVVDVSHEDPEVKFNWYVDGVEVHNAKTK |
| | | PREEQYNSTYRVVSVLTVLHQDWLNGKEYKCKVSNKA |
| | | LGAPIEKTISKAKGQPREPQVYTLPPSREEMTKNQVS |
| | | LTCLVKGFYPSDIAVEWESNGQPENNYKTTPPVLDSD |
| | | GSFFLYSKLTVDKSRWQQGNVFSCSVMHEALHNHYTQ |
| | | KSLSLSPGK |
| RSPO2RA-N-LC-ScFv-IgG | RSPO2RA- | RSPO2RA-F49 LC SEQ ID NO: 237 |
| | L6-F49 | 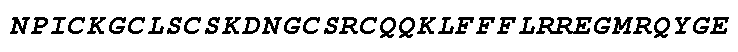 |
| | | 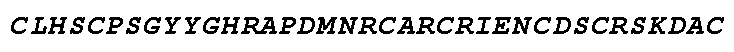 |
| | | 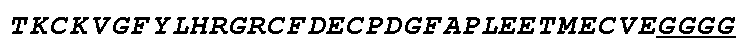 |
| | | SGGGGSGGGGSDIQMTQSPSSPSASVGDRVTITCRAS |
| | | QSISSYLNWYQQKPGKAPKLLIYAASSLQSGVPSRFS |
| | | GSGSGTDFTLTISSLQPEDFATYYCQQSYSTPLTFGG |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHKVYACEVTHQGLSSPVTKSFNR |
| | | GEC |
| | | L6-F49 HC SEQ ID NO: 238 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSQVQLVQSGAEVKKPGASVKVSCKASGGTFSSYA |
| | | ISWVRQAPGQGLEWMGWINAGNGNTTYAQKFQGRVTM |
| | | TRDTSTSTVYMELSSLRSEDTAVYYCARHYYGSGSYP |
| | | DWGQGTLVTVSSASTKGPSVFPLAPSSKSTSGGTAAL |
| | | GCLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPEAAGGPSVFLFPPKPKDTLMI |
| | | SRTPEVTCVVVDVSHEDPEVKFNWYDGVEVHNAKTK |
| | | PREEQYNSTYRVVSVLTVLHQDWLNGKEYKCKVSNKA |
| | | LGAPIEKTISKAKGQPREPQVYTLPPSREEMTKNQVS |
| | | LTCLVKGFYPSDIAVEWESNGQPENNYKTTPPVLDSD |
| | | GSFFLYSKLTVDKSRWQQGNVFSCSVMHEALHNHYTQ |
| | | KSLSLSPGK |
| RSPO2RA-N-LC-ScFv-IgG | RSPO2RA- | RSPO2RA-F49 LC SEQ ID NO: 239 |
| | L6-F49 | 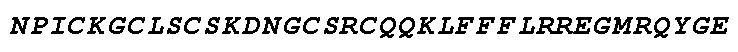 |
| | | 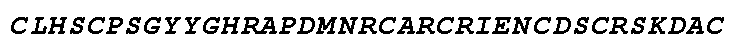 |
| | | 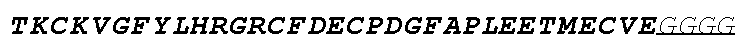 |
| | | SGGGGSGGGGSDIQMTQSPSSPSASVGDRVTITCRAS |
| | | QSISSYLNWYLQQKPGKAPKLLIYAASSLQSGVPSRFS |
| | | GSGSGTDFTLTISSLQPEDFATYYCQQSYSTPLTFGG |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHVYACEVTHQGLSSPVTKSFNR |
| | | GEC |
| | | L6-F49 HC SEQ ID NO: 240 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSQVQLVQSGAEVKKPGASVKVSCKASGGTFSSYA |
| | | ISWVRQAPGQGLEWMGWINAGNGNTTYAQKFQGRVTM |
| | | TRDTSTSTVYMELSSLRSEDTAVYYCARGYYGSGSYP |
| | | DWGQGTLVTVSSASTKGPSVFPLAPSSKSTSGGTAAL |
| | | GCLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPEAAGGPSVFLFPPKPKDTLMI |
| | | SRTPEVTCVVVDVSHEDPEVKFNWYVDGVEVHNAKTK |
| | | PREEQYNSTYRVVSVLTVLHQDWLNGKEYKCKVSNKA |
| | | LGAPIEKTISKAKGQPREPQVYTLPPSREEMTKNQVS |
| | | LTCLVKGFYPSDIAVEWESNGQPENNYKTTPPVLDSD |
| | | GSFFLYSKLTVDKSRWQQGNVFSCSVMHEALHNHYTQ |
| | | KSLSLSPGK |
| RSPO2RA-N-LC-ScFv-IgG | RSPO2RA- | RSPO2RA-F49 LC SEQ ID NO: 241 |
| | L6-F49 | 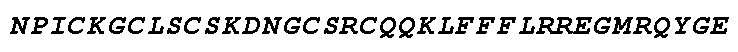 |
| | | 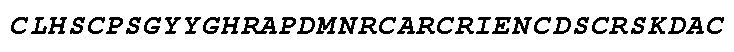 |
| | | 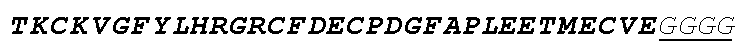 |
| | | SGGGGSGGGGSDIQMTQSPSSPSASVGDRVTITCRAS |
| | | QSISSYLNWYQQKPGKAPKLLIYAASSLQSGVPSRFS |
| | | GSGSGTDFTLTISSLQPEDFATYYCQQSYSTPLTFGG |
| | | GTKVEIKRTVAAPSVFIFPPSDEQLKSGTASVVCLLN |
| | | NFYPREAKVQWKVDNALQSGNQESVTEQDSKDSTYS |
| | | LSSTLTLSKADYEKHKVYACEVTHQGLGGPVTKSFNR |
| | | GEC |
| | | L6-F49 HC SEQ ID NO: 242 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSQVQLVQSGAEVKKPGASVKVSCKASGGTFSSYA |
| | | ISWVRQAPGQGLEWMGWINAGNGNTTYAQKFQGRVTM |
| | | TRDTSTSTVYMELSSLRSEDTAVYYCARHYYGSGSYP |
| | | DWGQGTLVTVSSASTKGPSVFPLAPSSKSTSGGTAAL |
| | | GCLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPEAAGGPSVFLFPPKPKDTLMI |
| | | SRTPEVTCVVVDVSHEDPEVKFNWYDGVEVHNAKTK |
| | | PREEQYNSTYRVVSVLTVLHQDWLNGKEYKCKVSNKA |
| | | LGAPIEKTISKAKGQPREPQVYTLPPSREEMTKNQVS |
| | | LTCLVKGFYPSDIAVEWESNGQPENNYKTTPPVLDSD |
| | | GSFFLYSKLTVDKSRWQQGNVFSCSVMHEALHNHYTQ |
| | | KSLSLSPGK |
| RSPO2RA-N-LC-ScFv-IgG | RSPO2RA- | RSPO2RA-F10 LC SEQ ID NO: 243 |
| | L6-F10 | 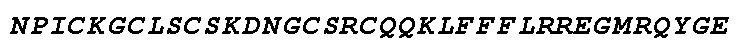 |
| | | 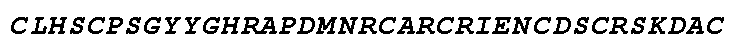 |
| | | 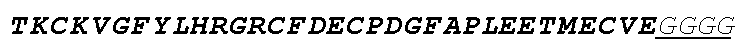 |
| | | SGGGGSGGGGSELTQPPSVSVSPGQTARITCSGDGSY |
| | | AGSYYYGWYQQKPGQAPVTLIYYNNKRPSGIPERFSG |
| | | SLSGSTNTLTISGVQAEDEADYYCGSADNSGAAFGGG |
| | | TKLTVLRTVAAPSVFIFPPSDEQLKSGTASVVCLLNN |
| | | FYPREAKVQWKVDNALQSGNSQESVTEQDSKDSTYSL |
| | | SSTLTLSKADYEKHKVYACEVTHQGLSSPVTKSFNRG |
| | | EC |
| | | L6-F10 HC SEQ ID NO: 244 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSEVQLVESGGGLVQPGGSLRLSCAASGFTFSSFN |
| | | MFWVRQAPGKGLEWVAGIDDDGSYPNYGSAVKGRATI |
| | | SRDNSKNTLYLQMNSLRAEDTAVYYCAKSGYGGSWGG |
| | | YIADDIDAWGQGTLVTVSSASTKGPSVFPLAPSSKST |
| | | SGGTAALGCLVKDYFPEPVTVSWNSGALTSGVHTFPA |
| | | VLQSSGLYSLSSVVTVPSSSLGTQTYICNVNHKPSNT |
| | | KVDKKVEPKSCDKTHTCPPCPAPEAAGGPSVFLFPPK |
| | | PKDTLMISRTPEVTCVVVDVSHEDPEVKFNWYDGVE |
| | | VHNAKTKPREEQYNSTYRVVSVLTVLHQDWLNGKEYK |
| | | CKVSNKALGAPIEKTISKAKGQPREPQVYTLPPSREE |
| | | MTKNQVSLTCLVKGFYPSDIAVEWESNGQPENNYKTT |
| | | PPVLDSDGSFFLYSKLTVDKSRWQQGNVFSCSVMHEA |
| | | LHNHTYQKSLSLSPGK |
| RSPO2RA-N-LC-ScFv-IgG | RSPO2RA- | RSPO2RA-F2I SEQ ID NO: 245 |
| | L6-F2I | 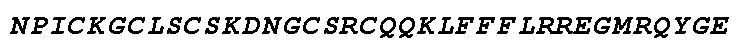 |
| | | 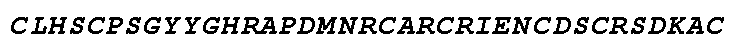 |
| | | 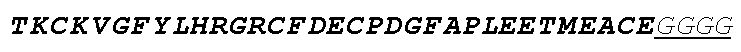 |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQGVYLFTFGQG |
| | | TKVEIKRTVAAPSVFIFPPSDEQLKSGTASVVCLLNN |
| | | FYPREAKVQWKVDNALQSGNSQESVTEQDSKDSTYSL |
| | | SSTLTLSKADYEKHKVYACEVTHQGLSSPVTKSFNRG |
| | | EC |
| | | L6-F2I HC SEQ ID NO: 246 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSGADIQMTQS |
| | | PSSLSASVGDRVTITCRASQDVSTAVAWYQQKPGKAP |
| | | KLLIYSASFLYSGVPSRFSGSGSGTDFTLTISSLQPE |
| | | DFATYYCQQSYTTPPTFGQGTKVEIKGGGGSGGGGSG |
| | | GGGSEVQLVESGGGLVQPGGSLRLSCAASGFNIHSSS |
| | | IHWVRQAPGKGLEWVAATYSSFGSITYADSVKGRFTI |
| | | SADTSKNTAYLQMNSLRAEDTAVYYCARYHHPFGYAL |
| | | DYWGQGTLVTVSSASTKGPSVFPLAPSSKSTSGGTAA |
| | | LGCLVKDYFPEPVTVSWNSGALTSGVHTFPAVLQSSG |
| | | LYSLSSVVTVPSSSLGTQTYICNVNHKPSNTKVDKKV |
| | | EPKSCDKTHTCPPCPAPEAAGGPSVFLFPPKPKDTLM |
| | | ISRTPEVTCVVVDVSHEDPEVKFNWYDGVEVHNAKT |
| | | KPREEQYNSTYRVVSVLTVLHQDWLNGKEYKCKVSNK |
| | | ALGAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHYT |
| | | QKSLSLSPGK |
| RSPO2RA-N-LC-ScFv-IgG | RSPO2RA- | RSPO2RA-F7B-LC SEQ ID NO: 247 |
| | L6-F7B | 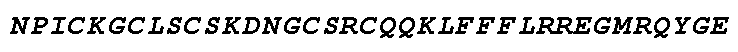 |
| | | 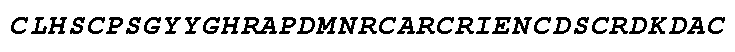 |
| | | 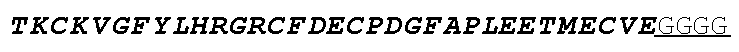 |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQYYSLFTFGQG |
| | | TKVEIKRTVAAPSVFIFPPSDEQLKSGTASVVCLLNN |
| | | FYPREAKVQWKVDNALQSGNSQESVTEQDSKDSTYSL |
| | | SSTLTLSKADYEKHKVYACEVTHQGLSSPVTKSFNRG |
| | | EC |
| | | L6-F7B HC SEQ ID NO: 248 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGSAASGSSGGSSSSGADIQMTQS |
| | | PSSLSASVGDRVTITCRASQDVSTAVAWYQQKPGKAP |
| | | KLLIYSASFLYSGVPSRFSGSGSGTYDFTLTISSLQPE |
| | | DFATYYCQQSYTTPPTFGQGTKVEIKGGGGSGGGGSG |
| | | GGGSEVQLVESGGGLVQPGGSLRLSCAASGFNFSSSS |
| | | IHWVRQAPGKGLEWVAYIYPSYDYTYYADSVKGRFTI |
| | | SADTSKNTAYLQMNSLRAEDTAVYYCARGYFYTWGGM |
| | | DYWGQGTLVTVSSASTKGPSVFPLAPSSKSTSGGTAA |
| | | LGCLVKDYFPEPVTVSWNSGALTSGVHTFPAVLQSSG |
| | | LYSLSSVVTVPSSSLGTQTYICNVNHKPSNTKVDKKV |
| | | EPKSCDKTHTCPPCPAPEAAGGPSVFLFPPKPKDTLM |
| | | ISRTPEVTCVVVDVSHEDPEVKFNWYVDGVEVHNAKT |
| | | KPREEQYNSTYRVVSVLTVLHQDWLNGKEYKCKVSNK |
| | | ALGAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHYT |
| | | QKSLSLSPGK |
| 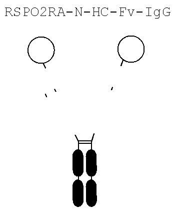 | RSPO2RA- L6-F12578 | RSPO2RA-L6-F12578 HC SEQ ID NO: 249 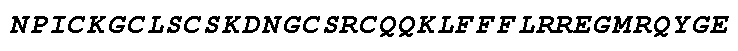 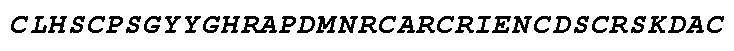 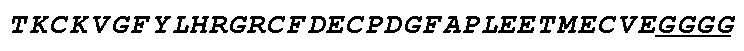 SGGGGSGGGGSEVQLVESGGGLVQPGGSLRLSCAASG FTFTSYYISWVRQAPGKGLEWVAEISPYSGSTYYADS VKGRFTISADTSKNTAYLQMNSLRAEDTAVYYCALRA RPPIRLHPRGSVMDYWGQGTLVTVSSGGGGSGGGGSG GGGSEVQLVESGGGLVQPGGGSLRLSCAASGFTFSHYT LSWVRQAPGKGLEWVSVISGDGSYTYYADSVKGRFTI SSDNDKNTLYLQMNSLRAEDTAVYYCARNFIKYVFAN |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLMIS |
| | | RTPEVTCVVVDVSHEDPEVKFNWYVDGVEVHNAKTKP |
| | | REEQYNSTYRVVSVLTVLHQDWLNGKEYKCKVSNKAL |
| | | GAPIEKTISKAKGQPREPQVYTLPPSREEMTKNQVSL |
| | | TCLVKGFYPSDIAVEWESNGQPENNYKTTPPVLDSDG |
| | | SFFLYSKLTVDKSRWQQGNVFSCSVMHEALHNHTYQK |
| | | SLSLSPGK |
| | | L6-F12578 LC SEQ ID NO: 250 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKGGGG |
| | | SGGGGSGGGGSDIELTQPPSVSVAPGQTARISCSGDN |
| | | IGSFYVHWYQQKPGQAPVLVIYDKSNRPSGIPERFSG |
| | | SNSGNTATLTISGTQAEDEADYYCQSYANTLSLVFGG |
| | | GTKLTVLGQPKAAPSVTLFPPSSEELQANKATLVCLI |
| | | SDFYPGAVTVAWKADSSPVKAGVETTTPSKQSNNKYA |
| | | ASSYLSLTPEQWKSHRSYSCQVTHEGSTVEKTVAPTE |
| | | CS |
| RSPO2RA-N-HC-Fv-IgG | RSPO2RA- | RSPO2RA-L6-F127 HC SEQ ID NO: 251 |
| | L6-F127 | 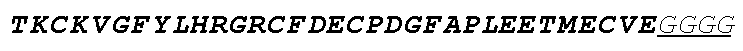 |
| | | 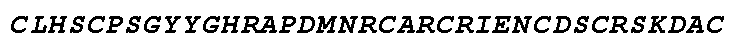 |
| | | 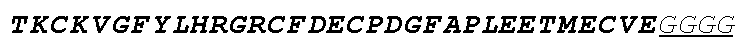 |
| | | SGGGGSGGGGSEVQLVESGGGLVQPGGSLRLSCAASG |
| | | FTFTSYYISWVRQAPGKGLEWVAEISPYSGSTYYADS |
| | | VKGRFTISADTSKNTAYLQMNSLRAEDTAVYYCALRA |
| | | RPPIRLHPRGSVMDYWGQGTLVTVSSGGGGSGGGGSG |
| | | GGGSEVQLVESGGGLVQPGGSLRLSCAASGFNISSSY |
| | | IHWVRQAPGKGLEWVAYIYSSYGSTYYADSVKGRFTI |
| | | SADTSKNTAYLQMNSLRAEDTAVYYCARASWYALDYW |
| | | GQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPK |
| | | SCDKTHTCPPCPAPEAAGGPSVFLFPPKPKDTLMISR |
| | | TPEVTCVVVDVSHEDPEVKFNWYVDGVEVHNAKTKPR |
| | | EEQYNSTYRVVSVLTVLHQDWLNGKEYKCKVSNKALG |
| | | APIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNHYTQKS |
| | | LSLSPGK |
| | | L6-F127 LC SEQ ID NO: 252 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKGGGG |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQYWYGVAPITF |
| | | GQGTKVEIKRTVAAPSVFIFPPSDEQLKSGTASVVCL |
| | | LNNFYPREAKVQWKVDNALQSGNSQESVTEQDSKDST |
| | | YSLSSTLTLSKADYEKHKVYACEVTHQGLSSPVTKSF |
| | | NRGEC |
| RSPO2RA-N-HC-Fv-IgG | RSPO2RA- | RSPO2RA-L6-F58 HC SEQ ID NO: 253 |
| | L6-F58 | 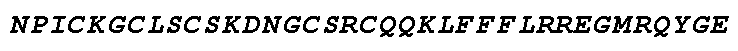 |
| | | 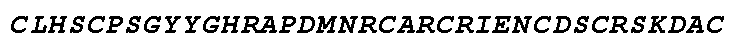 |
| | | 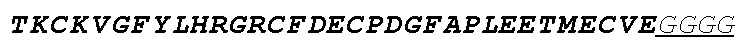 |
| | | SGGGGSGGGGSEVQLVESGGGLVQPGGSLRLSCAASG |
| | | FTFTSYYISWVRQAPGKGLEWVAEISPYSGSTYYADS |
| | | VKGRFTISADTSKNTAYLQMNSLRAEDTAVYYCALRA |
| | | RPPIRLHPRGSVMDYWGQGTLVTVSSGGGGSGGGGSG |
| | | GGGSEVQLVESGGGLVQPGGSLRLSCAASGFNISYSY |
| | | IHWVRQAPGKGLEWVASIYSSSGSTSYADSVKGRFTI |
| | | SADTSKNTAYLQMNSLRAEDTAVYYCARGAIDYWGQG |
| | | TLVTVSSASTKGPSVFPLAPSSKSTSGGTAALGCLVK |
| | | DYFPEPVTVSWNSGALTSGVHTFPAVLQSSGLYSLSS |
| | | VVTVPSSSLGTQTYICNVNHKPSNTKVDKKVEPKSCD |
| | | KTHTCPPCPAPEAAGGPSVFLFPPKPKDTLMISRTPE |
| | | VTCVVVDVSHEDPEVKFNWYVDGVEVHNAKTPREEQ |
| | | YNSTYRVVSVLTVLHQDWLNGKEYKCKVSNKALGAPI |
| | | EKTISKAKGQPREPQVYTLPPSREEMTKNQVSLTCLV |
| | | KGFYPSDIAVEWESNGQPENNYKTTPPVLDSDGSFFL |
| | | YSKLTVDKSRWQQGNVFSCSVMHEALHNHYTQKSLSL |
| | | SPGK |
| | | L6-F58 LC SEQ ID NO: 254 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKGGGG |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQWYSSGHVLIT |
| | | FGQGTKVEIKRTVAAPSVFIPPSDEQLKSGTASVVC |
| | | LLNNFYPREAKVQWKVDNALQSGNSQESVTEQDSKDS |
| | | TYSLSSTLTLSKADYEKHKVYACEVTHQGLSSPVTKS |
| | | FNRGEC |
| RSPO2RA-N-HC-Fv-IgG | RSPO2RA- | RSPO2RA-L6-F4-2 HC SEQ ID NO: 255 |
| | L6-F4-2 | 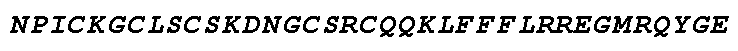 |
| | | 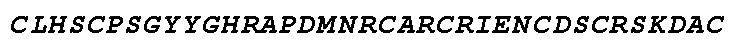 |
| | | 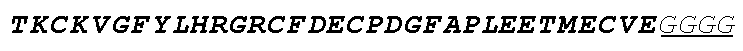 |
| | | SGGGGSGGGGSEVQLVESGGGLVQPGGSLRLSCAASG |
| | | FTFTSYYISWVRQAPGKGLEWVAEISPYSGSTYYADS |
| | | VKGRFTISADTSKNTAYLQMNSLRAEDTAVYYCALRA |
| | | RPPIRLHPRGSVMDYWGQGTLVTVSSGGGGSGGGGSG |
| | | GGGSEVQLVESGGGLVQPGGSLRLSCAASGFNISYYY |
| | | IHWVRQAPGKGLEWVASIYPSSGYTYYADSVKGRFTI |
| | | SADTSKNTAYLQMNSLRAEDTAVYYCARSSFYWAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLMIS |
| | | RTPEVTCVVVDVSHEDPEVKFNWYVDGVEVHNAKTKP |
| | | REEQYNSTYRVVSVLTVLHQDWLNGKEYKCKVSNKAL |
| | | GAPIEKTISKAKGQPREPQVYTLPPSREEMTKNQVSL |
| | | TCLVKGFYPSDIAVEWESNGQPENNYKTTPPVLDSDG |
| | | SFFLYSKLTVDKSRWQQGNVFSCSVMHEALHNHTYQK |
| | | SLSLSPGK |
| | | L6-F4-2 LC SEQ ID NO: 256 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKGGGG |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQSYAAYLFTFG |
| | | GTKVEIKRTVAAPSVFIFPPSDEQLKSGTASVVCLL |
| | | NNFYPREAKVQWKVDNALQSGNQESVTEQDSKDSTY |
| | | SLSSTLTLSKADYEKHDKVYACEVTHQGLSSPVTKSFN |
| | | RGEC |
| RSPO2RA-N-HC-Fv-IgG | RSPO2RA- | RSPO2RA-L6-F4 HC SEQ ID NO: 257 |
| | L6-F4 | 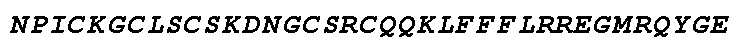 |
| | | 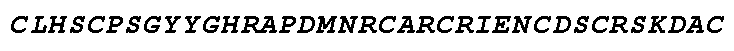 |
| | | 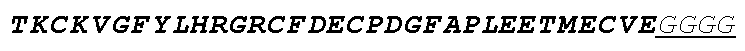 |
| | | SGGGGSGGGGSEVQLVESGGGLVQPGGSLRLSCAASG |
| | | FTFTSYYISWVRQAPGKGLEWVAEISPYSGSTYYADS |
| | | VKGRFTISADTSKNTAYLQMNSLRAEDTAVYYCALRA |
| | | RPPIRLHPRGSVMDYWGQGTLVTVSSGGGGSGGGGSG |
| | | GGGSEVQLVESGGGLVQPGGSLRLSCAASGFNLSSYS |
| | | MHWVRQAPGKGLEWVAYISSYYGYTYYADSVKGRFTI |
| | | SADTSKNTAYLQMNSLRAEDTAVYYCARPAPGHWGFD |
| | | YWGQGTLVTVSSASTKGPSVFPLAPSSKSTSGGTAAL |
| | | GCLVKDYFPEPVTVSWNSGALTSGHTFPAVLQSSGL |
| | | YSLSSVVTVPSSSLGTQTYICNVNHKPSNTKVDKKVE |
| | | PKSCDKTHTCPPCPAPEAAGGPSVFLFPPKPKDTLMI |
| | | SRTPEVTCVVVDVSHEDPEVKFNWYVDGVEVHNAKTK |
| | | PREEQYNSTYRVVSVLTVLHQDWLNGKEYKCKVSNKA |
| | | LGAPIEKTISKAKGQPREPQVYTLPPSREEMTKNQVS |
| | | LTCLVKGFYPSDIAVEWESNGQPENNYKTTPPVLDSD |
| | | GSFFLYSKLTVDKSRWQQGNVFSCSVMHEALHNHYTQ |
| | | KSLSLSPGK |
| | | L6-F4 LC SEQ ID NO: 258 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKGGGG |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQWYYAPITFGQ |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHKVYACEVTHQGLSSPVTKSFNR |
| | | GEC |
| RSPO2RA-N-HC-Fv-IgG | RSPO2RA- | RSPO2RA-L6-F49 HC SEQ ID NO: 259 |
| | L6-F49 | 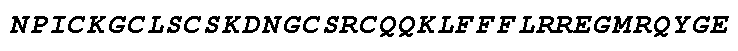 |
| | | 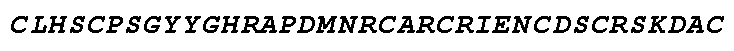 |
| | | 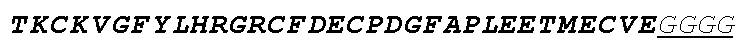 |
| | | SGGGGSGGGGSEVQLVESGGGLVQPGGSLRLSCAASG |
| | | FTFTSYYISWVRQAPGKGLEWVAEISPYSGSTYYADS |
| | | VKGRFTISADTSKNTAYLQMNSLRAEDTAVYYCALRA |
| | | RPPIRLHPRGSVMDYWGQGTLVTVSSGGGGSGGGGSG |
| | | GGGSEVQLVQSGAEVKKPGASVKVSCKASGGTFSSYA |
| | | ISWVRQAPGQGLEWMGWINAGNGNTTYAQKFQGRVTM |
| | | TRDTSTSTVYMELSSLRSEDTAVYYCARHYYGSGSYP |
| | | DWGQGTLVTVSSASTKGPSVFPLAPSSKSTSGGTAAL |
| | | GCLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPEAAGGPSVFLFPPKPKDTLMI |
| | | SRTPEVTCVVVDVSHEDPEVKFNWYDGVEVHNAKTK |
| | | PREEQYNSTYRVVSVLTVLHQDWLNGKEYKCKVSNKA |
| | | LGAPIEKTISKAKGQPREPQVYTLPPSREEMTKNQVS |
| | | LTCLVKGFYPSDIAVEWESNGQPENNYKTTPPVLDSD |
| | | GSFFLYSKLTVDKSRWQQGNVFSCSVMHEALHNHYTQ |
| | | KSLSLSPGK |
| | | L6-F49 LC SEQ ID NO: 260 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKRGGG |
| | | GSGGGGSGGGGSDIQMTQSPSSPSASVGDRVTITCRA |
| | | SQSISSYLNWYQQKPGKAPKLLIYAASSLQSGVPSRF |
| | | SGSGSGTDFTLTISSLQPEDFATYYCQQSYSTPLTFG |
| | | GGTKVEIKRTVAAPSVFIFPPSDEQLKSGTASVVCLL |
| | | NNFYPREAKVQWKVDNALQSGNSQESVTEQDSKDSTY |
| | | SLSSTLTLSKADYEKHKVYACEVTHQGLSSPVTKSFN |
| | | RGEC |
| RSPO2RA-N-HC-Fv-IgG | RSPO2RA- | RSPO2RA-L6-F10 HC SEQ ID NO: 261 |
| | L6-F10 | 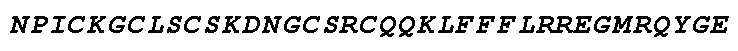 |
| | | 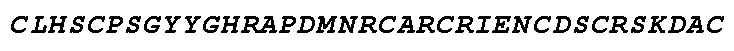 |
| | | 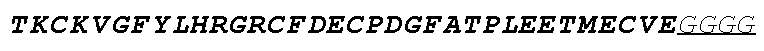 |
| | | SGGGGSGGGGSEVQLVESGGGLVQPGGSLRLSCAASG |
| | | FTFTSYYISWVRQAPKGLEWVAEISPYSGSTYYADS |
| | | VKGRFTISADTSKNTAYLQMNSLRAEDTAVYYCALRA |
| | | RPPIRLHPRGSVMDYWGQGTLVTVSSGGGGSGGGGSG |
| | | GGGSEVQLVESGGGLVQPGGSLRLSCAASGFTFSSFN |
| | | MFWVRQAPGKGLEWVAGIDDDGSYPNYGSAVKGRATI |
| | | SRDNSKNTLYLQMNSLRAEDTAVYYCAKSGYGGSWGG |
| | | YIADDIDAWGQGTLVTVSSASTKGPSVFPLAPSSKST |
| | | SGGTAALGCLVKDYFPEPVTVSWNSGALTSGVHTFPA |
| | | VLQSSGLYSLSSVVTVPSSSLGTQTYICNVNHKPSNT |
| | | KVDKKVEPKSCDKTHTCPPCPAPEAAGGPSVFLFPPK |
| | | PKDTLMISRTPEVTCVVVDVSHEDPEVKFNWYVDGVE |
| | | VHNAKTKPREEQYNSTYRVVSVLTVLHQDWLNGKEYK |
| | | CKVSNKALGAPIEKTISKAKGQPREPQVYTLPPSREE |
| | | MTKNQVSLTCLVDKGFYPSDIAVEWESNGQPENNYKTT |
| | | PPVLDSDGSFFLYSKLTVDKSRWQQGNVFSCSVMHEA |
| | | LHNHYTQKSLSLSPGK |
| | | L6-F10 LC SEQ ID NO: 262 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKGGGG |
| | | SGGGGSGGGGSELTQPPSVSVSPGQTARITCSGDGSY |
| | | AGSYYYGWYQQKPGQAPVTLIYYNNKRPSGIPERFSG |
| | | SLSGSTNTLTISGVQAEDEADYYCGSADNSGAAFGGG |
| | | TKLTVLRTVAAPSVFIFPPSDEQLKSGTASVVCLLNN |
| | | FYPREAKVQWKVDNALQSGNSQESVTEQDSKDSTYSL |
| | | SSTLTLSKADYEKHKVYACEVTHQGLSSPVTKSFNRG |
| | | EC |
| RSPO2RA-N-HC-Fv-IgG | RSPO2RA- | RSPO2RA-L6-F2I HC SEQ ID NO: 263 |
| | L6-F2I | 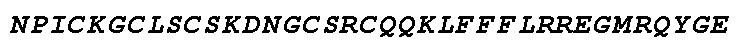 |
| | | 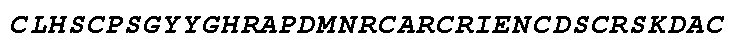 |
| | | 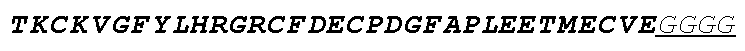 |
| | | SGGGGSGGGGSEVQLVESGGGLVQPGGSLRLSCAASG |
| | | FTFTSYYISWVRQAPGKGLEWVAEISPYSGSTYYADS |
| | | VKGRFTISADTSKNTAYLQMNSLRAEDTAVYYCALRA |
| | | RPPIRLHPRGSVMDYWGQGTLVTVSSGGGGSGGGGSG |
| | | GGGSEVQLVESGGGLVQPGGSLRLSCAASGFNIHSSS |
| | | IHWVRQAPGKGLEWVAATYSSFGSITYADSVKGRFTI |
| | | SADTSKNTAYLQMNSLRAEDTAVYYCARYHHHPFGYAL |
| | | DYWGQGTLVTVSSASTKGPSVFPLAPSSKSTSGGTAA |
| | | LGCLVKDYFPEPVTVSWNSGALTSGVHTFPAVLQSSG |
| | | LYSLSSVVTVPSSSLGTQTYICNVNHKPSNTKVDKKV |
| | | EPKSCDKTHTCPPCPAPEAAGGPSVFLFPPKPKDTLM |
| | | ISRTPEVTCVVVDVSHEDPEVKFNWYVDGVEVHNAKT |
| | | KPREEQYNSTYRVVSVLTVLHQDWLNGKEYKCKVSNK |
| | | ALGAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHYT |
| | | QKSLSLSPGK |
| | | L6-F2I LC SEQ ID NO: 264 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKRGGG |
| | | GSGGGGSGGGGSDIQMTQSPSSLSASVGDRVTITCRA |
| | | SQSVSSAVAWYQQKPGKAPKLLIYSASSLYSGVPSRF |
| | | SGSRSGTDFTLTISSLQPEDFATYYCQQGVYLFTFGQ |
| | | GTKVEIKRTVAAPSVFIFPPSDEQLKSGTASVVCLLN |
| | | NFYPREAKVQWKVDNALQSGNASQESVTEQDSKDSTYS |
| | | LSSTLTLSKADYEKHKVYACEVTHQGLSSPVTKSFNR |
| | | GEC |
| RSPO2RA-N-HC-Fv-IgG | RSPO2RA- | RSPO2RA-L6-F7B HC SEQ ID NO: 265 |
| | L6-F7B | 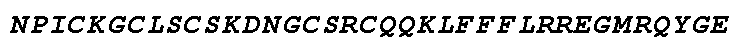 |
| | | 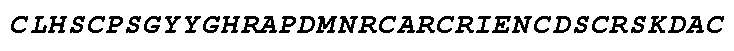 |
| | | 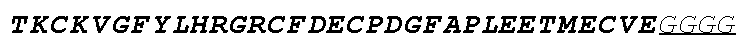 |
| | | SGGGGSGGGGSEVQLVESGGGLVQPGGSLRLSCAASG |
| | | FTFTSYYISWVRQAPGKGLEWVAEISPYSGSTYYADS |
| | | VKGRFTISADTSKNTAYLQMNSLRAEDTAVYYCALRA |
| | | RPPIRLHPRGSVMDYWGQGTLVTVSSGGGGSGGGGSG |
| | | GGGSEVQLVESGGGLVQPGGSLRLSCAASGFNFSSSS |
| | | IHWVRQAPGKGLEWVAYIYPSYDYTYYADSVKGRFTI |
| | | SADTSKNTAYLQMNSLRAEDTAVYYCARGYFYTWGGM |
| | | DYWGQGTLVTVSSASTKGPSVFPLAPSSKSTSGGTAA |
| | | LGCLVKDYFPEPVTVSWNSGALTSGVHTFPAVLQSSG |
| | | LYSLSSVVTVPSSSLGTQTYICNVNHKPSNTKVDKKV |
| | | EPKSCDKTHTCPPCPAPEAAGGPSVFLFPPKPKDTLM |
| | | ISRTPEVTCVVVDVSHEDPEVKFNWYVDGVEVHNAKT |
| | | KPREEQYNSTYRVVSVLTVLHQDWLNGKEYKCKVSNK |
| | | ALGAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHYT |
| | | QKSLSLSPGK |
| | | L6-F7B LC SEQ ID NO: 266 |
| | | DIQMTQSPSSLSASVGDRVTITCRASQDVSTAVAWYQ |
| | | QKPGKAPKLLIYSASFLYSGVPSRFSGSGSGTDFTLT |
| | | ISSLQPEDFATYYCQQSYTTPPTFGQGTKVEIKRGGG |
| | | GSGGGGSGGGGSDIQMTQSPSSLSASVGDRVTITCRA |
| | | SQSVSSAVAWYQQKPGKAPKLLIYSASSLYSGVPSRF |
| | | SGSRSGTDFTLTISSLQPEDFATYYCQQYYSLFTFGQ |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHKVYACEVTHQGLSSPVTKSFNR |
| | | GEC |
| 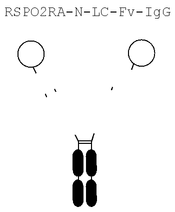 | RSPO2RA- L6-F12578 | RSPO2RA-L6-F12578 LC SEQ ID NO: 267 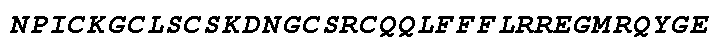 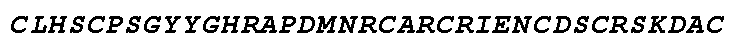 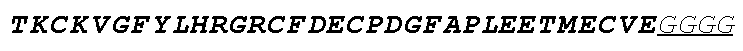 SGGGGSGGGGSDIQMTQSPSSLSASVGDRVTITCRAS QDVSTAVAWYQQKPGKAPKLLIYSASFLYSGVPSRFS GSGSGTDFTLTISSLQPEDFATYYCQQSYTTPPTFGQ GTKVEIKGGGGSGGGGSGGGGSDIELTQPPSVSVAPG QTARISCSGDNIGSFYVHWYQQKPGQAPVLVIYDKSN RPSGIPERFSGSNSGNTATLTISGTQAEDEADYYCQS YANTLSLVFGGGTKLTVLGQPKAAPSVTLFPPSSEEL |
| | | QANKATLVCLISDFYPGAVTVAWKADSSPVKAGVETT |
| | | TPSKQSNNKYAASSYLSLTPEQWKSHRSYSCQVTHEG |
| | | STVEKTVAPTECS |
| | | L6-F12578 HC SEQ ID NO: 268 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGGLVQPGGSLRLSCAASGFTFSHYTLSWVRQAPGKG |
| | | LEWVSVISGDGSYTYYADSVKGRFTISSDNSKNTLYL |
| | | QMNSLRAEDTAVYYCARNFIKYVFANWGQGTLVTVSS |
| | | ASTKGPSVFPLAPSSKSTSGGTAALGCSLVKDYFPEPV |
| | | TVSWNSGALTSGVHTFPAVLQSSGLYSLSSVVTVPSS |
| | | SLGTQTYICNVNHKPSNTKVDKKVEPKSCDKTHTCPP |
| | | CPAPEAAGGPSVFLFPPKPKDTLMISRTPEVTCVVVD |
| | | VSHEDPEVKFNWYVDGVEVHNAKTKPREEQYNSTYRV |
| | | VSVLTVTLHQDWLNGKEYKCKVSNKALGAPIEKTISDKA |
| | | KGQPREPQVYTLPPSREEMTKNQVSLTCLVKGFYPSD |
| | | IAVEWESNGQPENNYKTTPPVLDSDGSFFLYSKLTVD |
| | | KSRWQQGNVFSCSVMHEALHNHYTQKSLSLSPGK |

| | | |
|---|---|---|
| RSPO2RA-N-LC-Fv-IgG | LSPO2RA- | RSPO2RA-L6-F127 LC SEQ ID NO: 269 |
| | L6-F127 | 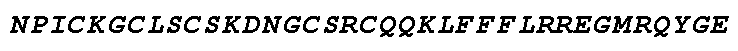 |

| | | |
|---|---|---|
| | | 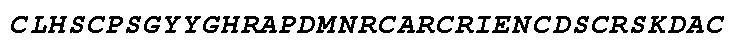 |
| | | 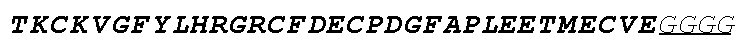 |
| | | SGGGGSGGGGSDIQMTQSPSSLSASVGDRVTITCRAS |
| | | QDVSTAVAWYQQKPGKAPKLLIYSASFLYSGVPSRFS |
| | | GSGSGTDFTLTISSLQPEDFATYYCQQSYTTPPTFGQ |
| | | GTKVEIKGGGGSGGGGSGGGGSDIQMTQSPSSLSASV |
| | | GDRVTITCRASQSVSSAVAWYQQKPGKAPKLLIYSAS |
| | | SLYSGVPSRFSGSRSGTDFTLTISSLQPEDFATYYCQ |
| | | QYWYGVAPITFGQGTKVEIKRTVAAPSVFIFPPSDEQ |
| | | LKSGTASVVCLLNNFYPREAKVQWKVDNALQSGNSQE |
| | | SVTEQDSKDSTYSLSSTLTLSKADYEKHKVYACEVTH |
| | | QGLSSPVTKSFNRGEC |
| | | L6-F127 HC SEQ ID NO: 270 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNISSSYIHWVRQAPGKG |
| | | LEWVAYIYSSYGSTYYADSVKGRFTISADTSKNTAYL |
| | | QMNSLRAEDTAVYYCARASWYALDYWGQGTLVTVSSA |
| | | STKGPSVFPLAPSSKSTSGGTAALGCLVKDYFPEPVT |
| | | VSWNSGALTSGVHTFPAVLQSSGLYSLSSVVTVPSSS |
| | | LGTQTYICNVNHKPSNTKVDKKVEPKSCDKTHTCPPC |
| | | PAPEAAGGPSVFLFPPKPKDTLMISRTPEVTCVVVDV |
| | | SHEDPEVKFNWYVDGVEVHNAKTKPREEQYNSTYRVV |
| | | SVLTVLHQDWLNGKEYKCKVSNKALGAPIEKTISKAK |
| | | GQPREPQVYTLPPSREEMTKNQVSLTCLVKGFYPSDI |
| | | AVEWESNGQPENNYKTTPPVLDSDGSFFLYSLKLTVDK |
| | | SRWQQGNVFSCSVMHEALHNHYTQKSLSLSPGK |
| RSPO2RA-N-LC-Fv-IgG | RSPO2RA- | RSPO2RA-L6-F58 LC SEQ ID NO: 271 |
| | L6-F58 | 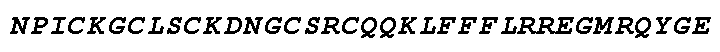 |
| | | 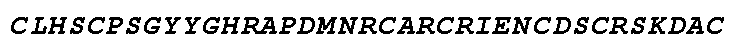 |
| | | 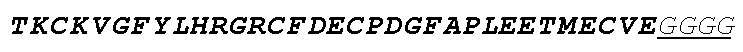 |
| | | SGGGGSGGGGSDIQMTQSPSSLSASVGDRVTITCRAS |
| | | QDVSTAVAWYQQKPGKAPKLLIYSASFLYSGVPSRFS |
| | | GSGSGTDFTLTISSLQPEDFATYYCQQSYTTPPTFGQ |
| | | GTKVEIKGGGGSGGGGSGGGGSDIQMTQSPSSLSASV |
| | | GDRVTITCRASQSVSSAVAWYQQKPGKAPKLLIYSAS |
| | | SLYSGVPSRFSGSRSGTDFTLTISSLQPEDFATYYCQ |
| | | QWYSSGHVLITFGQGTKVEIKRTVAAPSVFIFPPSDE |
| | | QLKSGTASVVCLLNNFYPREAKVQWKVDNALQSGNSQ |
| | | ESVTEQDSKDSTYSLSSTLTLSKADYEKHKVYACEVT |
| | | HQGLSSPVTKSFNRGEC |
| | | L6-F58 HC SEQ ID NO: 272 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVEQPGGSLRLSCAASGFNISYSYIHWVRQAPGKG |
| | | LEWVASIYSSSGSTSYADSVKGRFTISADTSKNTAYL |
| | | QMNSLRAEDTAVYYCARGAIDYWGQGTLVTVSSASTK |
| | | GPSVFPLAPSSKSTSGGTAALGCLVKDYFPEPVTVSW |
| | | NSGALTSGVHTFPAVLQSSGLYSLSSVVTVPSSSLGT |
| | | QTYICNVNHKPSNTKVDKKVEPKSCDKTHTCPPCPAP |
| | | EAAGGPSVFLFPPKPKDTLMISRTPEVTCVVVDVSHE |
| | | DPEVKFNWYDGVEVHNAKTKPREEQYNSTYRVVSVL |
| | | TVLHQDWLNGKEYKCKVSNKALGAPIEKTISKAKGQP |
| | | REPQVYTLPPSREEMTKNQVSLTCLVKGFYPSDIAVE |
| | | WESNGQPENNYKTTPPVLDSDGSFFLYSKLTVDKSRW |
| | | QQGNVFSCSVMHEALHNHYTQKSLSLSPGK |
| RSPO2RA-N-LC-Fv-IgG | RSPO2RA- | RSPO2RA-L6-F4-2 LC SEQ ID NO: 273 |
| | L6-F4-2 | 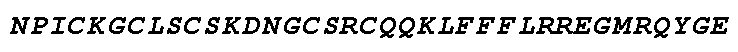 |
| | | 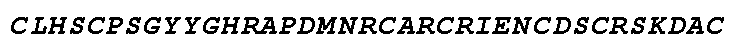 |
| | | 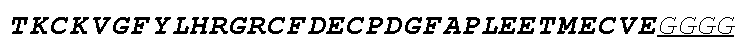 |
| | | SGGGGSGGGGDIQMTQSPSSLSASVGDRVTITCRASQ |
| | | DVSTAVAWYQQKPGKAPKLLIYSASFLYSGVPSRFSG |
| | | SGSGTDFTLTISSLQPEDFATYYCQQSYTTPPTFGQG |
| | | TKVEIKGGGGSGGGGSGGGGSDIQMTQSPSSLSASVG |
| | | DRVTITCRASQSVSSAVAWYGGKPGKAPKLLIYSASS |
| | | LYSGVPSRFSGSRSGTDFTLTISSLQPEDFATYYCQQ |
| | | SYAAYLFTFGQGTKVEIKRTVAAPSVFIFPPSDEQLK |
| | | SGTASVVCLLNNFYPREAKVQWKVDNALQSGANSQESV |
| | | TEQDSKDSTYSLSSTLTLSKADYEKHKVYACEVTHQG |
| | | LSSPVTKSFNRGEC |
| | | L6-F4-2 HC SEQ ID NO: 274 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNISYYYIHWVRQAPGKG |
| | | LEWVASIYPSSGYTYYADSVKGRFTISADTSKNTAYL |
| | | QMNSLRAEDTAVYYCARSSFYWAMDYWGQGTLVTVSS |
| | | ASTKGPSVFPLAPSSKSTSGGTAALGCLVKDYFPEPV |
| | | TVSWNSGALTSGVHTFPAVLQSSGLYSLSSVVTVPSS |
| | | SLGTQTYICNVNHKPSNTKVDKKVEPKSCDKTHTCPP |
| | | CPAPEAAGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALGAPIEKTISKA |
| | | KGQPREPQVYTLPPSREEMTKNQVSLTCLVKGFYPSD |
| | | IAVEWESNGQPENNYKTTPPVLDSDGSFFLYSKLTVD |
| | | KSRWQQGNVFSCSVMHEALHNHYTQKSLSLSPGK |
| RSPO2RA-N-LC-Fv-IgG | RSPO2RA- | RSPO2RA-L6-F4 LC SEQ ID NO: 275 |
| | L6-F4 | 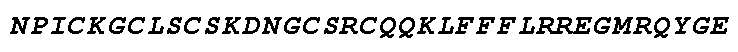 |
| | | 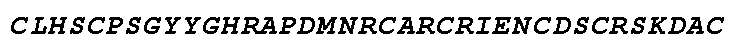 |
| | | 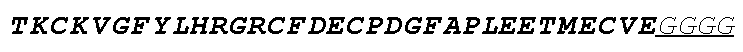 |
| | | SGGGGSGGGGSDIQMTQSPSSLSASVGDRVTITCRAS |
| | | QDVSTAVAWYQQKPGKAPKLLIYSASFLYSGVPSRFS |
| | | GSGSGTDFTLTISSLQPEDFATYYCQQSYTTPPTFGQ |
| | | GTKVEIKGGGGSGGGGSGGGGSDIQMTQSPSSLSASV |
| | | GDRVTITCRASQSVSSAVAWYQQKPGKAPKLLIYSAS |
| | | SLYSGVPSRFSGSRSGTDFTLTISSLQPEDFATYYCQ |
| | | QWYYAPITFGQGTKVEIKRTVAAPSVFIFPPSDEQLK |
| | | SGTASVVCLLNNFYPREAKVQWKVDNALQSGNSQESV |
| | | TEQDSKDSTYSLSSTLTLSKADYEKHKVYACEVTHQG |
| | | LSSPVTKSFNRGEC |
| | | L6-F4 HC SEQ ID NO: 276 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNLSSYSMHWVRQAPGKG |
| | | LEWVAYISSYYGYTYYADSVKGRFTISADTSKNTAYL |
| | | QMNSLRAEDTAVYYCARPAPGHWGFDYWGQGTLVTVS |
| | | SASTKGPSVFPLAPSSKSTSGGTAALGCLVKDYFPEP |
| | | VTVSWNSGALTSGVHTFPAVLQSSGLYSLSSVVTVPS |
| | | SSLGTQTYICNVNHKPSNTKVDKKVEPKSCDKTHTCP |
| | | PCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| RSPO2RA-N-LC-Fv-IgG | RSPO2RA- | RSPO2RA-L6-F49 LC SEQ ID NO: 277 |
| | L6-F49 | 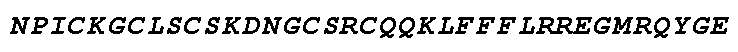 |
| | | 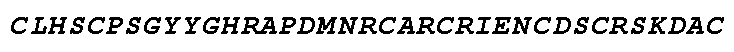 |
| | | 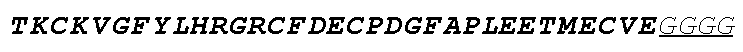 |
| | | SGGGGSGGGGSDIQMTQSPSSLSASVGDRVTITCRAS |
| | | QDVSTAVAWYQQKPGKAPKLLIYSASFLYSGVPSRFS |
| | | GSGSGTDFTLTISSLQPEDFATYYCQQSYTTPPTFGQ |
| | | GTKVEIKRGGGGSGGGGSGGGGSDIQMTQSPSSPSAS |
| | | VGDRVTITCRASQSISSYLNWYQQKPGKAPKLLIYAA |
| | | SSLQSGVPSRFSGSGSGTDFTLTISSLQPEDFATYYC |
| | | QQSYSTPLTFGGGGTKVEIKRTVAAPSVFIFPPSDEQL |
| | | KSGTASVVCLLNNFYPREAKVQWKVDNALQSGNSQES |
| | | VTEQDSKDSTYSLSSTLTLSKADYEKHKVYACEVTHQ |
| | | GLSSPVTKSFNRGEC |
| | | L6-F49 HC SEQ ID NO: 278 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVQS |
| | | GAEVKKPGASVKVSCKASGGTFSSYAISWVRQAPGQG |
| | | LEWMGWINAGNGNTTYAQKFQGRVTMTRDTSTSTVYM |
| | | ELSSLRSEDTAVYYCARGYYGSGSYPDWGQGTLVTVS |
| | | SASTKGPSVFPLAPSSKSTSGGTAALGCLVKDYFPEP |
| | | VTVSWNSGALTSGVHTFPAVLQSSGLYSLSSVVTVPS |
| | | SSLGTQTYICNVNHKPSNTKVDKKVEPKSCDKTHTCP |
| | | PCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGK |
| RSPO2RA-N-LC-Fv-IgG | RSPO2RA- | RSPO2RA-L6-F10 LC SEQ ID NO: 279 |
| | L6-F10 | 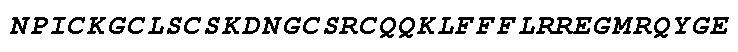 |
| | | 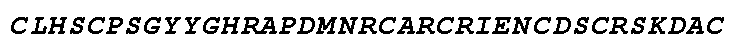 |
| | | 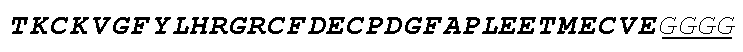 |
| | | SGGGGSGGGGSDIQMTQSPSSLSASVGDRVTITCRAS |
| | | QDVSTAVAWYQQKPGKAPKLLIYSASFLYSGVPSRFS |
| | | GSGSGTDFTLTISSLQPEDFATYYCQQSYTTPPTFGQ |
| | | GTKVEIKGGGGSGGGGSGGGGSELTQPPSVSVSPGQT |
| | | ARITCSGDGSYAGSYYYGWYQQKPGQAPVTLIYYNNK |
| | | RPSGIPERFSGSLSGSTNTLTISGVQAEDEADYYCGS |
| | | ADNSGAAFGGGTKLTVLRTVAAPSVFIFPPSDEQLKS |
| | | GTASVVCLLNNFYPREAKVQWKVDNALQSGNGQESVT |
| | | EQDSKDSTYSLSSTLTLSKADYEKHKVYACEVTHQGL |
| | | SSPVTKSFNRGEC |
| | | L6-F10 HC SEQ ID NO: 280 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFTFSSFNMFWVRQAPGKG |
| | | LEWVAGIDDDGSYPNYGSAVKGRATISRDNSKNTLYL |
| | | QMNSLRAEDTAVYYCAKSGYSSSWGGYIADDIDAWGQ |
| | | GTLVTVSSASTKGPSVFPLAPSSKSTSGGTAALGCLV |
| | | KDYFPEPVTVSWNSGALTSGVGTFPAVLQSSGLYSLS |
| | | SVVTVPSSSLGTQTYICNVNHKPSNTKVDKKVEPKSC |
| | | DKTHTCPPCPAPEAAGGPSVFLFPPKPKDTLMISRTP |
| | | EVTCVVVDVSHEDPEVKFNWYVDGVEVHNAKTKPREE |
| | | QYNSTYRVVSVLTVLHQDWLNGKEYKCKVSNKALGAP |
| | | IEKTISKAKGQPREPQVYTLPPSREEMTKNQVSLTCL |
| | | VKGFYPSDIAVEWESNGQPENNYKTTPPVLDSDGSFF |
| | | LYSKLTVDKSRWQQGNVFSCSVMHEALHNHYTQKSLS |
| | | LSPGK |
| RSPO2RA-N-LC-Fv-IgG | RSPO2RA- | RSPO2RA-L6-F2I LC SEQ ID NO: 281 |
| | L6-F7I | 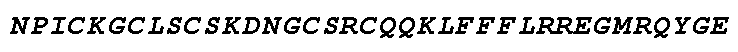 |
| | | 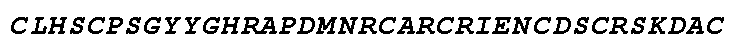 |
| | | 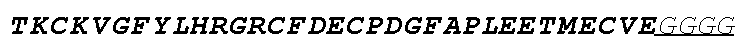 |
| | | SGGGGSGGGGSDIQMTQSPSSLSASVGDRVTITCRAS |
| | | QDVSTAVAWYQQKPGKAPKLLIYSASFLYSGVPSRFS |
| | | GSGSGTDFTLTISSLQPEDFATYYCQQSYTTPPTFGQ |
| | | GTKVEIKRGGGGSGGGGSGGGGSDIQMTQSPSSLSAS |
| | | VGDRVTITCRASQXVXXAVAWYQQKPGKAPKLLIYSA |
| | | SSLYSGVPSRFSGSRSGTDFTLTISSLQPEDFATYYC |
| | | QQGVYLFTFGQGTKVEIKRTVAAPSVFIFPPSDEQLK |
| | | SGTASVVCLLNNFYPREAKVQWKVDNALQSGNSQESV |
| | | TEQDSKDSTYSLSSTLTLSKADYEKHKVYACEVTHQG |
| | | LSSPVTKSFNRGEC |
| | | L6-F2I HC SEQ ID NO: 282 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNIHSSSIHWVRQAPGKG |
| | | LEWVAATYSSFGSITYADSVKGRFTISADTSKNTAYL |
| | | QMNSLRAEDTAVYYCARYHHPFGYALDYWGQGTLVTV |
| | | SSASTKGPSVFPLAPSSKSTSGGTAALGCLVDKYFPE |
| | | PVTVSWNSGALTSGVHTFPAVLQSSGLYSLSSVVTVP |
| | | SSSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYDGVEVHNAKTKPREEQYNSTY |
| | | RVVSVLTVLHQDWLNGKEYKCKVSNKALGAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQSLSLSPGK |
| RSPO2RA-N-LC-Fv-IgG | RSPO2RA- | RSPO2RA-L6-F7B LC SEQ ID NO: 283 |
| | L6-F7B | 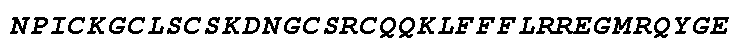 |
| | | 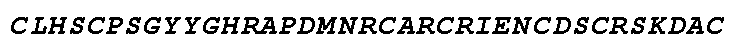 |
| | | 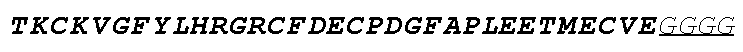 |
| | | SGGGGSGGGGSDIQMTQSPSSLSASVGDRVTITCRAS |
| | | QDVSTAVAWYQQKPGKAPKLLIYSASFLYSGVPSRFS |
| | | GSGSGTDFTLTISSLQPEDFATYYCQQSYTTPPTFGQ |
| | | GTKVEIKRGGGGSGGGGSGGGGSDIQMTQSPSSLSAS |
| | | VGDRVTITCRASQSVSSAVAWYQQKPGKAPKLLIYSA |
| | | SSLYSGVPSRFSGSRSGTDFTLTISSLQPEDFATYYC |
| | | QQYYSLFTFGQGTKVEIKRTVAAPSVFIFPPSDEQLK |
| | | SGTASVVCLLNNFYPREAKVQWKVDNALQSGNSQESV |
| | | TEQDSKDSTYSLSSTLTLSKADYEKHKVYACEVTHQG |
| | | LSSPVTKSFNRGEC |
| | | L6-F7B HC SEQ ID NO: 284 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNFSSSSIHWVRQAPGKG |
| | | LEWVAYIYPSYDTYYADSVKGRFTISADTSKNTAYL |
| | | QMNSLRAEDTAVYYCARGYFYTWGGMDYWGQGTLVTV |
| | | SSASTKGPSVFPLAPSSKSTSGGTAALGCLVKDYFPE |
| | | PVTVSWNSGALTSGVHTFPAVLQSSGLYSLSSVVTVP |
| | | SSSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALGAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 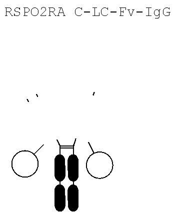 | L6-F4-2- RSPO2RA | L6-F4-2-RSPORA LC SEQ ID NO: 285 DIQMTQSPSSLSASVGDRVTITCRASQDVSTAVAWYQ QKPGKAPLLIYSASFLYSGVPSRFSGSGSGTDFTLT ISSLQPEDFATYYCQQSYTTPPTFGQGTKVEIKGGGG SGGGGSGGGGSDIQMTQSPSSLSASVGDRVTITCRAS QSVSSAVAWYQQKPGKAPKLLIYSASSLYSGVPSRFS GSRSGTDFTLTISSLQPEDFATYYCQQSYAAYLFTFG QGTKVDIKRTVAAPSVFIFPPSDEQLKSGTASVVCLL NNFYPREAKVQWKVDNALQSGNSQESVTEQDSKDSTY SLSSTLTLSKADYEKHKVYACEVTHQGLSSPVTKSFN RGECGGGGSGSGGSGGGGSNPICKGCLSCSKDNGCSR CQQKLFFFLRREGMRQYGECLHSCPSGYYGHRAPDMN RCARCRIENCDSCRDKDACTKCKVGFYLHRGRCFDEC PDGFAPLEETMECVE |
| | | L6-F4-2-5063 HC SEQ ID NO: 286 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNISYYYIHWVRQAPGKG |
| | | LEWVASIYPSSGYTYYADSVKGRFTISADTSKNTAYL |
| | | QMNSLRAEDTAVYYCARSSFYWAMDYWGQGTLVTVSS |
| | | ASTKGPSVFPLAPSSKSTSGGTAALGCLVKDYFPEPV |
| | | TVSWNSGALTSGVHTFPAVLQSSGLYSLSSVVTVPSS |
| | | SLGTQTYICNVNHKPSNTKVDKKVEPKSCDKTHTCPP |
| | | CPAPEAAGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALGAPIEKTISKA |
| | | KGPREPQVYTLPPSREEMTKNQVSLTCLVKGFYPSD |
| | | IAVEWESNGQPENNYKTTPPVLDSDGSFFLYSKLTVD |
| | | KSRWQQGNVFSCSVMHEALHNHYTQKSLSLSPGK |
| RSPO2RA C-LC-Fv-IgG | L6-F4- | L6 F4 RSPORA LC SEQ ID NO: 287 |
| | RSPO2RA | DIQMTQSPSSLSASVGDRVTITCRASQDVSTAVAWYQ |
| | | QKPKAPKLLIYSASFLYSGVPSRFSGSGSGTDFTLT |
| | | ISSLQPEDFATYYCQQSYTTPPTFGQGTKVEIKGGGG |
| | | SGGGGSGGGGSDIQMTQSPSSLSASVGDRVTITCRAS |
| | | QSVSSAVAWYQQKPGKAPKLLIYSASSLYSGVPSRFS |
| | | GSRSGTDFTLTISSLQPEDFATYYCQQWYYAPITFGQ |
| | | GTKVEIKRTVAAPSVFIFPPSDEQLKSGTASVVCLLN |
| | | NFYPREAKVQWKVDNALQSGNSQESVTEQDSKDSTYS |
| | | LSSTLTLSKADYEKHKVYACEVTHQGLSSPVTKSFNT |
| | | 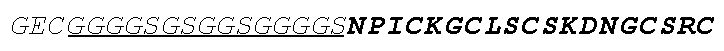 |
| | | 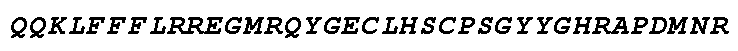 |
| | | 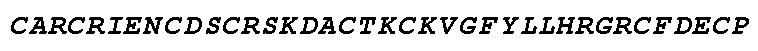 |
| | | 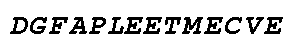 |
| | | L6-F4 HC SEQ ID NO: 288 |
| | | EVQLVESGGGLVQPGGSLRLSCAASGFTFTSYYISWV |
| | | RQAPGKGLEWVAEISPYSGSTYYADSVKGRFTISADT |
| | | SKNTAYLQMNSLRAEDTAVYYCALRARPPIRLHPRGS |
| | | VMDYWGQGTLVTVSSGGGGSGGGGSGGGGSEVQLVES |
| | | GGGLVQPGGSLRLSCAASGFNLSSYSMHWVRQAPGKG |
| | | LEWVAYISSYYGYTYYADSVKGRFTISADTSKNTAYL |
| | | QMNSLRAEDTAVYYCARPAPGHWGFDYWGQGTLVTVS |
| | | SASTKGPSVFPLAPSSKSTSGGTAALGCLVKDYFPEP |
| | | VTVSWNSGALTSGVHTFPAVLQSSGLYSLSSVVTVPS |
| | | SSLGTQTYICNVNHKPSNTKVDKKVEPKSCDKTHTCP |
| | | PCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISK |
| | | AKGQPREPQVYTLPPSREEMTKNQVSLTCLVKGFYPS |
| | | DIAVEWESNGQPENNYKTTPPVLDSDGSFFLYSKLTV |
| | | DKSRWQQGNVFSCSVMHEALHNHYTQKSLSLSPGK |

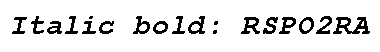

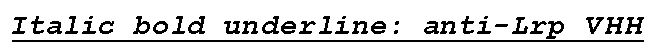

Italic underline: linker
Bold: VH or VL
“F” indicates Fzd binder and “aGFP” indicates anti-GFP antibody sequence

Additional constructs with RSPO2RA fusions with other FZD and LRP binders (e.g., R2M3 (“F6) and 26 (“L2”)) were made having the RSPO2RA fusions at different locations on the IgG molecule. For example, the RSPO2RA protein was fused to C terminus of either the heavy or light chain of the IgG. As shown in FIGS. 3H and 31, all of these FZD-RSPO2RA fusions resulted in RSPO mimetic activity, and additional fusion of a LRP binder resulted in super agonist activity. Therefore, these results demonstrated an approach to generate both RSPO mimetic as well as WNT super-agonists molecules that can target specific subsets of FZD receptors.

To further evaluate format and stoichiometry between different component, another set of molecules were generated between FZD and LRP binders and RSPO mutant as depicted in FIG. 4A, FIGS. 7 and 8. The activities of these various molecules are shown in FIGS. 4B and 4C.

### Example 3

### WNT Super Agonist Molecules Replace Both WNT and R-Spondin in Organoid Culture Expansion

Multiple tissues in the mammalian body are maintained by WNT-driven adult stem cells. Short-range Wnt signals are often further enhanced by local secretion of R-spondins. In this role, R-spondin acts as stem cell growth factor, but only in the presence of WNT. This pivotal interplay of WNT agonists in the stem cell niche is recapitulated in adult stem cell-derived organoid cultures. By providing niche signals in the culture medium, organoids can be maintained and expanded as self-organizing structures. Most of the media for epithelial organoid cultures is supplemented with R-spondin. The addition of R-spondin alone is sufficient if organoid cells secrete their own WNT proteins, such as, for example, Paneth cells in murine small intestinal organoids (Sato et al., 2009). Organoid cultures without endogenous WNT source, such as human intestinal organoids, require the addition of WNT's or WNT mimetics (Sato et al., 2011, Janda et al., 2017). Obtaining high quality WNT proteins and/or R-spondins for organoid medium can be laborious and costly. To test whether a single WNT super agonist molecule can replace both WNT and R-spondin in organoid medium, we tested the outgrowth efficiency of several different organoid cultures in the presence of L6-F12578-RSPO2RA.

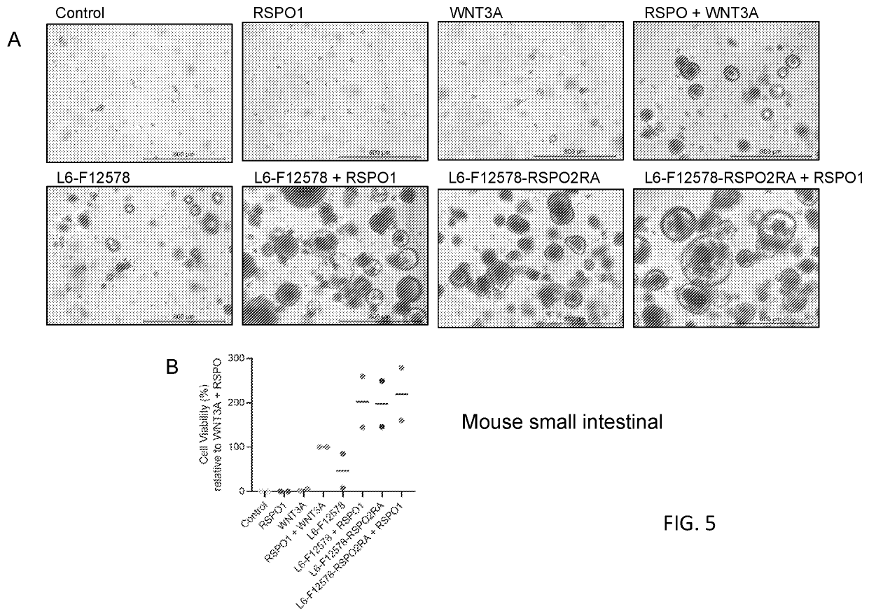

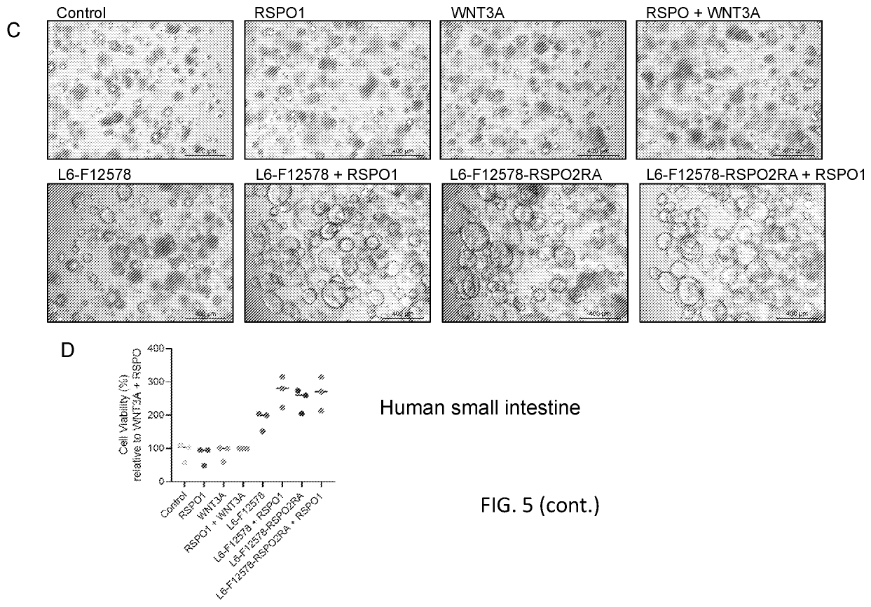

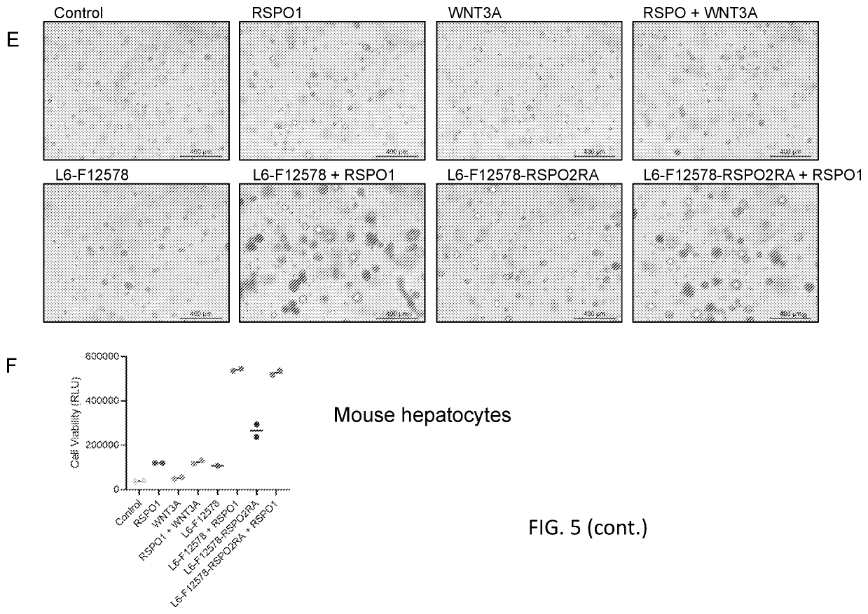

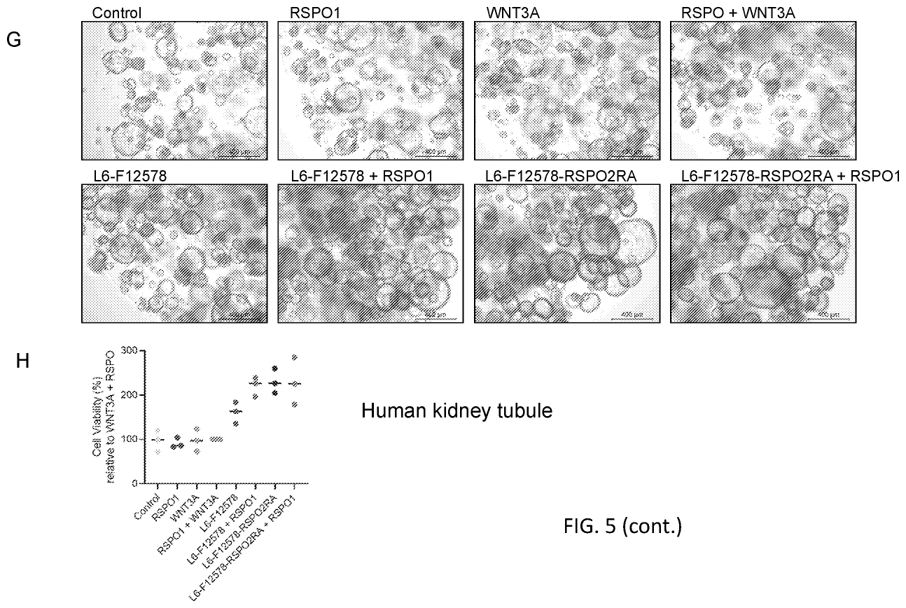

We first examined the applicability of WNT super agonist in the expansion of mouse small intestinal organoids. Murine cells were grown in the presence of porcupine inhibitor C59 to block any endogenous WNT secretion. The addition of RSPO1, WNT3A or L6-F12578 alone had little to no effect on the growth of these cells (FIG. 5A, 5B). As expected, WNT3A plus RSPO1 and L6-F12578 plus RSPO1 stimulated the outgrowth of large cystic organoids in seven days. As shown in FIG. 5A-B, the WNT super agonist L6-F12578-RSPO2RA alone at 0.1 nM was enough to stimulate maximal outgrowth. The addition of recombinant RSPO1 did not further enhance proliferation. Next, we looked at human small intestinal organoids, which require both exogenous WNT and RSPO1 in their expansion medium. While human small intestinal organoids had no response to recombinant WNT3A, a slight increase in outgrowth was observed with L6-F12578 alone (FIG. 5C, 5D). Similar as in mouse, the addition of L6-F12578-RSPO2RA alone at 1 nM displayed an activity level that is equivalent to L6-F12578 and RSPO1 combination (FIG. 5C-D). To further investigate the applicability of WNT super agonist in other cell types and tissues, we tested the expansion of mouse hepatocytes and human tubuloids, two culture systems that depend on the presence of R-spondin in their medium (Hu et al., 2018, Schutgens et al., 2019). Similar to the experimental setup for mouse and human intestine, porcupine inhibitor C59 was added to block any endogenous WNT signal and for mouse hepatocytes we additionally removed GSK3 inhibitor CHIR99021 from the base medium. Over the course of 14 days, murine hepatocytes cultured in L6-F12578-RSPO2RA expanded at a higher rate compared to L6-F12578 alone. The addition of recombinant RSPO1 to both conditions further enhanced outgrowth (FIG. 5E, 5F). The added effect of RSPO1 to L6-F12578-RSPO2RA can be due to a differential sensitivity to RSPO1 and RSPO2 but needs further investigation. Human kidney tubuloids cultured in L6-F12578-RSPO2RA alone rapidly expanded within one week to similar levels of the L6-F12578 and RSPO1 combination and outperforming recombinant WNT3A plus RSPO1 or surrogate WNT alone (FIG. 5G, 5H). Taken together, WNT super agonist can replace WNT3A and RSPO in organoids cultures from intestine, liver and kidney for both mouse and human. We expect L6-F12578-RSPO2RA and other WNT super agonists to outperform recombinant WNT, RSPO and WNT- and RSPO-conditioned media for a wide variety of other organoid models.

### Example 4

### In Vivo Effects of WNT Mimetic Molecules

The effects of WNT mimetic of different FZD specificity has not been fully explored previously. To test the in vivo effect of WNT mimetics with different FZD specificity, the panel of WNT mimetics described in FIG. 1 were dosed at 3 mg per kg intraperitoneally on day 0, 3, 7 and 10 in C57Bl/6J mice. Since we have previously established the effects of FZD1,2,7 WNT mimetics on bone formation in vivo (PCT Publication WO 2019/126398), we tested whether other FZD specificity could also impact bone formation. Compared to baseline at day 0, the whole-body bone mineral densities (BMD) were increased by 39%, 38%, 29% and 11% in the groups of L6-F12578, L6-F127, L6-F58, and L6-F4 at day 13 (P<0.001), respectively (FIG. 6A). Similarly, the BMDs of femur and lumbar were increased up to 60% in the groups of L6-F12578, L6-F127, L6-F58, and L6-F4 at day 13 (P<0.001) (FIG. 6B, C). These data not only confirmed our previous finding that FZD_{1,2,7} specific WNT mimetics induce bone formation, but also suggest that FZD_{5,8} specific WNT mimetics and FZD₄ specific WNT mimetics can also induce bone formation.

The body weights of the various treatment groups were also evaluated, and animals treated with L6-F12578 and L6-F127 groups showed significant reduction (FIG. 6D). This weight reduction may be predominately contributed by decreased body fat content as seen on day 7 and 13 by DEXA analysis (FIG. 6E). The other significant changes observed in the treated animals were the significant increases in salivary gland weight in the groups of L6-F12578, L6-F127, L6-F4, and L6-F10 compared with Vehicle group on day 14, by 101% (P<0.001), 114% (P<0.001), 29% (p<0.01) and 22% (P<0.05) respectively (FIG. 6F), where the effects of L6-F12578 and L6-F127 being the most pronounced. We observed wet fur and fur color change (brown) during day 7-14 of the mice in the groups of L6-F12578, L6-F127. Significant increase in liver and intestine weights have also been observed in several treatment groups (FIG. 6G, 6H). The liver weight increased by 28% (P<0.001) in L6-F12578 group and by 48% (P<0.001) in L6-F4 group, and the small intestine weight increased by 21% (P<0.05), 31% (P<0.001), 30% (p<0.01) and 24% (P<0.05) in the group of L6-F12578, L6-F58, L6-F4, and L6-F49, respectively.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention, and the various embodiments described above can be combined to provide further embodiments. Accordingly, the invention is not limited except as by the appended claims. Aspects of the embodiments can be modified, if necessary to employ concepts of the various patents, applications and publications disclosed herein to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Chen, H., Lu, C., Ouyang, B., Zhang, H., Huang, Z., Bhatia, D., Lee, S. J., Shah, D., Sura, A., Yeh, W. C., et al. (2020). Development of Potent, Selective Surrogate WNT Molecules and Their Application in Defining Frizzled Requirements. Cell Chem Biol 27, 598-609 e594.
Gurney, A., Axelrod, F., Bond, C. J., Cain, J., Chartier, C., Donigan, L., Fischer, M., Chaudhari, A., Ji, M., Kapoun, A. M., et al. (2012). Wnt pathway inhibition via the targeting of Frizzled receptors results in decreased growth and tumorigenicity of human tumors. Proc Natl Acad Sci USA 109, 11717-11722.
Hao, H. X., Xie, Y., Zhang, Y., Charlat, O., Oster, E., Avello, M., Lei, H., Mickanin, C., Liu, D., Ruffner, H., et al. (2012). ZNRF3 promotes Wnt receptor turnover in an R-spondin-sensitive manner. Nature 485, 195-200.
Hu, H., Gehart, H., Artegiani, B., C, L. O.-I., Dekkers, F., Basak, O., van Es, J., Chuva de Sousa Lopes, S. M., Begthel, H., Korving, J., et al. (2018). Long-Term Expansion of Functional Mouse and Human Hepatocytes as 3D Organoids. Cell 175, 1591-1606 e1519.
Janda, C. Y., Dang, L. T., You, C., Chang, J., de Lau, W., Zhong, Z. A., Yan, K. S., Marecic, O., Siepe, D., Li, X., et al. (2017). Surrogate Wnt agonists that phenocopy canonical Wnt and beta-catenin signalling. Nature 545, 234-237.
Koo, B. K., Spit, M., Jordens, I., Low, T. Y., Stange, D. E., van de Wetering, M., van Es, J. H., Mohammed, S., Heck, A. J., Maurice, M. M., et al. (2012). Tumour suppressor RNF43 is a stem-cell E3 ligase that induces endocytosis of Wnt receptors. Nature 488, 665-669.
Sato, T., Stange, D. E., Ferrante, M., Vries, R. G., Van Es, J. H., Van den Brink, S., Van Houdt, W. J., Pronk, A., Van Gorp, J., Siersema, P. D., et al. (2011). Long-term expansion of epithelial organoids from human colon, adenoma, adenocarcinoma, and Barrett's epithelium. Gastroenterology 141, 1762-1772.
Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
Schutgens, F., Rookmaaker, M. B., Margaritis, T., Rios, A., Ammerlaan, C., Jansen, J., Gijzen, L., Vormann, M., Vonk, A., Viveen, M., et al. (2019). Tubuloids derived from human adult kidney and urine for personalized disease modeling. Nat Biotechnol 37, 303-313.
Xie, Y., Zamponi, R., Charlat, O., Ramones, M., Swalley, S., Jiang, X., Rivera, D., Tschantz, W., Lu, B., Quinn, L., et al. (2013). Interaction with both ZNRF3 and LGR4 is required for the signalling activity of R-spondin. EMBO Rep 14, 1120-1126.

What is claimed is:

1. A WNT super agonist molecule, comprising:
a) a Frizzled (FZD) binding domain;
b) an LRP5/6 binding domain; and
c) an E3 ligase binding domain,
wherein the super agonist molecule activates the canonical WNT signaling pathway in a cell.

2. The super agonist molecule of claim 1, wherein:
a) the FZD binding domain binds one or more FZD receptor;
b) the LRP5/6 binding domain binds one or more of LRP5 and/or LRP6; and
c) the E3 ligase binding domain binds ZNRF3 and/or RNF43.

3. The super agonist molecule of claim 1 or claim 2, comprising one or more polypeptides, wherein at least one polypeptide comprises a FZD binding domain fused to an LRP5/6 binding domain, and wherein at least one polypeptide comprises an E3 ligase binding domain fused to a FZD binding domain or an LRP5/6 binding domain.

4. The super agonist molecule of claim 3, wherein the fused binding domains are fused directly together and/or fused via a peptide linker.

5. The super agonist molecule of claim 4, wherein the peptide linker is about 1 amino acid in length to about 30 amino acids in length.

6. The super agonist molecule of claim 5, wherein the peptide linker is about 5 amino acids in length to about 15 amino acids in length, optionally 5, 6, 7, 8, 9, 10, 11, 12, 13, 14 or 15 amino acids in length.

7. The super agonist molecule of any one of claims 4-6, wherein the peptide linker comprises one or more glycine and/or serine residues.

8. The super agonist molecule of any one of claims 1-7, wherein at least one of the binding domains is selected from the group consisting of: an scFv, a VHH/sdAb, a Fab fragment, a Fab′2 fragment, a diabody, and an Fv fragment.

9. The super agonist of any one of claims 1-8, wherein at least one of the binding domains is fused to an Fc fragment, optionally wherein the Fc fragment is from an IgG, IgM, IgA, IgD or IgE antibody isotype or an α, δ, ε, γ, or μ antibody heavy chain.

10. The super agonist molecule of claim 9, having a structure depicted in Table 3 or Table 4.

11. The super agonist molecule of claim 10, having the Fv-IgG structure.

12. The super agonist of any one of claims 1-10, wherein the WNT enhancer comprises an E3 ligase binding domain selected from the group consisting of: a mutant R-spondin (RSPO) protein and an antibody or functional fragment thereof.

13. The super agonist molecule of claim 12, wherein the mutant RSPO protein has reduced binding to Leucine-rich repeat-containing G-protein receptors 4-6 (LGR4-6) as compared to wild type RSPO.

14. The super agonist molecule of claim 12, wherein the E3 ligase binding domain binds a Zinc and Ring Finger 3 (ZNRF3) and/or a Ring Finger Protein 43 (RNF43).

15. The super agonist molecule of claim 14, wherein the E3 ligase binding domain is selected from the group consisting of: an scFv, a VHH/sdAb, a Fab fragment, a Fab′2 fragment, a diabody, and an Fv fragment.

16. The super agonist molecule of claim 15, wherein the E3 ligase binding domain is fused to a C-terminus of an Fc fragment of an Fv-IgG, either directly or via a linker, optionally wherein the linker is a peptide linker of about 1 amino acid in length to about 30 amino acids in length, or about 5 amino acids in length to about 15 amino acids in length, or 5, 6, 7, 8, 9, 10, 11, 12, 13, 14 or 15 amino acids in length.

17. The super agonist of claim 15, wherein the E3 ligase binding domain is fused to a C-terminus of:
a) a light chain or fragment thereof of a FZD binding domain;
b) a heavy chain or fragment thereof of a FZD binding domain;
c) a light chain or fragment thereof of a LRP5/6 binding domain; or
b) a heavy chain or fragment thereof of a LRP5/6 binding domain,
either directly or via a linker, optionally wherein the linker is a peptide linker of about 1 amino acid in length to about 30 amino acids in length, or about 5 amino acids in length to about 15 amino acids in length, or 5, 6, 7, 8, 9, 10, 11, 12, 13, 14 or 15 amino acids in length.

18. The super agonist of claim 15, wherein the binding domain that binds an E3 ubiquitin ligase is fused to a N-terminus of:
a) a light chain or fragment thereof of a FZD binding domain;
b) a heavy chain or fragment thereof of a FZD binding domain;
c) a light chain or fragment thereof of a LRP5/6 binding domain; or
b) a heavy chain or fragment thereof of a LRP5/6 binding domain,
either directly or via a linker, optionally wherein the linker is a peptide linker of about 1 amino acid in length to about 30 amino acids in length, or about 5 amino acids in length to about 15 amino acids in length, or 5, 6, 7, 8, 9, 10, 11, 12, 13, 14 or 15 amino acids in length.

19. The super agonist of any of claims 1-18, comprising a polypeptide having at least 90% or 95% sequence identity to a sequence provided in Table 3 or Table 4, or a combination of polypeptides, each having at least 90% or 95% sequence identity to a sequence provided in Table 3 or Table 4.

20. A pharmaceutical composition comprising the WNT super agonist molecule according to any of claims 1-19 and a pharmaceutically acceptable diluent, excipient, or carrier.

21. A method for treating a subject having a disease or disorder associated with reduced WNT signaling, comprising administering to the subject an effective amount of the WNT super agonist molecule according to any of claims 1-19 or the pharmaceutical composition of claim 20.

22. The method of claim 21, wherein the disease or disorder is selected from the group consisting of: oral mucositis, short bowel syndrome, inflammatory bowel diseases (IBD), other gastrointestinal disorders; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; myocardial infarction, coronary artery disease, heart failure; immunodeficiencies, graft versus host diseases, acute kidney injuries, chronic kidney diseases, chronic obstructive pulmonary diseases (COPD), idiopathic pulmonary fibrosis (IPF), cirrhosis, acute liver failure, chronic liver diseases with hepatitis C or B virus infection or post-antiviral drug therapies, alcoholic liver diseases, alcoholic hepatitis, non-alcoholic liver diseases with steatosis or steatohepatitis, treatment of hearing loss, including internal and external loss of auditory hair cells, vestibular hypofunction, macular degeneration, treatment of various retinopathies, including but not limited to vitreoretinopathy, diabetic retinopathy, other diseases of retinal degeneration, wet age-related macular degeneration (AMD), dry AMD), Fuchs' dystrophy, other corneal diseases, stroke, traumatic brain injury, Alzheimer's disease, multiple sclerosis and other conditions affecting the blood brain barrier; bone diseases, spinal cord injuries, other spinal cord diseases, and alopecia.

23. A method of generating, culturing, or maintaining an organ, tissue, cell, or organoid culture, comprising contacting the organ, tissue, cell, or organoid culture with:
a) the WNT super agonist molecule of any one of claims 1-19; or
b) the pharmaceutical composition of claim 20.

24. The method of claim 23 for maintaining viability of the organ or tissue ex vivo, comprising:
a) contacting an organ or tissue obtained from a donor ex vivo with a composition comprising the WNT super agonist molecule or the pharmaceutical composition, optionally by perfusion; or
b) contacting a donor organ or tissue in vivo with a composition comprising the WNT super agonist molecule or the pharmaceutical composition.

25. The method of claim 23 for generating or maintaining the organoid culture, comprising contacting the organoid culture, optionally by culturing the organoid culture in a medium comprising the WNT super agonist molecule.

26. A method for inducing bone formation or increasing bone density in a subject, comprising administering to the subject an effective amount of the WNT super agonist molecule according to any of claims 1-19 or the pharmaceutical composition of claim 20.

27. The method of claim 26, wherein the WNT super agonist molecule binds FZD5, FZD8, and FZD9.

28. A method for regenerating a salivary gland or inducing salivary gland growth in a subject, comprising administering to the subject an effective amount of the WNT super agonist molecule according to any of claims 1-19 or the pharmaceutical composition of claim 20.

29. The method of claim 28 for treating hyposalivation in the subject.

30. The method of claim 28 or claim 29, wherein the WNT super agonist molecule binds FZD1, FZD2, and FZD7.

31. An R-spondin (RSPO) mimetic comprising a first binding composition that binds a WNT receptor and a second binding composition that binds an E3 ubiquitin ligase.

32. The RSPO mimetic of claim 31, wherein the first binding composition binds a FZD receptor or an LRP receptor, optionally LRP5 and/or LRP6.

33. The RPSO mimetic of claim 31 or claim 32, wherein the first binding composition is selected from the group consisting of: an scFv, a VHH/sdAb, a Fab fragment, a Fab′2 fragment, a diabody, and an Fv fragment.

34. The RSPO mimetic of claim 31 or claim 32, wherein the second binding composition is an RSPO protein, optionally a mutant RSPO protein, or an antibody or fragment thereof that binds an E3 ubiquitin ligase.

35. The RSPO mimetic of any one of claims 31-34, wherein the binding compositions are fused directly together or via a peptide linker.

36. The RSPO mimetic of claim 35, wherein the peptide linker is about 1 amino acid in length to about 30 amino acids in length.

37. The RSPO mimetic of claim 36, wherein the peptide linker is about 5 amino acids in length to about 15 amino acids in length, optionally 5, 6, 7, 8, 9, 10, 11, 12, 13, 14 or 15 amino acids in length.

38. The RSPO mimetic of any one of claims 35-37, wherein the peptide linker comprises one or more glycine and/or serine residues.

39. The RSPO mimetic of any of claims 31-38, comprising a polypeptide having at least 90% or 95% sequence identity to a sequence provided in Table 3 or Table 4, or a combination of polypeptides, each having at least 90% or 95% sequence identity to a sequence provided in Table 3 or Table 4.

40. A pharmaceutical composition comprising the RSPO mimetic according to any of claims 31-39 and a pharmaceutically acceptable diluent, excipient, or carrier.

41. A method for treating a subject having a disease or disorder associated with reduced WNT signaling, comprising administering to the subject an effective amount of the RSPO according to any of claims 31-39 or the pharmaceutical composition of claim 40.

42. The method of claim 41, wherein the disease or disorder is selected from the group consisting of: oral mucositis, short bowel syndrome, inflammatory bowel diseases (IBD), other gastrointestinal disorders; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; myocardial infarction, coronary artery disease, heart failure; immunodeficiencies, graft versus host diseases, acute kidney injuries, chronic kidney diseases, chronic obstructive pulmonary diseases (COPD), idiopathic pulmonary fibrosis (IPF), cirrhosis, acute liver failure, chronic liver diseases with hepatitis C or B virus infection or post-antiviral drug therapies, alcoholic liver diseases, alcoholic hepatitis, non-alcoholic liver diseases with steatosis or steatohepatitis, treatment of hearing loss, including internal and external loss of auditory hair cells, vestibular hypofunction, macular degeneration, treatment of vitreoretinopathy, diabetic retinopathy, other diseases of retinal degeneration, Fuchs' dystrophy, other corneal diseases, stroke, traumatic brain injury, Alzheimer's disease, multiple sclerosis and other conditions affecting the blood brain barrier; spinal cord injuries, bone diseases, other spinal cord diseases, and alopecia.

43. A WNT surrogate comprising:
a) a Frizzled (FZD) binding domain; and
b) an LRP5/6 binding domain,
wherein the super agonist molecule activates the canonical WNT signaling pathway in a cell.

44. The WNT surrogate of claim 43, wherein
a) the FZD binding domain binds one or more FZD receptor; and
b) the LRP5/6 binding domain binds LRP5 and/or LRP6.

45. The WNT surrogate of claim 43 or claim 44, wherein the FZD binding domain is selected from the group consisting of: an scFv, a VHH/sdAb, a Fab fragment, a Fab′2 fragment, a diabody, and an Fv fragment.

46. The WNT surrogate of any one of claims 43-45, wherein the LRP5/6 binding domain is selected from the group consisting of: an scFv, a VHH/sdAb, a Fab fragment, a Fab′2 fragment, a diabody, and an Fv fragment.

47. The WNT surrogate of any one of claims 43-46, wherein the binding domains are fused directly together or via a peptide linker.

48. The WNT surrogate of claim 47, wherein the peptide linker is about 1 amino acid in length to about 30 amino acids in length.

49. The WNT surrogate of claim 48, wherein the peptide linker is about 5 amino acids in length to about 15 amino acids in length, optionally 5, 6, 7, 8, 9, 10, 11, 12, 13, 14 or 15 amino acids in length.

50. The WNT surrogate of any one of claims 47-49, wherein the peptide linker comprises one or more glycine and/or serine residues.

51. The WNT surrogate of any of claims 43-50, comprising a polypeptide having at least 90% or 95% sequence identity to a sequence provided in Table 3 or Table 4, or a combination of polypeptides, each having at least 90% or 95% sequence identity to a sequence provided in Table 3 or Table 4.

52. A pharmaceutical composition comprising the RSPO mimetic according to any of claims 43-51 and a pharmaceutically acceptable diluent, excipient, or carrier.

53. A method for treating a subject having a disease or disorder associated with reduced WNT signaling, comprising administering to the subject an effective amount of the WNT surrogate according to any of claims 43-51 or the pharmaceutical composition of claim 52.

54. The method of claim 53, wherein the disease or disorder is selected from the group consisting of: oral mucositis, short bowel syndrome, inflammatory bowel diseases (IBD), other gastrointestinal disorders; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; myocardial infarction, coronary artery disease, heart failure; immunodeficiencies, graft versus host diseases, acute kidney injuries, chronic kidney diseases, chronic obstructive pulmonary diseases (COPD), idiopathic pulmonary fibrosis (IPF), cirrhosis, acute liver failure, chronic liver diseases with hepatitis C or B virus infection or post-antiviral drug therapies, alcoholic liver diseases, alcoholic hepatitis, non-alcoholic liver diseases with steatosis or steatohepatitis, treatment of hearing loss, including internal and external loss of auditory hair cells, vestibular hypofunction, macular degeneration, treatment of vitreoretinopathy, diabetic retinopathy, other diseases of retinal degeneration, Fuchs' dystrophy, other corneal diseases, stroke, traumatic brain injury, Alzheimer's disease, multiple sclerosis and other conditions affecting the blood brain barrier; bone diseases, spinal cord injuries, other spinal cord diseases, and alopecia.

55. A method of generating, culturing, or maintaining an organ, tissue, cell, or organoid culture, comprising contacting the organ, tissue, cell, or organoid culture with:
a) the WNT surrogate molecule of any of claims 43-51; or
b) or the pharmaceutical composition of claim 52.

56. The method of claim 55 for maintaining viability of the organ or tissue ex vivo, comprising:
a) contacting an organ or tissue obtained from a donor ex vivo with a composition comprising the WNT surrogate molecule or the pharmaceutical composition, optionally by perfusion; or
b) contacting a donor organ or tissue in vivo with a composition comprising the WNT surrogate molecule or the pharmaceutical composition.

57. The method of claim 55 for generating or maintaining the organoid culture, comprising contacting the organoid culture, optionally by culturing the organoid culture in a medium comprising the WNT surrogate molecule.

58. A method for inducing bone formation or increasing bone density in a subject, comprising administering to the subject an effective amount of the WNT surrogate molecule of any of claims 43-51 or the pharmaceutical composition of claim 52.

59. The method of claim 58, wherein the WNT surrogate molecule binds FZD5, FZD8, and FZD9.

60. A method for regenerating a salivary gland or inducing salivary gland growth in a subject, comprising administering to the subject an effective amount of the WNT surrogate molecule according to any of claims 43-51 or the pharmaceutical composition of claim 52.

61. The method of claim 60 for treating hyposalivation in the subject.

62. The method of claim 60 or claim 61, wherein the WNT surrogate molecule binds FZD1, FZD2, and FZD7.
